SEQUENCE LISTING (86 sequences)

SEQ ID NO: 1 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 2 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 3 - protein, 379 aa, Clostridium acetobutylicum
MDFNLTREQELVRQMVREFAENEVKPIAAEIDETERFPMENVKKMGQYGMMGIPFSKEYGGAGGDVLSYIIAVEELSKVCGTTGVILSAHTSLCASLINEHGTEEQKQKYLVPLAKGEKIGAYGLTEPNAGTDSGAQQTVAVLEGDHYVINGSKIFITNGGVADTFVIFAMTDRTKGTKGISAFIIEKGFKGFSIGKVEQKLGIRASSTTELVFEDMIVPVENMIGKEGKGFPIAMKTLDGGRIGIAAQALGIAEGAFNEARAYMKERKQFGRSLDKFQGLAWMMADMDVAIESARYLVYKAAYLKQAGLPYTVDAARAKLHAANVAMDVTTKAVQLFGGYGYTKDYPVERMMRDAKITEIYEGTSEVQKLVISGKIFR

SEQ ID NO: 4 - protein, 337 aa, Clostridium acetobutylicum
MNKADYKGVWVFAEQRDGELQKVSLELLGKGKEMAEKLGVELTAVLLGHNTEKMSKDLLSHGADKVLAADNELLAHFSTDGYAKVICDLVNERKPEILFIGATFIGRDLGPRIAARLSTGLTADCTSLDIDVENRDLLATRPAFGGNLIATIVCSDHRPQMATVRPGVFFEKLPVNDANVSDDKIEKVAIKLTASDIRTKVSKVVKLAKDIADIGEAKVLVAGGRGVGSKENFEKLEELASLLGGTIAASRAAIEKEWVDKDLQVGQTGKTVRPTLYIACGISGAIQHLAGMQDSDYIIAINKDVEAPIMKVADLAIVGDVNKVVPELIAQVKAANN

SEQ ID NO: 5 - protein, 252 aa, Clostridium acetobutylicum
MNIVVCLKQVPDTAEVRIDPVKGTLIREGVPSIINPDDKNALEEALVLKDNYGAHVTVISMGPPQAKNALVEALAMGADEAVLLTDRAFGGADTLATSHTIAAGIKKLKYDIVFAGRQAIDGDTAQVGPEIAEHLGIPQVTYVEKVEVDGDTLKIRKAWEDGYEVVEVKTPVLLTAIKELNVPRYMSVEKIFGAFDKEVKMWTADDIDVDKANLGLKGSPTKVKKSSTKEVKGQGEVIDKPVKEAADMLSQN

SEQ ID NO: 6 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKTNDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 7 - protein, 405 aa, Euglena gracilis
MAMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 8 - protein, 397 aa, Aeromonas hydrophila
MIIKPKVRGFICTTTHPVGCEANVRRQIAYTKAKGTIENGPKKVLVIGASTGYGLASRIAAAFGSGAATLGVFFEKAGSETKTATAGWYNSAAFDKAAKEAGLYAKSINGDAFSNECRAKVIELIKQDLGQIDLVVYSLASPVRKLPDTGEVVRSALKPIGEVYTTTAIDTNKDQIITATVEPANEEEIQNTITVMGGQDWELWMAALRDAGVLADGAKSVAYSYIGTDLTWPIYWHGTLGRAKEDLDRAAAAIRGDLAGKGGTAHVAVLKSVVTQASSAIPVMPLYISMAFKIMKEKGIHEGCMEQVDRMMRTRLYAADMALDDQARIRMDDWELREDVQQTCRDLWPSITSENLCELTDYTGYKQEFLRLFGFGLEEVDYDADVNPDVKFDVVEL

SEQ ID NO: 9 - protein, 318 aa, Clostridium acetobutylicum
MNLLNLFTYVIPIAICIILPIFIIVTHFQIKSLNKAVTSFNKGDRSNALEILSKLVKSPIKNVKANAYITRERIYFYSRDFELSLRDLLQAIKLRPKTINDVYSFALSYHILGEPERALKYFLRAVELQPNVGISYENLAWFYYLTGKYDKAIENFEKAISMGSTNSVYRSLGITYAKIGDYKKSEEYLKKALDAEPEKPSTHIYFSYLKRKTNDIKLAKEYALKAIELNKNNFDGYKNLAEVNLAEDDYDGFYKNLEIFLEKINFVTNGEDFNDEVYDKVKDNEKFKELIAKTKVIKFKDLGIEIDDKKILNGKFLV

SEQ ID NO: 10 - protein, 389 aa, Clostridium acetobutylicum ATCC 824
MLSFDYSIPTKVFFGKGKIDVIGEEIKKYGSRVLIVYGGGSIKRNGIYDRATAILKENNIAFYELSGVEPNPRITTVKKGIEICRENNVDLVLAIGGGSAIDCSKVIAAGVYYDGDTWDMVKDPSKITKVLPIASILTLSATGSEMDQIAVISNMETNEKLGVGHDDMRPKFSVLDPTYTFTVPKNQTAAGTADIMSHTFESYFSGVEGAYVQDGIREAILRTCIKYGKIAMEKTDDYEARANLMWASSLAINGLLSLGKDRKWSCHPMEHELSAYYDITHGVGLAILTPNWMEYILNDDTLHKFVSYGINVWGIDKNKDNYEIAREAIKNTREYFNSLGIPSKLREVGIGKDKLELMAKQAVRNSGGTIGSLRPINAEDVLEIFKKSY

SEQ ID NO: 11 - protein, 390 aa, Clostridium acetobutylicum ATCC 824
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 12 - protein, 552 aa, Citrobacter freundii
MSQFFFNQRTHLVSDVIDGTIIASPWNNLARLESDPAIRIVVRRDLNKNNVAVISGGGSGHEPAHVGFIGKGMLTAAVCGDVFASPSVDAVLTAIQAVTGEAGCLLIVKNYTGDRLNFGLAAEKARRLGYNVEMLIVGDDISLPDNKHPRGIAGTILVHKIAGYFAERGYNLATVLREAQYAANNTFSLGVALSSCHLPQEADAAPRHHPGHAELGMGIHGEPGASVIDTQNSAQVVNLMVDKLMAALPETGRLAVMINNLGGVSVAEMAIITRELASSPLHPRIDWLIGPASLVTALDMKSFSLTAIVLEESIEKALLTEVETSNWPTPVPPREISCVPSSQRSARVEFQPSANAMVAGIVELVTTTLSDLETHLNALDAKVGDGDTGSTFAAGAREIASLLHRQQLPLDNLATLFALIGERLTVVMGGSSGVLMSIFFTAAGQKLEQGASVAESLNTGLAQMKFYGGADEGDRTMIDALQPALTSLLTQPQNLQAAFDAAQAGAERTCLSSKANAGRASYLSSESLLGNMDPGAHAVAMVFKALAESELG

SEQ ID NO: 13 - protein, 364 aa, Candida boidinii
MKIVLVLYDAGKHAADEEKLYGCTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 14 - protein, 549 aa, Klebsiella pneumoniae
MSQFFFNQRASLVNDVIEGTIIASPWNNLARLESDPAIRVVVRRDLNKNNVAVISGGGAGHEPAHVGFIGKGMLTAAVCGDLFASPSVDAVLTAIQAVTGEAGCLLIVKNYTGDRLNFGLAAEKARRLGYNVEMLIVGDDISLPDNKQPRGIAGTILVHKVAGYFAERGFNLATVLREAQYAASHTASIGVALASCHLPQEADSAPRHQAGHAELGMGIHGEPGASTIATQNSAEIVNLMVEKLTAALPETGRLAVMLNNLGGVSVAEMAILTRELANTPLQARIDWLIGPASLVTALDMKGFSLTAIVLEESIEKALLSDVETASWQKPVQPRTINAVPSTLDSARVDFTPSANPQVGDYVAQVTGALIDLEEHLNALDAKVGDGDTGSTFAAGAREIAERLERQQLPLNDLPTLFALIGERLTVVMGGSSGVLMSIFFTAAGQKLGQGASVAEALNAGLEQMKFYGGADEGDRTMIDALQPALAALLAEPENLQAAFAAAQAGADRTCQSSKAGAGRASYLNSDSLLGNMDPGAHAVAMVFKALAER

SEQ ID NO: 15 - protein, 584 aa, Saccharomyces cerevisiae
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 16 - protein, 591 aa, Saccharomyces cerevisiae
MSHKQFKSDGNIVTPYLLGLARSNPGLTVIKHDRVVFRTASAPNSGNPPKVSLVSGGGSGHEPTHAGFVGEGALDAIAAGAIFASPSTKQIYSAIKAVESPKGTLIIVKNYTGDIIHFGLAAERAKAAGMKVELVAVGDDVSVGKKKGSLVGRRGLGATVLVHKIAGAAASHGLELAEVAEVAQSVVDNSVTIAASLDHCTVPGHKPEAILGENEYEIGMGIHNESGTYKSSPLPSISELVSQMLPLLLDEDEDRSYVKFEPKEDVVLMVNNMGGMSNLELGYAAEVISEQLIDKYQIVPKRTITGAFITALNGPGFGITLMNASKAGGDILKYFDYPTTASGWNQMYHSAKDWEVLAKGQVPTAPSLKTLRNEKGSGVKADYDTFAKILLAGIAKINEVEPKVTWYDTIAGDGDCGTTLVSGGEALEEAIKNHTLRLEDAALGIEDIAYMVEDSMGGTSGGLYSIYLSALAQGVRDSGDKELTAETFKKASNVALDALYKYTRARPGYRTLIDALQPFVEALKAGKGPRAAAQAAYDGAEKTRKMDALVGRASYVAKEELRKLDSEGGLPDPGAVGLAALLDGFVTAAGY

SEQ ID NO: 17 - DNA, 2253 bp, Artificial Sequence
  Description of Artificial Sequence Synthetic polynucleotide
ctcgagtccctatcagtgatagagattgacatccctatcagtgatagagatactgagcacatcagcaggacgcactgaccgaattcattaaagaggagaaaggtaccgggccccccctcgaggtcgacggtatcgataagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtctaagaaaccattattatcatgacattaacctataaaaataggcgtatcacgaggccctttcgtcttcac

SEQ ID NO: 18 - DNA, 3068 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagataaatgtgagcggataacattgacattgtgagcggataacaagatactgagcacatcagcaggacgcactgaccgaattcattaaagaggagaaaggtaccatgtcagttttcgtttcaggtgctaacgggttcattgcccaacacattgtcgatctcctgttgaaggaagactataaggtcatcggttctgccagaagtcaagaaaaggccgagaatttaacggaggcctttggtaacaacccaaaattctccatggaagttgtcccagacatatctaagctggacgcatttgaccatgttttccaaaagcacggcaaggatatcaagatagttctacatacggcctctccattctgctttgatatcactgacagtgaacgcgatttattaattcctgctgtgaacggtgttaagggaattctccactcaattaaaaaatacgccgctgattctgtagaacgtgtagttctcacctcttcttatgcagctgtgttcgatatggcaaaagaaaacgataagtctttaacatttaacgaagaatcctggaacccagctacctgggagagttgccaaagtgacccagttaacgcctactgtggttctaagaagtttgctgaaaaagcagcttgggaatttctagaggagaatagagactctgtaaaattcgaattaactgccgttaacccagtttacgtttttggtccgcaaatgtttgacaaagatgtgaaaaaacacttgaacacatcttgcgaactcgtcaacagcttgatgcatttatcaccagaggacaagataccggaactatttggtggatacattgatgttcgtgatgttgcaaaggctcatttagttgccttccaaaagagggaaacaattggtcaaagactaatcgtatcggaggccagatttactatgcaggatgttctcgatatccttaacgaagacttccctgttctaaaaggcaatattccagtggggaaaccaggttctggtgctacccataacacccttggtgctactcttgataataaaaagagtaagaaattgttaggtttcaagttcaggaacttgaaagagaccattgacgacactgcctcccaaattttaaaatttgagggcagaatataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 19 - DNA, 3231 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgggaattcttattatttaggaggagtaaaacatgagagatgtagtaatagtaagtgctgtaagaactgcaataggagcatatggaaaaacattaaaggatgtacctgcaacagagttaggagctatagtaataaaggaagctgtaagaagagctaatataaatccaaatgagattaatgaagttatttttggaaatgtacttcaagctggattaggccaaaacccagcaagacaagcagcagtaaaagcaggattacctttagaaacacctgcgtttacaatcaataaggtttgtggttcaggtttaagatctataagtttagcagctcaaattataaaagctggagatgctgataccattgtagtaggtggtatggaaaatatgtctagatcaccatatttgattaacaatcagagatggggtcaaagaatgggagatagtgaattagttgatgaaatgataaaggatggtttgtgggatgcatttaatggatatcatatgggagtaactgcagaaaatattgcagaacaatggaatataacaagagaagagcaagatgaattttcacttatgtcacaacaaaaagctgaaaaagccattaaaaatggagaatttaaggatgaaatagttcctgtattaataaagactaaaaaaggtgaaatagtctttgatcaagatgaatttcctagattcggaaacactattgaagcattaagaaaacttaaacctattttcaaggaaaatggtactgttacagcaggtaatgcatccggattaaatgatggagctgcagcactagtaataatgagcgctgataaagctaacgctctcggaataaaaccacttgctaagattacttcttacggatcatatggggtagatccatcaataatgggatatggagctttttatgcaactaaagctgccttagataaaattaatttaaaacctgaagacttagatttaattgaagctaacgaggcatatgcttctcaaagtatagcagtaactagagatttaaatttagatatgagtaaagttaatgttaatggtggagctatagcacttggacatccaataggtgcatctggtgcacgtattttagtaacattactatacgctatgcaaaaaagagattcaaaaaaaggtcttgctactctatgtattggtggaggtcagggaacagctctcgtagttgaaagagactaaggatccgatccgatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 20 - DNA, 2908 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccggaattcattgatagtttctttaaatttagggaggtctgtttaatgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataaggatccgatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 21 - DNA, 3285 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagataaatgtgagcggataacattgacattgtgagcggataacaagatactgagcacatcagcaggacgcactgaccgaattcgctcaattacacaacggaggtataataatgggcaaagaaagtagttttagctgtgcatgtcgtacagccatcggaacaatgggtggatctcttagcacaattcctgcagtagatttaggtgctatcgttatcaaagaggctcttaaccgcgcaggtgttaaacctgaagatgttgatcacgtatacatgggatgcgttattcaggcaggacagggacagaacgttgctcgtcaggcttctatcaaggctggtcttcctgtagaagtacctgcagttacaactaacgttgtatgtggttcaggtcttaactgtgttaaccaggcagctcagatgatcatggctggagatgctgatatcgttgttgccggtggtatggaaaacatgtcacttgcaccatttgcacttcctaatggccgttacggatatcgtatgatgtggccaagccagagccagggtggtcttgtagacactatggttaaggatgctctttgggatgctttcaatgattatcatatgatccagacagcagacaacatctgcacagagtggggtcttacacgtgaagagctcgatgagtttgcagctaagagccagaacaaggcttgtgcagcaatcgaagctggcgcattcaaggatgagatcgttcctgtagagatcaagaagaagaaagagacagttatcttcgatacagatgaaggcccaagacagggtgttacacctgaatctctttcaaagcttcgtcctatcaacaaggatggattcgttacagctggtaacgcttcaggtatcaacgacggtgctgcagcactcgtagttatgtctgaagagaaggctaaggagctcggcgttaagcctatggctacattcgtagctggagcacttgctggtgttcgtcctgaagttatgggtatcggtcctgtagcagctactcagaaggctatgaagaaggctggtatcgagaacgtatctgagttcgatatcatcgaggctaacgaagcattcgcagctcagtctgtagcagttggtaaggatcttggaatcgacgtccacaagcagctcaatcctaacggtggtgctatcgctcttggacacccagttggagcttcaggtgctcgtatccttgttacacttcttcacgagatgcagaagaaagacgctaagaagggtcttgctacactttgcatcggtggcggtatgggatgcgctactatcgttgagaagtacgaataattaaactttcagagggtgtgaaggtcatataagatcaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 22 - DNA, 2877 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagataaatgtgagcggataacattgacattgtgagcggataacaagatactgagcacatcagcaggacgcactgaccgaattcccacaccctcttaatactgctaataattggaggacgaatcaatgagttttgttttatatgaacagaaagataagatcgctgttgtaactatcaaccgtccggaagcacttaatgctcttaactcagcagttctcgatgagcttaatgaagttctcgataacgttgatcttaatacagttagagcactcgttcttaccggtgctggagataagtcttttgtagctggtgctgatattggagagatgtccacacttacaaaggctgaaggtgaagcttttggtaagaagggtaacgatgtattccgtaagcttgagacacttcctatccctgtaattgcagctgttaacggctttgcacttggcggcggatgtgagatctctatgagctgcgatatccgtatctgctcagacaacgctatgttcggtcagcctgaagttggtcttggaattactcctggattcggcggaacacagagacttgcaagaacagttggtgttggtatggctaaacagcttatctacacagctcgtaatatcaaagctgacgaagcacttcgtatcggccttgtaaacgctgtatacactcaggaagagcttcttcctgcagctgagaagcttgcaacaacaatcgctggtaacgctcctatagctgttcgtgcttgtaagaaagctatcaacgatggtcttcagactgatatcgacagcgcacttgtaatcgaagaaaagctctttggttcatgcttcgagtcagaagatcaggtagaaggaatggctaacttccttcgtaagaaagatgatcctaagaaggttaagcacgtagatttcaagaatgcttaatatcgatctttgatgtgatattcggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 23 - DNA, 2994 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagataaatgtgagcggataacattgacattgtgagcggataacaagatactgagcacatcagcaggacgcactgaccgaattctacaaggtgagtattacagtcaaataatcggggattaaatagacatatatcatttaacggaaaataatagataaaatatatctaaggaggatttacaatgaaagtagctgtaattggtgcaggaacaatgggttctggtattgcacaggcattcgcacagtgtgacgctgttgagacagtttatctttgcgatatcaagcaggagttcgctgatggcggtaagagcaagatcgagaagaatcttggacgtcttgttaagaaggaaaagatgactcaggaagctgctgatgcaatcgtagcaaaggttaagacaggtcttaacacaatcgctacagatcctgatctcgtagttgaggctgcacttgaagttatggatatcaagaaagcttgcttcaaggaacttcaggagaacatcgttaagaatcctgattgtatctatgcttcaaacacatcatctctttcaatcacagagatcggtgcaggtcttaagactcctatcatcggaatgcacttgttcaacccagctcctgttatgaagctcatcgaggttatctcaggcgctaacacacctaaggagacaacagagaaggttatcgagatctccaagactcttggtaagacacctgtacaggttaacgaggctcctggattcgttgttaaccgtattcttattccacttatcaacgaaggtatcttcgtatattcagaaggaatttctgatatcgaaggcatcgatacagctatgaagcttggatgtaaccatcctatgggaccccttgaactgggtgactatgtaggtcttgatatcgttcttgctatcatggatgtactttacaatgagactaaggattccaagtatcgtgcatgcggactccttcgtaagatggttcgtgcaggtcaccttggcgttaagtcaggaatcggtttctacaagtacaacgaagacagaacaaagactcctgttgacaagctttaaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 24 - DNA, 2855 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattcattaaagaggagaaaggtaccaaaataagcaagtttgaaggaggtccttagaatggaattaaaaaatgttattcttgaaaaagaagggcatttagctattgttacaatcaatagaccaaaggcattaaatgcattgaattcagaaacactaaaagatttaaatgttgttttagatgatttagaagcagacaacaatgtgtatgcagttatagttacaggtgctggtgagaaatcttttgttgctggagcagatatttcagaaatgaaagatcttaatgaagaacaaggtaaagaatttggtattttaggaaacaatgtcttcagaagattagaaaaattggataagccagttatcgcagctatatcaggatttgctcttggtggtggatgtgaacttgctatgtcatgtgacataagaatagcttcagttaaagctaaatttggtcaaccagaagcaggacttggaataactccaggatttggtggaactcaaagattagctagaattgtagggccaggaaaagctaaagaattaatttatacttgtgaccttataaatgcagaagaagcttatagaataggtttagttaataaagtagttgaattagaaaaattgatggaagaagcaaaagcaatggctaacaagattgcagctaatgctccaaaagcagttgcatattgtaaagatgctatagacagaggaatgcaagttgatatagatgcagctatattaatagaagcagaagactttggaaagtgctttgcaacagaagatcaaacagaaggaatgactgcgttcttagaaagaagagcagaaaagaattttcaaaataaataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 25 - DNA, 2891 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattcaaaagatttagaggaggaataattcatgaaaaagatttttgtacttggagcaggaacaatgggtgctggtatcgttcaagcattcgctcaaaaaggttgtgaagtaattgtaagagacataaaggaagaatttgttgacagaggaatagctggaatcactaaaggattagaaaagcaagttgctaaaggaaaaatgtctgaagaagataaagaagctatactttcaagaatttcaggaacaactgatatgaaattagctgctgactgtgatttagtagttgaagctgcaatcgaaaacatgaaaattaagaaggaaatcttcgctgaattagatggaatttgtaagccagaagcgattttagcttcaaacacttcatctttatcaattactgaagttgcttcagctacaaagagacctgataaagttatcggaatgcatttctttaatccagctccagtaatgaagcttgttgaaattattaaaggaatagctacttctcaagaaacttttgatgctgttaaggaattatcagttgctattggaaaagaaccagtagaagttgcagaagctccaggattcgttgtaaacagaatattaatcccaatgattaacgaagcttcatttatcctacaagaaggaatagcttcagttgaagatattgatacagctatgaaatatggtgctaaccatccaatgggacctttagctttaggagatcttattggattagacgtttgcttagctatcatggatgttttattcactgaaacaggtgataacaagtacagagctagcagcatattaagaaaatatgttagagctggatggcttggaagaaaatcaggaaaaggattctatgattattctaaataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 26 - DNA, 5125 bp, Artificial Sequence
  Description of Artificial Sequence Synthetic polynucleotide
aattctaaactaactatacgctaaggagagtggaacatcatggattttaacttaacagatattcagcaagacttcctgaagctggcacacgactttggtgaaaagaaactggcccctactgttaccgaacgcgaccacaaaggtatctacgataaagaactgattgacgaactgctgtctctgggtatcaccggcgcatacttcgaagaaaaatacggcggtagcggtgacgacggtggcgatgtactgtcttatatcctggccgtagaagaactggcgaaatacgacgctggtgttgctatcactctgtctgccaccgtaagcctgtgtgcgaatccgatttggcagtttggtactgaggctcagaaagaaaagtttctggttccactggtcgaaggtactaaactgggtgcgtttggtctgaccgaaccgaacgcgggcactgatgcgagcggccagcaaactattgctactaaaaacgatgacggcacgtacaccctgaacggtagcaaaatcttcatcaccaacggtggcgctgccgatatctacatcgtatttgcgatgaccgacaaaagcaagggtaaccatggcatcaccgcgttcatcctggaagatggcactccgggtttcacctacggcaaaaaggaagataaaatgggtatccacacctctcagactatggaactggttttccaggacgttaaggtcccggccgagaacatgctgggcgaagaaggcaaaggcttcaagattgcaatgatgaccctggacggcggtcgcattggcgttgcggcccaggcactgggcatcgcagaggcagcgctggccgacgctgttgaatacagcaaacagcgtgttcagtttggcaaacctctgtgcaaattccaatccattagctttaagctggccgatatgaaaatgcagatcgaagccgcacgcaacctggtatataaagctgcatgcaagaaacaagaaggtaaaccgttcaccgtagacgctgcgatcgcgaaacgtgtagccagcgatgtggcaatgcgcgtgactaccgaagcagttcagattttcggtggctatggttactctgaagaatacccggtggctcgccacatgcgcgacgcaaaaatcactcagatctacgagggtacgaacgaagtgcagctgatggtcaccggcggtgctctgttaagttaattaaagtttatgctcggcctgccctttgctgggcccgttacataaaaaaagattttaggaggcaaaacgtaaatggaaatattggtatgtgtcaaacaagtgccggatactgcagaagtcaaaattgatccggttaaacacaccgtgattcgtgcgggtgtgccgaatatcttcaacccgttcgaccaaaacgcgctggaagcggcgctggcgctgaaggacgcggataaagacgttaagattactctgctgtctatgggcccggaccaggcaaaagatgttctgcgtgaaggcctggccatgggcgctgatgacgcgtacctgctgtccgatcgtaaactgggtggctccgacactctggccaccggttatgctctggcccaggctattaagaaactggctgcggacaagggtattgagcaattcgacatcatcctgtgtggtaagcaagcgattgacggtgataccgctcaggtaggtccacagatcgcttgtgagctgggcatcccgcagatcacttatgctcgtgacatcaaggttgagggcgataaggttactgtgcagcaggaaaacgaagagggttacatcgtgaccgaagcgcagttcccggttctgatcaccgcggttaaagacctgaacgaacctcgtttcccgaccatccgtggcaccatgaaggcgaagcgtcgtgaaatcccgaacctggacgcagctgcagttgccgcggacgacgcgcagatcggcctgtccggttctccgaccaaagtacgcaaaattttcaccccaccgcagcgttccggcggtctggtactgaaagtggaagacgacaacgaacaggccattgtcgaccaggttatggaaaaactggttgcccagaaaatcatttaatctaaggaggaacagtgaaaatggatttagcagaatacaaaggcatctacgtgatcgcagagcagttcgaaggtaaactgcgtgacgtttctttcgaactgctgggtcaagcgcgcatcctggcggacacgatcggcgacgaagtaggcgcaatcctgattggcaaagatgtaaaaccactggcgcaggaactgatcgcgcatggtgctcataaagtgtacgtctatgacgacccgcagctggaacattacaacacgactgcctatgccaaagtgatttgcgacttctttcatgaagagaaaccaaacgttttcctggttggtgcaactaacatcggtcgtgacctgggtccacgtgtagcgaacagcctgaaaaccggtctgactgcggattgtacccagctgggtgttgatgatgataagaaaaccatcgtttggacccgtccggcactgggcggcaacatcatggcggaaattatctgtccagataaccgcccgcagatgggcactgtgcgtcctcatgtcttcaaaaagccggaagccgacccgagcgcaactggtgaagtcattgaaaagaaagcgaacctgtctgacgctgatttcatgactaagttcgtagaactgatcaaactgggtggtgaaggcgttaaaatcgaggatgccgatgttattgttgctggtggccgtggcatgaatagcgaagagccttttaaaaccggtatcctgaaagagtgcgcggacgtactgggcggtgctgtcggtgccagccgtgccgccgtggacgcgggctggatcgacgctctgcaccaggtcggccagactggcaaaaccgttggtccgaaaatctacattgcttgtgcgattagcggtgctatccagccgctggcaggcatgacgggctctgattgtattatcgcaattaacaaagatgaagacgcgcctattttcaaggtgtgcgactatggcattgtgggcgatgtgttcaaagtgctgccactgctgactgaggcgatcaagaaacagaaaggcattgcataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccg

SEQ ID NO: 27 - DNA, 2982 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagataaatgtgagcggataacattgacattgtgagcggataacaagatactgagcacatcagcaggacgcactgaccgaattcattaaagaggagaaaggtaccaagaattatttaaagcttattatgccaaaatacttatatagtattttggtgtaaatgcattgatagtttctttaaatttagggaggtctgtttaatgaaaaaggtatgtgttataggcgcgggaaccatgggtagcggtattgcccaggcatttgctgcaaaaggtttcgaagtggttctgcgtgatatcaaggacgagtttgtcgatcgcggcttagacttcattaataaaaacctgtctaaactggtaaagaaagggaaaatcgaagaggcgacgaaggtggaaattttaactcggatcagtggaacagttgatctgaatatggccgctgactgcgatctggtcattgaagcggccgtagagcgtatggatatcaaaaaacaaatttttgcagacttagataacatctgtaagccggaaaccattctggcttcaaatacgtcctcgctgagcatcactgaggtggcgtctgccacaaaacgcccagacaaagttattggcatgcatttctttaaccctgcaccggtcatgaagttagtggaagtaatccgtgggattgctaccagtcaggaaacgttcgatgcggttaaagagacctcaatcgccattggaaaagacccagtggaagtcgcagaggcgcctggctttgttgtaaatcgcattctgatcccgatgattaacgaagctgtgggaatcctggccgaaggaattgcatccgtcgaggatatcgacaaggcgatgaaattaggcgctaatcacccgatgggtccactggaactgggcgacttcattggtctggatatctgcttagccattatggacgttctgtattcggagactggggatagcaaataccggcctcatacactgttaaagaaatatgtgcgtgcaggatggctgggccgcaaatctggtaagggtttctacgattattcaaaataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 28 - DNA, 5125 bp, Artificial Sequence; identical to SEQ ID NO: 26
  Description of Artificial Sequence Synthetic polynucleotide

SEQ ID NO: 29 - DNA, 2836 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgggaattcctatctatttttgaagccttcaatttttcttttctctatgaaagctgtcattgcatccttttgatcctctgttgaaaagcattctccaaatgcttctgattcaaatgctaaagcagtatcaatatcacactgcattcctctattaatagcctgtttgcttaacttaacagctactggagcattgctcacaattttgtttgcaatttcttttgctgtattcattaattcactaggttctactaccttatttacaagtccgattcttaatgcttcatctgcctttatattttgtgcagtaaatataagctgctttgccatgcccattccaactaatcttgaaagtctttgtgtaccaccaaaaccaggtgttattccgagacctacttctggttgaccaaatcttgcgttgcttgaagctattcttatatcacaagacatagctatttcgcatccgcctcctaaagcaaaaccattaacagctgctattacaggcttttcaagaagttctaatcttctaaacactttatttccaagtatcccgaattttctaccttcaatggtattcatttccttcatctcagaaatatctgctcctgctacaaatgatttttctcctgctccagttaaaattactgcaagtacttcgctatcattttcaatttcacctataacataatccatttcttttagtgtatcactatttaacgcatttaatgctttaggtctgttaatggtaactacagcaactttaccttccttttcaaggatgacattgtttagttccatgactaatcctcctaaaatattggatccgatccgatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 30 - DNA, 2018 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaattgtgagcggataacaattgacattgtgagcggataacaagatactgagcacatcagcaggacgcactgaccgaattcggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 31 - DNA, 3258 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagataaatgtgagcggataacaattgacattgtgagcggataacaagatactgagcacatcagcaggacgcactgaccgaattcattaaagaggagaaaggtaccatggccatgttcaccactaccgccaaggttattcagccgaaaatccgtggttttatctgtacgaccacccacccgattggctgtgaaaaacgcgtgcaggaagaaattgcttacgcacgtgcacatccaccgaccagcccgggtccgaaacgtgtcctggtcatcggctgttccactggctacggcctgtctactcgtatcaccgcagctttcggctatcaggcggctactctgggcgtgttcctggctggtccgccgactaaaggtcgcccggctgcggccggttggtataacaccgtagctttcgaaaaagcggccctggaagccggtctgtatgcccgctccctgaacggtgacgcttttgactctactaccaaagcacgcaccgtggaagctatcaaacgtgacctgggcaccgttgacctggtggtttatagcattgcagctccgaaacgtaccgatccggctaccggcgtgctgcacaaagcgtgtctgaaaccgatcggtgcgacctacaccaaccgtacggtaaatactgacaaagctgaagttacggacgtgtccatcgaaccggcgagcccagaagaaattgcagacactgtgaaagtaatgggtggcgaagactgggaactgtggattcaggctctgtctgaagccggcgttctggcagaaggcgcgaaaaccgtcgcatactcttatatcggtccggagatgacctggccggtgtactggtccggcaccattggtgaagccaaaaaggatgttgaaaaagccgctaaacgtattacccagcagtacggctgtccggcatacccggttgtggcaaaagcactggtgacgcaggcatcctccgcgatcccggtcgtcccgctgtatatttgtctgctgtaccgtgtaatgaaagaaaaaggcactcacgaaggttgcatcgaacaaatggtgcgtctgctgaccacgaaactgtacccggaaaacggtgccccgatcgttgatgaagcgggccgtgttcgtgtggacgattgggaaatggcagaagacgttcagcaagccgttaaagacctgtggagccaggtgagcacggcaaacctgaaagatatttccgacttcgccggttaccaaaccgagttcctgcgcctgtttggttttggtatcgatggcgtggactatgaccagccggttgacgtagaggcagacctgccgagcgcagctcagcagtaaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 32 - DNA, 3233 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagataaatgtgagcggataacattgacattgtgagcggataacaagatactgagcacatcagcaggacgcactgaccgaattcattaaagaggagaaaggtaccatgatcattaaaccgaaagttcgtggcttcatttgtaccaccactcatccggttggctgtgaagctaatgtacgccgccagatcgcgtataccaaagcaaaaggcactatcgaaaacggccctaagaaagtgctggtgattggtgcgagcaccggttacggtctggcgtcccgcattgcagcggcgttcggtagcggcgccgcgaccctgggtgttttcttcgaaaaagcgggctccgaaactaaaaccgcgaccgcaggttggtacaactctgccgcgtttgacaaagccgccaaagaggctggcctgtatgcgaaatctattaacggtgacgcgttcagcaacgaatgccgtgctaaagtgatcgaactgatcaaacaggatctgggccaaattgatctggttgtttattctctggcctccccggttcgtaaactgccggataccggcgaagttgtgcgcagcgctctgaaacctattggtgaagtgtacaccacgaccgcaattgatactaataaggaccagattatcaccgcaaccgtcgagccggccaacgaggaagagatccagaataccatcactgtgatgggcggtcaagactgggaactgtggatggcagcactgcgcgacgcaggtgttctggcagacggtgcaaagagcgtcgcttactcttacatcggcactgacctgacttggccgatctactggcatggcaccctgggtcgcgcgaaagaggatctggatcgcgcagcggcagcgatccgcggtgatctggccggtaagggcggtactgcgcacgttgccgttctgaaatccgtggtcacccaggcatcttctgcaatcccggtgatgccgctgtatatttctatggcctttaaaatcatgaaagagaagggtatccacgaaggctgtatggagcaagtggaccgcatgatgcgtactcgcctgtacgcggcggacatggcactggatgaccaggcgcgtatccgtatggacgattgggaactgcgtgaagatgttcagcagacttgccgtgatctgtggccgtccattacctccgaaaacctgtgcgagctgaccgattacactggttacaaacaggaatttctgcgtctgttcggtttcggtctggaagaagtagactacgatgcagacgttaacccggacgttaaatttgatgttgtcgaactgtgaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 33 - DNA, 2908 bp, Artificial Sequence; identical to SEQ ID NO: 20
  Description of Artificial Sequence Synthetic polynucleotide

SEQ ID NO: 34 - DNA, 3278 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaattgtgagcggataacaattgacattgtgagcggataacaagatactgagcacatcagcaggacgcactgaccgaattcaacaataaaaaccgtatcaaaatttaggaggttagttagaatgaaagaagttgtaatagctagcgcggtgcgtaccgccattggctcttatggtaaaagtctgaaggatgttccggcagtcgacttaggggctacggcgatcaaagaagccgtaaaaaaggcaggaattaaaccagaggatgtgaatgaagttatcctgggcaacgtcctgcaggctggtttagggcaaaatcctgcgcgccaggcctcatttaaagcaggactgccggtagagattccagctatgactatcaacaaggtgtgcggctccggtctgcggacagtttcgttagcggcccaaattatcaaagcaggcgacgctgatgtcattatcgcgggtgggatggaaaatatgagccgtgccccttacctggcaaacaatgcgcgctggggatatcgtatgggcaacgctaaattcgtggacgaaatgattaccgatggtctgtgggatgcctttaatgactaccatatgggcatcacggcagagaacattgcggaacgctggaatatctctcgggaggaacaggatgagttcgctttagccagtcagaagaaagcagaggaagcgattaaatcaggtcaatttaaggacgagatcgtaccggttgtgattaaagggcgtaaaggagaaactgtcgttgatacagacgaacacccgcgcttcggctccaccattgagggtctggctaagctgaaaccagcctttaaaaaggatgggacggtaaccgcaggcaacgcgtcgggtttaaatgattgtgccgcagtgctggtcatcatgagcgcggaaaaagctaaagagctgggagttaagcctctggccaaaattgtgtcttatggcagtgcgggtgtagacccggctatcatggggtacggcccgttctatgcaactaaagccgcgattgaaaaggctggttggacagtcgatgaattagacctgatcgagtcaaacgaagcatttgccgcgcagtccctggctgttgcaaaagatttaaaattcgatatgaataaggtgaacgtaaatggaggcgccattgcgctgggtcatccaatcggggcttcgggagcacgtattctggttacgttagtgcacgccatgcaaaaacgcgacgcgaaaaagggcctggctaccctgtgcatcggtgggggccagggtactgcaatattgctagaaaagtgctagacttaattaacaataatcgatgggcccaaggtacctaagcttggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 35 - DNA, 2863 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaattgtgagcggataacattgacattgtgagcggataacaagatactgagcacatcagcaggacgcactgaccgaattcagtattaattaacaataatcgatatattttaggaggattagtcatggaactaaacaatgtcatcctggaaaaagagggcaaggtggcggttgtcaccattaatcgtccgaaagccttaaacgcactgaatagcgatacgctgaaagaaatggactatgtaatcggtgagattgaaaacgattctgaagtgttagctgttatcctgactggggcgggagagaagagttttgtcgccggcgcagacatttcagaaatgaaagagatgaatacaatcgaaggtcgcaaattcgggattctgggaaacaaggtatttcggcgtttagaactgctggagaaaccagtgatcgctgcggttaatggcttcgccttaggtggcggttgcgaaattgcaatgtcctgtgatatccgcattgcttcgagcaacgcgcgttttgggcagcctgaggtcggactgggcatcacaccgggtttcggcggtacgcaacgcctgtctcggttagtggggatgggaatggccaaacagctgatttttactgcacaaaatatcaaggctgacgaagcgctgcgtattggcctggtaaacaaagttgtggaaccaagtgagttaatgaatacagccaaagaaatcgcaaacaagattgtctcaaatgcgcctgttgctgtaaaactgtccaaacaggccattaaccgcggtatgcagtgcgatatcgacaccgcactggcgttcgagtcggaagcttttggggaatgtttcagcacggaggaccaaaaggatgccatgaccgcatttattgaaaaacgtaaaattgaaggcttcaaaaatagataggataggtacctaagcttggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 36 - DNA, 7813 bp, Artificial Sequence
  Description of Artificial Sequence Synthetic polynucleotide
ctcgagtccctatcagtgatagagattgacatccctatcagtgatagagatactgagcacatcagcaggacgcactgaccgaattcacaataaaaaccgtatcaaaatttaggaggttagttagaatgaaagaagttgtaatagctagcgcggtgcgtaccgccattggctcttatggtaaaagtctgaaggatgttccggcagtcgacttaggggctacggcgatcaaagaagccgtaaaaaaggcaggaattaaaccagaggatgtgaatgaagttatcctgggcaacgtcctgcaggctggtttagggcaaaatcctgcgcgccaggcctcatttaaagcaggactgccggtagagattccagctatgactatcaacaaggtgtgcggctccggtctgcggacagtttcgttagcggcccaaattatcaaagcaggcgacgctgatgtcattatcgcgggtgggatggaaaatatgagccgtgccccttacctggcaaacaatgcgcgctggggatatcgtatgggcaacgctaaattcgtggacgaaatgattaccgatggtctgtgggatgcctttaatgactaccatatgggcatcacggcagagaacattgcggaacgctggaatatctctcgggaggaacaggatgagttcgctttagccagtcagaagaaagcagaggaagcgattaaatcaggtcaatttaaggacgagatcgtaccggttgtgattaaagggcgtaaaggagaaactgtcgttgatacagacgaacacccgcgcttcggctccaccattgagggtctggctaagctgaaaccagcctttaaaaaggatgggacggtaaccgcaggcaacgcgtcgggtttaaatgattgtgccgcagtgctggtcatcatgagcgcggaaaaagctaaagagctgggagttaagcctctggccaaaattgtgtcttatggcagtgcgggtgtagacccggctatcatggggtacggcccgttctatgcaactaaagccgcgattgaaaaggctggttggacagtcgatgaattagacctgatcgagtcaaacgaagcatttgccgcgcagtccctggctgttgcaaaagatttaaaattcgatatgaataaggtgaacgtaaatggaggcgccattgcgctgggtcatccaatcggggcttcgggagcacgtattctggttacgttagtgcacgccatgcaaaaacgcgacgcgaaaaagggcctggctaccctgtgcatcggtgggggccagggtactgcaatattgctagaaaagtgctagacttaattaaattttataaaggagtgtatataaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaaatcgatatattttaggaggattagtcatggaactaaacaatgtcatcctggaaaaagagggcaaggtggcggttgtcaccattaatcgtccgaaagccttaaacgcactgaatagcgatacgctgaaagaaatggactatgtaatcggtgagattgaaaacgattctgaagtgttagctgttatcctgactggggcgggagagaagagttttgtcgccggcgcagacatttcagaaatgaaagagatgaatacaatcgaaggtcgcaaattcgggattctgggaaacaaggtatttcggcgtttagaactgctggagaaaccagtgatcgctgcggttaatggcttcgccttaggtggcggttgcgaaattgcaatgtcctgtgatatccgcattgcttcgagcaacgcgcgttttgggcagcctgaggtcggactgggcatcacaccgggtttcggcggtacgcaacgcctgtctcggttagtggggatgggaatggccaaacagctgatttttactgcacaaaatatcaaggctgacgaagcgctgcgtattggcctggtaaacaaagttgtggaaccaagtgagttaatgaatacagccaaagaaatcgcaaacaagattgtctcaaatgcgcctgttgctgtaaaactgtccaaacaggccattaaccgcggtatgcagtgcgatatcgacaccgcactggcgttcgagtcggaagcttttggggaatgtttcagcacggaggaccaaaaggatgccatgaccgcatttattgaaaaacgtaaaattgaaggcttcaaaaatagataggataggtaccaagaattatttaaagcttattatgccaaaatacttatatagtattttggtgtaaatgcattgatagtttctttaaatttagggaggtctgtttaatgaaaaaggtatgtgttataggcgcgggaaccatgggtagcggtattgcccaggcatttgctgcaaaaggtttcgaagtggttctgcgtgatatcaaggacgagtttgtcgatcgcggcttagacttcattaataaaaacctgtctaaactggtaaagaaagggaaaatcgaagaggcgacgaaggtggaaattttaactcggatcagtggaacagttgatctgaatatggccgctgactgcgatctggtcattgaagcggccgtagagcgtatggatatcaaaaaacaaatttttgcagacttagataacatctgtaagccggaaaccattctggcttcaaatacgtcctcgctgagcatcactgaggtggcgtctgccacaaaacgcccagacaaagttattggcatgcatttctttaaccctgcaccggtcatgaagttagtggaagtaatccgtgggattgctaccagtcaggaaacgttcgatgcggttaaagagacctcaatcgccattggaaaagacccagtggaagtcgcagaggcgcctggctttgttgtaaatcgcattctgatcccgatgattaacgaagctgtgggaatcctggccgaaggaattgcatccgtcgaggatatcgacaaggcgatgaaattaggcgctaatcacccgatgggtccactggaactgggcgacttcattggtctggatatctgcttagccattatggacgttctgtattcggagactggggatagcaaataccggcctcatacactgttaaagaaatatgtgcgtgcaggatggctgggccgcaaatctggtaagggtttctacgattattcaaaataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 37 - DNA, 7814 bp, Artificial Sequence
  Description of Artificial Sequence Synthetic polynucleotide
ctcgagtccctatcagtgatagagattgacatccctatcagtgatagagatactgagcacatcagcaggacgcactgaccgaattcacaataaaaaccgtatcaaaatttaggaggttagttagaatgaaagaagttgtaatagctagcgcggtgcgtaccgccattggctcttatggtaaaagtctgaaggatgttccggcagtcgacttaggggctacggcgatcaaagaagccgtaaaaaaggcaggaattaaaccagaggatgtgaatgaagttatcctgggcaacgtcctgcaggctggtttagggcaaaatcctgcgcgccaggcctcatttaaagcaggactgccggtagagattccagctatgactatcaacaaggtgtgcggctccggtctgcggacagtttcgttagcggcccaaattatcaaagcaggcgacgctgatgtcattatcgcgggtgggatggaaaatatgagccgtgccccttacctggcaaacaatgcgcgctggggatatcgtatgggcaacgctaaattcgtggacgaaatgattaccgatggtctgtgggatgcctttaatgactaccatatgggcatcacggcagagaacattgcggaacgctggaatatctctcgggaggaacaggatgagttcgctttagccagtcagaagaaagcagaggaagcgattaaatcaggtcaatttaaggacgagatcgtaccggttgtgattaaagggcgtaaaggagaaactgtcgttgatacagacgaacacccgcgcttcggctccaccattgagggtctggctaagctgaaaccagcctttaaaaaggatgggacggtaaccgcaggcaacgcgtcgggtttaaatgattgtgccgcagtgctggtcatcatgagcgcggaaaaagctaaagagctgggagttaagcctctggccaaaattgtgtcttatggcagtgcgggtgtagacccggctatcatggggtacggcccgttctatgcaactaaagccgcgattgaaaaggctggttggacagtcgatgaattagacctgatcgagtcaaacgaagcatttgccgcgcagtccctggctgttgcaaaagatttaaaattcgatatgaataaggtgaacgtaaatggaggcgccattgcgctgggtcatccaatcggggcttcgggagcacgtattctggttacgttagtgcacgccatgcaaaaacgcgacgcgaaaaagggcctggctaccctgtgcatcggtgggggccagggtactgcaatattgctagaaaagtgctagacttaattaaaattttataaaggagtgtatataaatgaaagttacaaatcaaaaagaactgaaacagaagttaaatgagctgcgtgaggcgcaaaaaaaatttgccacctatacgcaggaacaagtggataagattttcaaacagtgcgcaatcgctgcggccaaagaacgcattaacctggcaaagttagctgttgaagagactggcatcggtctggtcgaggacaaaattatcaaaaatcattttgcggccgagtacatttataacaagtacaaaaacgagaaaacctgtgggatcattgaccacgatgatagcctgggaatcacaaaggtagcagaaccgattggcatcgtggctgcgattgttccaacgactaatcctacatctaccgccatcttcaaaagtttaatttcactgaaaacgcggaatgcaatctttttctccccgcatccacgtgctaagaaatcgaccattgcggccgcaaaactgattttagacgcggctgtcaaggccggtgcacctaaaaacatcattgggtggatcgacgaaccgagcattgaactgtctcaggatctgatgagtgaggcggacatcattttagctactggaggcccgtcaatggtaaaagccgcatattcctcgggtaagccagcgatcggcgtgggtgctgggaatactcctgccattatcgacgaaagcgcagacattgatatggcggtttctagtatcattctgtcaaaaacgtacgacaacggagtcatctgcgcctccgaacagtcgattctggtgatgaatagcatctatgagaaagtaaaggaagagtttgttaaacgcggctcttacattctgaaccagaatgaaattgcaaaaatcaaggaaaccatgttcaaaaacggtgcgattaatgctgatatcgtgggcaaaagtgcctatattatcgcgaagatggctggtattgaggtcccgcaaactacaaaaatcttaattggggaagttcagtcagtagaaaaatccgagctgtttagccacgaaaagctgtcgccggtgttagcaatgtataaagtcaaagatttcgacgaggccctgaagaaagcgcagcgtctgatcgaattaggaggctctggtcataccagttcactgtacattgatagccaaaacaataaagacaaggttaaagaatttgggctggctatgaaaacgtcccgcacctttatcaacatgccatcgtctcagggcgcaagtggtgatttatataatttcgccattgcgcctagctttactctgggatgtggcacatggggtgggaactcagtgtcccaaaatgtagagccgaagcatctgctgaacatcaaatcggtcgctgaacggcgtgagaatatgttatggttcaaagttccacagaagatttactttaaatatggctgcctgcgcttcgcactgaaagaattaaaggatatgaacaaaaaacgtgcctttatcgtgacggacaaggatctgttcaaactgggttacgtaaataaaattaccaaggttttagacgaaattgatatcaaatattctatttttactgacatcaaaagcgatccgacaattgatagtgtgaagaaaggagcgaaagagatgctgaacttcgaacctgacacgatcatttcaatcggcggtgggtccccgatggatgctgcaaaggtcatgcatctgttatacgagtatccagaagccgaaattgagaatctggcgatcaactttatggacattcgcaaacggatctgtaattttccgaaactgggaaccaaggctattagcgttgcaatccctactacggccggcaccggttcggaagcgacaccgttcgctgtgattaccaacgatgagactgggatgaaatatccactgacatcttacgaattaacgccgaatatggcaatcattgataccgaactgatgctgaacatgcctcgtaaattaactgccgcgacgggcattgacgcactggtacacgccatcgaggcgtatgtcagtgttatggcaaccgattacacagacgaactggcgttacgcgctattaagatgatctttaaatatctgccacgtgcctacaaaaatggtactaacgatattgaagcgcgcgagaagatggctcatgcatcaaatatcgccggaatggcgttcgctaacgcatttctgggcgtgtgccacagcatggcccataaattaggtgcgatgcaccatgtaccgcatgggattgcttgtgcagtcctgatcgaagaggttattaaatataatgccacggactgccctaccaagcagacagcgttcccgcaatacaaatccccaaacgctaaacggaagtatgcagaaatcgccgaatatctgaatctgaaaggcacttcggatacggagaaagtgaccgcgttaattgaagctatctctaagctgaaaattgatctgagtatcccgcagaacatttcagcagccggtattaataaaaaggacttttacaacaccttagataaaatgagcgagctggcgttcgacgatcaatgtacaactgctaatcctcgttatccgctgatctccgaattaaaagatatctatataaaatcattttaaatcgatatattttaggaggattagtcatggaactaaacaatgtcatcctggaaaaagagggcaaggtggcggttgtcaccattaatcgtccgaaagccttaaacgcactgaatagcgatacgctgaaagaaatggactatgtaatcggtgagattgaaaacgattctgaagtgttagctgttatcctgactggggcgggagagaagagttttgtcgccggcgcagacatttcagaaatgaaagagatgaatacaatcgaaggtcgcaaattcgggattctgggaaacaaggtatttcggcgtttagaactgctggagaaaccagtgatcgctgcggttaatggcttcgccttaggtggcggttgcgaaattgcaatgtcctgtgatatccgcattgcttcgagcaacgcgcgttttgggcagcctgaggtcggactgggcatcacaccgggtttcggcggtacgcaacgcctgtctcggttagtggggatgggaatggccaaacagctgatttttactgcacaaaatatcaaggctgacgaagcgctgcgtattggcctggtaaacaaagttgtggaaccaagtgagttaatgaatacagccaaagaaatcgcaaacaagattgtctcaaatgcgcctgttgctgtaaaactgtccaaacaggccattaaccgcggtatgcagtgcgatatcgacaccgcactggcgttcgagtcggaagcttttggggaatgtttcagcacggaggaccaaaaggatgccatgaccgcatttattgaaaaacgtaaaattgaaggcttcaaaaatagataggataggtaccaagaattatttaaagcttattatgccaaaatacttatatagtattttggtgtaaatgcattgatagtttctttaaatttagggaggtctgtttaatgaaaaaggtatgtgttataggcgcgggaaccatgggtagcggtattgcccaggcatttgctgcaaaaggtttcgaagtggttctgcgtgatatcaaggacgagtttgtcgatcgcggcttagacttcattaataaaaacctgtctaaactggtaaagaaagggaaaatcgaagaggcgacgaaggtggaaattttaactcggatcagtggaacagttgatctgaatatggccgctgactgcgatctggtcattgaagcggccgtagagcgtatggatatcaaaaaacaaatttttgcagacttagataacatctgtaagccggaaaccattctggcttcaaatacgtcctcgctgagcatcactgaggtggcgtctgccacaaaacgcccagacaaagttattggcatgcatttctttaaccctgcaccggtcatgaagttagtggaagtaatccgtgggattgctaccagtcaggaaacgttcgatgcggttaaagagacctcaatcgccattggaaaagacccagtggaagtcgcagaggcgcctggctttgttgtaaatcgcattctgatcccgatgattaacgaagctgtgggaatcctggccgaaggaattgcatccgtcgaggatatcgacaaggcgatgaaattaggcgctaatcacccgatgggtccactggaactgggcgacttcattggtctggatatctgcttagccattatggacgttctgtattcggagactggggatagcaaataccggcctcatacactgttaaagaaatatgtgcgtgcaggatggctgggccgcaaatctggtaagggtttctacgattattcaaaataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 38 - DNA, 3126 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattcaggaggaatttaaaatgaagatcgttttagtcttatatgatgctggtaaacacgctgccgatgaagaaaaattatacggttgtactgaaaacaaattaggtattgccaattggttgaaagatcaaggacatgaattaatcaccacgtctgataaagaaggcggaaacagtgtgttggatcaacatataccagatgccgatattatcattacaactcctttccatcctgcttatatcactaaggaaagaatcgacaaggctaaaaaattgaaattagttgttgtcgctggtgtcggttctgatcatattgatttggattatatcaaccaaaccggtaagaaaatctccgttttggaagttaccggttctaatgttgtctctgttgcagaacacgttgtcatgaccatgcttgtcttggttagaaattttgttccagctcacgaacaaatcattaaccacgattgggaggttgctgctatcgctaaggatgcttacgatatcgaaggtaaaactatcgccaccattggtgccggtagaattggttacagagtcttggaaagattagtcccattcaatcctaaagaattattatactacgattatcaagctttaccaaaagatgctgaagaaaaagttggtgctagaagggttgaaaatattgaagaattggttgcccaagctgatatagttacagttaatgctccattacacgctggtacaaaaggtttaattaacaaggaattattgtctaaattcaagaaaggtgcttggttagtcaatactgcaagaggtgccatttgtgttgccgaagatgttgctgcagctttagaatctggtcaattaagaggttatggtggtgatgtttggttcccacaaccagctccaaaagatcacccatggagagatatgagaaacaaatatggtgctggtaacgccatgactcctcattactctggtactactttagatgctcaaactagatacgctcaaggtactaaaaatatcttggagtcattctttactggtaagtttgattacagaccacaagatatcatcttattaaacggtgaatacgttaccaaagcttacggtaaacacgataagaaataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 39 - DNA, 2106 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccttaacgatcggttggcgccttaggattcccgggagatccccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 40 - DNA, 3311 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaattgtgagcggataacaattgacattgtgagcggataacaagatactgagcacatcagcaggacgcactgaccgaattctgaggagaagtcgacttggaagcggccgcttaggatccttgaggagattggtaccatggccatgttcaccactaccgccaaggttattcagccgaaaatccgtggttttatctgtacgaccacccacccgattggctgtgaaaaacgcgtgcaggaagaaattgcttacgcacgtgcacatccaccgaccagcccgggtccgaaacgtgtcctggtcatcggctgttccactggctacggcctgtctactcgtatcaccgcagctttcggctatcaggcggctactctgggcgtgttcctggctggtccgccgactaaaggtcgcccggctgcggccggttggtataacaccgtagctttcgaaaaagcggccctggaagccggtctgtatgcccgctccctgaacggtgacgcttttgactctactaccaaagcacgcaccgtggaagctatcaaacgtgacctgggcaccgttgacctggtggtttatagcattgcagctccgaaacgtaccgatccggctaccggcgtgctgcacaaagcgtgtctgaaaccgatcggtgcgacctacaccaaccgtacggtaaatactgacaaagctgaagttacggacgtgtccatcgaaccggcgagcccagaagaaattgcagacactgtgaaagtaatgggtggcgaagactgggaactgtggattcaggctctgtctgaagccggcgttctggcagaaggcgcgaaaaccgtcgcatactcttatatcggtccggagatgacctggccggtgtactggtccggcaccattggtgaagccaaaaaggatgttgaaaaagccgctaaacgtattacccagcagtacggctgtccggcatacccggttgtggcaaaagcactggtgacgcaggcatcctccgcgatcccggtcgtcccgctgtatatttgtctgctgtaccgtgtaatgaaagaaaaaggcactcacgaaggttgcatcgaacaaatggtgcgtctgctgaccacgaaactgtacccggaaaacggtgccccgatcgttgatgaagcgggccgtgttcgtgtggacgattgggaaatggcagaagacgttcagcaagccgttaaagacctgtggagccaggtgagcacggcaaacctgaaagatatttccgacttcgccggttaccaaaccgagttcctgcgcctgtttggttttggtatcgatggcgtggactatgaccagccggttgacgtagaggcagacctgccgagcgcagctcagcagtaaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 41 - DNA, 3620 bp, Artificial Sequence
  Description of Artificial Sequence Synthetic polynucleotide
ctcgagtccctatcagtgatagagattgacatccctatcagtgatagagatactgagcacatcagcaggacgcactgaccgaattcattaaagaggagaaaggtaccatgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagtactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaattggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtctaagaaaccattattatcatgacattaacctataaaaataggcgtatcacgaggccctttcgtcttcac

SEQ ID NO: 42 - DNA, 3620 bp, Artificial Sequence
  Description of Artificial Sequence Synthetic polynucleotide
ctcgagtccctatcagtgatagagattgacatccctatcagtgatagagatactgagcacatcagcaggacgcactgaccgaattcattaaagaggagaaaggtaccatgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaattggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtctaagaaaccattattatcatgacattaacctataaaaataggcgtatcacgaggccctttcgtcttcac

SEQ ID NO: 43 - DNA, 4244 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagataaatgtgagcggataacattgacattgtgagcggataacaagatactgagcacatcagcaggacgcactgaccgaattcattaaagaggagaaaggtaccatggccatgttcaccactaccgccaaggttattcagccgaaaatccgtggttttatctgtacgaccacccacccgattggctgtgaaaaacgcgtgcaggaagaaattgcttacgcacgtgcacatccaccgaccagcccgggtccgaaacgtgtcctggtcatcggctgttccactggctacggcctgtctactcgtatcaccgcagctttcggctatcaggcggctactctgggcgtgttcctggctggtccgccgactaaaggtcgcccggctgcggccggttggtataacaccgtagctttcgaaaaagcggccctggaagccggtctgtatgcccgctccctgaacggtgacgcttttgactctactaccaaagcacgcaccgtggaagctatcaaacgtgacctgggcaccgttgacctggtggtttatagcattgcagctccgaaacgtaccgatccggctaccggcgtgctgcacaaagcgtgtctgaaaccgatcggtgcgacctacaccaaccgtacggtaaatactgacaaagctgaagttacggacgtgtccatcgaaccggcgagcccagaagaaattgcagacactgtgaaagtaatgggtggcgaagactgggaactgtggattcaggctctgtctgaagccggcgttctggcagaaggcgcgaaaaccgtcgcatactcttatatcggtccggagatgacctggccggtgtactggtccggcaccattggtgaagccaaaaaggatgttgaaaaagccgctaaacgtattacccagcagtacggctgtccggcatacccggttgtggcaaaagcactggtgacgcaggcatcctccgcgatcccggtcgtcccgctgtatatttgtctgctgtaccgtgtaatgaaagaaaaaggcactcacgaaggttgcatcgaacaaatggtgcgtctgctgaccacgaaactgtacccggaaaacggtgccccgatcgttgatgaagcgggccgtgttcgtgtggacgattgggaaatggcagaagacgttcagcaagccgttaaagacctgtggagccaggtgagcacggcaaacctgaaagatatttccgacttcgccggttaccaaaccgagttcctgcgcctgtttggttttggtatcgatggcgtggactatgaccagccggttgacgtagaggcagacctgccgagcgcagctcagcagtaaggatccaggaggaatttaaaatgaagatcgttttagtcttatatgatgctggtaaacacgctgccgatgaagaaaaattatacggttgtactgaaaacaaattaggtattgccaattggttgaaagatcaaggacatgaattaatcaccacgtctgataaagaaggcggaaacagtgtgttggatcaacatataccagatgccgatattatcattacaactcctttccatcctgcttatatcactaaggaaagaatcgacaaggctaaaaaattgaaattagttgttgtcgctggtgtcggttctgatcatattgatttggattatatcaaccaaaccggtaagaaaatctccgttttggaagttaccggttctaatgttgtctctgttgcagaacacgttgtcatgaccatgcttgtcttggttagaaattttgttccagctcacgaacaaatcattaaccacgattgggaggttgctgctatcgctaaggatgcttacgatatcgaaggtaaaactatcgccaccattggtgccggtagaattggttacagagtcttggaaagattagtcccattcaatcctaaagaattattatactacgattatcaagctttaccaaaagatgctgaagaaaaagttggtgctagaagggttgaaaatattgaagaattggttgcccaagctgatatagttacagttaatgctccattacacgctggtacaaaaggtttaattaacaaggaattattgtctaaattcaagaaaggtgcttggttagtcaatactgcaagaggtgccatttgtgttgccgaagatgttgctgcagctttagaatctggtcaattaagaggttatggtggtgatgtttggttcccacaaccagctccaaaagatcacccatggagagatatgagaaacaaatatggtgctggtaacgccatgactcctcattactctggtactactttagatgctcaaactagatacgctcaaggtactaaaaatatcttggagtcattctttactggtaagtttgattacagaccacaagatatcatcttattaaacggtgaatacgttaccaaagcttacggtaaacacgataagaaataacctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 44 - DNA, 1395 bp, Artificial Sequence
  Description of Artificial Sequence Synthetic polynucleotide
atgaatcgttccgcaatcggcgtctcctctatggtgggtaacctggttttctctgttatctccgttaaacgtgagatcacgggccagtctggtactttccgtgcccgtccgccagccatcggctgcttcctgtacaacgcacgcgatttctccgatttccgcccgtctccgccgtttcgtcaggaagtatctatgatcatcaaacctcgcgttcgtggcttcatctgcgttaccacccacccagttggctgtgaggcgaacgttaaagaacagatcgactacgttacgagccacggcccgattgcaaacggtccgaaaaaggtactggtaattggtgcgagcaccggttacggcctggccgctcgcatcagcgccgctttcggtagcggcgcagacactctgggtgttttcttcgaacgtgcaggtagcgaaaccaagccgggcaccgcgggttggtacaactccgccgccttcgaaaaattcgctgcggaaaagggcctgtacgctcgttccatcaatggcgatgcgttcagcgacaaagtaaaacaggtgaccatcgacaccattaagcaggacctgggtaaggtggacctggttgtttattctctggctgcgccacgccgtacccatccgaagacgggtgaaaccatctccagcaccctgaagcctgtgggtaaagcggttactttccgcggcctggatacggacaaagaggttatccgcgaagtatccctggaaccggcaacccaagaagagattgacggcaccgtggcagttatgggcggcgaggattggcagatgtggatcgacgctctggatgaggcaggcgtactggccgacggcgctaaaactaccgctttcacttacctgggtgaacagatcacccatgacatctattggaacggcagcattggcgaagctaaaaaggacctggacaagaaagtgctgagcattcgcgacaagctggccgcgcacggcggcgatgctcgcgtaagcgtcctgaaagcagtcgtgacccaagcgtcttctgcaatcccgatgatgccgctgtatctgagcctgctgttcaaagtgatgaaggagactggcactcatgaaggttgtatcgaacaggtgtacggcctgctgaaagacagcctgtatggtgctactccacacgtagacgaagagggccgtctgcgtgctgactataaagaactggacccgcaggtacaagataaagtggtagctatgtgggataaagttaccaacgaaaatctgtacgaaatgactgacttcgcgggttacaaaaccgaatttctgcgcctgttcggctttgaaatcgcaggtgttgattatgatgccgacgttaatcctgatgttaagattccgggcattattgatactacggtttga

SEQ ID NO: 45 - DNA, 1221 bp, Artificial Sequence
  Description of Artificial Sequence Synthetic polynucleotide
atgatcgtccagccgaaagttcgcggttttatctgcactaccgcacacccagaaggctgcgcgcgtcacgttggtgagtggatcaattatgctaagcaggagccttccctgaccggcggtccgcagaaagtactgattatcggtgcgagcacgggctttggtctggcgtctcgtatcgtggctgccttcggtgcgggtgctaaaacgattggtgtgtttttcgaacgtccggcttctggcaaacgcaccgcgtcccctggttggtacaatactgcagcgttcgagaagaccgctctggcggctggcctgtacgcgaaatctatcaacggcgacgcgttcagcgacgaaattaaacagcaaaccatcgacctgatccagaaagattggcagggcggtgttgacctggtaatttactctatcgcgagcccgcgtcgcgtacacccgcgtactggtgaaatcttcaactctgtcctgaaacctattggtcagacctaccacaacaaaactgtggacgtaatgaccggcgaagtttccccggtatctattgagccggcaacggaaaaggaaatccgcgacactgaagcggtaatgggtggcgacgactgggcgctgtggatcaacgcgctgttcaaatacaactgcctggccgaaggcgtcaaaaccgttgcgttcacctatattggtccggaactgacccacgcggtatatcgtaacggcactatcggccgtgcgaaactgcacctggaaaagactgctcgcgaactggatacccagctggagagcgcgctgtctggtcaggctctgatttctgttaacaaagccctggtgacccaggcttccgcagctatcccggtagttccgctgtatatctccctgctgtataaaatcatgaaagagaaaaacatccacgagggttgcatcgagcagatgtggcgtctgtttaaggagcgcctgtactctaaccagaacatccctactgactccgaaggccgcatccgtattgatgactgggaaatgcgcgaagacgtacaagcggaaatcaaacgtctgtgggaatccatcaacaccggtaacgttgaaactgtctctgatatcgctggctatcgtgaggacttctataaactgttcggtttcggtctgaacggtatcgactacgaacgtggcgttgaaattgaaaaggctatcccgtccatcactgttactcctgaaaacccggaataa

SEQ ID NO: 46 - DNA, 1179 bp, Artificial Sequence
  Description of Artificial Sequence Synthetic polynucleotide
atgatcattaaaccgaaggtgcgtggctttatctgcactactgctcatccggtcggctgtgcagagaatgttcaacagcagatcgactacgtagcagcccagaacgccccgtctagcggcccgaaaaatgtactggtcatcggttgcagcaacggttacggtctggcgtcccgcatcaccagcgcattcggctttggtgcgaacaccctgggcgtcatgttcgaaaaagaaccgaccgaacgccgtccggcatctgccggttggtataacacccgtgcgctggagaaagcggctcaggaaaaaggtctgtacgcgcaatctctgaatgtggatgcgttctccgatgaagctaaaaccgcagtaatcgaggctgtgaaagctaacatgggtaaaattgatctggtcgtttacagcctgggtgcaccgcgtcgtaaagatccggaaaccggcactgtctactccagcacgctgaaacctattggcaaagctgtgacccgtaaaaacctgaacactgacacccgtgaggtaggtgaagtgactctggaaccagcgaccgaagaagaaattttcaacacggtgaaagtaatgggcggtgaagactgggaacgctggatgaccgctctggacgacgctggcgtgctggcagacggcgttaaaactaccgcgtatacctacattggtaaagagctgacctggccgatctacggcggtgcgaccatcggcaaggctaaagaagatctggatcgcgcatccgttgctattaacaagaaactggcagacaaatatcagggtgttagctacgtcgcagtgctgaaagcgctggtaactcagtcttcttccgccatcccagtaatgccgctgtacatttctgctctgtatcgtgttatgaaggaagaaggcacgcacgaaggctgcatcgagcagatcacgggcctgtttttcgaccagctgttctctgaaaacgccctgaacctggatgataccggccgtatccgcatggaagataacgaactgaaagcgtctgtacaggagaaagttgctgcgatctgggaacaggttaacacggaaaatctggacgagctgaccgacttcaaaggttaccaggaagaatttttcaaactgttcggtttcggcttcgaaggtgttgattacgacgcagacgtagatccagtggtgtga

SEQ ID NO: 47 - DNA, 1203 bp, Artificial Sequence
  Description of Artificial Sequence Synthetic polynucleotide
atgattatcaaaccgaaaacgcgtggctttatctgcactaccacccacccggttggttgtgaagccaacgttctggaacaaatcaacaccactaaagccaaaggcccgatcaccaatggtccaaaaaaagttctggttattggcagctccagcggttacggtctgtcttcccgtatcgctgcggcgtttggttccggtgcagcgaccctgggtgtattcttcgaaaaaccgggcaccgagaagaaacctggcaccgctggttggtataacagcgctgctttcgataaattcgctaaggcagatggcctgtactctaaatctattaacggtgacgcgttctcccacgaagccaaacagaaagcgatcgacctgatcaaagcggatctgggccaaattgacatggttgtgtactctctggcttctccggttcgtaaactgccggattccggcgaactgattcgttctagcctgaaaccaatcggcgaaacttacaccgctactgctgttgacacgaacaaagacctgatcattgaaacgagcgttgaaccagcgagcgaacaggaaatccaagatactgtaaccgtaatgggcggtgaagactgggaactgtggctggccgcgctgagcgatgctggtgtcctggcggatggctgcaaaaccgttgcgtactcttacattggtacggaactgacctggccgatctactggcacggcgctctgggcaaggcaaaaatggacctggaccgtgccgcaaaagcgctggacgaaaaactgagcacgaccggtggctctgcaaatgtggctgtgctgaaatctgtagtgacccaggcgtcctccgctatcccggtgatgccgctgtacatcgccatggtattcaaaaagatgcgcgaagaaggtctgcacgaaggctgcatggaacagatcaaccgtatgttcgcggaacgtctgtaccgtgaagatggtcaggctccgcaggtcgatgatgcaaatcgtctgcgcctggacgattgggaactgcgcgaggagatccagcagcactgccgtgatctgtggccgtctgtgactactgagaacctgagcgagctgaccgactaccgtgaatataaagatgagttcctgaaactgttcggtttcggcgttgaaggtgtagattacgacgccgacgttaacccggaagtaaacttcgacgtagaacagttctaa

SEQ ID NO: 48 - DNA, 1194 bp, Artificial Sequence
  Description of Artificial Sequence Synthetic polynucleotide
atgatcgtaaagcctatggttcgtaacaatatttgcctgaacgctcatccgcagggttgcaagaaaggtgtcgaggatcagattgaatacaccaagaaacgtattaccgctgaagttaaagcaggtgctaaagcgccgaaaaacgtgctggttctgggctgttccaacggctacggcctggcgtctcgcatcactgctgcgtttggttatggtgcggctactatcggtgtttcttttgaaaaagcgggctccgaaaccaaatatggcaccccaggttggtacaacaacctggcgttcgatgaagcggctaaacgcgagggcctgtactctgtgactatcgacggtgacgccttcagcgatgaaatcaaagcacaggttatcgaggaagccaaaaagaaaggcattaagtttgacctgattgtgtactctctggctagcccggtgcgtaccgatccggataccggcatcatgcacaaatccgtcctgaaaccgttcggcaaaactttcaccggtaaaacggtagatccgttcactggtgagctgaaagaaatctctgccgagccagctaacgatgaagaggcagctgctactgtcaaagtcatgggtggtgaagattgggaacgttggatcaaacagctgtctaaagaaggtctgctggaggaaggctgcattaccctggcatactcctacattggtccagaggccactcaggcgctgtatcgtaaaggtactatcggtaaagctaaagaacacctggaagctacggctcaccgtctgaacaaagaaaacccgtccatccgtgcattcgtttccgtcaacaagggcctggtcacccgtgcatccgcagttatcccggtcatccctctgtatctggcttccctgttcaaggttatgaaggaaaaaggtaaccatgagggttgtatcgaacagatcacccgtctgtacgccgaacgtctgtaccgcaaggatggcaccatcccggttgatgaggaaaaccgcattcgtatcgacgactgggaactggaagaagatgttcaaaaagctgtgtctgcgctgatggaaaaagtgaccggcgaaaatgcggaatccctgacggacctggcgggctatcgtcatgactttctggcgtccaacggttttgatgttgagggcatcaactatgaagcggaagtagagcgttttgaccgcatttaa

SEQ ID NO: 49 - DNA, 1386 bp, Artificial Sequence
  Description of Artificial Sequence Synthetic polynucleotide
atgcgtctgctgttcgaagcagttcacgcgcgtaagcgttggcatcgtactgcgccggctgccgcattcactcgttttcacaccgctgcatgcgtgactcatcaggcagtttcccgtgctccacacgccctgcgttgtcgccagcacctggcagatcaggagtccacgctgatcattcacccgaaagtacgtggtttcatctgcacgaccactcaccctctgggttgcgaacgtaacgtcctggaacagatcgcggctactcgtgctcgcggtgttcgtaacgatggtccgaagaaagttctggtgatcggcgcgtctagcggttacggtctggccagccgcattaccgccgcattcggtttcggtgcggataccctgggtgttttcttcgaaaaaccgggtactgcctctaaagctggcacggcgggttggtacaactccgcagcattcgacaagcacgcaaaagcggctggtctgtactctaaatctatcaatggtgatgcgttcagcgatgcggcgcgtgcacaggtgatcgaactgatcaaaactgagatgggtggtcaagttgacctggttgtttactctctggcctccccggtacgtaaactgccgggctctggtgaagttaaacgttctgcgctgaagccaatcggccagacctacaccgcaacggcgatcgacaccaacaaggacactatcatccaggcttccattgaacctgcttctgcgcaggaaatcgaggataccatcaccgtgatgggcggccaagactgggaactgtggatcgacgcactggaaggtgcaggcgtactggcagatggcgctcgttctgtagcgttctcctatatcggcaccgaaatcacttggccgatctactggcatggcgcactgggcaaagcaaaagtggacctggaccgtaccgctcaacgtctgaatgcccgtctggcaaaacacggtggtggcgcaaacgtggcagttctgaagagcgtagtgacccaagcttctgccgctattccggttatgccgctgtacatttccatggtgtataaaatcatgaaagaaaaaggtctgcatgagggtactatcgaacagctggatcgcctgtttcgtgaacgtctgtaccgccaggacggtcagccggcagaagtagatgaagttgatgaacagaaccgtctgcgcctggacgattgggaactgcgcgacgatgtacaggacgcctgcaaggctctgtggccgcaggtaactactgaaaatctgttcgagctgaccgattacgcgggctacaaacatgagttcctgaaactgtttggcttcggccgtaccgacgttgattacgatgcggatgttgcaactgacgtggctttcgattgtatcgaactggcctga

SEQ ID NO: 50 - DNA, 1200 bp, Artificial Sequence
  Description of Artificial Sequence Synthetic polynucleotide
atgatcattaaaccgcgtgttcgtggctttatctgtgttaccgctcatccgaccggctgcgaagcgaacgtcaaaaagcagatcgactacgttaccactgaaggcccgatcgctaacggccctaaacgcgttctggtaattggcgcttctaccggttacggcctggcggcacgtatcaccgccgcgtttggttgcggcgctgacaccctgggtgtgttcttcgaacgtccgggtgaagaaggcaaaccgggcacttctggctggtacaactccgcagcgtttcacaaatttgccgctcagaaaggtctgtacgcaaaatctatcaacggcgacgctttcagcgacgaaatcaaacagctgaccattgacgcgatcaaacaggacctgggccaggtagatcaggtgatctactccctggcctctccgcgtcgcacccaccctaaaaccggtgaagtattcaattccgccctgaagccgatcggtaacgcagtaaacctgcgcggcctggataccgacaaggaggtgatcaaagaaagcgtgctgcagccggcaacccagtctgaaattgactccactgttgcggtgatgggtggcgaagattggcagatgtggatcgacgcgctgctggatgcaggcgtactggcagaaggcgctcagactaccgcgttcacgtacctgggcgaaaagatcacccatgacatttattggaacggttccattggcgctgccaaaaaggacctggatcagaaagttctggctatccgtgaatccctggctgctcacggtggtggcgatgcacgtgtctccgtgctgaaagcagtcgtcacccaggcgtcctccgcgattccaatgatgccgctgtatctgagcctgctgtttaaagtcatgaaggaaaaaggcacccacgagggctgcattgaacaggtgtactctctgtataaagattctctgtgtggtgatagcccacatatggaccaggaaggtcgtctgcgtgctgactataaagagctggacccggaagtgcagaaccaggttcagcagctgtgggatcaagttactaacgacaacatttaccagctgacggatttcgtaggctacaaatctgagtttctgaacctgttcggtttcggtatcgacggtgtggactatgatgccgatgtcaacccggatgtaaagattccgaacctgatccaaggttaa

SEQ ID NO: 51 - DNA, 1188 bp, Artificial Sequence
  Description of Artificial Sequence Synthetic polynucleotide
atggttatttctcctaaggttcgcggctttatttgcactaatgcgcacccggttggttgtgcgaaaagcgtggaaaaccagatcgcttacgttaaagcgcagggtctgtctgctgaggcggcagatgcaccgaaaaacgtgctggttctgggctgttccaccggctatggtctggcgtctcgtatcactgcgtcctttggctatggtgccaacactgtaggcgtttgtttcgaaaaagctccgacggaacgcaaaaccggtactgcgggttggtataacacggcggcgttccacagcgaagcaaaagccgcaggcgttcaggcccataccctgaatggcgacgcattctccaacgaactgaaagcacagaccatcgaaaccctgaagaacaccatcggtaaagttgacctggtggtgtactctctggcgtccccgcgtcgtaccgacccggaaactggtgaagtgtataagagcaccctgaaaccggttggtcaggcatatgagaccaagacctacgacactgacaaagatctgatccacacggtggctctggaaccggcttctcaggatgaaattgataacaccatcaaagtgatgggtggtgaagactgggaactgtggatcaaagcgctggcggaagcggatctgctggcggagggtgctaaaaccaccgcttacacctacatcggcaaaaagctgacctggccgatctacggctccgccactatcggcaaagcaaaagaagacctggatcgcgctgccaccgcgatcaacaccacctacgcaaacctgaacgttgatgctcacgtatctagcctgaaagccctggtgacccaagcctcttccgctatcccggtcatgcctctgtatatcagcctgatttacaaagttatgaaagaagagggcactcacgaaggttgtatcgaacagatcgttggtctgtttactcagtgcctgctgaacgacggcgcgactctggatgaagttaaccgttatcgtatggatggtaaagaaactaacgacgccactcaggctaaaattgaagagctgtggcaccaggtgacccaggacaactttcacgaactgtccgactacgctggttataacgctgatttcctgaacctgtttggttttggcatcgaaggtgttgattacgaagcggacgttgatccgcaggtgtcctggtaa

SEQ ID NO: 52 - DNA, 1198 bp, Artificial Sequence
  Description of Artificial Sequence Synthetic polynucleotide
ggtaccatgattattgaacctaagatgcgtggctttatttgtctgacctcccacccgacgggttgtgaacagaacgttatcaaccagatcaactacgtgaaaagcaaaggcgttattaatggcccgaagaaagttctggttattggcgcatccactggcttcggcctggcgtctcgtatcacttctgctttcggtagcaatgctgcgacgatcggtgtcttcttcgaaaaaccggcgcaggagggtaaaccgggctctccgggctggtataacaccgtagctttccagaatgaggccaaaaaggctggcatttacgctaaaagcatcaacggtgatgccttttccactgaagtaaagcagaaaaccatcgacctgattaaagctgatctgggtcaagtggacctggttatctacagcctggcaagccctgttcgtaccaacccggtaaccggtgtaacccaccgctctgtactgaaaccgattggtggtgcgttctctaacaaaactgttgacttccataccggcaacgtaagcaccgttaccatcgaaccagcgaacgaagaagatgttaccaacaccgtcgctgttatgggtggtgaggattggggcatgtggatggacgcgatgctggaagcaggcgttctggccgaaggcgcaactacggttgcatattcctacatcggtccggctctgaccgaagcggtgtatcgtaagggcactatcggccgtgcgaaagaccacctggaggcatctgctgcaaccattactgataaactgaaatctgttaaaggtaaagcctacgtgtctgtgaacaaagcgctggtcacccaggcttccagcgcaattccggttattccgctgtacatctctctgctgtacaaggttatgaaagcagagggcattcacgaaggttgtatcgaacagattcagcgtctgtacgctgaccgtctgtacacgggcaaagctatcccaacggacgagcagggccgtatccgtatcgacgattgggaaatgcgtgaagatgtccaggcgaacgttgcagcactgtgggaacaagttacttctgaaaacgtttccgacatctctgacctgaaaggttataagaacgactttctgaacctgttcggtttcgcggttaacaaagttgattatctggctgacgtgaacgaaaacgttacgatcgaaggtctggtatgag

SEQ ID NO: 53 - DNA, 1203 bp, Artificial Sequence
  Description of Artificial Sequence Synthetic polynucleotide
atgatcattaaacctcgtatccgtggctttatctgcaccacgactcacccggtaggttgcgaagctaacgtcaaagaacaaatcgcatacactaaagctcagggcccgatcaaaaacgcccctaaacgtgttctggttgttggtgcctcctccggttatggtctgtcttctcgtatcgcggcagcgtttggcggcggtgcttccaccatcggcgtgttcttcgaaaaggaaggcaccgaaaagaaacctggtactgctggcttctacaacgctgcggcgttcgaaaaactggcgcgtgaagagggcctgtacgccaagagcctgaacggcgatgcattctccaacgaggcgaaacagaaaaccattgaactgatcaaagaagacctgggtcaaattgatatggtggtttacagcctggcatccccggtgcgcaaaatgccggaaaccggtgaactggtgcgcagcgcactgaaaccgattggtgagacttatacctctaccgcggtcgatacgaataaggatgtgatcattgaagcgagcgttgaaccggcgaccgaagaggaaatcaaagataccgtgactgtaatgggtggtgaggattgggaactgtggatcaatgcgctgagcgatgcaggcgtgctggctgaaggttgcaaaactgttgcttatagctacattggcaccgaactgacctggcctatctactgggacggtgcactgggtaaagctaaaatggatctggatcgtgcagccaaagcactgaacgacaaactggcggcaaccggtggctctgcgaatgtcgctgttctgaaatccgttgtaacccaagcttcctccgcaatcccggttatgccgctgtatatcgcaatggtgttcaagaaaatgcgcgaagaaggtgtacacgaaggctgcatggaacagatttaccgtatgttctctcagcgtctgtacaaggaagacggctctgctgccgaggttgatgaaatgaaccgtctgcgtctggacgattgggagctgcgcgacgacattcagcagcactgccgtgaactgtggccgcagattaccaccgaaaatctgaaagaactgaccgattacgttgaatataaggaagagttcctgaaactgttcggtttcggtgttgagggcgttgattacgaagcagacgtgaacccggctgtggaagccgatttcatccagatctaa

SEQ ID NO: 54 - DNA, 3487 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatgaatcgttccgcaatcggcgtctcctctatggtgggtaacctggttttctctgttatctccgttaaacgtgagatcacgggccagtctggtactttccgtgcccgtccgccagccatcggctgcttcctgtacaacgcacgcgatttctccgatttccgcccgtctccgccgtttcgtcaggaagtatctatgatcatcaaacctcgcgttcgtggcttcatctgcgttaccacccacccagttggctgtgaggcgaacgttaaagaacagatcgactacgttacgagccacggcccgattgcaaacggtccgaaaaaggtactggtaattggtgcgagcaccggttacggcctggccgctcgcatcagcgccgctttcggtagcggcgcagacactctgggtgttttcttcgaacgtgcaggtagcgaaaccaagccgggcaccgcgggttggtacaactccgccgccttcgaaaaattcgctgcggaaaagggcctgtacgctcgttccatcaatggcgatgcgttcagcgacaaagtaaaacaggtgaccatcgacaccattaagcaggacctgggtaaggtggacctggttgtttattctctggctgcgccacgccgtacccatccgaagacgggtgaaaccatctccagcaccctgaagcctgtgggtaaagcggttactttccgcggcctggatacggacaaagaggttatccgcgaagtatccctggaaccggcaacccaagaagagattgacggcaccgtggcagttatgggcggcgaggattggcagatgtggatcgacgctctggatgaggcaggcgtactggccgacggcgctaaaactaccgctttcacttacctgggtgaacagatcacccatgacatctattggaacggcagcattggcgaagctaaaaaggacctggacaagaaagtgctgagcattcgcgacaagctggccgcgcacggcggcgatgctcgcgtaagcgtcctgaaagcagtcgtgacccaagcgtcttctgcaatcccgatgatgccgctgtatctgagcctgctgttcaaagtgatgaaggagactggcactcatgaaggttgtatcgaacaggtgtacggcctgctgaaagacagcctgtatggtgctactccacacgtagacgaagagggccgtctgcgtgctgactataaagaactggacccgcaggtacaagataaagtggtagctatgtgggataaagttaccaacgaaaatctgtacgaaatgactgacttcgcgggttacaaaaccgaatttctgcgcctgttcggctttgaaatcgcaggtgttgattatgatgccgacgttaatcctgatgttaagattccgggcattattgatactacggtttgaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 55 - DNA, 3313 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatgatcgtccagccgaaagttcgcggttttatctgcactaccgcacacccagaaggctgcgcgcgtcacgttggtgagtggatcaattatgctaagcaggagccttccctgaccggcggtccgcagaaagtactgattatcggtgcgagcacgggctttggtctggcgtctcgtatcgtggctgccttcggtgcgggtgctaaaacgattggtgtgtttttcgaacgtccggcttctggcaaacgcaccgcgtcccctggttggtacaatactgcagcgttcgagaagaccgctctggcggctggcctgtacgcgaaatctatcaacggcgacgcgttcagcgacgaaattaaacagcaaaccatcgacctgatccagaaagattggcagggcggtgttgacctggtaatttactctatcgcgagcccgcgtcgcgtacacccgcgtactggtgaaatcttcaactctgtcctgaaacctattggtcagacctaccacaacaaaactgtggacgtaatgaccggcgaagtttccccggtatctattgagccggcaacggaaaaggaaatccgcgacactgaagcggtaatgggtggcgacgactgggcgctgtggatcaacgcgctgttcaaatacaactgcctggccgaaggcgtcaaaaccgttgcgttcacctatattggtccggaactgacccacgcggtatatcgtaacggcactatcggccgtgcgaaactgcacctggaaaagactgctcgcgaactggatacccagctggagagcgcgctgtctggtcaggctctgatttctgttaacaaagccctggtgacccaggcttccgcagctatcccggtagttccgctgtatatctccctgctgtataaaatcatgaaagagaaaaacatccacgagggttgcatcgagcagatgtggcgtctgtttaaggagcgcctgtactctaaccagaacatccctactgactccgaaggccgcatccgtattgatgactgggaaatgcgcgaagacgtacaagcggaaatcaaacgtctgtgggaatccatcaacaccggtaacgttgaaactgtctctgatatcgctggctatcgtgaggacttctataaactgttcggtttcggtctgaacggtatcgactacgaacgtggcgttgaaattgaaaaggctatcccgtccatcactgttactcctgaaaacccggaataaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 56 - DNA, 3271 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatgatcattaaaccgaaggtgcgtggctttatctgcactactgctcatccggtcggctgtgcagagaatgttcaacagcagatcgactacgtagcagcccagaacgccccgtctagcggcccgaaaaatgtactggtcatcggttgcagcaacggttacggtctggcgtcccgcatcaccagcgcattcggctttggtgcgaacaccctgggcgtcatgttcgaaaaagaaccgaccgaacgccgtccggcatctgccggttggtataacacccgtgcgctggagaaagcggctcaggaaaaaggtctgtacgcgcaatctctgaatgtggatgcgttctccgatgaagctaaaaccgcagtaatcgaggctgtgaaagctaacatgggtaaaattgatctggtcgtttacagcctgggtgcaccgcgtcgtaaagatccggaaaccggcactgtctactccagcacgctgaaacctattggcaaagctgtgacccgtaaaaacctgaacactgacacccgtgaggtaggtgaagtgactctggaaccagcgaccgaagaagaaattttcaacacggtgaaagtaatgggcggtgaagactgggaacgctggatgaccgctctggacgacgctggcgtgctggcagacggcgttaaaactaccgcgtatacctacattggtaaagagctgacctggccgatctacggcggtgcgaccatcggcaaggctaaagaagatctggatcgcgcatccgttgctattaacaagaaactggcagacaaatatcagggtgttagctacgtcgcagtgctgaaagcgctggtaactcagtcttcttccgccatcccagtaatgccgctgtacatttctgctctgtatcgtgttatgaaggaagaaggcacgcacgaaggctgcatcgagcagatcacgggcctgtttttcgaccagctgttctctgaaaacgccctgaacctggatgataccggccgtatccgcatggaagataacgaactgaaagcgtctgtacaggagaaagttgctgcgatctgggaacaggttaacacggaaaatctggacgagctgaccgacttcaaaggttaccaggaagaatttttcaaactgttcggtttcggcttcgaaggtgttgattacgacgcagacgtagatccagtggtgtgaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 57 - DNA, 3295 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatgattatcaaaccgaaaacgcgtggctttatctgcactaccacccacccggttggttgtgaagccaacgttctggaacaaatcaacaccactaaagccaaaggcccgatcaccaatggtccaaaaaaagttctggttattggcagctccagcggttacggtctgtcttcccgtatcgctgcggcgtttggttccggtgcagcgaccctgggtgtattcttcgaaaaaccgggcaccgagaagaaacctggcaccgctggttggtataacagcgctgctttcgataaattcgctaaggcagatggcctgtactctaaatctattaacggtgacgcgttctcccacgaagccaaacagaaagcgatcgacctgatcaaagcggatctgggccaaattgacatggttgtgtactctctggcttctccggttcgtaaactgccggattccggcgaactgattcgttctagcctgaaaccaatcggcgaaacttacaccgctactgctgttgacacgaacaaagacctgatcattgaaacgagcgttgaaccagcgagcgaacaggaaatccaagatactgtaaccgtaatgggcggtgaagactgggaactgtggctggccgcgctgagcgatgctggtgtcctggcggatggctgcaaaaccgttgcgtactcttacattggtacggaactgacctggccgatctactggcacggcgctctgggcaaggcaaaaatggacctggaccgtgccgcaaaagcgctggacgaaaaactgagcacgaccggtggctctgcaaatgtggctgtgctgaaatctgtagtgacccaggcgtcctccgctatcccggtgatgccgctgtacatcgccatggtattcaaaaagatgcgcgaagaaggtctgcacgaaggctgcatggaacagatcaaccgtatgttcgcggaacgtctgtaccgtgaagatggtcaggctccgcaggtcgatgatgcaaatcgtctgcgcctggacgattgggaactgcgcgaggagatccagcagcactgccgtgatctgtggccgtctgtgactactgagaacctgagcgagctgaccgactaccgtgaatataaagatgagttcctgaaactgttcggtttcggcgttgaaggtgtagattacgacgccgacgttaacccggaagtaaacttcgacgtagaacagttctaaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 58 - DNA, 3286 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatgatcgtaaagcctatggttcgtaacaatatttgcctgaacgctcatccgcagggttgcaagaaaggtgtcgaggatcagattgaatacaccaagaaacgtattaccgctgaagttaaagcaggtgctaaagcgccgaaaaacgtgctggttctgggctgttccaacggctacggcctggcgtctcgcatcactgctgcgtttggttatggtgcggctactatcggtgtttcttttgaaaaagcgggctccgaaaccaaatatggcaccccaggttggtacaacaacctggcgttcgatgaagcggctaaacgcgagggcctgtactctgtgactatcgacggtgacgccttcagcgatgaaatcaaagcacaggttatcgaggaagccaaaaagaaaggcattaagtttgacctgattgtgtactctctggctagcccggtgcgtaccgatccggataccggcatcatgcacaaatccgtcctgaaaccgttcggcaaaactttcaccggtaaaacggtagatccgttcactggtgagctgaaagaaatctctgccgagccagctaacgatgaagaggcagctgctactgtcaaagtcatgggtggtgaagattgggaacgttggatcaaacagctgtctaaagaaggtctgctggaggaaggctgcattaccctggcatactcctacattggtccagaggccactcaggcgctgtatcgtaaaggtactatcggtaaagctaaagaacacctggaagctacggctcaccgtctgaacaaagaaaacccgtccatccgtgcattcgtttccgtcaacaagggcctggtcacccgtgcatccgcagttatcccggtcatccctctgtatctggcttccctgttcaaggttatgaaggaaaaaggtaaccatgagggttgtatcgaacagatcacccgtctgtacgccgaacgtctgtaccgcaaggatggcaccatcccggttgatgaggaaaaccgcattcgtatcgacgactgggaactggaagaagatgttcaaaaagctgtgtctgcgctgatggaaaaagtgaccggcgaaaatgcggaatccctgacggacctggcgggctatcgtcatgactttctggcgtccaacggttttgatgttgagggcatcaactatgaagcggaagtagagcgttttgaccgcatttaaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 59 - DNA, 3479 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatgcgtctgctgttcgaagcagttcacgcgcgtaagcgttggcatcgtactgcgccggctgccgcattcactcgttttcacaccgctgcatgcgtgactcatcaggcagtttcccgtgctccacacgccctgcgttgtcgccagcacctggcagatcaggagtccacgctgatcattcacccgaaagtacgtggtttcatctgcacgaccactcaccctctgggttgcgaacgtaacgtcctggaacagatcgcggctactcgtgctcgcggtgttcgtaacgatggtccgaagaaagttctggtgatcggcgcgtctagcggttacggtctggccagccgcattaccgccgcattcggtttcggtgcggataccctgggtgttttcttcgaaaaaccgggtactgcctctaaagctggcacggcgggttggtacaactccgcagcattcgacaagcacgcaaaagcggctggtctgtactctaaatctatcaatggtgatgcgttcagcgatgcggcgcgtgcacaggtgatcgaactgatcaaaactgagatgggtggtcaagttgacctggttgtttactctctggcctccccggtacgtaaactgccgggctctggtgaagttaaacgttctgcgctgaagccaatcggccagacctacaccgcaacggcgatcgacaccaacaaggacactatcatccaggcttccattgaacctgcttctgcgcaggaaatcgaggataccatcaccgtgatgggcggccaagactgggaactgtggatcgacgcactggaaggtgcaggcgtactggcagatggcgctcgttctgtagcgttctcctatatcggcaccgaaatcacttggccgatctactggcatggcgcactgggcaaagcaaaagtggacctggaccgtaccgctcaacgtctgaatgcccgtctggcaaaacacggtggtggcgcaaacgtggcagttctgaagagcgtagtgacccaagcttctgccgctattccggttatgccgctgtacatttccatggtgtataaaatcatgaaagaaaaaggtctgcatgagggtactatcgaacagctggatcgcctgtttcgtgaacgtctgtaccgccaggacggtcagccggcagaagtagatgaagttgatgaacagaaccgtctgcgcctggacgattgggaactgcgcgacgatgtacaggacgcctgcaaggctctgtggccgcaggtaactactgaaaatctgttcgagctgaccgattacgcgggctacaaacatgagttcctgaaactgtttggcttcggccgtaccgacgttgattacgatgcggatgttgcaactgacgtggctttcgattgtatcgaactggcctgaggcgccttaggattcccgggagatccccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 60 - DNA, 3292 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatgatcattaaaccgcgtgttcgtggctttatctgtgttaccgctcatccgaccggctgcgaagcgaacgtcaaaaagcagatcgactacgttaccactgaaggcccgatcgctaacggccctaaacgcgttctggtaattggcgcttctaccggttacggcctggcggcacgtatcaccgccgcgtttggttgcggcgctgacaccctgggtgtgttcttcgaacgtccgggtgaagaaggcaaaccgggcacttctggctggtacaactccgcagcgtttcacaaatttgccgctcagaaaggtctgtacgcaaaatctatcaacggcgacgctttcagcgacgaaatcaaacagctgaccattgacgcgatcaaacaggacctgggccaggtagatcaggtgatctactccctggcctctccgcgtcgcacccaccctaaaaccggtgaagtattcaattccgccctgaagccgatcggtaacgcagtaaacctgcgcggcctggataccgacaaggaggtgatcaaagaaagcgtgctgcagccggcaacccagtctgaaattgactccactgttgcggtgatgggtggcgaagattggcagatgtggatcgacgcgctgctggatgcaggcgtactggcagaaggcgctcagactaccgcgttcacgtacctgggcgaaaagatcacccatgacatttattggaacggttccattggcgctgccaaaaaggacctggatcagaaagttctggctatccgtgaatccctggctgctcacggtggtggcgatgcacgtgtctccgtgctgaaagcagtcgtcacccaggcgtcctccgcgattccaatgatgccgctgtatctgagcctgctgtttaaagtcatgaaggaaaaaggcacccacgagggctgcattgaacaggtgtactctctgtataaagattctctgtgtggtgatagcccacatatggaccaggaaggtcgtctgcgtgctgactataaagagctggacccggaagtgcagaaccaggttcagcagctgtgggatcaagttactaacgacaacatttaccagctgacggatttcgtaggctacaaatctgagtttctgaacctgttcggtttcggtatcgacggtgtggactatgatgccgatgtcaacccggatgtaaagattccgaacctgatccaaggttaaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 61 - DNA, 3280 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatggttatttctcctaaggttcgcggctttatttgcactaatgcgcacccggttggttgtgcgaaaagcgtggaaaaccagatcgcttacgttaaagcgcagggtctgtctgctgaggcggcagatgcaccgaaaaacgtgctggttctgggctgttccaccggctatggtctggcgtctcgtatcactgcgtcctttggctatggtgccaacactgtaggcgtttgtttcgaaaaagctccgacggaacgcaaaaccggtactgcgggttggtataacacggcggcgttccacagcgaagcaaaagccgcaggcgttcaggcccataccctgaatggcgacgcattctccaacgaactgaaagcacagaccatcgaaaccctgaagaacaccatcggtaaagttgacctggtggtgtactctctggcgtccccgcgtcgtaccgacccggaaactggtgaagtgtataagagcaccctgaaaccggttggtcaggcatatgagaccaagacctacgacactgacaaagatctgatccacacggtggctctggaaccggcttctcaggatgaaattgataacaccatcaaagtgatgggtggtgaagactgggaactgtggatcaaagcgctggcggaagcggatctgctggcggagggtgctaaaaccaccgcttacacctacatcggcaaaaagctgacctggccgatctacggctccgccactatcggcaaagcaaaagaagacctggatcgcgctgccaccgcgatcaacaccacctacgcaaacctgaacgttgatgctcacgtatctagcctgaaagccctggtgacccaagcctcttccgctatcccggtcatgcctctgtatatcagcctgatttacaaagttatgaaagaagagggcactcacgaaggttgtatcgaacagatcgttggtctgtttactcagtgcctgctgaacgacggcgcgactctggatgaagttaaccgttatcgtatggatggtaaagaaactaacgacgccactcaggctaaaattgaagagctgtggcaccaggtgacccaggacaactttcacgaactgtccgactacgctggttataacgctgatttcctgaacctgtttggttttggcatcgaaggtgttgattacgaagcggacgttgatccgcaggtgtcctggtaaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 62 - DNA, 3283 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatgattattgaacctaagatgcgtggctttatttgtctgacctcccacccgacgggttgtgaacagaacgttatcaaccagatcaactacgtgaaaagcaaaggcgttattaatggcccgaagaaagttctggttattggcgcatccactggcttcggcctggcgtctcgtatcacttctgctttcggtagcaatgctgcgacgatcggtgtcttcttcgaaaaaccggcgcaggagggtaaaccgggctctccgggctggtataacaccgtagctttccagaatgaggccaaaaaggctggcatttacgctaaaagcatcaacggtgatgccttttccactgaagtaaagcagaaaaccatcgacctgattaaagctgatctgggtcaagtggacctggttatctacagcctggcaagccctgttcgtaccaacccggtaaccggtgtaacccaccgctctgtactgaaaccgattggtggtgcgttctctaacaaaactgttgacttccataccggcaacgtaagcaccgttaccatcgaaccagcgaacgaagaagatgttaccaacaccgtcgctgttatgggtggtgaggattggggcatgtggatggacgcgatgctggaagcaggcgttctggccgaaggcgcaactacggttgcatattcctacatcggtccggctctgaccgaagcggtgtatcgtaagggcactatcggccgtgcgaaagaccacctggaggcatctgctgcaaccattactgataaactgaaatctgttaaaggtaaagcctacgtgtctgtgaacaaagcgctggtcacccaggcttccagcgcaattccggttattccgctgtacatctctctgctgtacaaggttatgaaagcagagggcattcacgaaggttgtatcgaacagattcagcgtctgtacgctgaccgtctgtacacgggcaaagctatcccaacggacgagcagggccgtatccgtatcgacgattgggaaatgcgtgaagatgtccaggcgaacgttgcagcactgtgggaacaagttacttctgaaaacgtttccgacatctctgacctgaaaggttataagaacgactttctgaacctgttcggtttcgcggttaacaaagttgattatctggctgacgtgaacgaaaacgttacgatcgaaggtctggtatgaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 63 - DNA, 3295 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattctgaggagaagtcgacttggaagcggccgcttaggatccttgaggagattggtaccatgatcattaaacctcgtatccgtggctttatctgcaccacgactcacccggtaggttgcgaagctaacgtcaaagaacaaatcgcatacactaaagctcagggcccgatcaaaaacgcccctaaacgtgttctggttgttggtgcctcctccggttatggtctgtcttctcgtatcgcggcagcgtttggcggcggtgcttccaccatcggcgtgttcttcgaaaaggaaggcaccgaaaagaaacctggtactgctggcttctacaacgctgcggcgttcgaaaaactggcgcgtgaagagggcctgtacgccaagagcctgaacggcgatgcattctccaacgaggcgaaacagaaaaccattgaactgatcaaagaagacctgggtcaaattgatatggtggtttacagcctggcatccccggtgcgcaaaatgccggaaaccggtgaactggtgcgcagcgcactgaaaccgattggtgagacttatacctctaccgcggtcgatacgaataaggatgtgatcattgaagcgagcgttgaaccggcgaccgaagaggaaatcaaagataccgtgactgtaatgggtggtgaggattgggaactgtggatcaatgcgctgagcgatgcaggcgtgctggctgaaggttgcaaaactgttgcttatagctacattggcaccgaactgacctggcctatctactgggacggtgcactgggtaaagctaaaatggatctggatcgtgcagccaaagcactgaacgacaaactggcggcaaccggtggctctgcgaatgtcgctgttctgaaatccgttgtaacccaagcttcctccgcaatcccggttatgccgctgtatatcgcaatggtgttcaagaaaatgcgcgaagaaggtgtacacgaaggctgcatggaacagatttaccgtatgttctctcagcgtctgtacaaggaagacggctctgctgccgaggttgatgaaatgaaccgtctgcgtctggacgattgggagctgcgcgacgacattcagcagcactgccgtgaactgtggccgcagattaccaccgaaaatctgaaagaactgaccgattacgttgaatataaggaagagttcctgaaactgttcggtttcggtgttgagggcgttgattacgaagcagacgtgaacccggctgtggaagccgatttcatccagatctaaggcgccttaggattcccgggagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 64 - DNA, 3234 bp, Artificial Sequence
  Description of Artificial Sequence Synthetic polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagataaatgtgagcggataacaattgacattgtgagcggataacaagatactgagcacatcagcaggacgcactgaccgaattcattaaagaggagaaaggtaccatgatcgtaaagcctatggttcgtaacaatatttgcctgaacgctcatccgcagggttgcaagaaaggtgtcgaggatcagattgaatacaccaagaaacgtattaccgctgaagttaaagcaggtgctaaagcgccgaaaaacgtgctggttctgggctgttccaacggctacggcctggcgtctcgcatcactgctgcgtttggttatggtgcggctactatcggtgtttcttttgaaaaagcgggctccgaaaccaaatatggcaccccaggttggtacaacaacctggcgttcgatgaagcggctaaacgcgagggcctgtactctgtgactatcgacggtgacgccttcagcgatgaaatcaaagcacaggttatcgaggaagccaaaaagaaaggcattaagtttgacctgattgtgtactctctggctagcccggtgcgtaccgatccggataccggcatcatgcacaaatccgtcctgaaaccgttcggcaaaactttcaccggtaaaacggtagatccgttcactggtgagctgaaagaaatctctgccgagccagctaacgatgaagaggcagctgctactgtcaaagtcatgggtggtgaagattgggaacgttggatcaaacagctgtctaaagaaggtctgctggaggaaggctgcattaccctggcatactcctacattggtccagaggccactcaggcgctgtatcgtaaaggtactatcggtaaagctaaagaacacctggaagctacggctcaccgtctgaacaaagaaaacccgtccatccgtgcattcgtttccgtcaacaagggcctggtcacccgtgcatccgcagttatcccggtcatccctctgtatctggcttccctgttcaaggttatgaaggaaaaaggtaaccatgagggttgtatcgaacagatcacccgtctgtacgccgaacgtctgtaccgcaaggatggcaccatcccggttgatgaggaaaaccgcattcgtatcgacgactgggaactggaagaagatgttcaaaaagctgtgtctgcgctgatggaaaaagtgaccggcgaaaatgcggaatccctgacggacctggcgggctatcgtcatgactttctggcgtccaacggttttgatgttgagggcatcaactatgaagcggaagtagagcgttttgaccgcatttaaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 65 - DNA, 5241 bp, Artificial Sequence
  Description of Artificial Sequence Synthetic polynucleotide
taagaaaccattattatcatgacattaacctataaaaataggcgtatcacgaggccctttcgtcttcacctcgagaattgtgagcggataacaattgacattgtgagcggataacaagatactgagcacatcagcaggacgcactgaccgaattcattaaagaggagaaaggtaccatgtctcaattcttttttaatcaacgcacccatctcgtgagcgacgtcatcgacggtacgattatcgccagcccgtggaataacctggcgcgtctggaaagcgatccggccattcgcatcgtggtccgtcgtgacctcaacaaaaataacgtggcggtaatttccggcggtggttcagggcacgaacccgcgcacgttgggtttatcggtaaaggcatgctaaccgctgcggtttgcggcgacgttttcgcttccccgagcgtggatgcggtactgaccgccatccaggcggtaaccggtgaggcgggctgtttattgatcgtgaaaaattacaccggtgaccgtcttaatttcggtctcgccgccgagaaagcccgtcgccttggttacaacgttgaaatgctgattgttggcgacgacatctccctgcctgataacaaacacccacgcggcattgcgggaaccatcctggtgcataaaatcgcaggctattttgccgaacgcggctacaacctcgccaccgtcctgcgtgaagcgcagtacgcggccaataacaccttcagcctgggcgttgcgctttccagctgtcatctgccgcaagaagccgacgccgccccgcgtcatcatccgggccacgcggaactgggcatgggcattcacggcgaaccaggcgcatcggttatcgacacccagaacagtgcgcaggtggtgaacctgatggtggataagctgatggcagccctgcctgaaaccggccgtctggcggtgatgattaacaatcttggcggcgtttctgttgccgaaatggccatcattacccgcgaactggccagcagcccgctgcacccacgtatcgactggctgattggcccggcctcactggtcaccgctctggatatgaaaagcttttcactgacggccatcgtgctggaagaaagcatcgaaaaagcgttactcaccgaggtggaaaccagcaactggccgacgccggtcccgccgcgtgaaatcagttgtgtaccatcatctcagcgtagcgcacgcgtggaattccagccttcggcgaacgccatggtggccgggattgtggaacttgtcaccacaaccctttccgatctggagactcatcttaatgcgctggacgccaaagtcggcgatggcgataccggttcgacctttgccgctggcgcgcgtgaaattgccagtctgttgcatcgccagcagttgccgctggataaccttgccacgctgttcgcgctgattggcgaacgtctgaccgtagtgatgggtggttccagcggtgtgctgatgtctattttctttaccgctgcggggcagaaactggaacagggagctagcgttgccgaatccctgaatacgggactggcgcagatgaagttctacggcggcgcagacgaaggcgatcgcaccatgattgatgcgctgcaaccagccctgacttcgctgctcacgcagccgcaaaatctgcaggccgcattcgacgccgcgcaagcgggagccgaacgaacctgtttgtcgagcaaagccaatgccggtcgcgcatcgtatctcagcagcgaaagcctgctcggaaatatggaccccggcgcgcacgccgtagcgatggtgtttaaagcgctagcggagagtgagctgggctaatctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtc

SEQ ID NO: 66 - DNA, 2302 bp, Artificial Sequence
  Description of Artificial Sequence Synthetic polynucleotide
ctcgagagcttactccccatccccctgttgacaattaatcatcggctcgtataatgtgtggaattgtgagcggataacaattgaattcattaaagaggagaaagtcgacattatgcggccgcggatccataaggaggattaattaagacttcccgggtgatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 67 - DNA, 3384 bp, Artificial Sequence
  Description of Artificial Sequence Synthetic polynucleotide
ctcgagagcttactccccatccccctgttgacaattaatcatcggctcgtataatgtgtggaattgtgagcggataacaattgaattcattaaagaggagaaagtcgacatgaagatcgttttagtcttatatgatgctggtaaacacgctgccgatgaagaaaaattatacggttgtactgaaaacaaattaggtattgccaattggttgaaagatcaaggacatgaattaatcaccacgtctgataaagaaggcggaaacagtgtgttggatcaacatataccagatgccgatattatcattacaactcctttccatcctgcttatatcactaaggaaagaatcgacaaggctaaaaaattgaaattagttgttgtcgctggtgtcggttctgatcatattgatttggattatatcaaccaaaccggtaagaaaatctccgttttggaagttaccggttctaatgttgtctctgttgcagaacacgttgtcatgaccatgcttgtcttggttagaaattttgttccagctcacgaacaaatcattaaccacgattgggaggttgctgctatcgctaaggatgcttacgatatcgaaggtaaaactatcgccaccattggtgccggtagaattggttacagagtcttggaaagattagtcccattcaatcctaaagaattattatactacgattatcaagctttaccaaaagatgctgaagaaaaagttggtgctagaagggttgaaaatattgaagaattggttgcccaagctgatatagttacagttaatgctccattacacgctggtacaaaaggtttaattaacaaggaattattgtctaaattcaagaaaggtgcttggttagtcaatactgcaagaggtgccatttgtgttgccgaagatgttgctgcagctttagaatctggtcaattaagaggttatggtggtgatgtttggttcccacaaccagctccaaaagatcacccatggagagatatgagaaacaaatatggtgctggtaacgccatgactcctcattactctggtactactttagatgctcaaactagatacgctcaaggtactaaaaatatcttggagtcattctttactggtaagtttgattacagaccacaagatatcatcttattaaacggtgaatacgttaccaaagcttacggtaaacacgataagaaataaggatccataaggaggattaattaagacttcccgggtgatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 68 - DNA, 4570 bp, Artificial Sequence
  Description of Artificial Sequence Synthetic polynucleotide
ctcgagagcttactccccatccccctgttgacaattaatcatcggctcgtataatgtgtggaattgtgagcggataacaattgaattcattaaagaggagaaagtcgacatgaagatcgttttagtcttatatgatgctggtaaacacgctgccgatgaagaaaaattatacggttgtactgaaaacaaattaggtattgccaattggttgaaagatcaaggacatgaattaatcaccacgtctgataaagaaggcggaaacagtgtgttggatcaacatataccagatgccgatattatcattacaactcctttccatcctgcttatatcactaaggaaagaatcgacaaggctaaaaaattgaaattagttgttgtcgctggtgtcggttctgatcatattgatttggattatatcaaccaaaccggtaagaaaatctccgttttggaagttaccggttctaatgttgtctctgttgcagaacacgttgtcatgaccatgcttgtcttggttagaaattttgttccagctcacgaacaaatcattaaccacgattgggaggttgctgctatcgctaaggatgcttacgatatcgaaggtaaaactatcgccaccattggtgccggtagaattggttacagagtcttggaaagattagtcccattcaatcctaaagaattattatactacgattatcaagctttaccaaaagatgctgaagaaaaagttggtgctagaagggttgaaaatattgaagaattggttgcccaagctgatatagttacagttaatgctccattacacgctggtacaaaaggtttaattaacaaggaattattgtctaaattcaagaaaggtgcttggttagtcaatactgcaagaggtgccatttgtgttgccgaagatgttgctgcagctttagaatctggtcaattaagaggttatggtggtgatgtttggttcccacaaccagctccaaaagatcacccatggagagatatgagaaacaaatatggtgctggtaacgccatgactcctcattactctggtactactttagatgctcaaactagatacgctcaaggtactaaaaatatcttggagtcattctttactggtaagtttgattacagaccacaagatatcatcttattaaacggtgaatacgttaccaaagcttacggtaaacacgataagaaataaggatccataaggaggattaattaaatgatcgtaaagcctatggttcgtaacaatatttgcctgaacgctcatccgcagggttgcaagaaaggtgtcgaggatcagattgaatacaccaagaaacgtattaccgctgaagttaaagcaggtgctaaagcgccgaaaaacgtgctggttctgggctgttccaacggctacggcctggcgtctcgcatcactgctgcgtttggttatggtgcggctactatcggtgtttcttttgaaaaagcgggctccgaaaccaaatatggcaccccaggttggtacaacaacctggcgttcgatgaagcggctaaacgcgagggcctgtactctgtgactatcgacggtgacgccttcagcgatgaaatcaaagcacaggttatcgaggaagccaaaaagaaaggcattaagtttgacctgattgtgtactctctggctagcccggtgcgtaccgatccggataccggcatcatgcacaaatccgtcctgaaaccgttcggcaaaactttcaccggtaaaacggtagatccgttcactggtgagctgaaagaaatctctgccgagccagctaacgatgaagaggcagctgctactgtcaaagtcatgggtggtgaagattgggaacgttggatcaaacagctgtctaaagaaggtctgctggaggaaggctgcattaccctggcatactcctacattggtccagaggccactcaggcgctgtatcgtaaaggtactatcggtaaagctaaagaacacctggaagctacggctcaccgtctgaacaaagaaaacccgtccatccgtgcattcgtttccgtcaacaagggcctggtcacccgtgcatccgcagttatcccggtcatccctctgtatctggcttccctgttcaaggttatgaaggaaaaaggtaaccatgagggttgtatcgaacagatcacccgtctgtacgccgaacgtctgtaccgcaaggatggcaccatcccggttgatgaggaaaaccgcattcgtatcgacgactgggaactggaagaagatgttcaaaaagctgtgtctgcgctgatggaaaaagtgaccggcgaaaatgcggaatccctgacggacctggcgggctatcgtcatgactttctggcgtccaacggttttgatgttgagggcatcaactatgaagcggaagtagagcgttttgaccgcattcccgggtgatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 69 - DNA, 35 bp, Artificial Sequence
  Description of Artificial Sequence Synthetic primer
aattgaattcttattatttaggaggagtaaaacat

SEQ ID NO: 70 - DNA, 35 bp, Artificial Sequence
  Description of Artificial Sequence Synthetic primer
aattggatccttagtctctttcaactacgagagct

SEQ ID NO: 71 - DNA, 35 bp, Artificial Sequence
  Description of Artificial Sequence Synthetic primer
aattgaattcatattttagaaagaagtgtatattt

SEQ ID NO: 72 - DNA, 42 bp, Artificial Sequence
  Description of Artificial Sequence Synthetic primer
aattacgcgtttaaggttgttttttaaaacaatttatataca

SEQ ID NO: 73 - DNA, 36 bp, Artificial Sequence
  Description of Artificial Sequence Synthetic primer
aattgaattcattagatgcttgtattaaaataataa

SEQ ID NO: 74 - DNA, 36 bp, Artificial Sequence
  Description of Artificial Sequence Synthetic primer
aattggatccttacacagattttttgaatatttgta

SEQ ID NO: 75 - DNA, 35 bp, Artificial Sequence
  Description of Artificial Sequence Synthetic primer
aattgaattcattgatagtttctttaaatttaggg

SEQ ID NO: 76 - DNA, 35 bp, Artificial Sequence
  Description of Artificial Sequence Synthetic primer
aattggatccttattttgaataatcgtagaaacct

SEQ ID NO: 77 - DNA, 35 bp, Artificial Sequence
  Description of Artificial Sequence Synthetic primer
aattgaattcctatctatttttgaagccttcaatt

SEQ ID NO: 78 - DNA, 36 bp, Artificial Sequence
  Description of Artificial Sequence Synthetic primer
aattggatccaatattttaggaggattagtcatgga

SEQ ID NO: 79 - DNA, 37 bp, Artificial Sequence
  Description of Artificial Sequence Synthetic primer
aattggtaccttaattattagcagctttaacttgagc

SEQ ID NO: 80 - DNA, 40 bp, Artificial Sequence
  Description of Artificial Sequence Synthetic primer
aattggatccaaaattgaaggcttcaaaaatagataggag

SEQ ID NO: 81 - DNA, 44 bp, Artificial Sequence
  Description of Artificial Sequence Synthetic primer
aattgtcgacattttataaaggagtgtatataaatgaaagttac

SEQ ID NO: 82 - DNA, 36 bp, Artificial Sequence
  Description of Artificial Sequence Synthetic primer
ttaatctagattaaaatgattttatatagatatcct

SEQ ID NO: 83 - DNA, 20 bp, Artificial Sequence
  Description of Artificial Sequence Synthetic primer
ccgtgggtgaaacagttctt

SEQ ID NO: 84 - DNA, 20 bp, Artificial Sequence
  Description of Artificial Sequence Synthetic primer
cgtaagtgcgagcgtaatga

SEQ ID NO: 85 - DNA, 20 bp, Artificial Sequence
  Description of Artificial Sequence Synthetic primer
aaagctccacgctggtagaa

SEQ ID NO: 86 - DNA, 20 bp, Artificial Sequence
  Description of Artificial Sequence Synthetic primer
gtcacgcgtctgataagcaa

(19) United States
(12) Patent Application Publication
(10) Pub. No.: US 2009/0155869 A1
(43) Pub. Date: Jun. 18, 2009

(54) ENGINEERED MICROORGANISMS FOR PRODUCING N-BUTANOL AND RELATED METHODS

(75) Inventors: Thomas Buelter, Santa Monica, CA (US); Andrew C. Hawkins, Pasadena, CA (US); Kalib Kersh, LaVerne, CA (US); Peter Meinhold, Pasadena, CA (US); Matthew W. Peters, Pasadena, CA (US); Ezhilkani Subbian, Pasadena, CA (US)

Correspondence Address: PAUL, HASTINGS, JANOFSKY & WALKER LLP, 875 15th Street, NW, Washington, DC, 20005, US

(73) Assignee: Gevo, Inc., Pasadena, CA (US)

(21) Appl. No.: 11/949,724

(22) Filed: Dec. 3, 2007

Related U.S. Application Data
(60) Provisional application No. 60/868,326, filed on Dec. 1, 2006.
(60) Provisional application No. 60/940,877, filed on May 30, 2007.
(60) Provisional application No. 60/890,329, filed on Feb. 16, 2007.
(60) Provisional application No. 60/905,550, filed on Mar. 6, 2007.
(60) Provisional application No. 60/945,576, filed on Jun. 21, 2007.

(51) Int. Cl. C12P 7/16; C12N 1/21

(52) U.S. Cl. 435/160; 435/252.3

(57) ABSTRACT

A recombinant microorganism expressing at least a heterologous enzyme of an NADH-dependent pathway for conversion of a carbon source to n-butanol, metabolic intermediate and/or a derivative thereof and capable of producing n-butanol, a metabolic intermediate and/or a derivative thereof at a high yield and related methods. The recombinant microorganism engineered to inactivate a native enzyme of one or more pathways that compete with NADH-dependent heterologous pathway, and/or to balance the NADH-dependent heterologous pathway with respect to NADH production and consumption.

### CROSS REFERENCE TO RELATED APPLICATIONS

This application claims priority to U.S. Provisional Application Ser. No. 60/868,326 filed on Dec. 1, 2006, U.S. Provisional Application Serial Number No. 60/940,877 filed on May 30, 2007, U.S. Provisional Application Serial Number No. 60/890,329 filed on Feb. 16, 2007, U.S. Provisional Application Serial Number No. 60/905,550 filed on Mar. 6, 2007, and U.S. Provisional Application Serial Number No. 60/945,576 filed on Jun. 21, 2007, all incorporated herein by reference in their entirety.

### TECHNICAL FIELD

The present disclosure relates to engineered microorganisms. In particular, it relates to engineered microorganisms for producing biofuels such as n-butanol, metabolic intermediates thereof and/or derivatives thereof.

### BACKGROUND

The bioconversion of carbohydrates from biomass-derived sugars into n-butanol has been known and performed on a large scale for about 100 years. Its history goes back to Louis Pasteur, who observed in 1861 that certain bacteria produce n-butanol. In 1912, Chaim Weizmann discovered a microorganism called Clostridium acetobutylicum, which was able to ferment starch to acetone, n-butanol, and ethanol (hence ABE fermentation). This process is based on a unique set of metabolic pathways found in anaerobic gram positive bacteria of the genus Clostridium (see FIG. 1) which also provide production of by-products such as acetone and ethanol.

Recent instability of oil supplies from the Middle East, coupled with a readily available supply of renewable agriculturally based biomass in the U.S., have spurred a renewed interest in the production of n-butanol in Clostridium and prompted attempts to produce butanol in other microorganisms.

Engineered strains of Clostridium have been generated that optimize the production of n-butanol from treated biomass waste. Additionally, new n-butanol production processes using multiple Clostridium strains, optimized for either the conversion of carbohydrates into butyrate or the subsequent conversion of exogenous butyrate into n-butanol, have been developed.

Production of engineered strains of other microorganisms such as E. coli capable of producing a detectable amount of butanol has also been reported.

### SUMMARY

Recombinant microorganisms are herein disclosed that can provide n-butanol at high yields of greater than 70% of theoretical.

In particular, the recombinant microorganisms herein disclosed are engineered to activate a heterologous pathway for the production of n-butanol, to direct the carbon flux to n-butanol and possibly to balance said heterologous pathway with respect to NADH production and consumption to maximize the obtainable yield.

According to one embodiment a recombinant microorganism is described that is capable of producing n-butanol at a yield of at least 5 percent of theoretical. The recombinant microorganism is in particular obtainable by engineering the microorganism to activate an heterologous enzyme of an NADH-dependent pathway for conversion of a carbon source to n-butanol through production of one or more metabolic intermediates; engineering the microorganism to inactivate a native enzyme of one or more pathways for the conversion of a substrate to a product wherein the substrate is one of the one or more metabolic intermediates, and engineering the microorganism to activate at least one of an NADH-producing enzyme and an NADH-producing pathway to balance said NADH-dependent heterologous pathway.

According to another embodiment a recombinant microorganism is described that is capable of producing n-butanol at a yield of at least 2 percent of theoretical. The recombinant microorganism obtainable by engineering the microorganism to activate an heterologous enzyme of an NADH-dependent pathway for conversion of a carbon source to n-butanol through production of one or more metabolic intermediates; and engineering the microorganism to inactivate a native enzyme of one or more pathways for the conversion of a substrate to a product wherein the substrate is one of the one or more metabolic intermediates.

According to a further embodiment a recombinant microorganism is described that expresses a heterologous pathway for the conversion of a carbon source to n-butanol. The heterologous pathway comprising the following substrate to product conversions: acetyl-CoA to acetoacetyl-CoA; acetoacetyl-CoA to hydroxybutyryl-CoA; hydroxybutyryl-CoA to crotonoyl-CoA; crotonyl-CoA to butyryl-CoA; butyryl-CoA to butyraldehyde, and butyraldehyde to n-butanol. The recombinant microorganism is engineered to inactivate one or more native pathways for the conversion of a substrate to a product wherein the substrate is pyruvate or acetylCoA. The recombinant microorganism is further engineered to activate at least one of an anaerobically active pyruvate dehydrogenase, a NADH dependent formate dehydrogenase, and a heterologous pathway for the conversion of glycerol to pyruvate. The recombinant microorganism is capable of producing n-butanol at a yield of at least 5 percent of theoretical.

According to another embodiment aspect a recombinant microorganism is described that expresses a heterologous pathway for the conversion of a carbon source to n-butanol. The heterologous pathway comprising the following substrate to product conversions: acetyl-CoA to acetoacetyl-CoA; acetoacetyl-CoA to hydroxybutyryl-CoA; hydroxybutyryl-CoA to crotonoyl-CoA; crotonyl-CoA to butyryl-CoA; butyryl-CoA to butyraldehyde, and butyraldehyde to n-butanol. The recombinant microorganism is engineered to inactivate one or more native pathways for the conversion of a substrate to a product wherein the substrate is pyruvate or acetylCoA. The recombinant microorganism is capable of producing n-butanol at a yield of at least XX percent of theoretical.

The recombinant microorganisms herein described can produce n-butanol at high yields with a minimized production of by-products which is advantageous with respect to prior art systems wherein n-butanol is produced in Clostridium.

The recombinant microorganisms herein described can produce n-butanol at significantly higher yields than prior art systems wherein n-butanol is produced in microorganisms other than Clostridium.

According to another embodiment, a method for producing n-butanol is described the method comprising providing a recombinant microorganism herein described, and contacting the recombinant microorganism with a carbon source for a time and under conditions sufficient to allow n-butanol production, until a recoverable quantity of n-butanol is produced. The method can also include recovering the recoverable amount of n-butanol.

According to another embodiment a recombinant microorganism is described that is capable of producing butyrate at a yield of at least 5 percent of theoretical. The recombinant microorganism obtainable by engineering the microorganism to activate an NADH-dependent heterologous pathway for conversion of a carbon source to butyrate through production of one or more metabolic intermediates; and engineering the microorganism to inactivate a native pathway for the conversion of a substrate to a product wherein the substrate is one of the one or more metabolic intermediates.

According to another embodiment a recombinant microorganism is described that is capable of producing mixtures of butyrate and n-butanol at a yield of at least 5 percent of theoretical. The recombinant microorganism is obtainable by engineering the microorganism to activate an NADH-dependent heterologous pathway for conversion of a carbon source to butyrate through production of one or more metabolic intermediates; engineering the microorganism to activate an NADH-dependent heterologous pathway for conversion of a carbon source to n-butanol through production of one or more metabolic intermediates; engineering the microorganism to inactivate a native pathway for the conversion of a substrate to a product wherein the substrate is one of the one or more metabolic intermediates, and/or engineering the microorganism to activate at least one of an NADH-producing enzyme and an NADH-producing pathway to balance said NADH-dependent heterologous pathway.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and form a part of this specification, illustrate one or more embodiments of the present disclosure and, together with the detailed description, serve to explain the principles and implementations of the disclosure.

FIG. 1 illustrates the metabolic pathways involved in the conversion of glucose to acids and solvents in Clostridium acetobutylicum. Hexoses (e.g. glucose) and pentoses are converted to pyruvate, ATP and NADH. Subsequently, pyruvate is oxidatively decarboxylated to acetyl-CoA by a pyruvate-ferredoxin oxidoreductase. The reducing equivalents generated in this step are converted to hydrogen by an iron-only hydrogenase. Acetyl-CoA is the branch-point intermediate, leading to the production of organic acids (acetate and butyrate) and solvents (acetone, n-butanol and ethanol).

FIG. 2 illustrates a chemical pathway to produce n-butanol in microorganisms. Under ideal conditions, this pathway generates one molecule of n-butanol (maximum) per molecule of metabolized glucose. The depicted n-butanol-producing pathway is balanced with respect to NADH production and consumption, in that four (4) NADH are produced and consumed per glucose metabolized.

FIG. 3 illustrates mixed-acid fermentation in E. coli, the products of which include succinate, lactate, acetate, ethanol, formate, carbon dioxide and hydrogen gas. The enzymes which are boxed have been deleted or inactivated, either singly or in various combinations in accordance with the disclosure in one or more E. coli strains.

FIG. 4 illustrates a metabolic engineering strategy to produce anaerobically-active pyruvate dehydrogenase in E. coli. In this strategy, the enzymes in boxes are deleted/inactivated and the cells are grown anaerobically on minimal media and a carbon source such as glucose. Under those conditions, the only cells that grow are those that produce pyruvate dehydrogenase because they are capable of balancing NADH production and consumption via the pathway indicated in bold.

FIG. 5 depicts a 5614-bp EcoRI-BamHI restriction fragment showing the thl, adh, crt and hbd genes from C. acetobutylicum synthesized as a single transcript (seq tach, which is expressed from plasmid pGV1191.

FIG. 6 depicts a 3027-bp EcoRI-BamHI restriction fragment showing the bcd, etfA and etfB genes from C. acetobutylicum synthesized as a single transcript (seq Cbab, which is expressed from pGV1088.

FIG. 7 depicts a 3128-bp restriction fragment showing the bcd, etfA and etfB genes from M. elsdenii synthesized as a single transcript (seq Mbab, which is expressed from pGV1052.

FIG. 8 depicts the Seq tach-pZA11 (=pGV1191) plasmid containing thl, adhE2, crt, and hbd ORFS inserted at the EcoRI and BamHI sites in the vector MCS and downstream from a modified phage lambda tetO promoter (P_{L-tet}). The plasmid also carries a p15A origin of replication and an ampicillin resistance gene.

FIG. 9 depicts the Seq Cbab-pZE32 (=pGV1088) plasmid containing the bcd, elfA and etfB ORFS inserted at the EcoRI and BamHI sites in the vector MCS and downstream from a modified phage lambda LacO promoter (P_{L-lac}). The plasmid also carries the ColE1 origin of replication and a chloramphenicol resistance gene.

FIG. 10 shows a petri dish including GEVO1005 (E. coli W3110), GEVO922 (E. coli W3110 (ΔglpK, ΔglpD)), and GEVO926 (E. coli W3110 (ΔglpK, ΔglpD, evolved)). GEVO926 is labeled “GO2XKO-I” on the plate.

FIG. 11 shows a diagram illustrating the amount of glycerol consumed by a recombinant microorganism herein described (GEVO927) in comparison with the amount consumed by the corresponding wild-type microorganism (GEVO1005, pGV110) following anaerobic biotransformation under non-growing conditions.

FIG. 12 shows a diagram illustrating the amount of ethyl 3-hydroxybutyrate produced by a recombinant microorganism herein described (GEVO927) in comparison with the amount produced by the corresponding wild-type microorganism (GEVO1005, pGV1100) following anaerobic non-growing biocatalysis

FIG. 13 shows a diagram illustrating the carbon balance of a microorganism herein described (GEVO1005, pGV110) in terms of glycerol consumed and amount of acetate observed following anaerobic non-growing biocatalysis.

FIG. 14 shows a diagram illustrating the carbon balance of a recombinant microorganism herein described (GEVO927) in terms of glycerol consumed and amount of acetate observed following anaerobic non-growing biocatalysis.

FIG. 15 shows n-butanol formation over time in fermentations using E. coli strains expressing n-butanol production pathways utilizing TER from Euglena gracilis (pGV1191, pGV1113) and Aeromonas hydrophila (pGV1191, pGV1117) in comparison to E. coli expressing an n-butanol production pathways that does not contain a TER enzyme (pGV1191). Experiments were conducted using two biological replicates . . . .

FIG. 16 shows a diagram illustrating n-butanol fermentations performed with recombinant microorganisms herein disclosed expressing different TER homologues (pGV1340; pGV1344; pGV1345; pGV1346; pGV1347; pGV1348; pGV1349; pGV1272 (Control). pGV1344 contains the gene encoding the Treponema denticola TER. pGV1272 contains the gene encoding the Euglena gracilis TER. Experiments were conducted using two biological replicates.

FIG. 17 shows a diagram illustrating n-butanol fermentations with recombinant microorganisms containing the indicated plasmids expressing different TER homologues (pGV1341; pGV1342; pGV1343; pGV1272 (Control). pGV1272 contains the gene encoding the Euglena gracilis TER. Experiments were conducted using two biological replicates

FIG. 18 shows a diagram illustrating lactate production by recombinant microorganisms herein described (Strain A: GEVO1083, pGV1191, pGV1113; Strain B: GEVO1121, pGV1191, pGV1113) during the anaerobic bottle fermentation. Experiments were conducted using two biological replicates.

FIG. 19 shows a diagram illustrating n-butanol production by recombinant microorganisms according to embodiments herein described (Strain 1137: GEVO1137, pGV1190, pGV1113; Strain 1083: GEVO1083, pGV1190, pGV1113) engineered to inactivate the acetate fermentative pathway. Experiments were conducted using two biological replicates.

FIG. 20A shows a diagram illustrating n-butanol production by recombinant microorganisms according to embodiments of the present disclosure (Strain 1: GEVO1083, pGV1113, pGV1190; Strain 2: GEVO1083, pGV1281, pGV1190). Experiments were conducted using two biological replicates.

FIG. 20B shows a diagram illustrating glucose consumption by recombinant microorganism according to embodiments of the present disclosure. (rectangles: GEVO1083, pGV1113, pGV1190; triangles: GEVO1083, pGV1281, pGV1190). Experiments were conducted using two biological replicates.

FIG. 21A shows a diagram illustrating fermentations carried out with recombinant microorganisms according to embodiments herein described anaerobically without neutralization or feeding (circles: GEVO768, pGV1191, pGV1113; triangles: GEVO768). Experiments were conducted using two biological replicates.

FIG. 21B shows a diagram illustrating fermentations carried out with recombinant microorganisms of FIG. 21A, wherein the fermentation broth was neutralized and glucose was fed every 8 hours throughout the fermentation and wherein the fermentation was performed with an aerobic growth phase and an anaerobic biocatalysis phase (circles: GEVO768, pGV1191, pGV1113; triangles: GEVO768). Experiments were conducted using two biological replicates.

FIG. 22A shows a diagram illustrating n-butanol production during fermentations performed with recombinant microorganisms according to embodiments herein disclosed (GEVO1083, pGV1190, pGV1113) under different transitions from aerobic to anaerobic culture conditions. Fermenter 1 (F1) had a 2 hour transition, fermenter 2 (F2) had a 6 hour transition, fermenter 3 (F3) had a 12 hour transition and in fermenter 4 the transition was done in the time that it took the cells to consume the oxygen left in the fermenter after the oxygen supply was stopped.

FIG. 22B shows a diagram illustrating production during fermentations performed with recombinant microorganisms according to embodiments herein disclosed (GEVO1083, pGV1190, pGV1113) under different transitions from aerobic to anaerobic culture conditions. Fermenter 1 (F1) had a 2 hour transition, fermenter 2 (F2) had a 6 hour transition, fermenter 3 (F3) had a 12 hour transition and in fermenter 4 the transition was done in the time that it took the cells to consume the oxygen left in the fermenter after the oxygen supply was stopped.

FIG. 23A shows a diagram illustrating glucose consumption by recombinant microorganism according to embodiments of the present disclosure. (rectangles: GEVO1034, pGV1248; triangles: GEVO1034, pGV111). Experiments were conducted using two biological replicates.

FIG. 23B shows a diagram illustrating formate production by recombinant microorganism according to embodiments of the present disclosure. (rectangles: GEVO1034, pGV1248; triangles: GEVO1034, pGV111). Experiments were conducted using two biological replicates.

FIG. 23C shows a diagram illustrating ethanol production by recombinant microorganism according to embodiments of the present disclosure. (rectangles: GEVO1034, pGV1248; triangles: GEVO1034, pGV1111). Experiments were conducted using two biological replicates.

FIG. 23D shows a diagram illustrating acetate production by recombinant microorganism according to embodiments of the present disclosure. (rectangles: GEVO1034, pGV1248; triangles: GEVO1034, pGV1111). Experiments were conducted using two biological replicates.

FIG. 24A shows a diagram illustrating lactate production by recombinant microorganism according to embodiments of the present disclosure. (rectangles: GEVO1034, pGV1248; triangles: GEVO1034, pGV1111). Experiments were conducted using two biological replicates.

FIG. 24B shows a diagram illustrating succinate production by recombinant microorganism according to embodiments of the present disclosure. (rectangles: GEVO1034, pGV1248; triangles: GEVO1034, pGV1111). Experiments were conducted using two biological replicates.

FIG. 25A shows a diagram illustrating ethanol production by recombinant microorganism according to embodiments of the present disclosure. (rectangles: GEVO992, pGV1278; triangles: GEVO992, pGV1279; circles: GEVO992, pGV772). Experiments were conducted using two biological replicates.

FIG. 25B shows a diagram illustrating acetate production by recombinant microorganism according to embodiments of the present disclosure. (rectangles: GEVO992, pGV1278; triangles: GEVO992, pGV1279; circles: GEVO992, pGV772). Experiments were conducted using two biological replicates.

FIG. 26 shows a diagram illustrating glycerol metabolism in wild-type E. coli and an E. coli GEVO926 expressing a DHA kinase from plasmid pGV1563.

FIG. 27 shows a chemical pathway to produce mixtures of n-butanol and butyrate in microorganisms. The depicted n-butanol-producing pathway is balanced with respect to NADH production and consumption, in that four (4) NADH are produced and consumed per glucose metabolized.

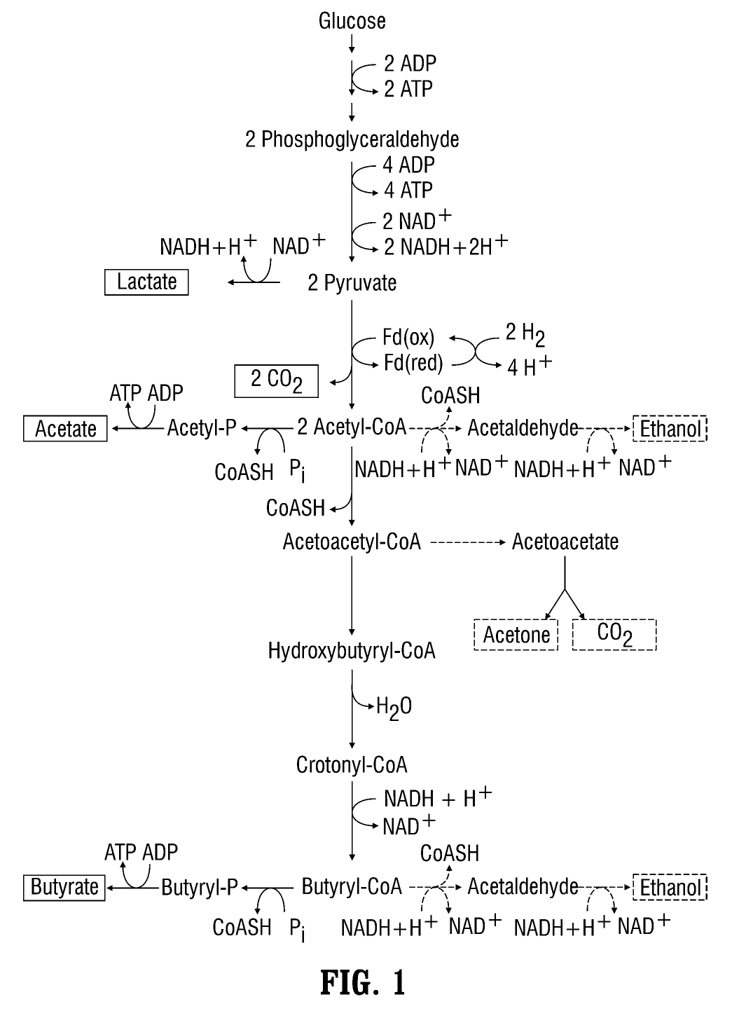

### DETAILED DESCRIPTION

Recombinant microorganisms are described that are engineered to convert a carbon source into n-butanol at high yield. In particular, recombinant microorganisms are described that are capable of metabolizing a carbon source for producing n-butanol at a yield of at least 5% percent of theoretical.

As used herein, the term “microorganism” includes prokaryotic and eukaryotic microbial species from the Domains Archaea, Bacteria and Eukaryote, the latter including yeast and filamentous fungi, protozoa, algae, or higher Protista. The terms “cell,” “microbial cells,” and “microbes” are used interchangeably with the term microorganism. In a preferred embodiment, the microorganism is E. coli or yeast (such as S. pombe or S. cerevisiae).

“Bacteria”, or “Eubacteria”, refers to a domain of prokaryotic organisms. Bacteria include at least 11 distinct groups as follows: (1) Gram-positive (Gram⁺) bacteria, of which there are two major subdivisions: (a) high G+C group (Actinomycetes, Mycobacteria, Micrococcus, others) (b) low G+C group (Bacillus, Clostridia, Lactobacillus, Staphylococci, Streptococci, Mycoplasmas); (2) Proteobacteria, e.g., Purple photosynthetic and non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Pseudomonas, Bacteroides, Acetobacter, Aerobacter, Agrobacterium, Azotobacter, Myxococcus, Spirilla, Serratia, Vibrio, Rhizobium, Chlamydia, Rickettsia, Treponema and Fusobacterium.

“Gram positive bacteria” include cocci, nonsporulating rods and sporulating rods. The genera of gram positive bacteria include, for example, Actinomyces, Bacillus, Clostridium, Corynebacterium, Erysipelothrix, Lactobacillus, Listeria, Mycobacterium, Nocardia, Staphylococcus, Streptococcus and Streptomyces.

The term “carbon source” generally refers to a substrate or compound suitable to be used as a source of carbon for prokaryotic or simple eukaryotic cell growth. Carbon sources may be in various forms, including, but not limited to polymers, carbohydrates, acids, alcohols, aldehydes, ketones, amino acids, peptides, etc. These include, for example, various monosaccharides such as glucose, oligosaccharides, polysaccharides, cellulosic material, saturated or unsaturated fatty acids, succinate, lactate, acetate, ethanol, etc., or mixtures thereof. The carbon source may additionally be a product of photosynthesis, including, but not limited to glucose. The term “carbon source” may be used interchangeably with the term “energy source,” since in chemoorganotrophic metabolism the carbon source is used both as an electron donor during catabolism as well as a source of carbon during cell growth.

Carbon sources which serve as suitable starting materials for the production of n-butanol products include, but are not limited to, biomass hydrolysates, glucose, starch, cellulose, hemicellulose, xylose, lignin, dextrose, fructose, galactose, corn, liquefied corn meal, corn steep liquor (a byproduct of corn wet milling process that contains nutrients leached out of corn during soaking), molasses, lignocellulose, and maltose. Photosynthetic organisms can additionally produce a carbon source as a product of photosynthesis. In a preferred embodiment, carbon sources may be selected from biomass hydrolysates and glucose. Glucose, dextrose and starch can be from an endogenous or exogenous source.

It should be noted that other, more accessible and/or inexpensive carbon sources, can be substituted for glucose with relatively minor modifications to the host microorganisms. For example, in certain embodiments, use of other renewable and economically feasible substrates may be preferred. These include: agricultural waste, starch-based packaging materials, corn fiber hydrolysate, soy molasses, fruit processing industry waste, and whey permeate, etc.

Five carbon sugars are only used as carbon sources with microorganism strains that are capable of processing these sugars, for example E. coli B. In some embodiments, glycerol, a three carbon carbohydrate, may be used as a carbon source for the biotransformations. In other embodiments, glycerin, or impure glycerol obtained by the hydrolysis of triglycerides from plant and animal fats and oils, may be used as a carbon source, as long as any impurities do not adversely affect the host microorganisms.

As used herein, the term “yield” refers to the molar yield. For example, the yield equals 100% when one mole of glucose is converted to one mole of n-butanol. In particular, the term “yield” is defined as the mole of product obtained per mole of carbon source monomer and may be expressed as percent. Unless otherwise noted, yield is expressed as a percentage of the theoretical yield. “Theoretical yield” is defined as the maximum mole of product that can be generated per a given mole of substrate as dictated by the stoichiometry of the metabolic pathway used to make the product. For example, the theoretical yield for one typical conversion of glucose to n-butanol is 100%. As such, a yield of n-butanol from glucose of 95% would be expressed as 95% of theoretical or 95% theoretical yield. For example, the theoretical yield for one typical conversion of glycerol to n-butanol is 50%. As such, a yield of n-butanol from glycerol of 45% would be expressed as 90% of theoretical or 90% theoretical yield.

The microorganisms herein disclosed are engineered, using genetic engineering techniques, to provide microorganisms which utilize heterologously expressed enzymes to produce n-butanol at high yield and in particular a yield of at least 5% of theoretical.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more nucleotides is also called nucleotidic oligomer or oligonucleotide.

The term “protein” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “heterologous” or “exogenous” as used herein with reference to molecules and in particular enzymes and polynucleotides, indicates molecules that are expressed in an organism other than the organism from which they originated or are found in nature, independently on the level of expression that can be lower, equal or higher than the level of expression of the molecule in the native microorganism.

On the other hand, the term “native” or “endogenous” as used herein with reference to molecules, and in particular enzymes and polynucleotides, indicates molecules that are expressed in the organism in which they originated or are found in nature, independently on the level of expression that can be lower equal or higher than the level of expression of the molecule in the native microorganism.

In certain embodiments, the native, unengineered microorganism is incapable of converting a carbon source to n-butanol or one or more of the metabolic intermediate(s) thereof, because, for example, such wild-type host lacks one or more required enzymes in a n-butanol-producing pathway.

In certain embodiments, the native, unengineered microorganism is capable of only converting minute amounts of a carbon source to n-butanol, at a yield of smaller than 0.1% of theoretical.

For instance, microorganisms such as E. coli or Saccharomyces sp. generally do not have a metabolic pathway to convert sugars such as glucose into n-butanol but it is possible to transfer a n-butanol producing pathway from a n-butanol producing strain, (e.g., Clostridium) into a bacterial or eukaryotic heterologous host, such as E. coli or Saccharomyces sp., and use the resulting recombinant microorganism to produce n-butanol.

Microorganisms, in general, are suitable as hosts if they possess inherent properties such as solvent resistance which will allow them to metabolize a carbon source in solvent containing environments.

The terms “host”, “host cells” and “recombinant host cells” are used interchangeably herein and refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Useful hosts for producing n-butanol may be either eukaryotic or prokaryotic microorganisms. While E. coli is one of the preferred hosts, other hosts include yeast strains such as Saccharomyces strains, which can be tolerant to n-butanol levels that are toxic to E. coli.

In certain embodiments, other suitable eukaryotic host microorganisms include, but are not limited to, Pichia, Hangeul, Yarrowia, Aspergillus, Kluyveromyces, Pachysolen, Rhodotorula,

Zygosaccharomyces, Galactomyces, Schizosaccharomyces, Penicillium, Torulaspora, Debaryomyces, Williopsis, Dekkera, Kloeckera, Metschnikowia and Candida species.

In another preferred embodiment, the hosts are bacterial hosts. In a more preferred embodiment the hosts include Arthrobacter, Bacillus, Brevibacterium, Clostridium, Corynebacterium, Escherichia, Gluconobacter, Nocardia, Pseudomonas, Rhodococcus, Streptomyces, Xanthomonas. In a more preferred embodiment, such hosts are E. coli or Pseudomonas. In an even more preferred embodiment, such hosts are E. coli (such as E. coli W3110 or E. coli B), Pseudomonas oleovorans, Pseudomonas fluorescens, or Pseudomonas putida.

In certain embodiments, the recombinant microorganism herein disclosed is resistant to certain levels of n-butanol in the growth medium, such that it is capable of growing in a medium with at least about 0.1%, 0.2%, 0.3%, 0.4%, 0.5%, 0.6%, 0.7%, 0.8%, 0.9%, 1%, 1.2%, 1.5%, 1.8%, 2%, 3%, 4%, 5%, 6%, 7%, 8% or more of n-butanol, at a rate substantially the same as that of the microorganism growing in the medium without n-butanol. As used herein, “substantially the same” refers to at least about 80%, 90%, 100%, 110%, or 120% of the wild-type growth rate.

In particular, the recombinant microorganisms herein disclosed are engineered to activate, and in particular express heterologous enzymes that can be used in the production of n-butanol. In particular, in certain embodiments, the recombinant microorganisms are engineered to activate heterologous enzymes that catalyze the conversion of acetyl-CoA to n-butanol.

The terms “activate” or “activation” as used herein with reference to a biologically active molecule, such as an enzyme, indicates any modification in the genome and/or proteome of a microorganism that increases the biological activity of the biologically active molecule in the microorganism. Exemplary activations include but are not limited to modifications that result in the conversion of the molecule from a biologically inactive form to a biologically active form and from a biologically active form to a biologically more active form, and modifications that result in the expression of the biologically active molecule in a microorganism wherein the biologically active molecule was previously not expressed. For example, activation of a biologically active molecule can be performed by expressing a native or heterologous polynucleotide encoding for the biologically active molecule in the microorganism, by expressing a native or heterologous polynucleotide encoding for an enzyme involved in the pathway for the synthesis of the biological active molecule in the microorganism, by expressing a native or heterologous molecule that enhances the expression of the biologically active molecule in the microorganism.

In some embodiments, the recombinant microorganism may express one or more heterologous genes encoding for enzymes that confer the capability to produce n-butanol. For example, the recombinant microorganism herein disclosed may express heterologous genes encoding one or more of: an anaerobically active pyruvate dehydrogenase (Pdh), NADH-dependent formate dehydrogenase (Fdh), acetyl-CoA-acetyltransferase (thiolase), hydroxybutyryl-CoA dehydrogenase, crotonase, butyryl-CoA dehydrogenase, butyraldehyde dehydrogenase, n-butanol dehydrogenase, bifunctional butyraldehyde/n-butanol dehydrogenase. Such heterologous DNA sequences are preferably obtained from a heterologous microorganism (such as Clostridium acetobutylicum or Clostridium beijerinckii), and may be introduced into an appropriate host using conventional molecular biology techniques. These heterologous DNA sequences enable the recombinant microorganism to produce n-butanol, at least to produce n-butanol or the metabolic intermediate(s) thereof in an amount greater than that produced by the wild-type counterpart microorganism.

In certain embodiments, the recombinant microorganism herein disclosed expresses a heterologous Thiolase or acetyl-CoA-acetyltransferase, such as one encoded by a thl gene from a Clostridium.

Thiolase (E.C. 2.3.1.19) or acetyl-CoA acetyltransferase, is an enzyme that catalyzes the condensation of an acetyl group onto an acetyl-CoA molecule. The enzyme is, in C. acetobutylicum, encoded by the gene thl (GenBank accession U08465, protein ID AAA82724.1), which was overexpressed, amongst other enzymes, in E. coli under its native promoter for the production of acetone (Bermejo et al., Appl. Environ. Mirobiol. 64: 1079-1085, 1998). Homologous enzymes have also been identified, and can easily be identified by one skilled in the art by performing a BLAST search against above protein sequence. These homologs can also serve as suitable thiolases in a heterologously expressed n-butanol pathway. Just to name a few, these homologous enzymes include, but are not limited to those from: C. acetobutylicum sp. (e.g., protein ID AAC26026.1), C. pasteurianum (e.g., protein ID ABA18857.1), C. beijerinckii sp. (e.g., protein ID EAP59904.1 or EAP59331.1), Clostridium perfringens sp. (e.g., protein ID ABG86544.1, ABG83108.1), Clostridium difficile sp. (e.g., protein ID CAJ67900.1 or ZP_{—}01231975.1), Thermoanaerobacterium thermosaccharolyticum (e.g., protein ID CAB07500.1), Thermoanaerobacter tengcongensis (e.g., AAM23825.1), Carboxydothermus hydrogenoformans (e.g., protein ID ABB13995.1), Desulfotomaculum reducens MI-1 (e.g., protein ID EAR45123.1), Candida tropicalis (e.g., protein ID BAA02716.1 or BAA02715.1), Saccharomyces cerevisiae (e.g., protein ID AAA62378.1 or CAA30788.1), Bacillus sp., Megasphaera elsdenii, or Butryivibrio fibrisolvens, etc. In addition, the endogenous E. coli thiolase could also be active in a heterologously expressed n-butanol pathway. E. coli synthesizes two distinct 3-ketoacyl-CoA thiolases. One is a product of the fadA gene, the second is the product of the atoB gene.

Homologs sharing at least about 55%, 60%, 65%, 70%, 75% or 80% sequence identity, or at least about 65%, 70%, 80% or 90% sequence homology, as calculated by NCBI's BLAST, are suitable thiolase homologs that can be used in the recombinant microorganisms herein disclosed. Such homologs include (without limitation): Clostridium beijerinckii NCIMB 8052 (ZP_{—}00909576.1 or ZP_{—}00909989.1), Clostridium acetobutylicum ATCC 824 (NP_{—}149242.1), Clostridium tetani E88 (NP_{—}781017.1), Clostridium perfringens str. 13 (NP_{—}563111.1), Clostridium perfringens SM101 (YP_{—}699470.1), Clostridium pasteurianum (ABA18857.1), Thermoanaerobacterium thermosaccharolyticum (CAB04793.1), Clostridium difficile QCD-32g58 (ZP_{—}01231975.1), Clostridium difficile 630 (CAJ67900.1), etc.

In certain embodiments, the recombinant microorganism herein disclosed expresses a heterologous 3-hydroxybutyryl-CoA dehydrogenase, such as one encoded by an hbd gene from a Clostridium.

The_{—}3-hydroxybutyryl-CoA dehydrogenase (BHBD) is an enzyme that catalyzes the conversion of acetoacetyl-CoA to 3-hydroxybutyryl-CoA. Different variants of this enzyme exist that produce either the (S) or the (R) isomer of 3-hydroxybutyryl-CoA. E. coli harboring an E. coli-C. acetobutylicum shuttle vector containing the C. acetobutylicum ATCC 824 gene for BHBD (hbd), amongst others, has been shown to functionally overexpress this enzyme. Many homologous enzymes have also been identified. Additional homologous enzymes can easily be identified by one skilled in the art by, for example, performing a BLAST search against afore-mentioned C. acetobutylicum BHBD. All these homologous enzymes could serve as a BHBD in a heterologously expressed n-butanol pathway. These homologous enzymes include, but are not limited the following: Clostridium kluyveri expresses two distinct forms of this enzyme (Miller et al., J. Bacteriol. 138: 99-104, 1979). Butyrivibrio fibrisolvens contains a bhbd gene which is organized within the same locus of the rest of its butyrate pathway (Asanuma et al., Current Microbiology 51: 91-94, 2005; Asanuma et al., Current Microbiology 47: 203-207, 2003). A gene encoding a short chain acyl-CoA dehydrogenase (SCAD) was cloned from Megasphaera elsdenii and expressed in E. coli. In vitro activity could be determined (Becker et al., Biochemistry 32: 10736-10742, 1993). Other homologues were identified in E. coli (fadB) where it is part of the fatty acid oxidation pathway (Pawar et al., J. Biol. Chem. 256: 3894-3899, 1981), and other Clostridium strains such as C. kluyveri (Hillmer et al., FEBS Lett. 21: 351-354, 1972; Madan et al., Eur. J. Biochem. 32: 51-56, 1973), C. beijerinckii, C. thermosaccharolyticum, C. tetani.

In certain embodiments, wherein a BHBD is expressed it may be beneficial to select an enzyme of the same organism that the upstream thiolase or the downstream crotonase originate from. This may avoid disrupting potential protein-protein interactions between proteins adjacent in the pathway when enzymes from different organisms are expressed.

In certain embodiments, the recombinant microorganism herein disclosed expresses a heterologous crotonase, such as one encoded by a crt gene from a Clostridium.

The crotonases or Enoyl-CoA hydratases are enzymes that catalyze the reversible hydration of cis and trans enoyl-CoA substrates to the corresponding β-hydroxyacyl CoA derivatives. In C. acetobutylicum, this step of the butanoate metabolism is catalyzed by EC 4.2.1.55, encoded by the crt gene (GenBank protein accession AAA95967, Kanehisa, Novartis Found Symp. 247: 91-101, 2002; discussion 01-3, 19-28, 244-52). The crotonase (Crt) from C. acetobutylicum has been purified to homogeneity and characterized (Waterson et al., J. Biol. Chem. 247: 5266-5271, 1972). It behaves as a homogenous protein in both native and denatured states. The enzyme appears to function as a tetramer with a subunit molecular weight of 28.2 kDa and 261 residues (Waterson et al. report a molecular mass of 40 kDa and a length of 370 residues). The purified enzyme lost activity when stored in buffer solutions at 4□C or when frozen (Waterson et al., J. Biol. Chem. 247: 5266-5271, 1972). The pH optimum for the enzyme is pH 8.4 (Schomburg et al., Nucleic Acids Res. 32: D431-433, 2004). Unlike the mammalian crotonases that have a broad substrate specificity, the bacterial enzyme hydrates only crotonyl-CoA and hexenoyl-CoA. Values of Vₘₐₓ and Kₘ of 6.5×10⁶ moles per min per mole and 3×10⁻⁵ M were obtained for crotonyl-CoA. The enzyme is inhibited at crotonyl-CoA concentrations of higher than 7×10⁵ M (Waterson et al., J. Biol. Chem. 247: 5252-5257, 1972; Waterson et al., J. Biol. Chem. 247: 5258-5265, 1972).

The structures of many of the crotonase family of enzymes have been solved (Engel et al., J. Mol. Biol. 275: 847-859, 1998). The crt gene is highly expressed in E. coli and exhibits a higher specific activity than seen in C. acetobutylicum (187.5 U/mg over 128.6 U/mg) (Boynton et al., J. Bacteriol. 178: 3015-3024, 1996). A number of different homologs of crotonase are encoded in eukaryotes and prokaryotes that functions as part of the butanoate metabolism, fatty acid synthesis, β-oxidation and other related pathways (Kanehisa, Novartis Found Symp. 247: 91-101, 2002; discussion 01-3, 19-28, 244-52; Schomburg et al., Nucleic Acids Res. 32: D431-433, 2003). A number of these enzymes have been well studied. Enoyl-CoA hydratase from bovine liver is extremely well-studied and thoroughly characterized (Waterson et al., J. Biol. Chem. 247: 5252-5257, 1972). A ClustalW alignment of 20 closest orthologs of crotonase from bacteria is generated. The homologs vary in sequence identity from 40-85%. The protein sequence of Crt and DNA sequence for the crt from C. acetobutylicum is available (see below, all sequences incorporated herein by reference). The crotonase (Crt) protein sequence (GenBank accession # AAA95967) is given in SEQ ID NO:2.

Homologs sharing at least about 45%, 50%, 55%, 60%, 65% or 70% sequence identity, or at least about 55%, 65%, 75% or 85% sequence homology, as calculated by NCBI's BLAST, are suitable Crt homologs that can be used in the recombinant microorganisms herein disclosed. Such homologs include (without limitation): Clostridium tetani E88 (NP_{—}782956.1), Clostridium perfringens SM101 (YP_{—}699562.1), Clostridium perfringens str. 13 (NP_{—}563217.1), Clostridium beijerinckii NCIMB 8052 (ZP_{—}00909698.1 or ZP_{—}00910124.1), Syntrophomonas wolfei subsp. wolfei str. Goettingen (YP_{—}754604.1), Desulfotomaculum reducens MI-1 (ZP_{—}01147473.1 or ZP_{—}01149651.1), Thermoanaerobacterium thermosaccharolyticum (CAB07495.1), Carboxydothermus hydrogenoformans Z-2901 (YP_{—}360429.1), etc.

Studies in Clostridia demonstrate that the crt gene that codes for crotonase is encoded as part of the larger BCS operon. However, studies on B. fibriosolvens, a butyrate producing bacterium from the rumen, show a slightly different arrangement. While Type I B. fibriosolvens have the thl, crt, hbd, bcd, etfA and etfB genes clustered and arranged as part of an operon, Type II strains have a similar cluster but lack the crt gene (Asanuma et al., Curr. Microbiol. 51: 91-94, 2005; Asanuma et al., Curr. Microbiol. 47: 203-207, 2003). Since the protein is well-expressed in E. coli and thoroughly characterized, the C. acetobutylicum enzyme is the preferred enzyme for the heterologously expressed n-butanol pathway. Other possible targets are homologous genes from Fusobacterium nucleatum subsp. Vincentii (Q7P3U9-Q7P3U9_FUSNV), Clostridium difficile (P45361-CRT_CLODI), Clostridium pasteurianum (P81357-CRT_CLOPA), and Brucella melitensis (Q8YDG2-Q8YDG2_BRUME).

In certain embodiments, the recombinant microorganism herein disclosed expresses a heterologous butyryl-CoA dehydrogenase and if necessary the corresponding electron transfer proteins, such as encoded by the bcd, etfA, and etfB genes from a Clostridium.

The C. acetobutylicum butyryl-CoA dehydrogenase (Bcd) is an enzyme that catalyzes the reduction of the carbon-carbon double bond in crotonyl-CoA to yield butyryl-CoA. This reduction is coupled to the oxidation of NADH. However, the enzyme requires two electron transfer proteins etfA and etfB (Bennett et al., Fems Microbiology Reviews 17: 241-249, 1995).

The Clostridium acetobutylicum ATCC 824 genes encoding the enzymes beta-hydroxybutyryl-coenzyme A (CoA) dehydrogenase, crotonase and butyryl-CoA dehydrogenase are clustered on the BCS operon, which GenBank accession number is U17110.

The butyryl-CoA dehydrogenase (Bcd) protein sequence (Genbank accession # AAA95968.1) is given in SEQ ID NO:3.

Homologs sharing at least about 55%, 60%, 65%, 70%, 75% or 80% sequence identity, or at least about 70%, 80%, 85% or 90% sequence homology, as calculated by NCBI's BLAST, are suitable Bcd homologs that can be used in the recombinant microorganisms herein disclosed. Such homologs include (without limitation): Clostridium tetani E88 (NP_{—}782955.1 or NP_{—}781376.1), Clostridium perfringens str. 13 (NP_{—}563216.1), Clostridium beijerinckii (AF494018_{—}2), Clostridium beijerinckii NCIMB 8052 (ZP_{—}00910125.1 or ZP_{—}00909697.1), Thermoanaerobacterium thermosaccharolyticum (CAB07496.1), Thermoanaerobacter tengcongensis MB4 (NP_{—}622217.1), etc.

The α-subunit of electron-transfer flavoprotein (EtfA) protein sequence (Genbank accession # AAA95970.1) is given in SEQ ID NO.4):

The β-subunit of electron-transfer flavoprotein (EtfB) protein sequence (Genbank accession # AAA95969.1) is given in SEQ ID NO:5.

The 3-hydroxybutyryl-CoA dehydrogenase (Hbd) protein sequence (Genbank accession # AAA95971.1) is given in SEQ ID NO:6.

Homologs sharing at least about 45%, 50%, 55%, 60%, 65% or 70% sequence identity, or at least about 60%, 70%, 80% or 90% sequence homology, as calculated by NCBI's BLAST, are suitable Hbd homologs that can be used in the recombinant microorganism herein described. Such homologs include (without limitation): Clostridium acetobutylicum ATCC 824 (NP_{—}349314.1), Clostridium tetani E88 (NP_{—}782952.1), Clostridium perfringens SM101 (YP_{—}699558.1), Clostridium perfringens str. 13 (NP_{—}563213.1), Clostridium saccharobutylicum (AAA23208.1), Clostridium beijerinckii NCIMB 8052 (ZP_{—}00910128.1), Clostridium beijerinckii (AF494018_{—}5), Thermoanaerobacter tengcongensis MB4 (NP_{—}622220.1), Thermoanaerobacterium thermosaccharolyticum (CAB04792.1), Alkaliphilus metalliredigenes QYMF (ZP_{—}00802337.1), etc.

The Kₘ of Bcd for butyryl-CoA is 5. C. acetobutylicum bcd and the genes encoding the respective ETFs have been cloned into an E. coli-C. acetobutylicum shuttle vector. Increased Bcd activity was detected in C. acetobutylicum ATCC 824 transformed with this plasmid (Boynton et al., Journal of Bacteriology 178: 3015-3024, 1996). The Km of the C. acetobutylicum P262 Bcd for butyryl-CoA is approximately 6 μM (DiezGonzalez et al., Current Microbiology 34: 162-166, 1997). Homologues of Bcd and the related ETFs have been identified in the butyrate-producing anaerobes Megasphaera elsdenii (Williamson et al., Biochemical Journal 218: 521-529, 1984), Peptostreptococcus elsdenii (Engel et al., Biochemical Journal 125: 879, 1971), Syntrophosphora bryanti (Dong et al., Antonie Van Leeuwenhoek International Journal of General and Molecular Microbiology 67: 345-350, 1995), and Treponema phagedemes (George et al., Journal of Bacteriology 152: 1049-1059, 1982). The structure of the M. elsdenii Bcd has been solved (Djordjevic et al., Biochemistry 34: 2163-2171, 1995). A BLAST search of C. acetobutylicum ATCC 824 Bcd identified a vast amount of homologous sequences from a wide variety of species, some of the homologs are listed herein above. Any of the genes encoding these homologs may be used for the subject invention. It is noted that expression and/or electron transfer issues may arise when heterologously expressing these genes in one microorganism (such as E. coli) but not in another. In addition, one homologous enzyme may have expression and/or electron transfer issues in a given microorganism, but other homologous enzymes may not. The availability of different, largely equivalent genes provides more design choices when engineering the recombinant microorganism.

One promising bcd that has already been cloned and expressed in E. coli is from Megasphaera elsdenii, and in vitro activity of the expressed enzyme could be determined (Becker et al., Biochemistry 32: 10736-10742, 1993). O'Neill et al. reported the cloning and heterologous expression in E. coli of the etfA and eftB genes and functional characterization of the encoded proteins from Megasphaera elsdenii (O'Neill et al., J. Biol. Chem. 273: 21015-21024, 1998). Activity was measured with the ETF assay that couples NADH oxidation to the reduction of crotonyl-CoA via Bcd. The activity of recombinant ETF in the ETF assay with Bcd is similar to that of the native enzyme as reported by Whitfield and Mayhew. Therefore, utilizing the Megasphaera elsdenii Bcd and its ETF proteins provides a solution to synthesize butyryl-CoA. The Kₘ of the M. elsdenii Bcd was measured as 5 μM when expressed recombinantly, and 14 μM when expressed in the native host (DuPlessis et al., Biochemistry 37: 10469-77, 1998). M. elsdenii Bcd appears to be inhibited by acetoacetate at extremely low concentrations (Kᵢ of 0.1 μM) (Vanberkel et al., Eur. J. Biochem. 178: 197-207, 1988). A gene cluster containing thl, crt, hbd, bcd, etfA, and etfB was identified in two butyrate producing strains of Butyrivibrio fibrisolvens. The amino acid sequence similarity of these proteins is high, compared to Clostridium acetobutylicum (Asanuma et al., Current Microbiology 51:91-94, 2005; Asanuma et al., Current Microbiology 47: 203-207, 2003). In mammalian systems, a similar enzyme, involved in short-chain fatty acid oxidation is found in mitochondria.

In certain embodiments, the recombinant microorganism herein disclosed expresses a heterologous “trans-2-enoyl-CoA reductase” or “TER”.

Trans-2-enoyl-CoA reductase or TER is a protein that is capable of catalyzing the conversion of crotonyl-CoA to butyryl-CoA. In certain embodiments, the recombinant microorganism expresses a TER which catalyzes the same reaction as Bcd/EtfA/EtfB from Clostridia and other bacterial species. Mitochondrial TER from E. gracilis has been described, and many TER proteins and proteins with TER activity derived from a number of species have been identified forming a TER protein family (U.S. Pat. Appl. 2007/0022497 to Cirpus et al.; Hoffmeister et al., J. Biol. Chem., 280: 4329-4338, 2005, both of which are incorporated herein by reference in their entirety). A truncated cDNA of the E. gracilis gene has been functionally expressed in E. coli. This cDNA or the genes of homologues from other microorganisms can be expressed together with the n-butanol pathway genes thl, crt, adhE2, and hbd to produce n-butanol in E. coli, S. cerevisiae or other hosts.

TER proteins can also be identified by bioinformatics methods known to those skilled in the art, such as BLAST. Examples of TER proteins include, but are not limited to, TERs from the following species:

Euglena spp. including but not limited to E. gracilis, Aeromonas spp. including but not limited to A. hydrophila, Psychromonas spp. including but not limited to P. ingrahamii, Photobacterium spp. including but not limited to P. profundum, Vibrio spp. including but not limited to V angustum, V cholerae, V alginolyticus, Vparahaemolyticus, V vulnificus, Vfischeri, V splendidus, Shewanella spp. including but not limited to S. amazonensis, S. woodyi, S. frigidimarina, S. paeleana, S. baltica, S. denitrificans, Oceanospirillum spp., Xanthomonas spp. including but not limited to X oryzae, X campestris, Chromohalobacter spp. including but not limited to C. salexigens, Idiomarina spp. including but not limited to I. baltica, Pseudoalteromonas spp. including but not limited to P. atlantica, Alteromonas spp., Saccharophagus spp. including but not limited to S. degradans, S. marine gamma proteobacterium, S. alpha proteobacterium, Pseudomonas spp. including but not limited to P. aeruginosa, P. putida, P. fluorescens, Burkholderia spp. including but not limited to B. phytofirmans, B. cenocepacia, B. cepacia, B. ambifaria, B. vietnamensis, B. multivorans, B. dolosa, Methylbacillus spp. including but not limited to M. flageliatus, Stenotrophomonas spp. including but not limited to S. maltophilia, Congregibacter spp. including but not limited to C. litoralis, Serratia spp. including but not limited to S. proteamaculans, Marinomonas spp., Xytella spp. including but not limited to X fastidiosa, Reinekea spp., Colwellia spp. including but not limited to C. psychrerythraea, Yersinia spp. including but not limited to Y. pestis, Y. pseudotuberculosis, Methylobacillus spp. including but not limited to M flagellatus, Cytophaga spp. including but not limited to C. hutchinsonii, Flavobacterium spp. including but not limited to F. johnsoniae, Microscilla spp. including but not limited to M marina, Polaribacter spp. including but not limited to P. irgensii, Clostridium spp. including but not limited to C. acetobutylicum, C. beijerenckii, C. cellulolyticum, Coxiella spp. including but not limited to C. burnetii.

In addition to the foregoing, the terms “trans-2-enoyl-CoA reductase” or “TER” refer to proteins that are capable of catalyzing the conversion of crotonyl-CoA to butyryl-CoA and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to either or both of the truncated E. gracilis TER as given in SEQ ID NO:7 or the full length A. hydrophila TER as given in SEQ ID NO: 8.

As used herein, “sequence identity” refers to the occurrence of exactly the same nucleotide or amino acid in the same position in aligned sequences. “Sequence similarity” takes approximate matches into account, and is meaningful only when such substitutions are scored according to some measure of “difference” or “sameness” with conservative or highly probably substitutions assigned more favorable scores than non-conservative or unlikely ones.

Another advantage of using TER instead of Bcd/EtfA/EtfB is that TER is active as a monomer and neither the expression of the protein nor the enzyme itself is sensitive to oxygen.

As used herein, “trans-2-enoyl-CoA reductase (TER) homologue” refers to an enzyme homologous polypeptides from other organisms, e.g., belonging to the phylum Euglena or Aeromonas, which have the same essential characteristics of TER as defined above, but share less than 40% sequence identity and 50% sequence similarity standards as discussed above. Mutations encompass substitutions, additions, deletions, inversions or insertions of one or more amino acid residues. This allows expression of the enzyme during an aerobic growth and expression phase of the n-butanol process, which could potentially allow for a more efficient biofuel production process.

In certain embodiments, the recombinant microorganism herein disclosed expresses a heterologous butyraldehyde dehydrogenase/n-butanol dehydrogenase, such as encoded by the bdhA/bdhB, aad, or adhE2 genes from a Clostridium.

The Butyraldehyde dehydrogenase (BYDH) is an enzyme that catalyzes the NADH-dependent reduction of butyryl-CoA to butyraldehyde. Butyraldehyde is further reduced to n-butanol by an n-butanol dehydrogenase (BDH). This reduction is also accompanied by NADH oxidation. Clostridium acetobutylicum contains genes for several enzymes that have been shown to convert butyryl-CoA to n-butanol.

One of these enzymes is encoded by aad (Nair et al., J. Bacteriol. 176: 871-885, 1994). This gene is referred to as adhE in C. acetobutylicum strain DSM 792. The enzyme is part of the sol operon and it encodes for a bifunctional BYDH/BDH (Fischer et al., Journal of Bacteriology 175: 6959-6969, 1993; Nair et al., J. Bacteriol. 176: 871-885, 1994). The protein sequence of this protein (GenBank accession # AAD04638.1) is given in SEQ ID NO:9.

The gene product of aad was functionally expressed in E. coli. However, under aerobic conditions, the resulting activity remained very low, indicating oxygen sensitivity. With a greater than 100-fold higher activity for butyraldehyde compared to acetaldehyde, the primary role of Aad is in the formation of n-butanol rather than of ethanol (Nair et al., Journal of Bacteriology 176: 5843-5846, 1994).

Homologs sharing at least about 50%, 55%, 60% or 65% sequence identity, or at least about 70%, 75% or 80% sequence homology, as calculated by NCBI's BLAST, are suitable homologs that can be used in the recombinant microorganisms herein disclosed. Such homologs include (without limitation): Clostridium tetani E88 (NP_{—}781989.1), Clostridium perfringens str. 13 (NP_{—}563447.1), Clostridium perfringens ATCC 13124 (YP_{—}697219.1), Clostridium perfringens SM101 (YP_{—}699787.1), Clostridium beijerinckii NCIMB 8052 (ZP_{—}00910108.1), Clostridium acetobutylicum ATCC 824 (NP_{—}149199.1), Clostridium difficile 630 (CAJ69859.1), Clostridium difficile QCD-32g58 (ZP_{—}01229976.1), Clostridium thermocellum ATCC 27405 (ZP_{—}00504828.1), etc.

Two additional NADH-dependent n-butanol dehydrogenases (BDH I, BDH II) have been purified, and their genes (bdhA, bdhB) cloned. The GenBank accession for BDH I is AAA23206.1, and the protein sequence is given in SEQ ID NO:10.

The GenBank accession for BDH II is AAA23207.1, and the protein sequence is given in SEQ ID NO:11.

These genes are adjacent on the chromosome, but are transcribed by their own promoters (Walter et al., Gene 134: 107-111, 1993). BDH I utilizes NADPH as the cofactor, while BDH II utilizes NADH. However, it is noted that the relative cofactor preference is pH-dependent. BDH I activity was observed in E. coli lysates after expressing bdhA from a plasmid (Petersen et al., Journal of Bacteriology 173: 1831-1834, 1991). BDH II was reported to have a 46-fold higher activity with butyraldehyde than with acetaldehyde and is 50-fold less active in the reverse direction. BDH I is only about two-fold more active with butyraldehyde than with acetaldehyde (Welch et al., Archives of Biochemistry and Biophysics 273: 309-318, 1989). Thus in one embodiment, BDH II or a homologue of BDH II is used in a heterologously expressed n-butanol pathway. In addition, these enzymes are most active under a relatively low pH of 5.5, which trait might be taken into consideration when choosing a suitable host and/or process conditions.

While the afore-mentioned genes are transcribed under solventogenic conditions, a different gene, adhE2 is transcribed under alcohologenic conditions (Fontaine et al., J. Bacteriol. 184: 821-830, 2002, GenBank accession # AF321779). These conditions are present at relatively neutral pH. The enzyme has been overexpressed in anaerobic cultures of E. coli and with high NADH-dependent BYDH and BDH activities. In certain embodiments, this enzyme is the preferred enzyme. The protein sequence of this enzyme (GenBank accession # AAK09379.1) is listed as SEQ ID NO:1.

Homologs sharing at least about 50%, 55%, 60% or 65% sequence identity, or at least about 70%, 75% or 80% sequence homology, as calculated by NCBI's BLAST, are suitable homologs that can be used in the recombinant microorganisms herein disclosed. Such homologs include (without limitation): Clostridium perfringens SM101 (YP_{—}699787.1), Clostridium perfringens str. 13 (NP_{—}563447.1), Clostridium perfringens ATCC 13124 (YP_{—}697219.1), Clostridium tetani E88 (NP_{—}781989.1), Clostridium beijerinckii NCIMB 8052 (ZP_{—}00910108.1), Clostridium difficile QCD-32g58 (ZP_{—}01229976.1), Clostridium difficile 630 (CAJ69859.1), Clostridium acetobutylicum ATCC 824 (NP_{—}149325.1), Clostridium thermocellum ATCC 27405 (ZP_{—}00504828.1), etc.

In certain embodiments, any homologous enzymes that are at least about 70%, 80%, 90%, 95%, 99% identical, or sharing at least about 60%, 70%, 80%, 90%, 95% sequence homology (similar) to any of the above polypeptides may be used in place of these wild-type polypeptides. These enzymes sharing the requisite sequence identity or similarity may be wild-type enzymes from a different organism, or may be artificial/recombinant enzymes.

In certain embodiments, any genes encoding for enzymes with the same activity as any of the above enzymes may be used in place of the genes encoding the above enzymes. These enzymes may be wild-type enzymes from a different organism, or may be artificial, recombinant or engineered enzymes.

Additionally, due to the inherent degeneracy of the genetic code, other nucleic acid sequences which encode substantially the same or a functionally equivalent amino acid sequence can also be used to clone and express the polynucleotides encoding such enzymes. As will be understood by those of skill in the art, it can be advantageous to modify a coding sequence to enhance its expression in a particular host. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.” Methodology for optimizing a nucleotide sequence for expression in a plant is provided, for example, in U.S. Pat. No. 6,015,891, and the references cited therein]

In certain embodiments, the recombinant microorganism herein disclosed has one or more heterologous DNA sequence(s) from a solventogenic Clostridia, such as Clostridium acetobutylicum or Clostridium beijerinckii. An exemplary Clostridium acetobutylicum is strain ATCC824, and an exemplary Clostridium beijerinckii is strain NCIMB 8052.

Expression of the genes may be accomplished by conventional molecular biology means. For example, the heterologous genes can be under the control of an inducible promoter or a constitutive promoter. The heterologous genes may either be integrated into a chromosome of the host microorganism, or exist as an extra-chromosomal genetic elements that can be stably passed on (“inherited”) to daughter cells. Such extra-chromosomal genetic elements (such as plasmids, BAC, YAC, etc.) may additionally contain selection markers that ensure the presence of such genetic elements in daughter cells.

In certain embodiments, the recombinant microorganism herein disclosed may also produce one or more metabolic intermediate(s) of the n-butanol-producing pathway, such as acetoacetyl-CoA, hydroxybutyryl-CoA, crotonyl-CoA, butyryl-CoA, or butyraldehyde, and/or derivatives thereof, such as butyrate.

In some embodiments, the recombinant microorganisms herein described engineered to activate one or more of the above mentioned heterologous enzymes for the production of n-butanol, produce n-butanol via a heterologous pathway.

As used herein, the term “pathway” refers to a biological process including one or more enzymatically controlled chemical reactions by which a substrate is converted into a product. Accordingly, a pathway for the conversion of a carbon source to n-butanol is a biological process including one or more enzymatically controlled reaction by which the carbon source is converted into n-butanol. A “heterologous pathway” refers to a pathway wherein at least one of the at least one or more chemical reactions is catalyzed by at least one heterologous enzyme. On the other hand, a “native pathway” refers to a pathway wherein the one or more chemical reactions is catalyzed by a native enzyme.

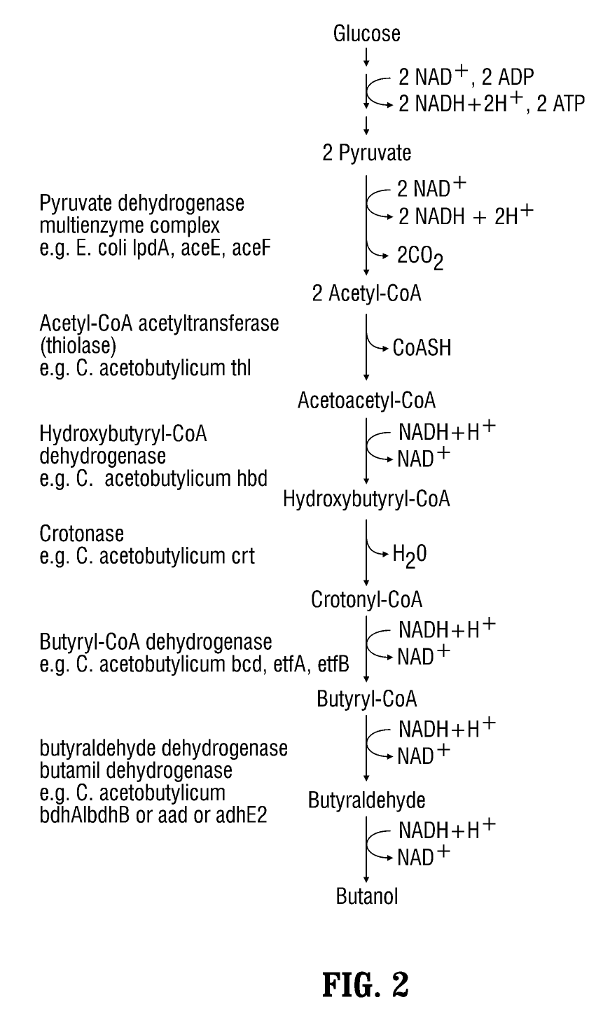

In certain embodiments, the recombinant microorganism herein disclosed are engineered to activate an n-butanol producing heterologous pathway (herein also indicated as n-butanol pathway) that comprises: (1) Conversion of 2 Acetyl-CoA to Acetoacetyl-CoA, (2) Conversion of Acetoacetyl CoA to Hydroxybutyryl-CoA, (3) Conversion of Hydroxybutyryl-CoA to Crotonyl-CoA, (4) Conversion of Crotonyl CoA to Butyryl-CoA, (5) Conversion of Butyraldehyde to n-butanol, (see the exemplary illustration of FIG. 2).

The conversion of 2 Acetyl-CoA to Acetoacetyl-CoA can be performed by expressing a native or heterologous gene encoding for an acetyl-CoA-acetyl transferase (thiolase) or Th1 in the recombinant microorganism. Exemplary thiolases suitable in the recombinant microorganism herein disclosed are encoded by thl from Clostridium acetobutylicum, and in particular from strain ATCC824 or a gene encoding a homologous enzyme from C. pasteurianum, C. beijerinckii, in particular from strain NCIMB 8052 or strain BA101, Candida tropicalis, Bacillus spp., Megasphaera elsdenii, or Butyrivibrio fibrisolvens, or an E. coli thiolase selected from fadA or atoB.

The conversion of Acetoacetyl CoA to Hydroxybutyryl-CoA can be performed by expressing a native or heterologous gene encoding for hydroxybutyryl-CoA dehydrogenase Hbd in the recombinant microorganism. Exemplary Hbd suitable in the recombinant microorganism herein disclosed are encoded by hbd from Clostridium acetobutylicum, and in particular from strain ATCC824, or a gene encoding a homologous enzyme from Clostridium kluyveri, Clostridium beijerinckii, and in particular from strain NCIMB 8052 or strain BA110, Clostridium thermosaccharolyticum, Clostridium tetani, Butyrivibrio fibrisolvens, Megasphaera elsdenii, or E. coli (fadB).

The conversion of Hydroxybutyryl-CoA to Crotonyl-CoA can be performed by expressing a native or heterologous gene encoding for a crotonase or Crt in the recombinant microorganism. Exemplary crt suitable in the recombinant microorganism herein disclosed are encoded by crt from Clostridium acetobutylicum, and in particular from strain ATCC824, or a gene encoding a homologous enzyme from B. fibriosolvens, Fusobacterium nucleatum subsp. Vincentii, Clostridium difficile, Clostridium pasteurianum, or Brucella melitensis.

The conversion of Crotonyl CoA to Butyryl-CoA can be performed by expressing a native or heterologous gene encoding for a butyryl-CoA dehydrogenase in the recombinant microorganism. Exemplary butyryl-CoA dehydrogenases suitable in the recombinant microorganism herein disclosed are encoded by bcd/etfA/etfB from Clostridium acetobutylicum, and in particular from strain ATCC824, or a gene encoding a homologous enzyme from Megasphaera elsdenii, Peptostreptococcus elsdenii, Syntrophosphora bryanti, Treponema phagedemes, Butyrivibrio fibrisolvens, or a mammalian mitochondria Bcd homolog.

The conversion of Butyraldehyde to n-butanol can be performed by expressing a native or heterologous gene encoding for a butyraldehyde dehydrogenase or a n-butanol dehydrogenase in the recombinant microorganism. Exemplary butyraldehyde dehydrogenase/n-butanol dehydrogenase suitable in the recombinant microorganism herein disclosed are encoded by bdhA, bdhB, aad, or adhE2 from Clostridium acetobutylicum, and in particular from strain ATCC824, or a gene encoding ADH-1, ADH-2, or ADH-3 from Clostridium beijerinckii, in particular from strain NCIMB 8052 or strain BA110.

In certain embodiments, the enzymes of the metabolic pathway from acetyl-CoA to n-butanol are (i) thiolase (Th1), (ii) hydroxybutyryl-CoA dehydrogenase (Hbd), (iii) crotonase (Crt), (iv) at least one of alcohol dehydrogenase (AdhE2), or n-butanol dehydrogenase (Aad) or butyraldehyde dehydrogenase (Ald) together with a monofunctional n-butanol dehydrogenase (BdhA/BdhB), and (v) trans-2-enoyl-CoA reductase (TER) (FIG. 2). In certain embodiments, the Th1, Hbd, Crt, AdhE2, Ald, BdhA/BdhB and Aad are from Clostridium. In certain embodiments, the Clostridium is a C. acetobutylicum. In certain embodiments, the TER is from Euglena gracilis or from Aeromonas hydrophila.

A recombinant microorganism that expresses an heterologous n-butanol pathway produces n-butanol at very low yields because most carbon is metabolized by native pathways. The n-butanol yield of a microorganism expressing a heterologous n-butanol pathway may be limited to levels of less than 2%. As exemplified in Example 19, wild-type E. coli W3110 expressing an n-butanol pathway on plasmids pGV1191 and pGV1113 converts glucose to n-butanol at a yield of about 1.4% of theoretical.

In order to provide the high yield of n-butanol, the recombinant microorganism including activated enzymes for the production of n-butanol, is further engineered to direct the carbon-flux originating from the metabolism of the carbon source to n-butanol. In particular, direction of carbon-flux to n-butanol can be performed by inactivating a metabolic pathway that competes with the n-butanol production.

A “competing pathway” with respect to the n-butanol production indicates a pathway for conversion of a substrate into a product wherein at least one of the substrates is a metabolic intermediate in the production of n-butanol. In certain embodiments, the competing pathway can also consume NADH (competing with respect to NADH consumption). Examplary pathways that compete with n-butanol production are endogenous fermentative pathways that lead to undesirable fermentation by-products and that possibly use or consume NADH.

The term “inactivated” or “inactivation” as used herein with reference to a pathway indicates a pathway in which any enzyme controlling a reaction in the pathway is biologically inactive, which includes but is not limited to inactivation of the enzyme is performed by deleting one or more genes encoding for enzymes of the pathway. The term “activated” or “activation”, as used herein with reference to a pathway, indicates a pathway in which any enzyme controlling a reaction in the pathway is biologically active. Accordingly, a pathway is inactivated when at least one enzyme controlling a reaction in the pathway is inactivated so that the reaction controlled by said enzyme does not occur. On the contrary, a pathway is activated when all the enzymes controlling a reaction in the pathway are activated.

In certain embodiments, inactivation of a competing pathway is performed by inactivating an enzyme involved in the conversion of a substrate to a product within the competing pathway. The enzyme that is inactivated may preferably catalyze the conversion of a metabolic intermediate for the production of n-butanol or may catalyze the conversion of a metabolic intermediate of the competing pathway. In certain embodiments, the enzyme also consumes NADH and therefore also competes with the n-butanol production also with respect to to NADH consumption.

The terms “inactivate” or “inactivation” as used herein with reference to a biologically active molecule, such as an enzyme, indicates any modification in the genome and/or proteome of a microorganism that prevents or reduces the biological activity of the biologically active molecule in the microorganism. Exemplary inactivations include but are not limited to modifications that results in the conversion of the molecule from a biologically active form to a biologically inactive form and from a biologically active form to a biologically less or reduced active form, and any modifications that result in a total or partial deletion of the biologically active molecule. For example, inactivation of a biologically active molecule can be performed by deleting or mutating a native or heterologous polynucleotide encoding for the biologically active molecule in the microorganism, by deleting or mutating a native or heterologous polynucleotide encoding for an enzyme involved in the pathway for the synthesis of the biologically active molecule in the microorganism, by activating a further a native or heterologous molecule that inhibits the expression of the biologically active molecule in the microorganism.

In particular, in some embodiments inactivation of a biologically active molecule such as an enzyme can be performed by deleting from the genome of the recombinant microorganism one or more endogenous genes encoding for the enzyme.

Accordingly, in certain embodiments the inactivation is performed by deleting from the microorganism's genome a gene coding for an enzyme involved in pathway that competes with the n-butanol production to make available the carbon/NADH to the one or more polypeptide(s) for producing n-butanol or metabolic intermediates thereof.

In certain embodiments, deletion of the genes encoding for these enzymes improves the n-butanol yield because more carbon and/or NADH is made available to one or more polypeptide(s) for producing n-butanol or metabolic intermediates thereof.

In certain embodiments, the DNA sequences deleted from the genome of the recombinant microorganism encode an enzyme selected from the group consisting of: D-lactate dehydrogenase, pyruvate formate lyase, acetaldehyde/alcohol dehydrogenase, phosphate acetyl transferase, acetate kinase A, fumarate reductase, pyruvate oxidase, and methylglyoxal synthase.

In particular when the microorganism is E. coli, the DNA sequences deleted from the genome can be selected from the group consisting of ldhA pflB, pflDC, adhE, pta, ackA, frd, poxB and mgsA.

Genes that are deleted or knocked out to produce the microorganisms herein disclosed are exemplified for E. coli. One skilled in the art can easily identify corresponding, homologous genes or genes encoding for enzymes which compete with the n-butanol producing pathway for carbon and/or NADH in other microorganisms by conventional molecular biology techniques (such as sequence homology search, cloning based on homologous sequences, etc.). Once identified, the target genes can be deleted or knocked-out in these host organisms according to well-established molecular biology methods.

In an embodiment, the deletion of a gene of interest occurs according to the principle of homologous recombination. According to this embodiment, an integration cassette containing a module comprising at least one marker gene is flanked on either side by DNA fragments homologous to those of the ends of the targeted integration site. After transforming the host microorganism with the cassette by appropriate methods, homologous recombination between the flanking sequences may result in the marker replacing the chromosomal region in between the two sites of the genome corresponding to flanking sequences of the integration cassette. The homologous recombination event may be facilitated by a recombinase enzyme that may be native to the host microorganism or be overexpressed.

The enzymes D-lactate dehydrogenase, pyruvate formate lyase, acetaldehyde/alcohol dehydrogenase, phosphate acetyl transferase, acetate kinase A, fumarate reductase, pyruvate oxidase, and/or methylglyoxal synthase, may be required for certain competing endogenous pathways that produce succinate, lactate, acetate, ethanol, formate, carbon dioxide and/or hydrogen gas.

In particular, the enzyme D-lactate dehydrogenase (encoded in E. coli by ldhA), couples the oxidation of NADH to the reduction of pyruvate to D-lactate. Deletion of ldhA has previously been shown to eliminate the formation of D-lactate in a fermentation broth (Causey, T. B. et al, 2003, Proc. Natl. Acad. Sci., 100, 825-32).

The enzyme Pyruvate formate lyase (encoded in E. coli by pflB), oxidizes pyruvate to acetyl-CoA and formate. Deletion of pflB has proven important for the overproduction of acetate (Causey, T. B. et al, 2003, Proc. Natl. Acad. Sci., 100, 825-32), pyruvate (Causey, T. B. et al, 2004, Proc. Natl. Acad. Sci., 101, 2235-40) and lactate (Zhou, S., 2005, Biotechnol. Lett., 27, 1891-96). Formate can further be oxidized to CO₂ and hydrogen by a formate hydrogen lyase complex, but deletion of this complex should not be necessary in the absence of pflB. pflDC is a homolog of pflB and can be activated by mutation. As indicated above, the pyruvate formate lyase may not need to be deleted for anaerobic fermentation of n-butanol. A (heterologous) NADH-dependent formate dehydrogenase may be provided, if not already available in the host, to effect the conversion of pyruvate to acetyl-CoA coupled with NADH production.

The enzyme acetaldehyde/alcohol dehydrogenase (encoded in E. coli by adhE) is involved the conversion of acetyl-CoA to acetaldehyde dehydrogenase and alcohol dehydrogenase. In particular, under aerobic conditions, pyruvate is also converted to acetyl-CoA, acetaldehyde dehydrogenase and alcohol dehydrogenase, but this reaction is catalyzed by a multi-enzyme pyruvate dehydrogenase complex, yielding CO₂ and one equivalent of NADH. Acetyl-CoA fuels the TCA cycle but can also be oxidized to acetaldehyde and ethanol by acetaldehyde dehydrogenase and alcohol dehydrogenase, both encoded by the gene adhE. These reactions are each coupled to the reduction of one equivalents NADH.

The enzymes phosphate acetyl transferase (encoded in E. coli by pta) and acetate kinase A (encoded in E. coli by ackA), are involved in the pathway which converts acetyl-CoA to acetate via acetyl phosphate. Deletion of ackA has previously been used to direct the metabolic flux away from acetate production (Underwood, S. A. et al, 2002, Appl. Environ. Microbiol., 68, 6263-72; Zhou, S. D. et al, 2003, Appl. Environ. Mirobiol., 69, 399-407), but deletion of pta should achieve the same result.

The enzyme fumarate reductase (encoded in E. coli by frd) is involved in the pathway which converts pyruvate to succinate. In particular, under anaerobic conditions, phosphoenolpyruvate can be reduced to succinate via oxaloacetate, malate and fumarate, resulting in the oxidation of two equivalents of NADH to NAD⁺. Each of the enzymes involved in those conversions could be inactivated to eliminate this pathway. For example, the final reaction catalyzed by fumarate reductase converts fumarate to succinate. The electron donor for this reaction is reduced menaquinone and each electron transferred results in the translocation of two protons. Deletion of frd has proven useful for the generation of reduced pyruvate products.

The enzyme pyruvate oxidase (encoded in E. coli by poxB) is involved in the pathway which converts pyruvate to acetate. This enzyme does not require NADH. However, upon decarboxylation of pyruvate, pyruvate oxidase transfers electrons from pyruvate to ubiquinone to form ubiquinol. Because of this electron transfer to the quinone pool, pyruvate oxidase indirectly increases the microorganism's need for oxygen. Removing pyruvate oxidase from the microorganism will prevent oxygen from being consumed by this pathway.

The enzyme methylglyoxal synthase (MGS, encoded in E. coli by mgsA) is involved in pathway which converts pyruvate to lactate. It has been discovered that even when the ldhA gene has been inactivated significant residual amounts of lactate are still produced. Much of the residual lactate can be attributed to the methylglyoxal bypass of the glycolytic pathway. In particular, the first step of the methyglyoxal bypass is catalyzed by methylglyoxal synthase (MGS) (E.C. 4.2.99.11), which in E. coli is encoded by the mgsA gene, alternatively known as yccG. Homologues of mgsA were identified by database searches in Haemophilus influenzae (D6411169), Bacillus subtilis (P42980), Brucella abortus (BAU21919_{—}2) and Synechocystis (SYCSLLLH_{—}17) (Tötemeyer et al., Molecular Microbiology 27: 553-562, 1998). MGS catalyzes the apparently irreversible conversion of dihydroxyacetone phosphate (DHAP) to methylglyoxal and orthophosphate. Methylglyoxal synthases have been identified in a variety of organisms including Pseudomonas saccarophila, Pseudomonas doudoroffi, Clostridium tetanomorphum, Clostridium pasteurianum, Desulfovibrio gigas and Proteus vulgaris (see, Saadat et al., Biochemistry 37: 10074-10086, 1998; Tötemeyer et al., Molecular Microbiology 27: 553-562, 1998). Methylglyoxal is extremely cytotoxic at millimolar concentrations. In E. coli the enzymes glyoxalase I and II are the primary enzymes used to detoxify methylglyoxal by catalyzing the glutathione dependent conversion of methylglyoxal to D(−)-lactate. D(−)-Lactate can be converted to pyruvate via flavin-linked dehydrogenases.

The expression of gene fnr is associated with a series of activities in E. coli. The pathways associated to the activity expressed by fnr are usually related to oxygen utilization that is down regulated as oxygen is depleted and in a reciprocal fashion, alternative anaerobic pathways for fermentation are upregulated by Fnr. An indication of those pathways can be found in Chrystala Constantinidou et al., “A Reassessment of the FNR Regulon and Transcriptomic Analysis of the Effects of Nitrate, Nitrite, NarXL, and NarQP as Escherichia coli K12 Adapts from Aerobic to Anaerobic Growth,” J. Biol. Chem., 2006, 281:4802-4815 Kirsty Salmon et al., “Global Gene Expression Profiling in Escherichia coli K12—The Effects Of Oxygen Availability And FNR “J. Biol. Chem. 2003, 278(32):29837-55” and Kirsty A. Salmon et al. “Global Gene Expression Profiling in Escherichia coli K12—the Effects of Oxygen Availability and ArcA” J. Biol. Chem., 2005, 280(15):15084-15096, all incorporated by reference in their entirety in the present application.

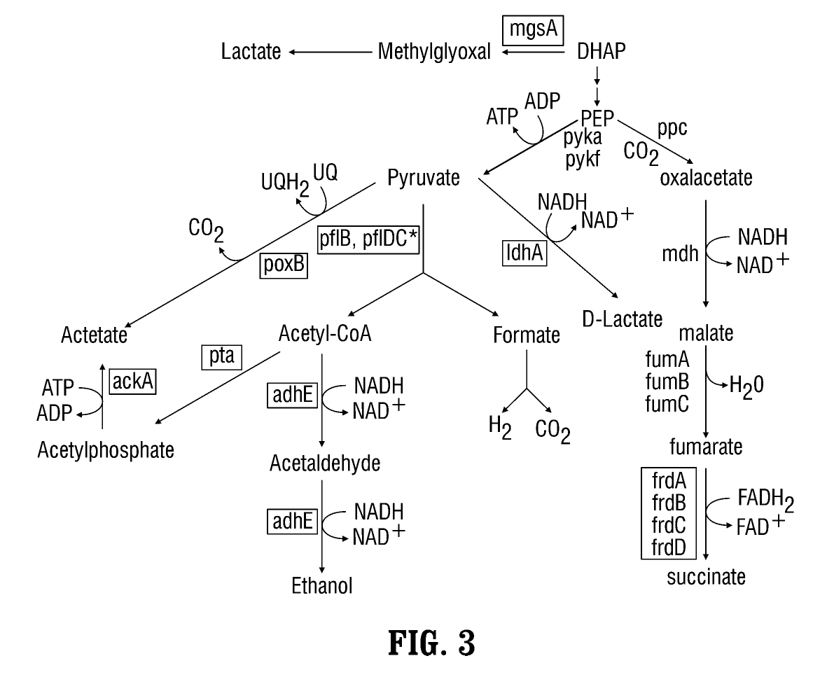

Pathways and conversions catalyzed by the some of the mentioned enzymes are schematically illustrated in the exemplary representation of FIG. 3.

In view of the above, and in particular of the pathways that are inactivated by the inactivation of said enzymes, recombinant microorganisms are herein disclosed engineered to activate one or more heterologous enzymes for the production of n-butanol, the recombinant microorganism further engineered to inactivate competing pathways including (1) Conversion of Pyruvate to Lactate (2) Conversion of Acetyl-CoA to acetate, (3) Conversion of Acetyl-CoA to Acethaldehyde, (4) Conversion of Pyruvate to Succinate, and (5) Conversion of Pyruvate to Acetate, and (6) any metabolic pathways associated with the expression of an fnr gene in the microorganism. A schematic representation of the above pathways is illustrated in FIG. 3

In particular, deletion of the conversion of pyruvate to lactate can be performed by inactivation of the competing enzymes D-lactate dehydrogenase and/or methylglyoxal synthase, in particular by inactivating a gene that encodes in the microorganism for D-lactate dehydrogenase and/or a gene in the microorganism that encodes for methylglyoxal synthase.

Deletion of the conversion of Acetyl-CoA to acetate can be performed by inactivation of the competing enzyme Acetaldehyde/alcohol dehydrogenase, in particular by inactivating a gene in the microorganism that encodes for the Acetaldehyde/alcohol dehydrogenase.

Deletion of the conversion of Acetyl-CoA to Acethaldehyde can be performed by inactivating the competing enzyme phosphate acetyl transferase and/or competing enzyme acetate kinase A, in particular by inactivating the gene in the microorganism that encodes for the phosphate acetyl transferase and/or acetate kinase A.

Deletion of the conversion of pyruvate to succinate can be performed by inactivating the competing enzyme fumarate reductase, in particular by inactivating a gene in the microorganism that encodes for fumarate reductas.

Deletion of the conversion of the conversion of Pyruvate to Acetate, can be performed by inactivating the competing enzyme pyruvate oxidase, in particular by inactivating a gene in the microorganism that encodes for pyruvate oxidase.

Deletion of any pathways associated to fnr gene can be performed by inactivating the relevant gene in the microorganism.

In some embodiments, the recombinant microorganism is engineered to inactivate one of these pathways. In some embodiments the recombinant microorganism is engineered to inactivate some or all of the above pathways. Thus it is contemplated that not all of these pathways are to be removed in all embodiments. One or more of the pathways may remain largely or partially intact. In addition, one or more of these pathways may be conditionally inactivated, such as by using an inducible promoter to direct the expression of one or more key enzymes in the pathways, or by using a temperature sensitive mutation of one or more key enzymes in the pathways. It is possible, though usually not necessary to disable all enzymes in the same pathway.

In some embodiments, the inactivation of lactate dehydrogenase and of the related conversion of pyruvate to lactate can increase the n-butanol yield to about 2%. For example, the n-butanol yield of GEVO1082 (E. coli W3110, ΔldhA) is expected to be about 2% of theoretical, which is 40% higher compared to the strain without any competing pathways removed. However, this strain produces mainly ethanol. In an attempt to remove ethanol production and further increase the n-butanol yield, the inactivation of a gene encoding for an alcohol dehydrogenase that converts acetyl-CoA to ethanol may be removed.

In some embodiments the inactivation of alcohol dehydrogenase and of the related conversion of acetyl-coA to ethanol can increase the n-butanol yield to about 6%. For example, the n-butanol yield of GEVO1054 (E. coli W3110, ΔadhE) is expected to be about 5 to 5.6% of theoretical.

In some embodiments the inactivation of lactate dehydrogenase and of the related conversion of pyruvate to lactate and the inactivation of alcohol dehydrogenase and of the related conversion of acetyl-CoA to ethanol may decrease the production of lactate and ethanol and may increases the n-butanol yield to about 7%. For example, the n-butanol yield of GEVO1084 (E. coli W3110, ΔldhA, ΔadhE) is expected to be about 7% of theoretical.

In some embodiments, the inactivation of lactate dehydrogenase, alcohol dehydrogenase, and fumarate reductase, and of the related conversions pyruvate to lactate, acetyl-CoA to ethanol, and pyruvate to succinate, respectively, may decrease the production of lactate, ethanol and succinate and may increase the n-butanol yield to about 21%. As exemplified in example 17, GEVO1083 (E. coli W3110, ΔldhA, ΔadhE, Δndh, Δfrd) may be about 20 to 22.4% of theoretical.

In some embodiments, the inactivation of lactate dehydrogenase, alcohol dehydrogenase, fumarate reductase, and methylglyoxal synthase and of the related conversion of pyruvate to lactate, acetyl-CoA to ethanol, pyruvate to succinate, and pyruvate to methylglyoxal, respectively, may decrease the production of lactate, ethanol, and succinate and increase the n-butanol yield to about 21%. As exemplified in example 16, the n-butanol yield of GEVO1121 (E. coli W3110, ΔldhA, ΔadhE, Δndh, Δfrd, ΔmgsA) may be about 19% higher compared to GEVO1083 (E. coli W3110, ΔldhA, ΔadhE, Δndh, Δfrd) and thus may be expected to give at least a yield of up to 25% of theoretical.

In some embodiments, the inactivation of a lactate dehydrogenase, alcohol dehydrogenase, fumarate reductase, and acetate kinase and of the related conversions of pyruvate to lactate, acetyl-CoA to ethanol, pyruvate to succinate, and acetyl-CoA to acetate, respectively, may decrease the production of lactate, ethanol, succinate and acetate and may increase the n-butanol yield to about 25%. As exemplified in example 17, the n-butanol yield of GEVO1121 (E. coli W3110, ΔldhA, ΔadhE, Δndh, Δfrd, ΔackA) is about 25% of theoretical.

In certain embodiments, production of n-butanol in the recombinant microorganisms herein disclosed occurs through an NADH-dependent pathway, i.e. a pathway wherein the conversion of the substrate to the product requires reducing equivalents provided by NAD(P)H at some catalytic step within said pathway or by some or one enzyme or biologically active molecule within said pathway.

In particular, in embodiments, wherein the n-butanol producing pathway includes conversion of acetyl-CoA to n-butanol (see e.g. the n-butanol pathway, FIG. 2), four molecules of NADH are required for the conversions of two molecules of acetyl-CoA to one molecule of n-butanol. During the conversion of glucose to acetyl-CoA under anaerobic conditions, however, only two molecules of NADH are generated.

Microorganisms providing only two molecules of NADH to the n-butanol pathway that requires four molecules of NADH are not balanced, and thus cannot produce n-butanol at a yield of greater than 50% of theoretical. The microorganism therefore may be engineered to increase the moles of NADH generated from one mole of glucose. Preferably, the four moles of NADH are generated from one mole of glucose.

Accordingly, in some embodiments, in order to provide the high yield of n-butanol, the recombinant microorganisms expression heterologous enzymes for the production of n-butanol, are further engineered to balance NADH production and consumption with respect to the production of n-butanol, i.e., the total number of NADH molecules produced (e.g., as produced during glycolysis and during conversion of pyruvate to acetyl-CoA) equals the total number of NADH molecules consumed by the n-butanol-producing pathway, thus leaving no extra NADH and having no NADH deficiency.

Accordingly in those embodiments, the conversion of a carbon source to n-butanol is balanced with respect to NADH production and consumption. NADH produced during the oxidation reactions of the carbon source equals the NADH utilized to convert acetyl-CoA to n-butanol. Only under these conditions is all the NADH recycled. Without recycling, the NADH/NAD⁺ ratio becomes imbalanced and will cause the organisms to ultimately die unless alternate metabolic pathways are available to maintain a balance.

In particular, in certain embodiments, the recombinant microorganism is engineered so that production of n-butanol occurs through a fermentative heterologous pathway, wherein the unengineered microorganism is unable to produce n-butanol via a balanced fermentation because the microorganism does not produce sufficient NADH to convert acetyl-CoA to n-butanol.

Thus, in certain embodiments, if necessary or desirable, pyruvate dehydrogenase is activated under culture conditions at which n-butanol is produced, preferably under anaerobic conditions. In certain embodiments, pyruvate dehydrogenase is engineered to be active under anaerobic conditions. Alternatively, a pyruvate dehydrogenase from a heterologous host that utilizes the enzyme under anaerobic conditions may be expressed in the microorganism.

In another embodiment, formate hydrogen lyase is replaced by an NADH-dependent formate dehydrogenase.

In yet another embodiment, the microorganism is engineered to utilize glycerol as a carbon source via an engineered metabolic pathway that produces sufficient NADH to convert acetyl-CoA to n-butanol.

For example, in an E. coli host microorganism, an n-butanol-producing pathway as depicted in FIG. 2 is balanced with respect to NADH production, since four total NADH molecules are generated and then consumed by the pathway enzymes. This can be achieved in several ways. In one embodiment, the host may functionally express the native pyruvate dehydrogenase under anaerobic conditions. In another embodiment, pyruvate dehydrogenases from other organisms may also be used for this purpose under anaerobic conditions. The polypeptides encoded by these E. coli or heterologous genes may be put under the control of an inducible promoter to effect functional expression.

In certain embodiments, the recombinant microorganism herein disclosed includes an activated NADH-dependent formate dehydrogenase which is active under anaerobic or microaerobic conditions.

NADH-dependent formate dehydrogenase (Fdh; EC 1.2.1.2) catalyzes the oxidation of formate to CO₂ and the simultaneous reduction of NAD⁺ to NADH. Fdh can be used in accordance with the present disclosure to increase the intracellular availability of NADH within the host microorganism and may be used to balance the n-butanol producing pathway with respect to NADH. In particular, a biologically active NADH-dependent Fdh can be activated and in particular overexpressed in the host microorganism. In the presence of this newly introduced formate dehydrogenase pathway, one mole of NADH will is formed when one mole of formate is converted to carbon dioxide. In certain embodiments, in the native microorganism a formate dehydrogenase converts formate to CO₂ and H₂ with no cofactor involvement.

In certain embodiments, such as in embodiments wherein the microorganism is E. coli, the host utilizes an endogenous pyruvate-formate-lyase (encoded in E. coli by pfl) to convert pyruvate to acetyl-CoA under anaerobic conditions, NADH is not produced by this reaction, since pyruvate-formate-lyase is not NADH-dependent. Under this circumstance, an NADH-dependent formate dehydrogenase may be activated in the microorganism, so that in combination with the endogenous non-NADH-dependent pyruvate-formate-lyase, the following reaction stoichiometry is similarly achieved under anaerobic or microaerobic conditions (Berrios-Rivera, S. J. et al, 2002, Metabol. Eng., 2002, 217-29):

Pyruvate+NAD⁺→acetyl-CoA+NADH+CO₂

In particular, a heterologous NADH-dependent formate dehydrogenase can be activated, so that the conversion of pyruvate results in the same net stoichiometry: for each mole of pyruvate, one mole of carbon dioxide is formed, generating the necessary equivalent of NADH. This allows the cells to retain the reducing power that otherwise will be lost by release of formate or hydrogen in the native pathway.

Examplary fdh suitable in the recombinant microorganisms herein described include, an NADH-dependent Fdh1 of Candida boidinii (GenBank Accession NO: AF004096), fdh from Candida methylica (GenBank Accession NO: CAA57036), Arabidopsis thaliana (GenBank Accession NO: AAF19436), Pseodomonas sp. 101 (GenBank Accession NO: P33160), and Staphylococcus aureus (GenBank Accession NO: BAB94016).

Additional exemplary fdh enzymes suitable in the recombinant microorganisms herein described comprise native fdh of the following microorganisms Saccharomyces servazzii, Saccharomyces bayanus, Zygosaccharomyces rouxii, Saccharomyces exiguus, Saccharomyces kluyveri, Kluyveromyces lactis, Kluyveromyces thermotolerans, Kluyveromyces marxianus, Debaryomyces hansenii, Pichia sorbitophila, Pichia angusta, Candida tropicalis and Yarrowia lipolytica.

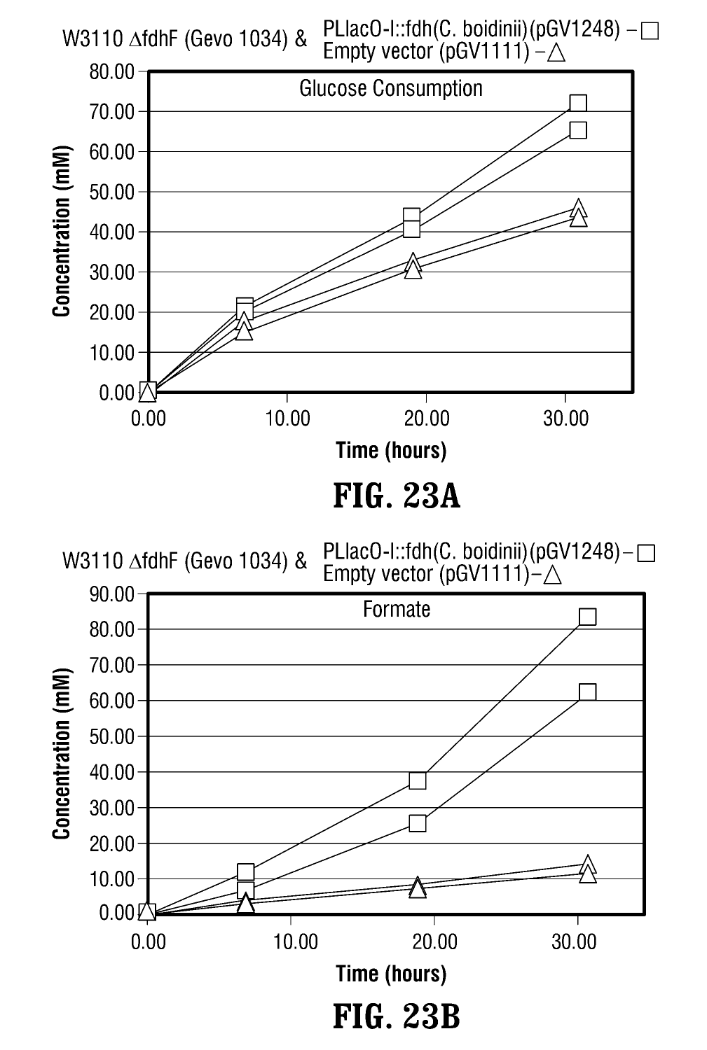

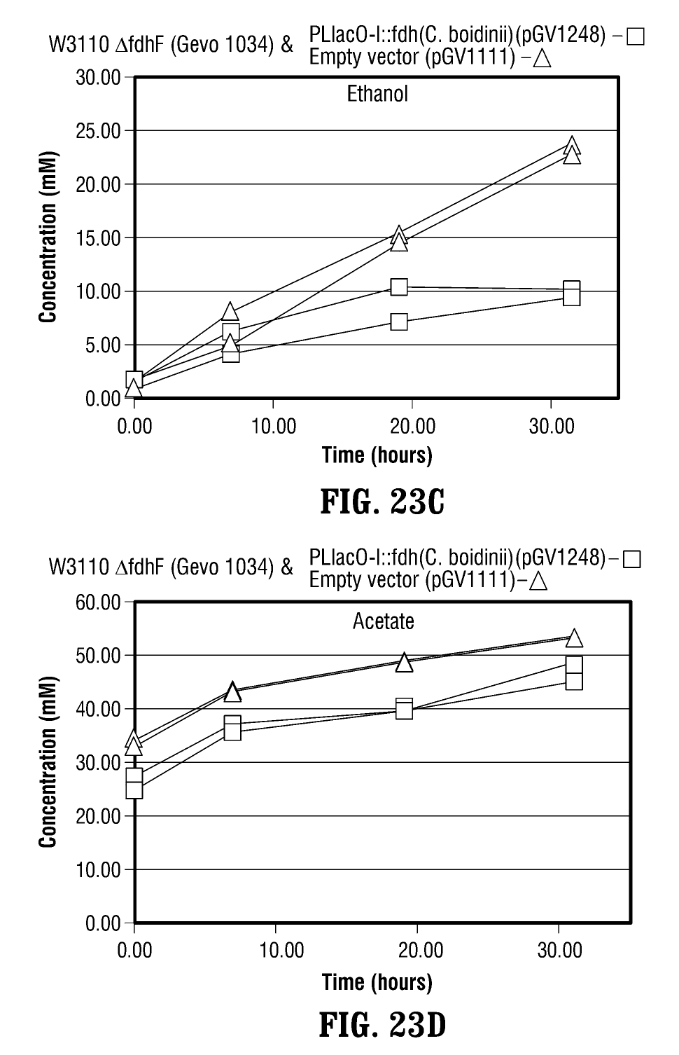

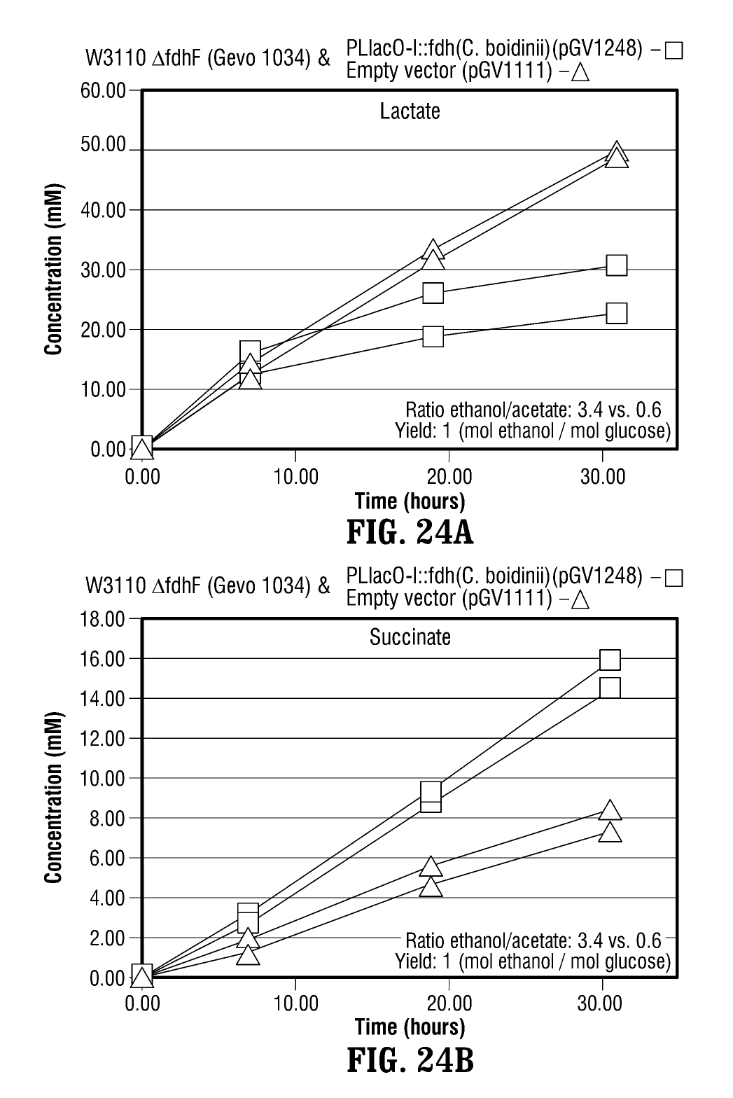

Activation of an fdh can be performed in the host using several approaches. For example, expression of Fdh from Candida boidinii (SEQ ID NO:13) in a strain with decreased pyruvate-formate-lyase activity increases ethanol production (see FIG. 23B) which indicates an intracellular NADH availability of at least three moles of NADH per mole of glucose consumed. Furthermore, an Fdh-dependent availability of up to 4 moles of NADH per glucose consumed has been described (Berrios-Rivera et al., Metabol. Eng., 4, 217, 2007; US 2003/0175903 A1; Example 8).

Thus, overexpression of an NADH-dependent formate dehydrogenase is expected to increase the moles of NADH available to the n-butanol pathway to 2.5, 3, 3.5, 4, and therefore to achieve balancing of an n-butanol pathway in a microorganism. As exemplified in example 21, E. coli strain GEVO1034 expressing Fdh from pGV1248 produces about 3 moles of NADH per mole of glucose. Expression of an n-butanol production pathway in a microorganism expressing Fdh is expected to result in n-butanol yields of greater than 1.4% if the n-butanol production pathway can compete with endogenous fermentative pathways. As exemplified in example 24, GEVO768 (E. coli W3110) expressing an NADH-dependent Fdh and an n-butanol production pathway from pGV1191 and pGV1583 produces n-butanol at a yield that is 30% higher (2% of theoretical) compared a control strain GEVO768 expressing an n-butanol production pathway from plasmids pGV1191 and pGV1435.

In certain embodiments, the recombinant microorganism herein disclosed include an active pyruvate dehydrogenase (Pdh) under anaerobic or microaerobic conditions. The pyruvate dehydrogenase or NADH-dependent formate dehydrogenase may be heterologous to the recombinant microorganism, in that the coding sequence encoding these enzymes is heterologous, or the transcriptional regulatory region is heterologous (including artificial), or the encoded polypeptides comprise sequence changes that renders the enzyme resistant to feedback inhibition by certain metabolic intermediates or substrates.

The enzyme pyruvate dehydrogenase (Pdh) catalyzes the conversion of pyruvate to acetyl-CoA with production of carbon dioxide. While catalyzing this reaction, Pdh produces one NADH and consumes one ATP. This enzyme is usually expressed under aerobic conditions, where ATP is plentiful, and NADH can easily be consumed by NADH dehydrogenase enzymes in the respiration pathways, resulting in a relatively low NADH/NAD⁺ ratio. Under anaerobic conditions when additional NADH is not needed, and when the NADH/NAD⁺ ratio is relatively high, pyruvate formate lyase is used by the cell to convert pyruvate to acetyl-CoA and formate. In this case, the electrons that are released by the Pdh reaction remain in formate, which is either secreted or converted into carbon dioxide and hydrogen gas by formate hydrogen lyase. To balance an n-butanol production pathway in E. coli, the conversion of pyruvate to acetyl-CoA must produce an NADH under anaerobic conditions.

Until recently, it was widely accepted that Pdh does not function under anaerobic conditions, but several recent reports have demonstrated that this is not the case (de Graef, M. et al, 1999, Journal of Bacteriology, 181, 2351-57; Vernuri, G. N. et al, 2002, Applied and Environmental Microbiology, 68, 1715-27). Moreover, other microorganisms such as Enterococcus faecalis exhibit high in vivo activity of the Pdh complex, even under anaerobic conditions, provided that growth conditions were such that the steady-state NADH/NAD⁺ ratio was sufficiently low (Snoep, J. L. et al, 1991, Fems Microbiology Letters, 81, 63-66). Instead of oxygen regulating the expression and function of Pdh, it has been shown that Pdh is regulated by NADH/NAD⁺ ratio (de Graef, M. et al, 1999, Journal of Bacteriology, 181, 2351-57). The Pdh from E. coli is generally inactivated by the increasing NADH levels that are associated with a switch to anaerobic metabolism, but if alternative electron acceptors are available to the cell to drop the NADH levels, Pdh may be used. If the n-butanol pathway expressed in E. coli consumes NADH fast enough to maintain a low NADH/NAD⁺ level inside the cell, the endogenous Pdh may remain active enough to balance the pathway, especially if the gene for pyruvate formate lyase is knocked out.

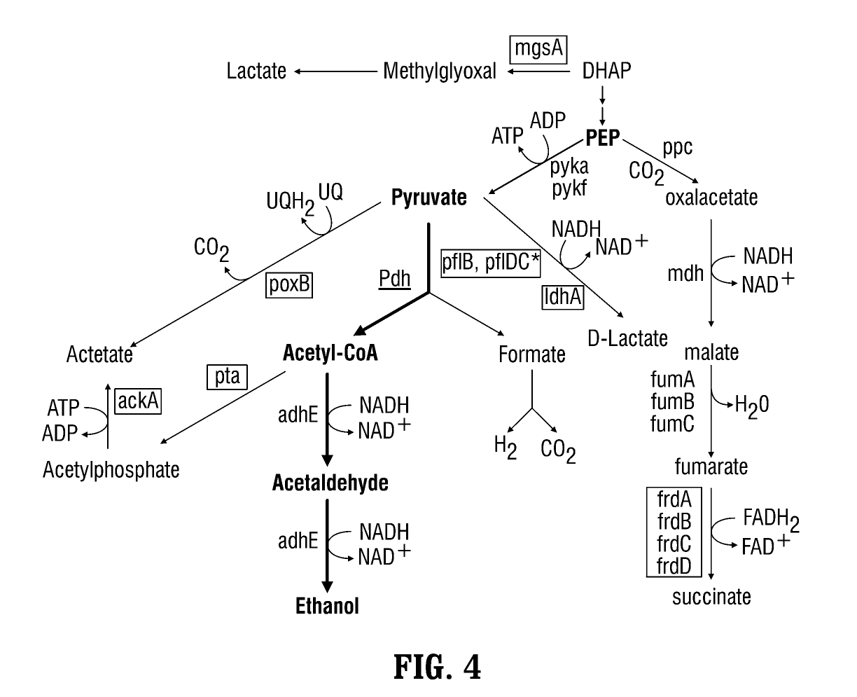

Thus in some embodiments, the recombinant microorganism expresses a functional endogenous Pdh in the n-butanol-producing pathway. Preferably, in those embodiments the enzyme pyruvate formate lyase is also inactivated. Alternatively, an evolutionary strategy may be used to increase Pdh activity under anaerobic conditions. This strategy relies upon utilizing an engineered E. coli variant that has all fermentative pathways but ethanol production removed (FIG. 4). This strain is fed glucose under anaerobic conditions. Under these conditions, the fermentation of glucose to ethanol is only possible if an additional equivalent of NADH is provided by a functionally expressed Pdh. Pdh with increased activity under anaerobic conditions may be generated using this method, and be used in the recombinant microorganism herein disclosed.

If embodiments wherein the native Pdh is not active under anaerobic conditions to drive n-butanol production (e.g. in E. coli), a Pdh from another organism can be expressed. For example, Pdh from Enterococcus faecalis is similar to the Pdh from E. coli but is inactivated at much lower NADH/NAD⁺ levels. Additionally, some organisms such as Bacillus subtilis and almost all strains of lactic acid bacteria use a Pdh in anaerobic metabolism. These Pdh enzymes can balance the n-butanol pathway in recombinant microorganism herein disclosed.

Expression of a Pdh that is functional under anaerobic conditions is expected increase the moles of NADH per mole of glucose. Evolution of Pdh as described supra may increase its activity under anaerobic conditions which is observable by increased ratios of ethanol to acetate produced from glucose. As exemplified in example 22, the ratio of ethanol to acetate may increase from 0.8 to 1.1, indicating that Pdh exhibits increased activity under anaerobic conditions. Kim et al. describe the a Pdh that makes available in E. coli up to four moles of NADH per mole of glucose consumed (Kim Y. et al. Appl. Environm. Microbiol., 2007, 73, 1766-1771). Thus, utilization of an anaerobically active Pdh is expected to increase the moles of NADH available to the n-butanol pathway to 2.5, 3, 3.5, 4, and therefore is expected to achieve balancing of an n-butanol pathway in a microorganism. Expression of an n-butanol production pathway in a microorganism expressing a Pdh that is functional under anaerobic conditions is expected to result in n-butanol yields of greater than 1.4% if the n-butanol production pathway can compete with endogenous fermentative pathways.

In certain embodiments, a carbon source that is more reduced than glucose can be used to balance the n-butanol pathway. In particular, said carbon source can be glycerol that is generally metabolized by its conversion into the glycolysis intermediate glyceraldehyde-3-phosphate (Lin, E. C. C., 1976, Annu. Rev. Microbiol., 30, 535-78). A yield of up to two molecules of NADH per glycerol converted to acetyl-CoA may be achieved, thus providing sufficient NADH for the conversion of acetyl-CoA to n-butanol.

In certain embodiments the recombinant microorganism is engineered to activate a heterologous pathway for converting glycerol to pyruvate.

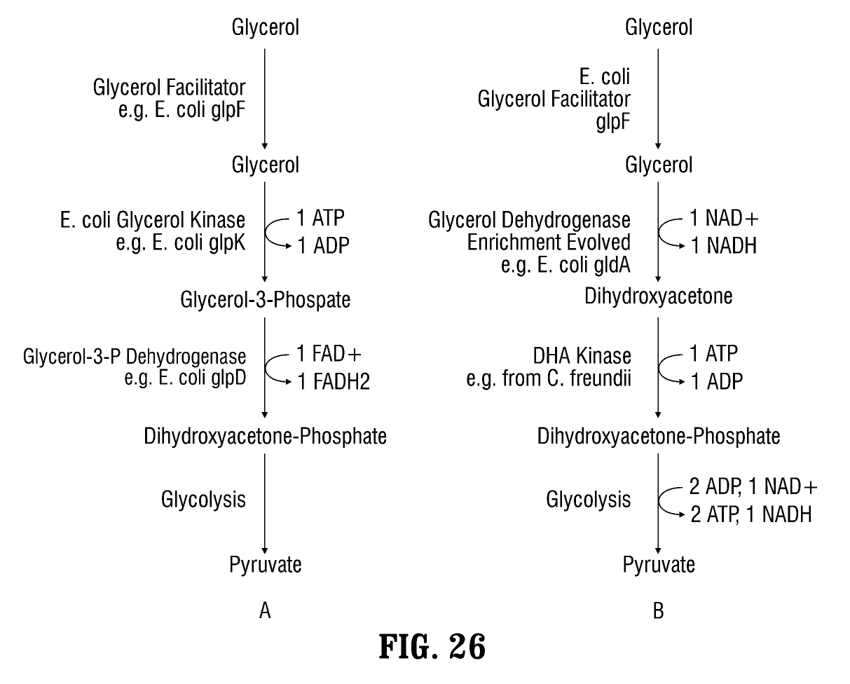

In particular, in some embodiments the carbon source to be converted to n-butanol comprises glycerol, and a glycerol degradation pathway is activated that avoids a glycerol-3-phosphate dehydrogenase catalyzed step that feeds electrons into the quinone pool. The glycerol degradation pathway can be activated by inactivating genes encoding glycerol kinase and glycerol-3-phosphate dehydrogenase (Jin, R. Z. et al, 1983, Journal of Molecular Evolution, 19, 429-36). The pathway is made more efficient by expressing a DHA kinase which may be from Citrobacter freundii, S. cerevisiae or other organisms (FIG. 26). The DHA kinase avoids the phosphorylation of DHA by a phosphotransferase system (PTS), which requires DHA to diffuse out of the cell and re-enter through the PTS while being phosphorylated (FIG. 26).

In some embodiments, the recombinant microorganism herein disclosed are engineered to complement the evolution-enhanced expression or overexpression of a glycerol dehydrogenase, wherein the native microorganism does not metabolize glycerol via the intermediate dihydroxyacetone (DHA). In particular, in certain embodiments host organisms have a native pathway that converts glycerol via the intermediate DHA, wherein conversion proceeds via the PEP-dependent PTS conversion of DHA to dihydroxyacetone-phosphate (DHAP). By expressing a soluble DHA kinase of, for example Citrobacter freundii, Klebsiella pneumonia, or Saccharomyces cerevisiae recombinantly, limitations of native DHA utilization pathways requiring PEP and the diffusion of DHA to the cell's membrane may be overcome, so that DHAP may be more efficiently available to the cell. Hence the subsequent metabolites of DHAP metabolism, such as pyruvate and acetyl-CoA, and NAD(P)H equivalents that may be utilized by the cell for a biotransformation, be they native or heterologously expressed enzymes, may be more efficiently available to the cell as well.

In one embodiment, a gene encoding DHA kinase from C. freundii, K. pneumoniae or S. cerevisiae is cloned by utilizing the polymerase chain reaction and primers appropriate to obtain linear double-stranded DNA of the complete gene by methods well known by those of skill in the art.

The sequence of the DHA kinase-encoding gene from C. freundii (Genbank accession # DQ473522.1), is given as SEQ ID NO:12. The sequence of the DHA kinase-encoding gene on the K. pneumoniae genomes is given as SEQ ID NO:14. The sequence of the DHA kinase-encoding gene Dak1 on the S. cerevisiae genomes is given as SEQ ID NO:15. The sequence of the DHA kinase-encoding gene Dak2 on the S. cerevisiae genomes is given as SEQ ID NO:16.

In one embodiment, the gene encoding DHA kinase is used without deleting the wild-type DHA operon of the host organism. In an alternative embodiment, the wild-type DHA operon of the host organism is deleted. In one embodiment, DHA kinase is overexpressed from a plasmid with one of many promoters and antibiotic resistance genes, appropriate to the expression level required for a given strain.

In one embodiment, a gene encoding DHA kinase is chromosomally integrated. Methods of chromosomally integrating a gene are known in the art. According to this embodiment, by using standard molecular biology techniques, the C. freundii, K. pneumonia, or S. cerevisae gene for DHA kinase is inserted into the microorganism genome.

The presence and integrity of the DHA kinase-encoding gene insertion into the chromosome may be verified by PCR using primers that are adjacent and outside the replaced gene as well as complementary to the internal DHA kinase-encoding gene sequence, so that PCR products of the expected size verify the presence of the inserted gene and the expected changes to the chromosomal DNA. In this way, the integrity of the edges of the modification, as well as the internal sequence may be verified.

In wild-type E. coli and other bacteria which metabolize glycerol via the intermediate glycerol-3-phosphate, the metabolism of dihydroxyacetone (DHA) depends on its phosphorylation by proteins of the DHA regulon that interact with proteins of the phosphotransfer system (PTS) (FIG. 26).

The PTS system phosphorylates DHA to DHAP (dihydroxyacetonephosphate). DHAP is an intermediate of glycolysis, and since it is common to the pathway of glycerol metabolism, it connects glycerol metabolism with central bacterial metabolism. The PTS system is membrane-bound. Therefore, DHA that is formed by a soluble glycerol dehydrogenase, such as the E. coli glycerol dehydrogenase, encoded by gldA, must diffuse to the membrane before it can be converted to DHAP, at such time that it may enter central metabolism, subsequently yielding additional NADH and ATP as well as acetyl-CoA, all of which may be utilized by a recombinant biocatalyst enzyme or pathway.

The PTS-mediated phosphorylation requires PEP, phosphoenolpyruvate. PEP donates its high-energy phosphoryl group to enzyme I of the PTS, and then the enzyme known in the art as HPr, both of which are located in the cytoplasm. However, the protein which specifically binds DHA is a homolog of the canonical enzyme II of the PTS, consisting of subunits IIA, IIB, and IIC, of which IIC is located in the cell membrane. In general, these IIA, B, and C proteins can be monomers or linked together covalently. IIA and IIB are hydrophilic, while IIC is a six or eight segment transmembrane protein. The phosphoryl group is believed to be transferred from P-HPr to IIA, then to IIB, and finally onto the subsequently phosphorylated sugar, without IIC ever being phosphorylated.

The pathway of DHA utilization similar in both C. freundii and K. pneumoniae involves a single ATP-dependent enzyme that is soluble in the cytoplasm, and bears some similarity to enzyme II of the PTS. Recombinant expression in a microorganism with a PTS-based route of DHA utilization, such as E. coli and other bacteria, may alleviate one or more limitations noted previously, such as a requirement of PEP, and diffusion of DHA to the membrane (even if the DHA is formed within the cytoplasm).

By way of example, in one embodiment, the reactions of the pathway from glycerol to pyruvate are as follows:

Glycerol→Dihydroxyacetone+NADH (1)

Dihydroxyacetone→Dihydroxyacetone-Phosphate+ADP (2)

Dihydroxyacetone-Phosphate→Pyruvate+NADH+2 ATP (3)

Where the net reaction is as follows:

Glycerol+2NAD⁺+2H⁺+1ADP→Pyruvate+1 ATP+2 NADH (4)

In one embodiment, an NADH-dependent glycerol dehydrogenase GldA enzyme catalyzes reaction (1) and the enzyme DHA Kinase derived from C. freundii or from K. pneumoniae catalyzing reaction (2). (see FIG. 26).

In one embodiment, the genes glpK (encoding glycerol kinase) and glpD (encoding G3P dehydrogenase) are deleted from a host microorganism's genome, and gldA (encoding an NADH-linked glycerol dehydrogenase) and a PEP (phosphoenolpyruvate)-dependent dihydroxyacetone (DHA) kinase emerge as the active route of glycerol degradation. In one embodiment, the host organism metabolizes glycerol through a conversion pathway that proceeds via a PEP-dependent PTS (phosphotransfer system) conversion of DHA to DHAP. In these hosts, by expressing the soluble DHA kinase of either Citrobacter freundii, Klebsiella pneumoniae or Saccharomyces cerevisiae recombinantly, limitations of native DHA utilization pathways requiring PEP and diffusion of the DHA to the cell's membrane may be overcome. DHAP may thereby be more efficiently available to the cell. Hence the subsequent metabolites of DHAP metabolism, such as acetyl-CoA, and NAD(P)H equivalents that may be utilized by the cell for biocatalysis, be they native or heterologously expressed enzymes, may also be more efficiently available to the cell.

Expression of a functional glycerol utilization pathway as herein described is expected to increase the moles of NADH per mole of glycerol. Specifically the moles of NADH per mole of glycerol may be increased to up to two moles of NADH per mole of glycerol. Thus, expression of a functional glycerol utilization pathway as herein described is expected to increase the moles of NADH available to the n-butanol pathway to 1.25, 1.5, 1.75, 2, and therefore to achieve balancing of an n-butanol pathway in a microorganism. As exemplified in example 4, GEVO926 produces about two moles of NADH per mole of glycerol. Expression of an n-butanol production pathway in a microorganism expressing a a functional glycerol utilization pathway as described supra may result in n-butanol yields of greater than 1.4% if the n-butanol production pathway can compete with endogenous fermentative pathways.

In certain embodiments, a recombinant microorganism herein described that express a heterologous enzyme for the production of n-butanol and in particular an NADH dependent heterologous pathway for the production of n-butanol such as the n-butanol pathway, is further engineered to inactivate a competing pathway and to balance NADH production and consumption in the microorganism with respect to the production of n-butanol.

In particular, in some embodiments, inactivation of lactate dehydrogenase and related conversion of pyruvate to lactate in addition to engineering for the microorganism for supplying sufficient NADH to the n-butanol production pathway by activating and in particular overexpressing Fdh, by activating an anerobically active Pdh, or by utilizing glycerol as the carbon source is expected to increase the n-butanol yield to about 5% of theoretical. In those embodiments, most of the carbon may still be diverted into ethanol. In particular, as exemplified in example 27, the n-butanol yield of GEVO1082 (and engineered to delete the gene coding for lactate dehydrogenase is expected to be about 5% of theoretical.

In some embodiments, in recombinant microorganism wherein alcohol dehydrogenase and the related conversion of acetyl-CoA to ethanol is inactivated, the activation, and in particular overexpression, of an NADH-dependent Fdh in addition to inactivation of competing metabolic pathways is expected to further increase the n-butanol yield to at least about 30%, 35%, 40%, 50%, 60%, 70%, 80%, 90%, and 95% with respect to theoretical, depending on the competing pathways that are inactivated in the microorganism. In particular, as exemplified in Example 18, the n-butanol yield expected by a recombinant microorganism such as of GEVO1083 expressing Fdh and having inactivated lactate dehydrogenase, alcohol dehydrogenase and fumarate reductase is about 42% higher compared to the strain not expressing Fdh (pGV1281 of Example 18). Fdh as expressed from a similar expression system as pGV1281 in GEVO1034 only resulted in three moles of NADH per mole of glucose which indicates that Fdh expression leads to an increase in NADH availability. However, this increase is not sufficient to allow balancing of the n-butanol pathway, thus limiting the expected yield to about 35%.

In some embodiments, wherein alcohol dehydrogenase and the related conversion of acetyl-CoA to ethanol is inactivated, the activation, and in particular the expression of an anaerobically active Pdh in addition to the inactivation of competing metabolic pathways is expected to further increase the n-butanol yield to at least about 30%, 35%, 40%, 50%, 60%, 70%, 80%, 90% and 95% of theoretical, depending on the competing pathways that are inactivated in the microorganism. In particular, as exemplified in example 23, the n-butanol yield of a recombinant microorganism such as GEVO1510, expressing Pdh under anaerobic conditions and having inactivated lactate dehydrogenase, alcohol dehydrogenase, fumarate reductase, methylglyoxal synthase and acetate kinase is expected to be about 73% of theoretical.

In some embodiments, wherein alcohol dehydrogenase and the related conversion of acetyl-CoA to ethanol is inactivated, the activation and in particular expression of a functional Fdh in addition to inactivation of competing metabolic pathways is expected to further increase the n-butanol yield to at least about 30%, 35%, 40%, 50%, 60%, 70%, 80%, 90% and 95% of theoretical, depending on the competing pathways that are inactivated in the microorganism. In particular, as exemplified in example 27, the n-butanol yield of a recombinant microorganism such as GEVO1507 (E. coli W3110, ΔldhA, ΔadhE, Δfrd, ΔackA, ΔmgsA) expressing Fdh and having inactivated lactate dehydrogenase, alcohol dehydrogenase, fumarate reductase, methylglyoxal synthase and acetate kinase, is expected to be about 70% of theoretical

In some embodiments, wherein alcohol dehydrogenase and the related conversion of acetyl-CoA to ethanol is inactivated, the activation and particular expression of a functional glycerol utilization pathway in addition to inactivation of competing metabolic pathways is expected to increase the n-butanol yield to levels of at least 50% 60%, 70%, 80%, 90% and 95% of theoretical, depending on the competing pathways that are inactivated in the microorganism. In particular, as exemplified in example the n-butanol yield of an E. coli W3110, ΔldhA, ΔadhE, Δndh, Δfrd, ΔackA, ΔmgsA) utilizing glycerol as a carbon source, and having inactivated lactate dehydrogenase, alcohol dehydrogenase, fumarate reductase, methylglyoxal synthase and acetate kinase is expected to be about 70% of theoretical.

In some embodiments, inactivation of an alcohol dehydrogenase that converts acetyl-CoA to ethanol in addition to engineering the microorganism for supplying sufficient NADH to the n-butanol production pathway by activating and in particular overexpressing Fdh, activating an anerobically active Pdh, or by utilizing glycerol as the carbon source is expected to increase the n-butanol yield to at least about 40% of theoretical. In particular, as exemplified in example 27 the n-butanol yield of GEVO1084 engineered to delete the gene coding for alcohol dehydrogenase, is expected to be about 40% of theoretical.

In some embodiments, the inactivation of lactate dehydrogenase and alcohol dehydrogenase and of the related conversion of pyruvate to lactate and acetyl-CoA to ethanol, respectively, in addition to supplying sufficient NADH to the n-butanol production pathway by activating and in particular overexpressing Fdh, activating an anerobically active Pdh, or by utilizing glycerol as the carbon source is expected to increase the n-butanol yield to about 50% of theoretical. In particular, as exemplified in example 27 the n-butanol yield of GEVO1084, (engineered to delete the gene coding for alcohol dehydrogenase and lactate dehydrogenase is expected to be about 50% of theoretical.

In some embodiments, the inactivation of a lactate dehydrogenase, alcohol dehydrogenase, and fumarate reductase, and of the related conversions of pyruvate to lactate, acetyl-CoA to ethanol, and fumarate to succinate respectively, in addition to engineering the microorganisms for supplying sufficient NADH to the n-butanol production pathway by acticating and in particular overexpressing Fdh, by activating an anerobically active Pdh, or by utilizing glycerol as the carbon source is expected to increase the n-butanol yield to about 55%. As exemplified in example 27 the n-butanol yield of a recombinant microorganism such as GEVO1508, ((engineered to delete the gene coding for alcohol dehydrogenase, lactate dehydrogenase and fumarate reductase is expected to be about 55% of theoretical.

In some embodiments, inactivation of a lactate dehydrogenase, alcohol dehydrogenase, fumarate reductase, and methylglyoxal synthase and of the related conversions of pyruvate to lactate, acetyl-CoA to ethanol, fumarate to succinate, and dihydroxy-acetone phosphate to methylglyoxal, respectively, in addition to engineering the microorganism for supplying sufficient NADH to the n-butanol production pathway by activating and in particular overexpressing Fdh, by activating an anerobically active Pdh, or by utilizing glycerol as the carbon source may increase the n-butanol yield to about 60%. In particular, as exemplified in example 27 the n-butanol yield of a recombinant microorganism such as GEVO1509, engineered to delete the genes coding for alcohol dehydrogenase, lactate dehydrogenase, fumarate reductase and methylglyoxal synthase, is expected to be about 60% of theoretical.

In some embodiments, inactivation of a lactate dehydrogenase, alcohol dehydrogenase, fumarate reductase, and acetate kinase and of the related conversion of pyruvate to lactate, acetyl-CoA to ethanol, fumarate to succinate, and acetyl-phosphate to acetate, respectively, in addition to engineering the microorganism for supplying sufficient NADH to the n-butanol production pathway by activating and in particular overexpressing Fdh, by activating an anerobically active Pdh, or by utilizing glycerol as the carbon source is expected to increase the n-butanol yield to about 65% of theoretical. As exemplified in example 27 the n-butanol yield of a recombinant microorganism such as GEVO1085, engineered to delete the gene coding for alcohol dehydrogenase, lactate dehydrogenase, fumarate reductase, and acetate kinase is expected to be about 65% of theoretical.

In some embodiments, inactivation of a lactate dehydrogenase, alcohol dehydrogenase, fumarate reductase, acetate kinase and methylgloxal synthase and of the related conversion of pyruvate to lactate, acetyl-CoA to ethanol, fumarate to succinate, acetyl-phosphate to acetate, and dihydroxy-acetone phosphate to methylglyoxal, respectively, in addition to engineering the microorganism for supplying sufficient NADH to the n-butanol production pathway by activating and in particular overexpressing Fdh, by activating an anerobically active Pdh, or by utilizing glycerol as the carbon source may increase the n-butanol yield to about 70%. In particular, as exemplified in example 27 the n-butanol yield of a recombinant microorganism such as GEVO1507, (engineered to delete the genes coding for alcohol dehydrogenase, lactate dehydrogenase, fumarate reductase, methylglyoxal synthase and acetate kinase is expected to be about 70% of theoretical.

Accordingly, in certain embodiments recombinant microorganisms herein disclosed includes recombinant microorganisms such as strains and derivatives thereof such as GEVO788, GEVO789, GEVO800, GEVO801, GEVO802, GEVO803, GEVO804, GEVO805, GEVO817, GEVO818, GEVO821, GEVO822, GEVO1054, GEVO1084, GEVO1085, GEVO1083, GEVO1493, GEVO1494, GEVO1495, GEVO1496, GEVO1497, GEVO1498, GEVO01499, GEVO1500, GEVO1501, GEVO1502, GEVO1503, GEVO1504, GEVO1505, GEVO1507, GEVO1508, GEVO1509, GEVO1510, GEVO1511 Preferred microorganisms include GEVO 1495, and, GEVO 1505. Those microorganisms their production and use are further described in the example section.

In certain embodiments, the n-butanol yield can be further raised by engineering the n-butanol producing pathway to increase its efficiency. In particular, this in embodiments wherein one or more heterolologously-expressed biocatalysts are not be initially optimized for use as a metabolic enzyme inside a host microorganism. However, these enzymes can usually be improved for example by using evolutionary approaches.

For example, using the engineered microorganisms described above, which contain the most effective variant of a desired n-butanol-producing pathway, selective pressure may be appliced to obtain improved biocatalysts. In this approach, the n-butanol producing pathway is transformed into a suitable host microorganism wherein the growth rate depends upon the efficiency of the pathway, i.e. wherein, the n-butanol pathway is the only means of re-oxidizing NADH. Microorganisms may be identified from this library which exhibit a detectable increase in growth rate that is not due to formation of another fermentation product. Other fermentation products may be identified by analyzing the fermentation broth via analytical methods known to those of skill in the art. This process may be repeated iteratively.

For example, using the engineered E. coli strains described above, which contain the most effective variant of a desired n-butanol-producing pathway, directed evolution can be performed to obtain improved biocatalysts. In this approach, an enzyme, preferably the rate limiting enzyme of the n-butanol producing pathway is mutated using methods known to those of skill in the art. The library of mutated genes is incorporated into the n-butanol producing pathway which is transformed into a suitable host microorganism wherein the growth rate depends upon the efficiency of the pathway, i.e. wherein, the n-butanol pathway is the only means of re-oxidizing NADH. Microorganisms may be identified from this library which exhibits an increased growth rate due to a beneficial mutation within the gene and not due to formation of another fermentation product. Other fermentation products may be identified by analyzing the fermentation broth via analytical methods known to those of skill in the art. This process may be repeated iteratively. For example, enzymes of the n-butanol producing pathway may be optimized by directed evolution according to methods of known to those of skilled in the art.

Metabolism of glucose through the heterologously expressed n-butanol pathway is the only way the engineered cells can generate ATP and also the only way they are able to maintain a steady NAD⁺/NADH ratio. Growth rates therefore depend on the rate of n-butanol formation. Selection for increased growth rate can easily be performed by serial dilution or chemostat evolution.

The same technique may be utilized to select for mutants with increased tolerance to n-butanol. N-butanol is a toxic substance to all microorganisms, mainly because it disrupts the cell membrane. E. coli has previously been engineered using an evolutionary strategy for increased ethanol resistance (Yomano, L. P. et al, 1998, Journal of Industrial Microbiology & Biotechnology, 20, 132-38). It is therefore expected that mutants displaying increased n-butanol resistance can be engineered in the same way.

Accordingly, in some embodiments, recombinant microorganism are described that are obtainable by providing a recombinant microorganism engineered to activate a heterologous pathway for conversion of a carbon source to n-butanol, and having a first growth rate that is dependent on the n-butanol production, the recombinant microorgranism also capable of producing butanol at a first production rate; identifying an enzyme in the heterologous pathway that is rate limiting with respect to the heterologous pathway; mutating said enzyme; contacting the recombinant microorganism comprising the mutated enzyme with a culture medium for a time and under condition to detect a second growth rate that is increased with respect to the first growth rate; and selecting the recombinant microorganism having the second growth rate, the selected recombinant microorganism capable of producing n-butanol at a second production rate, the second production rate greater than the first production rate.

Similar process may also be used to identify/isolate strains with a higher n-butanol yield per glucose metabolized.

In another embodiment, the microorganism is engineered to activate a metabolic pathway used to convert a carbon source to metabolic intermediates in the production of n-butanol or derivatives thereof. In particular in some embodiments, the recombinant microorganism is engineered to activate a metabolic pathway butyrate. In this pathway, genes are overexpressed to convert acetyl-CoA to butyryl-CoA. For example, genes encoding for thiolase, hydroxybutyryl-CoA-dehydrogenase, crotonase, and butyryl-CoA dehydrogenase may be expressed to convert acetyl-CoA to butyryl-CoA.

Butyryl-CoA is then converted to butyrate by two enzymes, phosphate butyryltransferase and butyrate kinase. Phosphate butyryltransferase, encoded for example by the gene ptb from C. acetobutylicum converts butyryl-CoA to butyryl-phosphate under release of CoA:

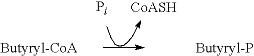

Butyryl-phosphate is then de-phosphorylated to butyrate by butyrate kinase, encoded for example by the gene buk from C. acetobutylicum under release of ATP:

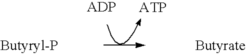

In an embodiment, E. coli is engineered to convert a carbon source to butyrate. In this pathway, genes encoding for thiolase, hydroxybutyryl-CoA dehydrogenase, crotonase, butyryl-CoA dehydrogenase, phosphate butyryltransferase, and butyrate kinase may be expressed to convert acetyl-CoA to butyrate.

In an embodiment, C. tyrobutyricum is used as a host organism to produce butyrate. In an embodiment, the C. tyrobutyricum utilizes a TER heterologous enzyme to catalyze the conversion of crotonyl-CoA to butyryl-CoA According to this embodiment, genes ack and pta encoding enzymes AK and PTA, involved in the competing acetate formation pathway, may be knocked-out, as described in X. Liu and S. T. Yang, Construction and Characterization of pta Gene Deleted Mutant of Clostridium tyrobutyricum for Butyric Acid Fermentation, Biotechnol. Bioeng., 90:154-166 (2005), Y. Yang, S. Basu, D. L. Tomasko, L. J. Lee, and S. T. Yang, which is incorporated herein by reference in its entirety.

Since only two moles of NADH are required to convert acetyl-CoA to butyrate, pyruvate formate lyase may be used to convert pyruvate to acetyl-CoA. Removal of competing pathways may increase the yield of the glucose to n-butyrate conversion and decrease the levels of by-products.

The removal of genes encoding for a lactate dehydrogenase, alcohol dehydrogenase, fumarate reductase, and acetate kinase which convert pyruvate to lactate, acetyl-CoA to ethanol, fumarate to succinate, and acetyl-phosphate to acetate, respectively, may decrease the production of lactate, ethanol, succinate and acetate and may increase the butyrate yield.

In another embodiment, the microorganism is engineered to convert a carbon source to a product wherein the product is a mixture of butyrate and n-butanol. The microorganism expresses genes for the conversion of acetyl-CoA to butyryl-CoA, genes for the conversion of butyryl-CoA to n-butanol, and genes for the conversion of butyryl-CoA to butyrate.

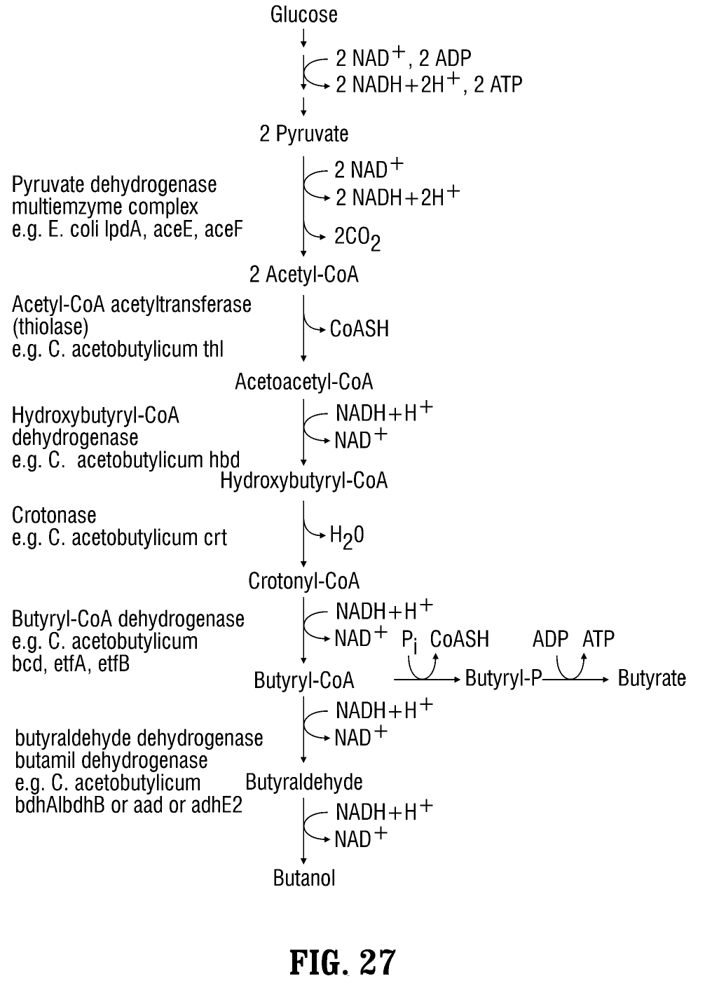

In an embodiment, genes expressed for the conversion of acetyl-CoA to butyryl-CoA may include those encoding thiolase, hydroxybutyryl-CoA dehydrogenase, crotonase, butyryl-CoA dehydrogenase, genes expressed for the conversion of butyryl-CoA to n-butanol may include those encoding butyraldehyde dehydrogenase and n-butanol dehydrogenase or a bifunctional butyraldehyde/butanol dehydrogenase, and genes for the conversion of butyryl-CoA to butyrate may include those encoding phosphate butyryltransferase, and butyrate kinase, as illustrated in FIG. 27.

The ratio of this mixture may depend on the availability of NADH since four molecules of NADH are required for the conversion of acetyl-CoA to n-butanol but only two molecules of NADH are required for the conversion of acetyl-CoA to butyrate. Therefore, to produce an equimolar mixture of butyrate and n-butanol, three molecules of NADH are generated per glucose converted to acetyl-CoA.

A method for producing n-butanol is further herein disclosed, the method comprising culturing a recombinant microorganism herein disclosed in a suitable culture medium.

In certain embodiments, the method further comprises isolating n-butanol from the culture medium. For example, n-butanol may be isolated from the culture medium by any of the art-recognized methods, such as pervaporation, liquid-liquid extraction, or gas stripping (see more details below).

In certain embodiments, the n-butanol yield is highest if the microorganism does not use aerobic or anaerobic respiration since carbon is lost in the form of carbon dioxide in these cases.

In certain embodiments, the microorganism produces n-butanol fermentatively under anaerobic conditions so that carbon is not lost in form of carbon dioxide.

The term “aerobic respiration” refers to a respiratory pathway in which oxygen is the final electron acceptor and the energy is typically produced in the form of an ATP molecule. The term “aerobic respiratory pathway” is used herein interchangeably with the wording “aerobic metabolism”, “oxidative metabolism” or “cell respiration”.

On the other hand, the term “anaerobic respiration” refers to a respiratory pathway in which oxygen is not the final electron acceptor and the energy is typically produced in the form of an ATP molecule, which includes a respiratory pathway wherein an organic or inorganic molecule other than oxygen (e.g. nitrate, fumarate, dimethylsulfoxide, sulfur compounds such as sulfate, and metal oxides) is the final electron acceptor. The wording “anaerobic respiratory pathway” is used herein interchangeably with the wording “anaerobic metabolism” and “anaerobic respiration”.

“Anaerobic respiration” has to be distinguishe by “fermentation”. In “fermentation”, NADH donates its electrons to a molecule produced by the same metabolic pathway that produced the electrons carried in NADH. For example, in one of the fermentative pathways of E. coli, NADH generated through glycolysis transfers its electrons to pyruvate, yielding lactate.

A microorganism operating under fermentative conditions can only metabolize a carbon source if the fermentation is “balanced.” A fermentation is said to be “balanced” when the NADH produced during the oxidation reactions of the carbon source equal the NADH utilized to convert acetyl-CoA to fermentation end products. Only under these conditions is all the NADH recycled. Without recycling, the NADH/NAD⁺ ratio becomes imbalanced which leads the organism to ultimately die unless alternate metabolic pathways are available to maintain a balance NADH/NAD⁺ ratio. According to White, 2000 #168, “a written fermentation is said to be ‘balanced’ when the hydrogens produced during the oxidations equal the hydrogens transferred to the fermentation end products. Only under these conditions is all the NADH and reduced ferredoxin recycled to oxidized forms. It is important to know whether a fermentation is balanced, because if it is not, then the overall written reaction is incorrect.

Anaerobic conditions are preferred for a high yield n-butanol producing microorganisms.

In some embodiments, a method for generating a recombinant microorganism herein disclosed, comprises: (1) generating a library of recombinant microorganisms by: (a) introducing into counterpart wild-type microorganisms one or more heterologous DNA sequence(s) encoding one or more polypeptide(s) capable of utilizing NADH to convert acetyl-CoA and one or more metabolic intermediate(s) of a n-butanol-producing pathway, (b) deleting from the genome of the counterpart wild-type microorganisms one or more endogenous DNA sequence(s) encoding an enzyme or enzymes which directly or indirectly consumes NADH and metabolic intermediates for (competing endogenous) anaerobic fermentation, wherein steps (a) and (b) are performed in either order, (2) selecting the recombinant microorganisms generated in step (1) for one or more recombinant microorganisms capable of growing anaerobically while producing n-butanol, wherein the counterpart wild-type microorganism is incapable of growing anaerobically while producing n-butanol.

In the method, one or more heterologous DNA sequence(s) encoding one or more polypeptide(s) capable of utilizing NADH to convert acetyl-CoA and one or more metabolic intermediate(s) of a n-butanol-producing pathway are introduced in a pre-selected host microorganism. Also in the host microorganism, one or more endogenous DNA sequence(s) encoding an enzyme or enzymes which compete with the n-butanol producing pathway for carbon and/or NADH are deleted to make available the carbon/NADH to the one or more polypeptide(s) for producing n-butanol or metabolic intermediates thereof. The recombinant microorganisms generated as such are then subject to selection pressure, so that those capable of growing faster anaerobically while producing n-butanol outgrow the population and are enriched for.

Optionally, the recombinant microorganisms may be randomly mutagenized through art-recognized means, such by addition of chemical mutagens such as ethyl methane sulfonate or N-methyl-N′-nitro-N-nitrosoguanidine to cultures. In addition, any n-butanol-producing microorganisms generated by the subject method may be subject to additional rounds of mutagenesis and selection so as to produce higher yield strains.

In certain embodiments, the method may also include steps to select for n-butanol-tolerant strains of microorganisms, either before or after the selection for recombinant microorganisms capable of surviving on produced n-butanol. For example, the method can include a step that selects for one or more recombinant microorganisms capable of growing anaerobically in a medium with at least about 0.1%, 0.2%, 0.3%, 0.4%, 0.5%, 0.6%, 0.7%, 0.8%, 0.9%, 1%, 1.2%, 1.5%, 1.8%, 2%, 3%, 4%, 5%, 6%, 7%, 8% or more of n-butanol, at a rate substantially the same as that of the counterpart wild-type microorganism growing in the medium without n-butanol.

In certain embodiments the method for producing n-butanol, comprises culturing a recombinant microorganism of the invention in a suitable culture medium under suitable culture conditions.

Suitable culture conditions depend on the temperature optimum, pH optimum, and nutrient requirements of the host microorganism and are known by those skilled in the art. These culture conditions may be controlled by methods known by those skilled in the art.

For example, E. coli cells are typically grown at temperatures of about 25° C. to about 40° C. and a pH of about pH4.0 to pH 8.0. Growth media used to produce n-butanol according to the present invention include common media such as Luria Bertani (LB) broth, EZ-Rich medium, and commercially relevant minimal media that utilize cheap sources of Nitrogen, mineral salts, trace elements and a carbon source as defined.

Fermentations may be performed under aerobic or anaerobic conditions, where anaerobic or microaerobic conditions are preferred during the n-butanol production phase.

In an embodiment, the fermentation consists of an aerobic phase and an anaerobic phase. Biomass is produced and the pathway enzymes are expressed under aerobic conditions more efficiently than under anaerobic conditions. The biotransformation, i.e. the conversion of glucose to n-butanol, occurs during the anaerobic phase.

Biomass production and protein expression are more efficient under aerobic conditions since the energy yield from a carbon source is higher. This allows for higher growth yield, growth rate, and protein expression rate. These advantages outweigh the cost of aerating the fermentation vessel.

The amount of 1-butanol produced in the fermentation medium can be determined using a number of methods known in the art, for example, high performance liquid chromatography or gas chromatography

In some embodiments, a method of producing n-butanol is provided which comprise culturing any of the recombinant microorganisms of the present disclosure for a time and under aerobic conditions or macroaerbic conditions, to produce a cell mass, in particular in the range of from about 1 to about 190 g dry cells liter, or preferably in the range of from about 1 to about 50 g dry cells liter⁻¹, then altering the culture conditions for a time and under conditions to produce one or more biofuels and/or biofuel precursors, in particular for a time and under conditions wherein the one or more biofuels are detectable in the culture, and recovering the one or more biofuels and/or biofuel precursors. In certain embodiments, the culture conditions are altered from aerobic or macroaerobic conditions to anaerobic conditions. In certain embodiments, the culture conditions are altered from aerobic conditions to macroaerobic conditions. In certain embodiments, the culture conditions are altered from aerobic conditions or macroaerobic conditions to microaerobic conditions.

The term “aerobic conditions” of a culture refers to conditions wherein the oxygen dissolved in the liquid fraction of the culture is 10% or higher relative to air saturation, taking into account the modifications due to equipment variability.

The term “microaerobic conditions” of a culture refers to conditions wherein the oxygen dissolved in the liquid fraction of the culture is from about 0.5% to about 5% relative to air saturation, taking into account the modifications due to equipment variability.

The term “macroaerobic conditions” of a culture refers to conditions wherein the oxygen dissolved in the liquid fraction of the culture is from about 5% to about 10% air saturation, taking into account the modifications due to equipment variability.

Productivity in batch reactors is often low due to downtime, long lag phase, and product inhibition. While downtime and lag phase can be eliminated using a continuous culture, the problem of product inhibition remains. This problem can be eliminated by the application of novel product removal techniques. In addition to continuous culture, fed-batch techniques can also be applied to the fermentation process. However, fermentation must be combined with a suitable product removal technique. Furthermore, application of immobilized cell culture and cell recycle reactors is known to increase reactor productivity 40-50 times as compared to batch reactors. An increase in productivity results in the reduction of process volume and reactor size, thus improving process economics.

One of the reasons for low reactor productivity is the low concentration of cells in the bioreactor. In a batch reactor, cell concentration over 3 g/L is rarely achieved. Therefore, reactor productivity can be improved by increasing the cell concentration in the reactor. An increased cell concentration can be achieved either by fixing cells onto supports or gel particles. Another option for increasing cell concentration is the application of a membrane that returns cells to the reactor while the aqueous solution containing the product permeates the membrane.

The following three sub-sections describe the different reactors that may be suitable for n-butanol production.

A) Batch, Fed-batch, and Free Cell Continuous Fermentation

The batch process is a simple method of fermentation for n-butanol production. During medium cooling, nitrogen or carbon dioxide is blown across the surface to keep the medium anaerobic. After inoculation, the medium is sparged with these gases to mix the inoculum.

Fed-batch fermentation is an industrial technique, which is applied to processes where a high substrate concentration is toxic to the culture. In such cases, the reactor is initiated in a batch mode with a low substrate concentration (noninhibitory to the culture) and a low medium volume, usually less than half the volume of the fermenter. As the substrate is used by the culture, it is replaced by adding a concentrated substrate solution at a slow rate, thereby keeping the substrate concentration in the fermenter below the toxic level for the culture. In this type of system, the culture volume increases in the reactor over time. The culture is harvested when the liquid volume is approximately 75% of the volume of the reactor.

Since n-butanol is toxic to the recombinant microorganisms, the fed-batch fermentation technique cannot be applied unless one of the novel product recovery techniques is applied for simultaneous separation of product. As a result of substrate reduction and reduced product inhibition, greater cell growth occurs and reactor productivity is improved.

The continuous culture technique can be used to improve reactor productivity and to study the physiology of the culture in a steady state. In such systems, the reactor is initiated in a batch mode and cell growth is allowed until the cells are in the exponential phase. As a precaution, fermentation is not allowed to enter the stationary phase because accumulation of n-butanol would kill the cells. While the cells are in the exponential phase, the reactor is fed continuously with the medium and a product stream is withdrawn at the same flow rate as the feed, thus keeping a constant volume in the reactor. Running fermentation in this manner eliminates downtime, thus improving reactor productivity. Additionally, fermentation runs much longer than in a typical batch process.

In a continuous culture, a serious problem may exist, in that solvent production may not be stable for long periods and may ultimately decline over time with a concomitant increase in acid production. In a single stage continuous system, high reactor productivity may be obtained, but this occurs at the expense of low product concentration when compared to that achieved in a batch process.

B) Immobilized Cell Continuous Reactors

High cell concentrations result in high reactor productivity. Such systems are continuous where feed is introduced into a tubular reactor at the bottom with product escaping at the top. These systems are often non-mixing reactors where product inhibition is significantly reduced. To improve reactor productivity, cells may be immobilized onto clay brick particles by adsorption and achieve a higher reactor productivity, resulting in economic advantage.

C) Membrane Cell Recycle Reactors

Membrane cell recycle reactors are another option for improving reactor productivity. In such systems, the reactor is initiated in a batch mode and cell growth is allowed. Before reaching the stationary phase, the fermentation broth is circulated through the membrane. The membrane allows the aqueous product solution to pass while retaining the cells. The reactor feed and product (permeate) removal are continuous and a constant volume is maintained in the reactor. In such cell recycle systems, cell concentrations of over 100 g/L can be achieved. However, to keep the cells productive, a small bleed should be withdrawn (<10% of dilution rate) from the reactor.

A) Distillation

The cost of recovering n-butanol by distillation is high because its concentration in the fermentation broth is low due to product inhibition. In addition to low product concentration, the boiling point of n-butanol is higher than that of water (118° C.). The usual concentration of total solvents in the fermentation broth is 18-33 g/L (using starch or glucose) of which n-butanol is only about 13-18 g/L. This makes n-butanol recovery by distillation energy intensive. A tremendous amount of energy can be saved if the n-butanol concentration in the fermentation broth can be increased from 10 to 40 g/L.

To reduce the cost of n-butanol recovery, a number of recovery techniques have been investigated including in situ gas stripping, liquid-liquid extraction, and pervaporation. Details of these techniques have been described elsewhere (see Maddox, Biotechnol. & Genetic Eng. Revs. 7: 190, 1989; Groot et al., Process Biochem. 27: 61, 1992; incorporated herein by reference). These techniques can be applied for in situ n-butanol removal, thus removing n-butanol from the reactor simultaneously with its production. The objective is to prevent the concentration of n-butanol from exceeding the tolerance level of the culture. The product is subsequently recovered either by condensation (gas stripping or pervaporation) or by distillation (extraction).

B) Alternative Economically Feasible Technologies

Gas Stripping

Gas stripping is a simple technique for recovering n-butanol (acetone or ethanol) from the fermentation broth. Either nitrogen or the fermentation gases (CO₂ and H₂) are bubbled through the fermentation broth followed by passing the gas (or gases) through a condenser. As the gas is bubbled through the fermenter, it captures the solvents (e.g., n-butanol). The solvents then condense in the condenser and are collected in a receiver. Once the solvents are condensed, the gas is recycled back to the fermenter to capture more solvents. This process continues until all the sugar in the fermenter is utilized by the culture. In some cases, a separate stripper can be used to strip off the solvents followed by the recycling of the stripper effluent that is low in solvents. Gas stripping has been successfully applied to remove solvents from a variety of reactors.

To reduce substrate inhibition, fed-batch fermentation may be integrated with gas stripping. For this purpose, a reactor may be initiated with 100 g/L glucose. As the sugar is consumed by the culture, the used glucose is replaced by adding a known volume of concentrated (500 g/L) sugar solution. The level of sugar inside the reactor is kept below the toxic level, preferably less than 80 g/L. Cellular inhibition that is caused by the solvents is reduced by removing them by gas stripping.

Liquid-Liquid Extraction

Liquid-liquid extraction is another technique that can be used to remove solvents (e.g., n-butanol) from the fermentation broth. In this process, an extraction solvent is mixed with the fermentation broth. N-butanol are extracted into the extraction solvent and recovered by back-extraction into another extraction solvent or by distillation.

Some of the requirements for extractive n-butanol fermentation are:

1. Non-toxic to the producing organism

2. High partition coefficient for the fermentation products

3. Immiscible and non-emulsion forming with the fermentation Broth

4. Inexpensive and easily available extraction solvent

5. The extraction solvent can be sterilized and does not pose health hazards.

For example, corn oil may be used as the extraction solvent. Many extraction solvents for n-butanol has also been reported in the literature. Among them, oleyl alcohol appears to meet some of the above requirements.

Extractant toxicity is a major problem with extractive fermentations. To avoid the toxicity problem brought about by the extraction solvent, a membrane may be used to separate the extraction solvent from the cell culture. For example, in a continuous fermentation cell recycle system, the fermentation broth may be circulated through the membrane and the bacteria are returned to the fermenter while the permeate is extracted with decanol to remove the n-butanol.

Another approach for reducing the toxicity and improving the partition coefficient has been to mix a high partition coefficient, high toxicity extractant with a low partition coefficient, low toxicity extractant. The resultant mixture is an extractant with an overall high partition coefficient and low toxicity. Oleyl alcohol may be used for this purpose.

Pervaporation

Pervaporation is a membrane-based process that is used to remove solvents from fermentation broth by using a selective membrane. The liquids or solvents diffuse through a solid membrane, leaving behind nutrients, sugar, and microbial cells. The concentration of solvents across the membrane depends upon membrane composition and membrane selectivity, which is a function of feed solvent concentration.

For example, a liquid membrane containing oleyl alcohol may be supported on a flat sheet of microporous polypropylene 25 mm thick. The liquids that diffused through the membrane show a selectivity of 180 as compared to the selectivity of a silicone membrane of approximately 45. It is estimated that if this pervaporation membrane is used as a pretreatment process for n-butanol separation, the energy requirements would be only 10% of that required by conventional distillation.

To develop a stable membrane having a high degree of selectivity, silicalite, an adsorbent, may be included in a silicone membrane. This may improve the selectivity level of the silicone-silicalite membrane. The working life of the membrane is several years. The membrane may be used with both n-butanol model solutions and fermentation broths.

### EXAMPLES

The present disclosure is also illustrated in the following examples, which are provided by way of illustration and are not intended to be limiting.

Certain strains, mentioned in the disclosure and in particular described in the following examples are listed in Table 1.

TABLE 1
Strains

| Strain | Genotype |
|---|---|
| GEVO709 (E. coli | E. coli B, gal-151, met-100, [malB + (LamS)], hsdR11, Δ46 |
| WA837) CGSC 90266 | |
| GEVO768 | E. coli W3110, attB::(Sp⁺ lacIq⁺ tetR⁺) |
| E. coli DHSα | E. coli F⁻ endA1 glnV44 thi-1 recA1 relA1 gyrA96 deoR nupG |
| | Φ80dlacZΔM15 Δ(lacZFA-argF)U169, hsdR17(r_{K}⁻ m_{K}⁺), λ− |
| GEVO788 | E. coli W3110, ΔldhA |
| GEVO789 | E. coli WA837, ΔldhA |
| GEVO800 | E. coli W3110, ΔadhE |
| GEVO801 | E. coli W3110, ΔpoxB |
| GEVO802 | E. coli W3110, ΔfocA-pflB |
| GEVO803 | E. coli WA837, ΔadhE |
| GEVO804 | E. coli WA837, ΔpoxB r |
| GEVO805 | E. coli WA837, ΔfocA-pflB |
| GEVO817 | E. coli W3110, ΔackA |
| GEVO818 | E. coli W3110, Δfrd |
| GEVO821 | E. coli WA837, ΔackA |
| GEVO822 | E. coli WA837, Dfrd |
| GEVO914 | E. coli W3110, Δldh, ΔpoxB, Δfrd |
| GEVO916 | E. coli W3110, ΔglpD |
| GEVO917 | E. coli W3110, ΔglpK |
| GEVO922 | E. coli W3110, ΔglpK, ΔglpD |
| GEVO926 | E. coli W3110, ΔglpD, ΔglpK* |
| GEVO927 | E. coli W3110, ΔglpD, ΔglpK*, pGV1010 |
| GEVO954 DSMZ 615 | E. coli B |
| GEVO992 | E. coli W3110, ΔldhA, Δfrd |
| GEVO1005 (E. coli | E. coli F-L-rph-1 INV(rrnD, rrnE) |
| W3110) DSMZ 5911 | |
| GEVO1007 | E. coli W3110, Δldh, ΔpoxB, ΔackA |
| GEVO1034 | E. coli W3110, ΔfdhF |
| GEVO1039 | E. coli W3110, Δndh, Δldh, ΔadhE, ΔfocA-pflB, Δfrd, |
| | Δfnr, attB::(Sp+ lacIq+ tetR+) |
| GEVO1043 | E. coli W3110, Δndh, Δldh, ΔadhE, ΔfocA-pflB, ΔackA, Δfrd, |
| | Δfnr, attB::(Sp+ lacIq+ tetR+) |
| GEVO1044 | E. coli W3110, Δndh, ΔpoxB, ΔackA, Δ(fnr-ldhA), attB::(Sp+ |
| | lacIq+ tetR+) |
| GEVO1047 | E. coli W3110, ΔldhA, Δfrd, attB::(Sp+ lacIq+ tetR+) |
| GEVO1054 | E. coli W3110, ΔadhE, attB::(Sp+ lacIq+ tetR+) |
| GEVO1082 | E. coli W3110, ΔldhA, attB::(Sp+ lacIq+ tetR+) |
| GEVO1083 | E. coli W3110, Δndh, Δldh, ΔadhE, Δfrd, attB::(Sp+ lacIq+ |
| | tetR+) |
| GEVO1084 | E. coli W3110, ΔldhA, ΔadhE, attB::(Sp+ lacIq+ tetR+) |
| GEVO1085 | E. coli W3110, ΔldhA, ΔadhE, Δfrd, ΔackA, attB::(Sp+ lacIq+ |
| | tetR+) |
| GEVO1086 | E. coli W3110, ΔldhA, Δfrd, ΔackA, attB::(Sp+ lacIq+ tetR+) |
| GEVO1121 | E. coli W3110, Δndh, Δldh, ΔadhE, Δfrd, ΔmgsA, attB::(Sp+ |
| | lacIq+ tetR+) |
| GEVO1137 | E. coli W3110, Δndh, Δldh, ΔadhE, Δfrd, attB::(Sp+ lacIq+ |
| | tetR+), ΔackA |
| GEVO1200 | E. coli W3110, ΔldhA, ΔackA |
| GEVO1227 | E. coli W3110, ΔlpdA |
| GEVO1228 | E. coli WA837, ΔlpdA |
| GEVO1229 | E. coli W3110, ΔlpdA::lpdAmut |
| GEVO1230 | E. coli W3110, ΔlpdA::lpdAN |
| GEVO1470 | E. coli W3110, Δndh, Δldh, ΔadhE, Δfrd, attB::(Sp+ lacIq+ |
| | tetR+)* |
| GEVO1493 | E. coli W3110, ΔldhA |
| GEVO1494 | E. coli W3110, ΔldhA, ΔackA |
| GEVO1495 | E. coli W3110, Δldh, ΔpoxB, ΔackA, ΔadhE |
| GEVO1496 | E. coli W3110, Δldh, ΔpoxB, ΔackA, ΔadhE, ΔfocApflB |
| GEVO1497 | E. coli W3110, ΔpflDC |
| GEVO1498 | E. coli W3110, Δldh, ΔpoxB, ΔackA, ΔadhE, ΔfocApflB, ΔpflDC |
| GEVO1499 | E. coli W3110, Δldh, ΔpoxB, ΔackA, ΔadhE, ΔfocApflB, Δfrd |
| GEVO1500 | E. coli W3110, Δldh, ΔpoxB, ΔackA, ΔfocApflB |
| GEVO1501 | E. coli W3110, Δldh, ΔpoxB, ΔackA, ΔpflDC |
| GEVO1502 | E. coli W3110, Δldh, ΔpoxB, ΔackA, ΔpflDC, Δfrd |
| GEVO1503 | E. coli W3110, Δfnr |
| GEVO1504 | E. coli W3110, Δldh, ΔpoxB, ΔackA, ΔpflDC, Δfnr |
| GEVO1505 | E. coli W3110, Δldh, ΔpoxB, ΔackA, ΔpflDC, Δfnr, attB::(Sp+ |
| | lacIq+ tetR+) |
| GEVO1507 | E. coli W3110, ΔldhA, ΔadhE ΔackA, ΔmgsA, ΔackA, Δfrd, |
| | attB::(Sp+ lacIq+ tetR+) |
| GEVO1508 | E. coli W3110, Δldh, ΔadhE, Δfrd, attB::(Sp+ lacIq+ tetR+) |
| GEVO1509 | E. coli W3110, Δldh, ΔadhE, Δfrd, ΔmgsA attB::(Sp+ lacIq+ |
| | tetR+) |
| GEVO1510 | E. coli W3110, Δldh, ΔadhE, ΔpflB, ΔpflDC, Δfrd, ΔmgsA |
| | attB::(Sp+ lacIq+ tetR+)* |
| GEVO1511 | E. coli W3110, Δldh, ΔadhE, ΔpflB, ΔpflDC, Δfrd, ΔmgsA |
| | attB::(Sp+ lacIq+ tetR+) |

*strain evolved

Certain plasmids mentioned in the disclosure and used in the experiments described in the following examples, are listed in the following Table 2.

TABLE 2
Plasmids

| | | |
|---|---|---|
| pGV772 | PltetO1, KanR, colE1 | SEQ ID NO: 17 |
| pGv1010 | PLlacOI::AA3, Cm^{R}, | SEQ ID NO: 18 |
| | colE1 | |
| pGV1035 | PLlacO1::thl(C.a.), CmR, | SEQ ID NO: 19 |
| | colE1 | |
| pGV1037 | PLlacO1::hbd(C.a.), Cm^{R}, | SEQ ID NO: 20 |
| | colE1 | |
| pGV1039 | PLlacO1::thl(B.f.), Cm^{R}, | SEQ ID NO: 21 |
| | colE1 | |
| pGV1040 | PLlacO1::crt(B.f.), Cm^{R}, | SEQ ID NO: 22 |
| | colE1 | |
| pGV1041 | PLlacO1::hbd(B.f.) Cm^{R}, | SEQ ID NO: 23 |
| | colE1 | |
| pGV1049 | PLlacO1::crt(C.b.), Cm^{R}, | SEQ ID NO: 24 |
| | colE1 | |
| pGV1050 | PLlacO1::hbd(C.b.), Cm^{R}, | SEQ ID NO: 25 |
| | colE1 | |
| pGV1052 | PLlacOI::bcd::etfB::etfA | SEQ ID NO: 26 |
| | (M. elsdenii), Cm^{R}, | |
| | colE1 | |
| pGV1054 | PLlacO1::thl(C.a.), Cm^{R}, | SEQ ID NO: 27 |
| | colE1 | |
| pGV1088 | PLlacOI::bcd::etfB::etfA | SEQ ID NO: 28 |
| | (C. acetobutylicum), Cm^{R}, | |
| | colE1 | |
| pGV1094 | PLlacO1::crt(C.a.), Cm^{R}, | SEQ ID NO: 29 |
| | colE1 | |
| pGV1111 | PLlacO1, Cm^{R}, | SEQ ID NO: 30 |
| | colE1 | |
| pGV1113 | PLlacO1::TER(E.g.), Cm^{R}, | SEQ ID NO: 31 |
| | colE1 | |
| pGV1117 | PLlacO1::TER(A.h.), Cm^{R}, | SEQ ID NO: 32 |
| | colE1 | |
| pGV1154 | PLlacO1::hbd(C.a.co), Cm^{R}, | SEQ ID NO: 33 |
| | colE1 | |
| pGV1188 | PLlacO1::thl(C.a.co), Cm^{R}, | SEQ ID NO: 34 |
| | colE1 | |
| pGV1189 | PLlacO1::crt(C.a.co), Cm^{R}, | SEQ ID NO: 35 |
| | colE1 | |
| pGV1190 | PLlacO1::thl(C.a.co)::adhE2 | SEQ ID NO: 36 |
| | (C.a.)::crt(C.a.co)::hbd | |
| | (C.a.co), Amp^{R}, | |
| | p15A | |
| pGV1191 | PLlacO1::thl(C.a.co)::adhE2 | SEQ ID NO: 37 |
| | (C.a.co)::crt(C.a.co)::hbd | |
| | (C.a.co), Amp^{R}, | |
| | p15A | |
| pGV1248 | PLlacO1::fdh(C.b.), Cm^{R}, | SEQ ID NO: 38 |
| | colE1 | |
| pGV1252 | PLlacO1::MCS, Cm^{R}, colE1 | SEQ ID NO: 39 |
| pGV1272 | PLlacO1::TER(E.g.), Cm^{R}, | SEQ ID NO: 40 |
| | colE1 | |
| pGV1278 | PLtetO1::lpdAmut(E.c.), | SEQ ID NO: 41 |
| | Kan^{R}, colE1 | |
| pGV1279 | PLtetO1::lpdAwt(E.c.), Kan^{R}, | SEQ ID NO: 42 |
| | colE1 | |
| pGV1281 | PLlacO1::TER(E.g.)::fdh(C.b.), | SEQ ID NO: 43 |
| | Cm^{R}, colE1 | |
| pGV1300 | TER (Bulkholderia | Contains SEQ |
| | cenocepacia) | ID NO: 44 |
| pGV1301 | TER (Coxiella burnetti) | Contains SEQ |
| | | ID NO: 45 |
| pGV1302 | TER (Reinekea) | Contains SEQ |
| | | ID NO: 46 |
| pGV1303 | TER (Shewanella woodyi) | Contains SEQ |
| | | ID NO: 47 |
| pGV1304 | TER (Treponema denticola) | Contains SEQ |
| | | ID NO: 48 |
| pGV1305 | TER (Xanthomonas orycae | Contains SEQ |
| | orycae KACC1033) | ID NO: 49 |
| pGV1306 | TER (Yersinia pestis) | Contains SEQ |
| | | ID NO: 50 |
| pGV1307 | TER (alpha proteobacterium | Contains SEQ |
| | HTCC2255) | ID NO: 51 |
| pGV1308 | TER (Cytophaga | Contains SEQ |
| | hutchinsonii) | ID NO: 52 |
| pGV1309 | TER (Vibrio Ex25) | Contains SEQ |
| | | ID NO: 53 |
| pGV1340 | PLlacO1::TER(Bulkholderia | SEQ ID NO: 54 |
| | cenocepacia), Cm^{R}, colE1 | |
| pGV1341 | PLlacO1::TER (Coxiella | SEQ ID NO: 55 |
| | burnetti), Cm^{R}, colE1 | |
| pGV1342 | PLlacO1::TER (Reinekea), | SEQ ID NO: 56 |
| | Cm^{R}, colE1 | |
| pGV1343 | PLlacO1::TER (Shewanella | SEQ ID NO: 57 |
| | woodyi), Cm^{R}, colE1 | |
| pGV1344 | PLlacO1::TER (Treponema | SEQ ID NO: 58 |
| | denticola), Cm^{R}, colE1 | |
| pGV1345 | PLlacO1::TER (Xanthomonas | SEQ ID NO: 59 |
| | orycae orycae KACC1033), | |
| | Cm^{R}, colE1 | |
| pGV1346 | PLlacO1::TER (Yersinia | SEQ ID NO: 60 |
| | pestis), Cm^{R}, | |
| | colE1 | |
| pGV1347 | PLlacO1::TER (alpha | SEQ ID NO: 61 |
| | proteobacterium HTCC2255), | |
| | Cm^{R}, colE1 | |
| pGV1348 | PLlacO1::TER (Cytophaga | SEQ ID NO: 62 |
| | hutchinsonii), Cm^{R}, | |
| | colE1 | |
| pGV1349 | PLlacO1::TER (Vibrio Ex25), | SEQ ID NO: 63 |
| | Cm^{R}, colE1 | |
| pGV1435 | PLlacO1::TER (Treponema | SEQ ID NO: 64 |
| | denticola), Cm^{R}, | |
| | colE1 | |
| pGV1563 | PLlacOI::DHA kinase | SEQ ID NO: 65 |
| | (Citrobacter freundii), | |
| | kanR, SC101 | |
| pGV1569 | Ptac, Amp^{R}, colE1, | SEQ ID NO: 66 |
| pGV1582 | Ptac::fdh (C. boidinii), | SEQ ID NO: 67 |
| | Amp^{R}, ColE1, | |
| pGV1583 | Ptac::fdh (C. boidinii)::TER | SEQ ID NO: 68 |
| | (Treponema denticola), | |
| | Amp^{R}, ColE1, | |

Certain primers mentioned in the present disclosure and used in the experiments described in this section, are listed in the following Tables 3.

TABLE 3
Primers

| | | |
|---|---|---|
| Cac_th1F | AATTGAATTCTTATTATTTAGGAGGAGTAAAACAT | (SEQ ID NO:69) |
| Cac_th1R | AATTGGATCCTTAGTCTCTTTCAACTACGAGAGCT | (SEQ ID NO:70) |
| Cac_aadF | AATTGAATTCATATTTTAGAAAGAAGTGTATATTT | (SEQ ID NO:71) |
| Cac_aadR | AATTACGCGTTTAAGGTTGTTTTTTAAAACAATTTATATACA | (SEQ ID NO:72) |
| Cac_bdhF | AATTGAATTCATTAGATGCTTGTATTAAAATAATAA | (SEQ ID NO:73) |
| Cac_bdhR | AATTGGATCCTTACACAGATTTTTTGAATATTTGTA | (SEQ ID NO:74) |
| Cac_hbdF | AATTGAATTCATTGATAGTTTCTTTAAATTTAGGG | (SEQ ID NO:75) |
| Cac_hbdR | AATTGGATCCTTATTTTGAATAATCGTAGAAACCT | (SEQ ID NO:76) |
| Cac_crtF | AATTGAATTCCTATCTATTTTTGAAGCCTTCAATT | (SEQ ID NO:77) |
| Cac_crtR | AATTGGATCCAATATTTTAGGAGGATTAGTCATGGA | (SEQ ID NO:78) |
| Cac_bcdF | AATTGGTACCTTAATTATTAGCAGCTTTAACTTGAGC | (SEQ ID NO:79) |
| Cac_bcdR | AATTGGATCCAAAATTGAAGGCTTCAAAAATAGATAGGAG | (SEQ ID NO:80) |
| Cac_adhF | AATTGTCGACATTTTATAAAGGAGTGTATATAAATGAAAGTTAC | (SEQ ID NO:81) |
| Cac_adhR | TTAATCTAGATTAAAATGATTTTATATAGATATCCT | (SEQ ID NO:82) |
| glpDchk_F | CCGTGGGTGAAACAGTTCTT | (SEQ ID NO:83) |
| glpDchk_R | CGTAAGTGCGAGCGTAATGA | (SEQ ID NO:84) |
| glpKchk_F | AAAGCTCCACGCTGGTAGAA | (SEQ ID NO:85) |
| glpKchk_R | GTCACGCGTCTGATAAGCAA | (SEQ ID NO:86) |

### Example 1

### Removal of Competing Metabolic Pathways from Host Microorganism Genome

This example illustrates the construction of n-butanol production host strains. Competing pathways of the host organism are fermentative pathways that couple the oxidation of NADH to the production of compounds such as succinate, lactate, ethanol, carbon dioxide and hydrogen gas and pathways that compete for the carbon from the carbon source such as the acetate pathway and the production of formate.

The strains listed in Table 1 were obtained by deletion of genes in the bacterial genome. The genes were deleted using homologous recombination techniques. The gene deletions were transferred from strain to strain using phage P1 transduction. The gene deletions were combined by sequential deletion of individual genes.

Parent strains used for the metabolic engineering of GEVO1005 (E. coli W3110 (DSMZ 5911)) and E. coli B (DSMZ 613). For the transfer of genomic deletions, insertions and gene disruptions from E. coli K12 to E. coli B strain, E. coli WA837 (CGSC 90266) was used as an intermediate host. During strain construction, cultures were grown on Luria-Bertani (LB) medium or agar (Sambrook and Russel, Molecular Cloning, A Laboratory Manual. 3rd ed. 2001, Cold Spring Harbor, N.Y.: Cold Spring Harbor Laboratory Press). Unless stated otherwise, standard methods were used, such as transduction with phage P1, PCR, and sequencing (Miller, A short Course in Bacterial Genetics: A Laboratory Manual and Handbook for Escherichia coli and Related Bacteria. 1992, Cold Spring Harbor, N.Y.: Cold Spring Harbor Press; Sambrook and Russel, Molecular Cloning, A Laboratory Manual. 3 ed. 2001, Cold Spring Harbor, N.Y.: Cold Spring Harbor Laboratory Press). DNA for the insertion of genes and expression cassettes into the E. coli chromosome was constructed with splicing by overlap extension method (SOE) of Horton, Mol. Biotechnol. 3: 93-99, 1995. Chromosomal integrations and deletions were verified with the appropriate markers and by PCR analysis, or, in the case of integrations, by sequencing.

D-lactate Dehydrogenase (encoded by ldhA): Most of the gene coding for the lactate dehydrogenase in E. coli (ldhA) was deleted (nucleotides 11-898 were deleted). The resulting strains containing the deletion of ldhA are:

The deletion of ldhA was combined with the deletions of nuoA_N and ndh. GEVO914 was transduced with a P1 lysate prepared from GEVO788 and the resulting strain is designated GEVO915. For the construction of the corresponding E. coli B strain, GEVO916 is transduced with a P1 lysate prepared from GEVO789 and the transduced strain is designated GEVO917.

Acetate Kinase A (encoded by ackA): The gene coding for acetate kinase in E. coli (ackA) was disrupted with a deletion (nucleotides 29-1062 are deleted). The strains containing the deletion of ackA are GEVO817 and GEVO821.

The deletion of ackA is combined with the deletion of ldhA. GEVO1493, is transduced with a P1 lysate prepared from GEVO817 and the resulting strain is designated GEVO1494.

Pyruvate Oxidase (encoded by poxB): The gene coding for pyruvate oxidase in E. coli (poxB) was disrupted with a deletion in poxB (nucleotides 30-1600 were deleted). The resulting strains are GEVO801 and GEVO804.

The deletion of poxB is combined with the deletions of ldhA and ackA. GEVO1494, is transduced with a P1 lysate prepared from GEVO801 and the resulting strain is designated GEVO1007.

Acetaldehyde/alcohol Dehydrogenase (encoded by adhE): The gene coding for the alcohol dehydrogenase in E. coli (adhE) was disrupted with a deletion (nucleotides-308-2577 were deleted). The resulting strains are GEVO800 and GEVO803.

The deletion of adhE is combined with the deletion of ldhA, ackA and poxB. GEVO1007, is transduced with a P1 lysate prepared from GEVO800 and the resulting strain is designated GEVO1495. For the construction of the corresponding E. coli B strain GEVO1211 is transduced with a P1 lysate prepared from GEVO803 and the transduced strain is designated GEVO1212.

In Saccharomyces, pyruvate is converted to acetaldehyde by pyruvate decarboxylase. At least five independent NADH-dependent alcohol dehydrogenases are known that then reduce acetaldehyde to ethanol. These are ADH1, ADH2, ADH3, ADH4, and ADH5.

Pyruvate Formate Lyase (encoded by pflB): The gene coding for the pyruvate formate lyase in E. coli (pflB) was disrupted by the deletion of focA and pflB (nucleotides -69(focA)-2240(pflB) were deleted). The resulting strains are GEVO802 and GEVO805.

The deletion of pflB is combined with the deletions of ldhA, ackA, poxB, and adhE. The resulting strain GEVO1495 is transduced with a P1 lysate prepared from GEVO802 and the resulting strain is designated GEVO1496.

Pyruvate Formate Lyase 2 (encoded by pflDC): The gene coding for the pyruvate formate lyase 2 in E. coli (pflDC) was disrupted by the deletion of pflDC (nucleotides -69(pflD) -2240(pflC) were deleted). The resulting strains are GEVO2000 and GEVO2001.

The deletion of pflDC is combined with the deletions of ldhA, ackA, poxB, adhE, and pflB. The resulting strain GEVO1496 is transduced with a P1 lysate prepared from GEVO1497 and the resulting strain is designated GEVO1498.

Fumarate Reductase (encoded by frd): The genes coding for the fumarate reductase in E. coli (frdABCD) were disrupted with a deletion of frdABCD (nucleotides -86(frdA)-178(frdD) were deleted). The resulting strains are GEVO818 and GEVO822.

The deletion of frdABCD is combined with the deletions of ldhA, ackA, poxB, adhE and focA-pflB. GEVO1496, is transduced with a P1 lysate prepared from GEVO818 and the resulting strain is designated GEVO1499.

### Example 2

### (Prophetic) Recombinant E. Coli Engineered to Use a Reduced Carbon Source (Glycerol) to Balance a N-Butanol Producing Heterologous Pathway

One method to balance the n-butanol pathway in E. coli is to use glycerol as a carbon source. For growth on glycerol, the alternative glycerol degradation pathway that avoids the glycerol phosphate dehydrogenasecatalyzed step that feeds electrons into the quinone pool has to be active.

The alternative pathway can be activated by inactivating genes encoding glycerol kinase and glycerol-3-phosphate dehydrogenase. The pathway is made more efficient by expressing a DHA kinase from C. freundii, K. pneumonia, S. cerevisiae or other organisms. The expression of a DHA kinase avoids the phosphotransferase system (PTS)-coupled phosphorylation of DHA, which requires DHA to diffuse out of the cell and re-enter through the pts while being phosphorylated.

The gene encoding DHA kinase is cloned from C. freundii utilizing the polymerase chain reaction and primers appropriate to obtain linear double-stranded DNA of the complete gene. The gene is cloned into an expression plasmid that is compatible with the n-butanol pathway expression plasmids.

The resulting construct is pGV1563. GEVO926 (E. coli W3110 (F-L-rph-1 INV(rrnD, rrnE)), ΔglpD, ΔglpK) is transformed with pGV1191, and pGV1113 for the expression of the n-butanol pathway (Strain A) and GEVO 926 is transformed with pGV1191, pGV1113, and pGV1563 for expression of the n-butanol pathway and the expression of DHA kinase from C. freundii. Strain A (GEVO926, pGV1191, pGV1113) and Strain B (GEVO926, pGV1191, pGV1113, pGV1563) are compared by n-butanol bottle fermentation.

The strains A and B are grown aerobically in medium B (EZ-Rich medium containing 0.4% glycerol, 100 mg/L Cm, and 200 mg/L Amp, and 50 mg/L Kan) in tubes overnight at 37° C. and 250 rpm. 60 mL of Medium B in shake flasks is inoculated at 2% from the overnight cultures and the cultures are grown to an OD₆₀₀ of 0.6. The cultures are induced with 1 mM IPTG and 100 ng/mL aTc and are incubated at 30° C., 250 rpm for 12 h. 50 mL of the culture are transferred into anaerobic flasks and incubated at 30° C., 250 rpm for 36 h. Samples are taken at different time points and the cultures are fed with glucose and neutralized with NaOH if necessary. The samples are analyzed with GC and HPLC.

The results show that Strain A produces n-butanol with a yield of 60% and strain B produces n-butanol with a yield of 70%. This example shows that a production strain with a deletion of the native glycerol degradation pathway provides enough NADH to reach n-butanol yields higher that 50% of the theoretical yield. In addition these results show that the expression of DHA kinase increases the yield of n-butanol production from glycerol in such a glycerol pathway deletion strain.

### Example 3

### Production of a recombinant E. coli able metabolize glycerol via dihydroxyacetone and dihydroxyacetone phosphate

This example demonstrates the generation of a strain which converts glycerol to acetyl-CoA while generating two molecules of NADH per molecule of glycerol.

Strain GEVO1005 (E. coli W3110 (F-L-rph-1 INV(rrnD, rrnE))) was used as the parent strain. The genes glpD and glpK were deleted from the host's genome. The double knockout glpD glpK was constructed by P1 transduction. The resulting strain was GEVO922.

GEVO922 was subjected to an enrichment evolution protocol, since it showed very poor growth on minimal glycerol media, compared to the wild-type parent strain. During the 4-week course of this enrichment evolution, which began with 2.4×10¹² cells, glycerol was used as the carbon source and was fed every other day. Glycerol was fed to a final concentration of 2 mM, every other day, for the first 2 weeks, 1 mM for the third week, and 0.5 mM for the fourth and final week. At the end of this process, several mutants were isolated.

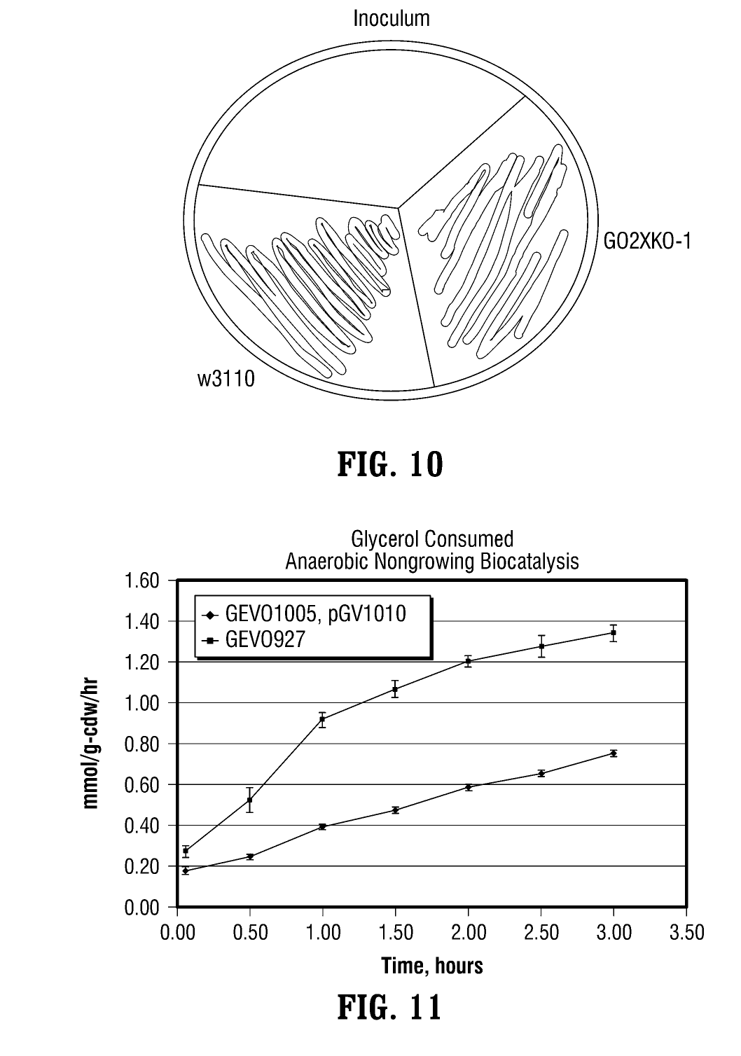

Consistent with the expected genotype, with glycerol as sole carbon and energy source, GEVO922, the glpD glpK double knockout, grew slowly compared to the parental, wild-type strain. Subsequent to the four week enrichment evolution, one clone (GEVO926) that grew fast on minimal M9 glycerol plates was selected for continued study. GEVO926 had a growth rate similar to wild-type levels, on minimal media plates with glycerol as carbon source (FIG. 10)

After the enrichment evolution process, the gene deletions in the evolved strain were verified by PCR, using the PCR primers listed in Table 4.

TABLE 4

PCR Primers Used to Verify the Maintenance of
Changes to Chromosomal DNA

| | | |
|---|---|---|
| Sequence | Primer | Description |

| | | |
|---|---|---|
| CCG TGG GTG | glpDchk_F | Primer binds upstream and outside of glpD |
| AAA CAG TTC TT | SEQ ID NO:83 | gene to verify gene knockout of glpD |
| CGT AAG TGC | glpDchk_R | Primer binds downstream and outside of |
| GAG CGT AAT GA | SEQ ID NO:84 | glpD gene to verify gene knockout of glpD |
| AAA GCT CCA CGC | glpKchk_F | Primer binds upstream and ouside of glpK |
| TGG TAG AA | SEQ ID NO:85 | gene to verify gene knockout of glpK |
| GTC ACG CGT CTG | glpKchk_R | Primer binds downstream and outside of |
| ATA AGC AA | SEQ ID NO:86 | glpK gene to verify gene knockout of glpK |

Finally, both wild-type GEVO1005 and the enrichment-evolved, double knockout, GEVO926, were transformed with pGV110, a plasmid containing the chloramphenicol antibiotic resistance genetic marker and the gene encoding an NADPH-dependent yeast ketoreductase/dehydrogenase, under control of a lac promoter. However, since GEVO1005 is a derivative of the E. coli K-12 strain, it only has a single lac repressor gene on the chromosome, and production of the ketoreductase in both strains is constitutive. No inducer was used in the growth of the biocatalytic cells, as it was shown that expression levels with and without inducer were about the same.

### Example 4

### Recombinant E. Coli Engineered to Use of a Reduced Carbon Source (Glycerol) to Balance a N-Butanol Producing Heterologous Pathway

This example demonstrates that an engineered microorganism converts one mole of glycerol to acetyl-CoA and yields two moles of NADH and meets the requirement with respect to NADH for utilizing glycerol to produce n-butanol using a balanced n-butanol production pathway. In contrast, a wild-type, unengineered and unmodified strain, only generates one mole of NADH.

The balanced n-butanol pathway requires four moles of NADH and two moles of acetyl-CoA for every mole of n-butanol produced. Redox balance of a pathway is critical to reaching the highest yields. The engineering described in Examples 2 and 3 effectively produces an E. coli biocatalyst that produces a total of two moles of NADH and one mole of acetyl-CoA for every mole of glycerol metabolized anaerobically under non-growing conditions; in contrast, the unengineered wild-type strain produces only one mole of NADH per acetyl-CoA generated anaerobically under non-growing conditions so it therefore cannot work as an efficient biocatalyst for n-butanol production using glycerol as a carbon source. The engineered E. coli produced as a result of Example 3, was verified to produce the metabolic intermediates required to function as a biocatalyst with a balanced n-butanol production pathway.

### Biocatalysis

GEVO1005 and GEVO926 were transformed with pGV1010 and plated on LB plates supplemented with 50 mg/mL chloramphenicol to ensure that cells retained the plasmid with chloramphenicol antibiotic resistance marker and the yeast AA3 ketoreductase-encoding gene. From single colonies three biological replicates of starter cultures of 3 mLs of M9Y+0.4% glycerol were inoculated for overnight growth in a shaking incubator at 37° C. and 250 rpm. Using 1.2 mLs of each starter culture as inoculum, a culture of 120 mLs of M9Y+0.4% glycerol was inoculated and grown to stationary phase at 37° C. and 250 rpm The cultures were harvested by centrifugation at 4000 g for 15 minutes, with OD₆₀₀ being measured at time of harvest. The cells were washed once with 60 mL of carbon source- and nitrogen-free media for biocatalysis (biocatalysis medium). This medium does not allow cell growth. The culture was centrifuged again at 4000 g for 15 minutes, and re-suspended in a volume of biocatalysis medium equal to 10 times the OD₆₀₀ at time of harvest. For the anaerobic biocatalyses, from the first washing step on, all work was performed under anaerobic conditions.

The growth phase prior to biocatalysis, was conducted aerobically in a rich medium, M9Y+0.4% glycerol, to promote high harvest ODs. With the rich medium, due to the presence of yeast extract, the cells did not have to synthesize all biomolecules de novo from glycerol as in the minimal medium. However, although glpK had been eliminated in the engineered strain, very small amounts of G3P may be synthesized via the GpsA enzyme via DHAP and NAD⁺ for triacylglycerol synthesis. Therefore the glpK gene deletion does not prevent the strain GEVO926 from producing triacylglycerol.

The biocatalysis phase was performed in anaerobic, biocatalysis medium with only glycerol as carbon source to accurately account for carbon consumed. The biocatalysis was conducted anaerobically to match the biocatalysis conditions of the n-butanol fermentation and to greatly simplify carbon accounting complicated by loss of carbon via carbon dioxide aerobically. Aerobically, more NADH is generated by metabolism of glycerol than may be used by the pathway, so the n-butanol pathway would not be balanced; acetyl-CoA is lost to the TCA cycle as CO₂. Anaerobically, the engineered strain, GEVO926, produces two moles of NADH, so the n-butanol pathway is balanced.

The ketoreductase reaction was used to monitor availability of NADH being generated by metabolism of glycerol since one ethyl 3-hydroxybutyrate molecule formed enzymatically requires 1 NAD(P)H and ethyl acetoacetate. It is assumed that the NAD(P)H transhydrogenases readily convert NADH to the NADPH preferentially utilized by the ketoreductase. The biocatalysis reaction was performed as follows. The re-suspended cells were stored on ice until ready to be used for anaerobic biocatalysis at 30° C. Substrate of the ketoreductase, ethyl acetoacetate, was added to 40 mM concentration, and the reaction was started with addition of filter-sterilized 10% glycerol to a concentration of 5.5 mM. Depending on the experiment, background reactions with substrate but no carbon source were also run in parallel to the experimental reactions to monitor any metabolites or product of the enzymatic reaction when no carbon source was fed. Samples were taken periodically, at least every half hour.

### Assays: Cell Dry Weight

The rates of glycerol consumption, product formation, and metabolite generation were normalized to cell dry weights. Cell dry weights were determined by taking triplicate 10 mL aliquots of the re-suspended cells in pre-weighed 15 mL conical tubes for each biological replicate, centrifugation at 4000 g for 15 minutes, and discarding the supernatant. The pellets were dried in an oven at 80° C., cooled, and the cell pellet weights were recorded.

### Assays: Protein Gels

Protein gels verified that similar cell masses had an abundant and similar quantity of the ketoreductase enzyme.

### Analytical Chromatography: Sample Preparation

Samples from the biocatalysis were prepared for liquid and gas chromatography. In particular, samples in all experiments were handled with care taken to minimize the exposure of samples to room temperature and air. Samples were frozen at −80° C. immediately after all of the samples of a given time-point were taken. Then, the samples were pelleted in a microcentrifuge for 15 minutes at 12000 g without prior defrosting once removed from −80° C. storage. The supernatant was transferred to individual wells of a multi-well filter-plate (Pall AcroPrep 96 Filter Plate, 0.2 micrometer GH Polypropylene) on top of a deep-well, multi-well plate. With an aspirator and a purpose-specific manifold, the samples were drawn through the filters and into the lower plate. Each sample was subsequently transferred to vials for liquid chromatographic (LC) analysis and gas chromatographic (GC) analysis. Typically, the samples were processed on the LC, then internal standard for GC analysis was added, and GC analysis was subsequently performed.

### Analytical Chromatography: LC Analysis of Mixed Acids Metabolites, Glycerol, Ethyl Acetoacetate, and Ethyl 3-Hydroxybutyrate

In order to determine the ratio of NADH available per glycerol metabolized, quantitation of glycerol, and the product of the NADH-dependent conversion, ethyl 3-hydroxybutyrate, was necessary. To account for all NADH generated, any possible other metabolites that were produced via NADH dependent conversions were quantitated, as well, since those compounds reflect NADH diverted from the ketoreductase. These metabolites include succinate and lactate. Formate and acetate are other metabolites that were quantitated. Acetate is of particular interest, since it indicates availability of acetyl-CoA.

The parameters of the LC analysis are performed as described in Table 5 below.

TABLE 5
Parameters for LC Analysis

| | |
|---|---|
| Column: | BioRad Aminex 87H (sulphate-derivatized column) |
| Mobile phase: | 0.04 N H₂SO₄ |
| Temperature: | 60° C. column temp |
| Detectors: | RID; UV at 210 nm |

Standards were prepared by independently weighing triplicate solid or volatile components into 10 mL volumetric flasks on an analytical balance, and then bringing the solution up to volume with HPLC-grade or milliQ water. The preparation of the standards was validated by agreement between the three individually prepared curves. Standards were prepared within several days of use and stored at 4° C. between uses.

### Analytical Chromatography: GC Analysis of Ethanol

The parameters of the GC analysis of ethanol are described in Table 6 below.

TABLE 6
Parameters for GC Analysis

| Column: | J & W DB-FFAP (Nitroterephthalic acid modified | |
|---|---|---|
| | polyethylene glycol) | |
| Column length: | 30 m; column diameter, 0.32 mm; film thickness: | |
| | 0.25 microM. | |
| Syringe volume: | 1 microL | |
| Runtime: | 14.7 minutes | |
| Temperature | Initial temp, 50° C. | 8° C./min to 80° C. |
| program: | 13° C./min to 170° C. | 50° C./min to 220° C. |

| | |
|---|---|
| Detector: | FID |

Standards for ethanol quantitation were prepared by weighing absolute ethanol into 10 mL volumetric flasks on an analytical balance and immediately capping the flasks. Then, the flask was filled to volume with HPLC-grade or milliQ-purified water. Three independently-prepared sets of dilutions were prepared and run to validate the standards. An internal standard of 1-pentanol was added, 50 μL, to each milliliter of sample prepared. The sample holder of the GC was recirculated with water cooled to 4° C. to prevent the evaporation of volatiles from the liquid phase.

Then, based on measured cell dry weights, the raw concentrations of products, metabolites, and glycerol consumption rates were normalized to mmol/g-cell dry weight.

Results: Anaerobic Biocatalysis—Determining NADH per glycerol, Derived From Rates

The yield of NAD(P)H-dependent products indicate that the engineered pathway produced two moles of NADH per glycerol versus the one mole of NADH per glycerol from the wild-type pathway. The following explains the first of two approaches that indicate that the engineered strain, GEVO 926, may provide the necessary metabolic intermediates to produce n-butanol with glycerol as a carbon source.

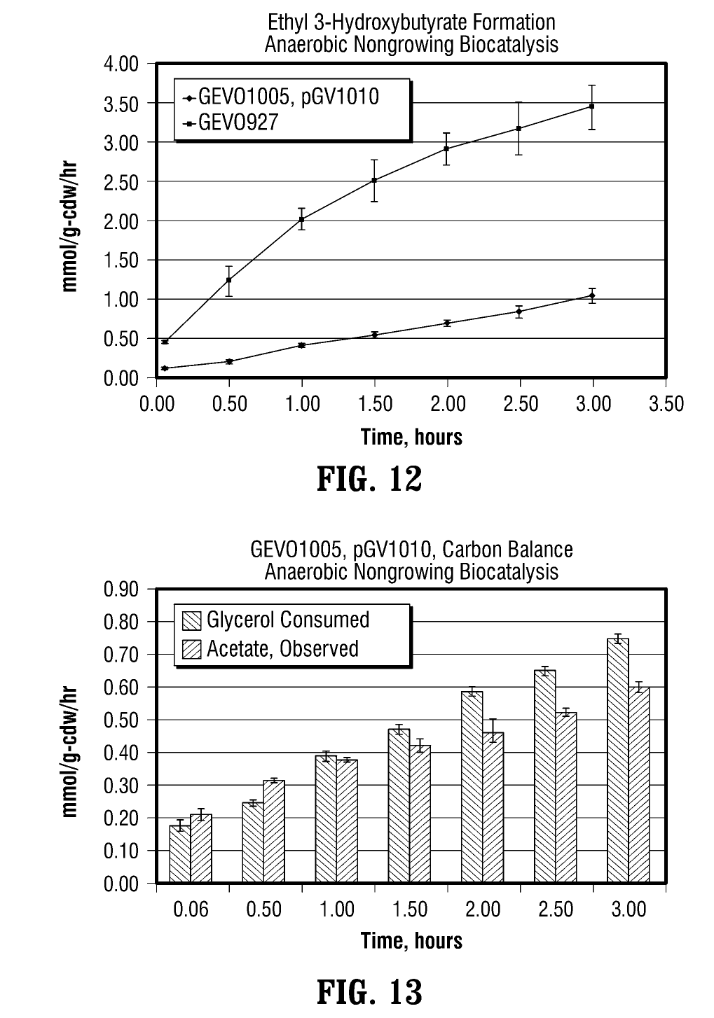

The concentration of the product of the biocatalyst formed per unit of glycerol consumed was used as the indicator of NAD(P)H made available by metabolism per glycerol consumed. FIG. 11 illustrates the glycerol consumed by anaerobic biocatalysis. FIG. 12 illustrates the amount of product formed over time. The rates of product formation and glycerol consumption over the first hour of the reaction were calculated by linear regression. During that period, the product formation and glycerol consumption were linear and neither carbon source nor substrate were limiting. Using the rates from those calculations for each strain, the product per glycerol ratio for each strain was evaluated. These ratio are listed in Table 8. Note that GEVO927 is the evolved, engineered strain GEVO926 containing the pGV110 plasmid, from which the ketoreductase gene is expressed. The rates for product formation and glycerol consumption were normalized to the cell dry weights of each of the individual replicate cell suspensions used for each biocatalysis.

Then, since essentially no other metabolites that indicate NADH availability were observed, it was concluded that almost all of the NADH made available by glycerol metabolism was utilized by the ketoreductase enzyme to form ethyl 3-hydroxybutyrate. Therefore, the product formed to glycerol consumed ratio of each strain is equivalent to the NADH per glycerol ratio. The engineered to the wild-type NADH per glycerol ratio was calculated to determine the ratio of increased NADH availability to the engineered strain over the wild-type. The engineered pathway as functional in GEVO926 did generate about nearly twice the amount of NAD(P)H per glycerol as compared to the wild-type pathway as functional in GEVO1005. With no oxygen available, the engineered pathway should theoretically yield one additional NADH over the wild-type pathway, as glycerol is metabolized to pyruvate. The elimination of the FADH2-linked GlpD enzyme leads to one reducing equivalent not being lost to the electron transport chain. In the engineered strain the NADH-dependent glycerol dehydrogenase (GldA) enzyme transfers the reducing equivalent available from glycerol to NADH.

The product per glycerol ratios for each strain were somewhat higher than theoretically expected. This may be a consequence of slight over-estimation of the concentration of product formed. Whatever the contribution to an under-estimation of glycerol consumed or an over-estimation of product formed, this systematic error cancels in the strain-to-strain ratio. Derived from rates, the strain-to-strain comparison indicates that two moles of NADH are available in GEVO 926, relative to the non engineered strain GEVO1005. The calculated ratio of 1.74+/−0.5 is within the error range of the expected ratio of 2.

A higher than theoretically expected product per glycerol ratio could also reflect carbon source other than the glycerol that was fed over the course of the biocatalysis, possibly autolyzed cells in the suspension or metabolism of intracellular carbon source. By using the comparison of both strains, contributions such as the ones postulated cancel out, assuming that the same processes are at work in each strain. If during the enrichment evolution, the engineered strain acquired an addition to differentiate itself in this way from the wild-type, this comparison would be subject to that caveat. Further discussion of the possible differences between the two strains that could invalidate this hypothesis are discussed later.

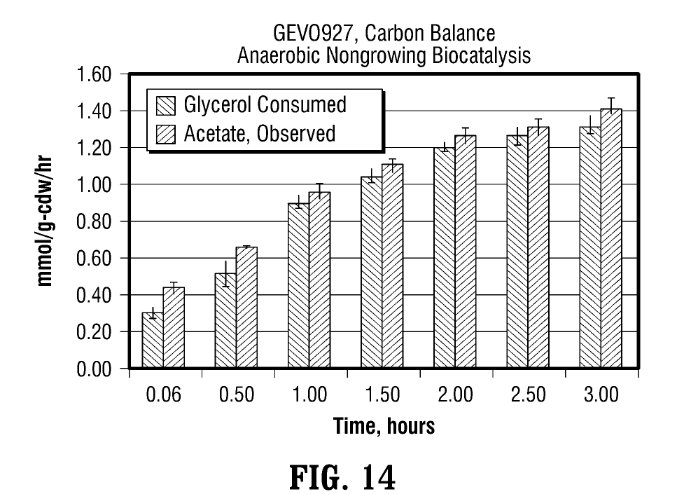

FIG. 13 and FIG. 14 compare the glycerol consumed to acetate produced by GEVO1005, pGV1010, and the engineered strain, GEVO 927. This shows that the evolved strain provide a quantitative amount of acetate per glycerol consumed. Provided that the n-butanol producing pathway is expressed in the cells, acetyl-CoA produced from glycerol may be converted to n-butanol instead of acetate.

TABLE 7
Parameters from Anaerobic Biocatalysis

| | GEVO1005, pGV1010 | GEVO 927 |
|---|---|---|
| From first hour of data | mmol/g-cdw/hr | mmol/g-cdw/hr |
| Product Formation Rate | 0.319 +/− 0.026 | 1.67 +/− 0.15 |
| Glycerol Consumption | 0.228 +/− 0.023 | 0.688 +/− 0.053 |
| Rate | | |

| | Product |
|---|---|
| Glycerol | GEVO 927/GEVO1005, pGV1010 |
| Strain-to-strain ratio, | 1.74 +/− 0.50 |
| derived from rates | |

| | | |
|---|---|---|
| P/G ratio, derived from | 1.40 +/− 0.29 | 2.42 +/− 0.47 |
| rates over first hour | | |
| Product/glycerol ratio, | 1.43 +/− 0.11 | 2.83 +/− 0.17 |
| from end-point | | |
| measurements | | |

| | |
|---|---|
| Strain-to-strain ratio, from | 1.98 +/− 0.19 |
| end-point measurements | |

### Results: Anaerobic Biocatalysis—End-Point Assay

In an independent experiment, an anaerobic biocatalysis was performed as described supra with the exception that a limiting amount of glycerol was fed to the biocatalysis. By doing this, independent of time, the amount of product formed per total glycerol consumed should reflect the same ratio calculated by the rates-based approach described supra. Using the absolute amount of product formed when all glycerol is consumed in an anaerobic biocatalysis, the product per glycerol ratio is consistent with the expected changes to glycerol metabolism. As shown in Table 7, the engineered strain GEVO927 produces NAD(P)H-dependent products, e.g. ethyl 3-hydroxybutyrate, relative to GEVO1005, pGV110, from the same amount of glycerol consumed.

If no other aspect of the system is limiting and the substrate available to the biocatalyst is in excess, even if all of the carbon source is consumed, the amount of NAD(P)H-dependent product formed should indicate the amount of NADH made available by metabolism of the carbon source. In order that the substrate never becomes limiting, the concentration of the carbon source should be smaller than the amount of substrate supplied to the reaction by the number of NADH equivalents expected per carbon source molecule. In that case, independent of time, if all carbon source is consumed, then the product formed indicates the quantity of NAD(P)H made available to the catalyst for a given carbon source amount. This assumes the conditions delineated above, for example, that no NAD(P)H equivalents are being diverted to other NAD(P)H-consuming pathways. This approach would be expected to confirm the results of the rates-derived determination, as it does.

If the carbon source is limiting, the amount of product formed by the biocatalyst is proportional to the NAD(P)H available to the cell by metabolism of that carbon source, regardless of the rates of product formation or glycerol consumption.

### Carbon Balance

The carbon balance calculations also confirm that most of the ethanol comes from the abiotic source, since including uncorrected ethanol concentrations would cause the carbon balance calculations to be impossibly high, 7.4 to 3.5 times higher for the wild-type, and 4.3 to 2.4 times higher for the engineered strain, in terms of % carbon recovered. (See FIGS. 13 and 14) The result that would invalidate the hypothesis that the engineered strain, GEVO926, is making more NADH per glycerol than the wild-type would be the observation that more reduced metabolites were being produced by the wild-type strain by diverting NADH to fermentative pathways, producing reduced products like ethanol, succinate, and lactate. However, the high % carbon recovered for the wild-type indicates that very little NADH is being diverted to reduced metabolites. The total amount of NADH-dependent metabolites between the two strains was not identical. However, the amount of NADH that was spent to form these metabolites is small compared with the amount that went to the biocatalyst. Under anaerobic metabolism, carbon recovered as metabolites should be equal to carbon consumed as glycerol. If all reducing equivalents go to the biocatalyst, then the carbon from metabolism would be expected to show up as unreduced products, acetate or formate, which may be decomposed into CO₂ and H₂ by the action of formate dehydrogenase. FIG. 13 is a bar graph of the carbon balance of GEVO1005, pGV110. FIG. 14 is a bar graph of carbon balance of GEVO927.

The rate of product formation by the NADH-dependent ketoreductase biocatalyst indicates the rate of NADH formation by conversion of glycerol consumed if the system meets certain requirements: (1) The catalyst and substrate are not limiting, so that the reaction is first-order with respect to NADH. This means there is sufficient catalyst, in terms of protein concentration and activity, to readily convert substrate to product, as the reduced cofactor becomes available in the cell, as it is formed by metabolism. If the catalyst is not sufficiently active, then the NADH made available will go to other NADH-utilizing enzymes, especially fermentation pathways. Even in this scenario, the metabolite profiles between the two strains should show increased amounts of reduced fermentation products in the strain producing more reducing equivalents.

However, the results indicate that almost all of the NAD(P)H is going to the ketoreductase, since any available NADH would show up as reduced metabolites or product of the NADH-dependent enzymatic conversion. The NAD(P)H being generated by metabolism is unlikely being used for biosynthetic purposes, since protein synthesis is inhibited by the lack of nitrogen in the media. NADH dehydrogenases are only active under respiratory conditions, so that potential sink is unlikely under the anaerobic conditions.

One example of a step in the wild-type metabolism of glycerol that would be hypothetically inhibited by the lack of FAD+ is the FADH2-linked dehydrogenation of glycerol-3-phosphate to dihydroxyacetone phosphate (DHAP) under anaerobic metabolism of glycerol without exogenous electron acceptor. Anaerobically grown E. coli do not metabolize glycerol and cannot grow without exogenous electron acceptor, such as fumarate or nitrate. However, interestingly, the anaerobic biocatalysis in this study reveals that even without addition of a known electron acceptor, somehow, the wild-type cells do consume glycerol and generate reducing equivalents as NAD(P)H, as reflected by formation of NADPH-dependent product and reduced metabolites, indicating that glycerol metabolism is functioning.

Note that due to nitrogen starvation of the cells in the non-growing medium, the cellular proteins are thought to be locked into that of the aerobic metabolic machinery, even though the cell is in an anaerobic environment. Since the NADH-generating step is subsequent to the FAD+-requiring step, it must be concluded that FAD+ is available for the conversion of G3P to DHAP, or that reducing equivalents through the Electron Transport Chain are being shuttled in some unknown manner. Other studies have reported cases in which it was not possible to determine how the cell was functioning under anaerobic conditions, since no terminal electron acceptor could be identified, but growth occurred regardless. (Anaerobic growth on glycerol enabled by K. pneumoniae genes)

Table 8 depicts the Media formulas used in the disclosed examples.

TABLE 8
Media formulas
M9Y + 0.4% glycerol, 1 L

| | | |
|---|---|---|
| 200 | mLs | M9 salts |
| 2 | mLs | MgSO₄, 1M |
| 0.1 | mL | CaCl₂, 1M |
| 20 | mLs | 20% glycerol |
| 100 | mLs | yeast extract (20 g/L) |
| 678 | mLs | milliQH₂O |

Biocatalysis medium: M9M (−carbon/−ammonium), 1 L

| | | |
|---|---|---|
| 200 | mLs | M9 salts w/o NH₄Cl |
| 2 | mL | 1M MgSO₄ |
| 10 | mL | VA Vitamin Solution |
| 5 | mLs | 0.0324% thiamine |
| 1 | mL | Micronutrient stock, 100X |
| 0.1 | mL | 1M CaCl₂ |

M9 salts

| | | |
|---|---|---|
| 64 | grams | Na₂HPO₄*7H₂O |
| 15 | grams | KH₂PO₄ |
| 2.5 | grams | NaCl |
| 5 | grams | NH₂Cl (Not included in nitrogen-free media) |

VA Vitamin Solution 100X, 500 mLs

| | | |
|---|---|---|
| 25 | mLs | 0.02 M thiamine |
| 25 | mLs | 0.02 M pantothenate |
| 25 | mLs | 0.02 M p-aminobenzoic acid |
| 25 | mLs | 0.02 M p-hydroxybenzoic acid |
| 25 | mLs | 0.02 M 2,3-dihydroxybenzoic acid |
| 375 | mLs | milliQH₂O |

Micronutrient stock, in 50 mLs total volume of milliQH₂O

| | |
|---|---|
| NH₄ molybdate*H₂O | 0.009 grams |
| Boric acid | 0.062 grams |
| Cobalt chloride | 0.018 grams |
| Cupric sulfate | 0.006 grams |
| Manganese chloride | 0.040 grams |
| Zinc sulfate | 0.007 grams |

### Example 5

### In vivo Evolution of E. coli for Functional Expression of Pyruvate Dehydrogenase under Anaerobic Conditions

One way to balance the n-butanol pathway in E. coli is to produce an anaerobically-active pdh gene product. To produce such strains, one can use a selection system which couples redox balance and therefore growth of that E. coli strain with anaerobic activity of Pdh. For example, a strain can constructed that contains knock outs in fermentation pathways to leave only the ethanol production pathway intact as outlined in FIG. 4. Such a strain can not grow anaerobically on glucose minimal medium since the redox balance can not be maintained. Two NADH per glucose are produced in glycolysis and four NADH have to be oxidized in the ethanol pathway. A mutation which leads to anaerobic Pdh activity balances the metabolism and allows anaerobic growth on glucose.

Strain construction for the selection system: GEVO1007 is suitable for this selection system. The strain grows very slowly on glucose minimal medium (M9). For strains that do not grow at all on glucose minimal medium, additional knock outs of frd and of pflB are added to these strains. In addition a silent Pfl encoded by pflDC in E. coli has to be deleted to avoid its mutational activation under selection pressure.

Pyruvate Formate Lyase (encoded by pflB): GEVO1007 is transduced with a P1 lysate prepared from GEVO802, and the resulting strain is designated GEVO1500.

Pyruvate Formate Lyase 2 (encoded by pflDC): GEVO1007 is transduced with a P1 lysate prepared from GEVO1497, and the resulting strain is designated GEVO1501.

Fumarate Reductase (encoded by frd): GEVO1501 is transduced with a P1 lysate prepared from GEVO818 and the resulting strain is designated GEVO1502. For the construction of the corresponding E. coli B strain, GEVO1225 is transduced with a P1 lysate prepared from GEVO822 and the transduced strain is designated GEVO1226.

Characterization of strains for selection: 3 mL LB cultures of GEVO1007 and GEVO1501 inoculated from LB plates, and incubated at 37° C. and 250 rpm over night. These cultures are used to inoculate 1^{st} pass M9 cultures (3 mL) at 5%. The M9 cultures are incubated at 37° C. and 250 rpm over day. The aerobic M9 over day cultures are used to inoculate 2^{nd} pass M9 over night cultures at 2%. The tubes are incubated at 37° C. and 250 rpm. The M9 over night cultures are used to inoculate 3^{rd} pass aerobic M9 cultures (3 mL) at 2%. The M9 over night cultures were also used to inoculate anaerobic tubes with M9 medium at 5%. The tubes were incubated at 37° C. and 250 rpm. In the anaerobic tube GEVO1007 shows slow growth to an OD of 0.2 after 2 days of incubation. GEVO1501 does not grow in the anaerobic tubes.

Strains GEVO1007, and 1501 were streaked onto M9 plates and the plates were incubated anaerobically in an anaerobic jar at 37° C. None of the strains produced visible colonies after 3 days of incubation.

In vivo evolution: Anaerobic cultures of GEVO1007 are transferred daily by diluting 1:100 into 10 ml of fresh broth containing glucose as the sole carbon source. The cultures are incubated for 24 hr at 37° C. without agitation. To enrich for anaerobic Pdh activity, cultures are diluted and spread on solid medium containing gluconate as the sole carbon source once a week. The plates are then incubated in an anaerobic environment. Colonies which grow most rapidly are scraped into fresh broth treated as described above. This process is repeated iteratively until no further increase in growth rate is observed.

### Example 6

### Site-Directed Mutagenesis and Directed Evolution of lpdA

Dehydrolipoate dehydrogenase (encoded by lpdA) is the subunit of the Pdh multienzyme complex which binds NADH. Its mutagenesis can lead to variants that alleviate the inhibition of Pdh at high NADH/NAD ratios typical for anaerobic metabolism. For this purpose, the lpdA gene on the E. coli chromosome is deleted and replaced by mutated lpdA, which is either expressed from a plasmid or from the chromosome. The lpdA gene was cloned into the pCRBlunt vector (Invitrogen) from genomic DNA prepared from E. coli W3110 and sequenced. The resulting plasmid pCRBlpdA was used as the template for site directed mutagenesis of codon 55, which is part of the NADH binding pocket. The lpdA sequence was mutagenized by SOE to produce the mutation A55V (Horton, supra).

In a parallel mutagenesis, PCR was carried out to produce the mutations A55V, I, L, F (Horton, supra).

The gene coding for the dehydrolipoate dehydrogenase in E. coli (lpdA) is disrupted by the deletion of nucleotides 107-1400 of the gene. The resulting strains are GEVO1227, and GEVO1228.

For the construction of the replacement of lpdA with mutated lpdA, the gene was amplified from pCRBlpdAmut or pCRBlpdAN using PCR primers. The mutated lpdA genes were inserted into the genome of GEVO1227 The resulting strain GEVO1229 contains mutated lpdA, lpdAmut, and the resulting strain GEVO1230 contains mutated lpdA, lpdAN, in place of the wild type lpdA gene.

### Example 7

### Deregulation of pdh Expression

The expression of the PDH multienzyme complex is regulated on the transcriptional level by the regulators ArcA and Fnr in response to anaerobicity. In order to avoid down regulation of pdh gene expression under anaerobic conditions, the gene coding for the regulator Fnr (fnr) is deleted from the E. coli genome.

### Transcriptional Dual Regulator Fnr:

The gene coding for the response regulator Fnr in E. coli (fnr) is disrupted with a deletion (nucleotides-87-646 are deleted), resulting in strain, GEVO1503. The deletion of fnr is combined with the deletion of ldhA, ackA, poxB, pflB, and frd.

Strain, GEVO1501, is transduced with a P1 lysate prepared from GEVO1503 and the resulting strain is designated GEVO1504.

### Optimization of the Expression Level of the N-Butanol Pathway

The expression level of the n-butanol pathway genes in the synthesized operon is modified by using the inducible promoter PLtetOI and PLlacOI. In wild type E. coli W3110, PLtetOI is constitutive since the repressor tetR is not present in the cell. The promoter PLlacOI is not completely repressed by the repressor encoded by the chromosomal lad gene, which limits the regulatory range of this promoter. Strain, GEVO1504, is transduced with a P1 lysate prepared from DH5αZ1, and the resulting strain is designated GEVO1505.

### Example 8

### (Prophetic) Heterologous Expression of Formate Dehydrogenase

The native cofactor-independent formate hydrogen lyase is replaced by an NADH-dependent Fdh as described (Berrios-Rivera et al., Metabol. Eng. 2002: 217-229, 2002).

### Example 9

### Heterologous Expression of Clostridium acetobutylicum Genes for the Conversion of Acetyl-CoA to N-Butanol

One set of genes that can be used for heterologous expression of the n-butanol fermentation pathway in E. coli encode thiolase (thl), hydroxybutyryl-CoA dehydrogenase (hbd), crotonase (crt), butyryl-CoA dehydrogenase (bcd), electron transfer proteins (etfA and etfB), and alcohol dehydrogenase (adhE2). The alcohol dehydrogenase-encoding gene (adhE2) can be substituted with either butyraldehyde dehydrogenase-encoding (bdhA/bdhB) or n-butanol dehydrogenase-encoding (aad) genes.

The expression of each protein in E. coli was then first tested and its activity calibrated.

Calibration of activity assays for each enzyme: The above genes are first cloned individually from the genomic DNA of Clostridium acetobutylicum ATCC 824 that was obtained commercially. Using the forward and reverse primer listed in Table 3, each gene is PCR amplified from the genomic DNA and cloned individually into the pZE32 vector using appropriate restriction enzyme sites. The genes together with their native ribosome binding sites are cloned under a modified phage lambda (P_{L-lac}) promoter (Lutz et al., Nucleic Acids Res. 25: 1203-1210, 1997). The genes are then expressed in E. coli cells and assayed for activity.

The pZE32 vector carrying the respective gene is transformed into electrocompetent E. coli-W3110 cells by electroporation. The transformed cells are grown either aerobically or anaerobically in 50 ml of Luria Bertani (LB) medium with 0.1 mg/ml Ampicillin. At mid-log phase of growth, the cells are induced with 0.1 mM of IPTG (isopropyl-beta-D-thiogalactopyranoside). After the cells have reached the stationary phase, transformants are harvested by centrifugation. The activity of the enzymes is monitored using enzyme specific assays (Boynton et al., J. Bacteriol. 178(11): 3015-3024, 1996; Bermejo et al., Applied and Environmental Microbiology 64: 1079-1085, 1998).

Cells grown under aerobic conditions are resuspended in 50 mM 4-morpholine-propanesulfonic acid (MOPS) buffer (pH 7.0) containing 1 mM 1,4-dithiothreitol. The cell suspension is sonicated at 60% power for 9-15 min. Cell debris is removed by centrifugation at 30,000 g for 30 min at 4° C. The supernatant is tested for enzyme activity. Cells grown under anaerobic conditions are resuspended in anaerobic MOPS buffer in the absence of 1,4-dithiothreitol. The cell suspensions is treated with lysozyme, and then disrupted by vigorous vortexing for 10 min. inside the anaerobic chamber at 0° C. The sample is centrifuged at 9000 g for 20mins to separate the lysate and pellet. The suspension is capped tightly during centrifugation. After centrifugation, the supernatant is transferred into ampoules and sealed tightly to prevent contact with air (Boynton et al., J. Bacteriol. 178: 3015-3024, 1996).

The cells are assayed for thiolase using the thiolysis reaction. The thiolysis reaction is coupled at room temperature to the arsenolysis of acetyl-CoA with the aid of phosphotransacetylase. Each assay contains 67 mM Tris hydrochloride (pH 8.0), 0.2 mM uncombined CoA, 0.2 mM acetoacetyl-CoA, 25 mM potassium arsenate (pH 8.1), and 2U of phosphotransacetylase. The reaction is initiated by the addition of acetoacet-CoA. The decrease in absorbance at 232 nm that results from the cleavage of the acyl-CoA bond is monitored. One unit of enzyme is defined as the amount of enzyme catalyzing the thiolytic cleavage of 1 μmol of acetoacetyl-CoA per min per mg of protein (Petersen et al., Applied and Environmental Microbiology 57: 2735-2741, 1991).

Hbd activity is determined by monitoring the rate of oxidation of NADH, as measured by the decrease in absorbance at 340 nm, with acetoacetyl-CoA as the substrate (Boynton et al., Journal of Bacteriology 178: 3015-3024, 1996). A control reaction is done in the absence of substrate to monitor background activity. Crotonase activity is analyzed by observing the decrease in absorbance of crotonyl-CoA in the specific absorption band at 263 nm (Boynton et al., Journal of Bacteriology 178: 3015-3024, 1996). The activity of Bcd is monitored by coupling the oxidation of NADH to the reduction of crotonyl-CoA. The assay will contain in a final volume of 1 ml, 30 μM crotonyl-CoA, 60 mM potassium phosphate pH 6.0, and 0.1 mM NADH. The decrease in absorbance at 340 nm of NADH is used to establish the activity of Bcd, EtfA and EtfB (Becker et al., Biochemistry 32: 10736-10742, 1993). Activity of Aad, AdhE2 and BdhA/B is determined by measuring the rate of oxidation of NADH in the presence of their respective substrates namely, butyraldehyde or butyryl CoA.

The protein concentration is measured by the dye-binding method of Bradford with bovine serum albumin (Bio-Rad) as the standard. For each enzyme, the units of activity in wildtype E. coli is established, where one unit is the amount of enzyme that converts 1 μmole of substrate to product in 1 min.

### Example 10

### Heterologous Expression of Codon-Optimized Clostridium acetobutylicum Genes for the Conversion of Acetyl-CoA to N-Butanol

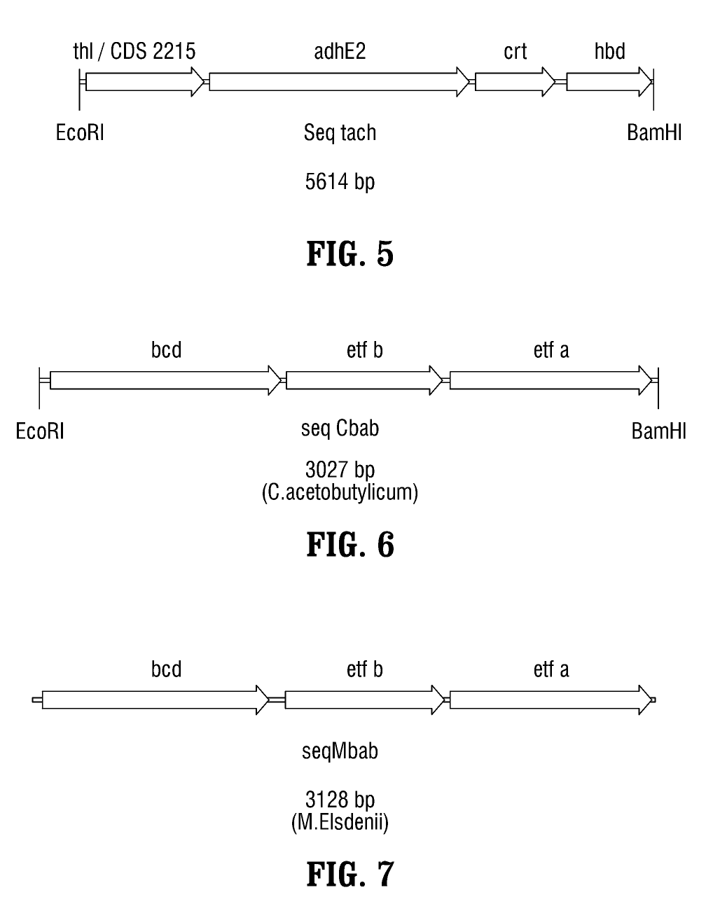

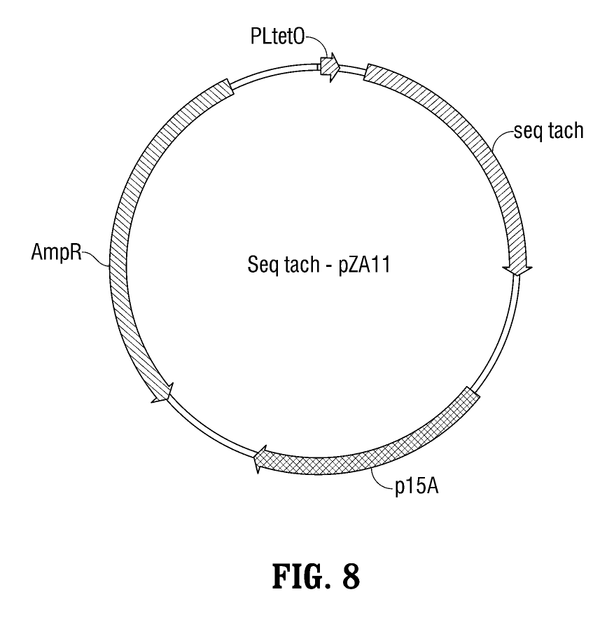

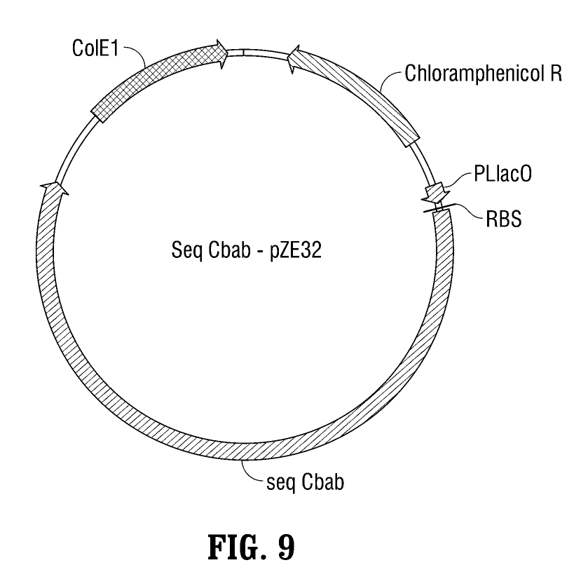

Codon optimization of genes for the expression host increases both protein expression and stability (Gustafsson et al., Trends Biotechnol. 22: 346-353, 2004). To enhance the expression of the genes (FIG. 2) from C. acetobutylicum, the genes were codon optimized for E. coli and synthesized commercially. For expression of the complete pathway in E. coli, the genes are expressed using a two-plasmid system. The thl, hbd, crt and adhE2 genes are expressed as a single transcript (FIG. 5), while the bcd, etfA and etfB genes are expressed together as a second transcript (FIGS. 6 and 7). The two plasmids (FIGS. 8 and 9) are transformed separately, and together, into E. coli cells and tested for activity.

Expression of thl, adhE2, crt and hbd: The thl, adh, crt and hbd genes from C. acetobutylicum are synthesized as a single transcript (seq tach) with unique restriction enzyme sites flanking each gene (FIG. 5). The genes are codon optimized using the proprietary codon optimization algorithm of Codon Devices, Inc. (Cambridge, Mass.). The native ribosome-binding site is located upstream of each gene. The fragment containing the four ORFs is cloned into the pZA11 (Lutz et al., Nucleic Acids Res. 25: 1203-1210, 1997, FIG. 8) vector using EcoRI and BamHI restriction enzyme sites available in the vector MCS.

This vector carries p15A-origin of replication, a modified phage lambda (P_{L-tet}) promoter and an ampicillin resistance gene. The seq tach fragment is cloned downstream of the P_{L-tet} promoter. The seq tach-pZA11 plasmid is transformed into E. coli-W3110 cells by electroporation. The transformants are grown aerobically or anaerobically in 50 ml of Luria Bertani (LB) media containing 0.1 mg/ml Ampicillin at 37° C. At mid-log phase, gene expression is induced using 100 ng/ml anhydrotetracylcine. The cells are harvested 24 hours after induction by centrifugation at 4000 g for 15mins. The harvested cells are re-suspended in 50 mM 4-morpholinepropanesulfonic acid (MOPS) buffer (pH 7.0) containing 1 mM 1,4-dithiothreitol. The cell suspension is sonicated at 60% power for 9 to 15 min. Cell debris is removed by centrifugation at 30,000 g for 30 min. at 4° C. The supernatant is tested for enzyme expression and activity.

The expression of each enzyme is monitored by SDS-PAGE electrophoresis {Sambrook, 2001 #172} by comparing culture samples taken before and after induction. The activity of Crt, Th1, Hbd and AdhE2 is determined using enzyme specific activity assays as outlined above.

Expression of bcd, etfA and etfB: The bcd, etfA and etfB genes from C. acetobutylicum (seq Cbab), and from M. elsdenii (seq Mbab), are synthesized in two separate constructs as outlined in FIGS. 6 and 7, respectively. The genes are codon optimized using the proprietary codon optimization algorithm of DNA 2.0, Inc. The ribosome binding site and inter-genic regions are maintained identical to the native Clostridium operon (Boynton et al., Applied and Environmental Microbiology 62: 2758-2766, 1996). Both sequences are cloned into the pZE32 (Lutz et al., Nucleic Acids Res. 25: 1203-1210, 1997, FIG. 9) vector using EcoRI and BamHI restriction enzyme sites available in the vector MCS. This vector carries ColE1-origin of replication, a modified phage lambda (P_{L-lac}) promoter and chloramphenicol resistance gene. The seqCbab and seqMbab fragments are cloned individually downstream of the P_{L-lac} promoter.

The seqCbab-pZE32 and seqMbab-pZE32 plasmids are transformed into E. coli-W3110 cells by electroporation. The transformants are grown anaerobically in 50 ml of Luria Bertani media containing 0.05 mg/ml chloramphenicol at 37° C. At mid-log phase, gene expression is induced using 1 mM IPTG (isopropyl-beta-D-thiogalactopyranoside). The cells are harvested 24 hours after induction by centrifugation at 4000 g for 15 min. and resuspended in anaerobic MOPS buffer in the absence of 1,4-dithiothreitol. The cell suspension is treated with lysozyme and then disrupted by vigorous vortexing for 10 min inside the anaerobic chamber at 0° C. The sample is centrifuged at 9000 g for 20 min. to separate the lysate and pellet. The suspension is capped tightly during centrifugation. After centrifugation, the supernatant is transferred into ampoules and sealed tightly to prevent contact with air.

The expression of bcd, etfA and etfB is monitored by SDS-PAGE electrophoresis {Sambrook, 2001 #172} by comparing culture samples taken before and after induction. The activity of Bcd is monitored by coupling the oxidation of NADH to the reduction of crotonyl-CoA. The assay will contain in a final volume of 1 ml, 30 μM crotonyl-CoA, 60 mM potassium phosphate pH 6.0 and 0.1 mM NADH. The decrease in absorbance at 340 nm of NADH is used to establish the activity of Bcd, EtfA and EtfB (Boynton et al., Applied and Environmental Microbiology 62: 2758-2766, 1996; O'Neill et al., J. Biol. Chem. 273(33): 21015-21024, 1998).

Expression of complete pathway: The seqCbab-pZE32 and seqtach-pZA11 plasmids are transformed into E. coli-W3110 cells by electroporation. The transformants are grown anaerobically in 250 ml of Luria Bertani media containing 0.05 mg/ml chloramphenicol and 0.1 mg/ml Ampicillin at 37° C. At mid-log phase, gene expression is induced using 1 mM IPTG (isopropyl-beta-D-thiogalactopyranoside) and 100 ng/ml anhydrotetracycline.

At 0, 2, 4, 6, 8, 10, 12 and 24 hrs after induction, samples are taken and analyzed for a variety of properties. 2.5 ml of the cells are harvested by centrifugation at 4000 g for 15 min. and resuspended in anaerobic MOPS buffer in the absence of 1,4-dithiothreitol. The cell suspension is treated with lysozyme and then disrupted by vigorous vortexing for 10 min. inside the anaerobic chamber at 0° C. The suspension is capped tightly during centrifugation. After centrifugation, the supernatant is transferred into ampoules and sealed tightly to prevent contact with air. The lysate is then tested for protein expression and enzyme activity as outlined above. The concentration of glucose and metabolites in the reaction medium is analyzed by high performance liquid chromatography (Causey et al., Proc. Natl. Acad. Sci. U.S.A. 100: 825-832, 2003) according to standard protocols. The concentration of n-butanol and other pathway intermediates is measured by high performance liquid chromatography (HPLC) according to established procedures {Fontaine, 2002 #5}. Ratios of n-butanol molecules formed per glucose molecule consumed are calculated from this data. The above expression, activity and product analysis is repeated in the engineered GEVO strains. With the fermentative pathways knocked out, the cells can grow only with an active n-butanol pathway.

### Example 11

### (Prophetic) Pathway Shuffling of Genes Homologous to Clostridium Acetobutylicum for the Conversion of Acetyl-CoA to N-Butanol

For each of the enzymes that catalyze the metabolic reactions leading from Acetyl-CoA to n-butanol several homologues from a variety of organisms were identified. In order to evaluate the suitability of these alternative enzymes and of all combinations of these enzymes for the production of n-butanol DNA all possible combinations of the pathway enzymes can be expressed from separate DNA constructs.

The n-butanol pathway is synthesized as two operons expressed first from two plasmids (pZE32 and pZA11). The genes thl, crt, adh, and hbd are expressed from pZA11 under control of the PLtetO promoter and the genes bcd, etfB and etfA are expressed from pZE32 under control of the PlacOI promoter. The library contains all combinations of the homologous genes described above with the exception of etfA and etfB which are always from the same organism. All homologous genes are codon optimized for the E. coli expression host. All genes are preceded by their native SD and UTR sequences. The plasmid libraries are transformed into GEVO1505.

The colonies from the selection plates of this transformation are washed from the plates and the resulting strain library is used to inoculate 9 LB cultures containing the inducers anhydrotetracyclin (aTc) and IPTG in different concentrations (0.01, 0.1, 1 mM IPTG×1, 10, 100 ng/ml aTc). After 24 h of incubation at 37° C. and 250 rpm in a shaking incubator, these cultures are used to inoculate 9 tubes containing defined medium with glucose as the sole carbon source. After 12 h of incubation at 37° C. and 250 rpm in a shaking incubator, the cultures are used to inoculate 100 mL of the same medium, and inducer levels in anaerobic tubes to a starting OD of 0.1. The tubes are incubated at 37° C. and 250 rpm in a shaking incubator.

The anaerobic growth rate of the strains depends on the functional expression of the n-butanol pathway. The members of the combinatorial pathway library that allow fastest growth under anaerobic conditions are selected for by serial dilution of the anaerobic tubes.

### Example 12

### (Prophetic) In Vivo Evolution of Recombinant E. Coli for Increasing the N-Butanol Production Rate

Anaerobic cultures of E. coli containing the complete n-butanol pathway are transferred daily by diluting 1:100 into 10 ml of fresh broth containing glucose as the sole carbon source. The cultures are incubated for 24 hr at 37° C. without agitation. Since growth rate correlates to n-butanol production rates, enrichment for increased n-butanol production rates is achieved by diluting cultures and spreading them onto solid medium containing glucose as the sole carbon source once a week. The plates are then incubated in an anaerobic environment. Colonies which grow most rapidly are scraped into fresh broth and treated as described above. This process is repeated iteratively until no further increase in growth rate is observed.

### Example 13

### Testing E. Coli for N-Butanol Resistance

Butanol inhibits cell growth the ultimate level of n-butanol production not only in Clostridium acetobutylicum but also in E. coli. Initial experiments were performed to determine the level of toxicity of n-butanol to E. coli cells. E. coli DH5a cells were used in these experiments.

Briefly, 50 mL of LB medium in 250 mL baffled Erlenmeyer flasks were supplemented with 0 to 5% n-butanol in 0.5% increments. Growth rates and max OD600 were determined after inoculation with 500 μL of an overnight culture. At 0.5% n-butanol, growth rate and max OD600 were approximately halved. At 1% n-butanol, growth rates could not be quantified, and the max OD600 was about 40-fold less.

### Example 14

### In Vivo Evolution of E. Coli for Increasing N-Butanol Resistance

To increase the level of n-butanol tolerance, anaerobic cultures of E. coli cutures are transferred daily by diluting 1:100 into 10 ml of fresh broth containing n-butanol and glucose. These cultures are incubated for 24 hr at 37° C. without agitation. As cultures increased in density during subsequent transfers, n-butanol concentrations are progressively increased to select for resistant mutants. Once a week, the cultures are diluted and spread onto solid medium to enrich for n-butanol-resistant mutants. The fastest growing colonies are scraped from these plates and used to inoculate fresh medium. These cultures are then treated as described above. The initial n-butanol concentration in the medium is 0.5%. Every week, this concentration is increased by 0.1%. This is repeated until no further increase in n-butanol tolerance becomes apparent.

### Example 15

### Recombinant Microorganisms Expressing an Optimized N-Butanol Pathway—BCD/CCR/Ter E. Gracilis/Treponema

Alternative enzymes for the butyrylCoA dehydrogenase step in the n-butanol pathway were tested. Bcd, EtfB, and EtfA from Megasphaera elsdenii and Bcd, EtfB, and EtfA from Clostridium acetobutylicum did not yield any n-butanol in fermentation experiments. Crotonyl-CoA reductase (Ccr) from Streptomyces collinus was functionally expressed and was active in n-butanol fermentation experiments. Trans-2-Enoyl-CoA Reductase (TER) from Euglena gracilis was more active in n-butanol fermentation experiments than Ccr from Streptomyces collinus.

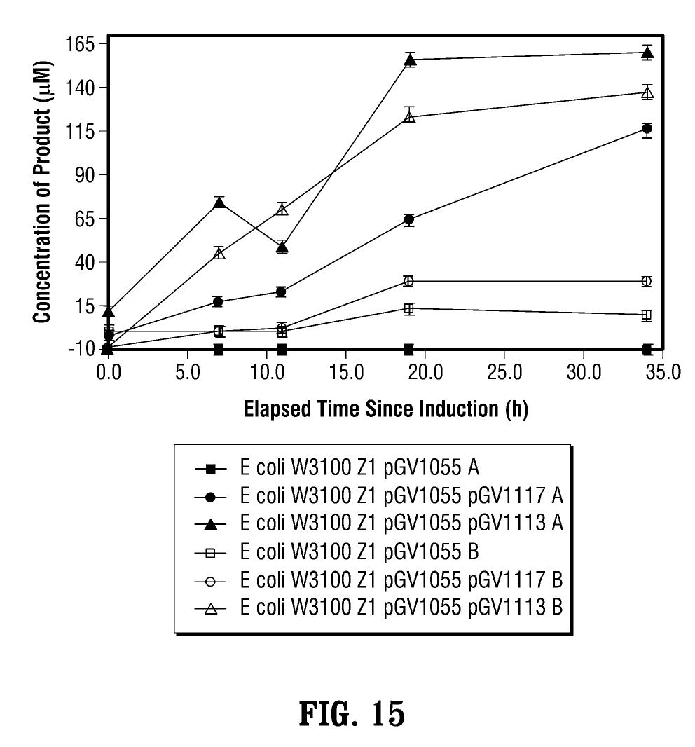

Also, TER from Euglena gracilis was more active in n-butanol fermentation experiments than TER from Aeromonas hydrophila. This was observed following experiments where GEVO768 (W3110Z1) was transformed with pGV1191 and pGV1113 (TEREg—Euglena gracilis) and pGV1117 (TERAh—Aeromonas hydrophila) respectively. The transformants were compared by n-butanol fermentation. The results are illustrated in FIG. 15. The average productivity of the strain with the TERAh was 1.6*10⁻⁴ g/L/h and the average productivity of the strain with the TEREg was 3.2*10⁻⁴ g/L/h.

Further the bacterial TER homologue from Treponema denticola was more active in n-butanol fermentations than TER from Euglena gracilis. This was observed following experiments wherein the 10 genes coding for bacterial TER homologues from Coxiella burnetti, alpha proteobacterium HTCC2255, Bulkholderia cenocepacia, Cytophaga hutchinsonii, Reinekea, Shewanella woodyi, Treponema denticola, Vibrio Ex25, Xanthomonas orycae KACC10331 and Yersinia pestis were codon optimized for expression in E. coli and synthesized. The TER genes were cloned into a vector pGV1252 that is compatible with the n-butanol pathway and ensures low expression of the TER relative to the other pathway genes. The pGV1252 derivatives pGV1272, pGV1300-1309 and pGV1190 were used as a modified 2-vector system which allowed the comparison of the TER genes under conditions that render TER activity limiting for the pathway. GEVO 1121 (E. coli W3110, Δndh,Δldh,ΔadhE,Δfrd, attB::(Sp+ lacIq+ tetR+), ΔmgsA) was used as the host strain for the fermentations to test the homologues. The 10 clones were tested in two independent bottle fermentation experiments with pGV1272 (TER—Euglena gracilis) as control.

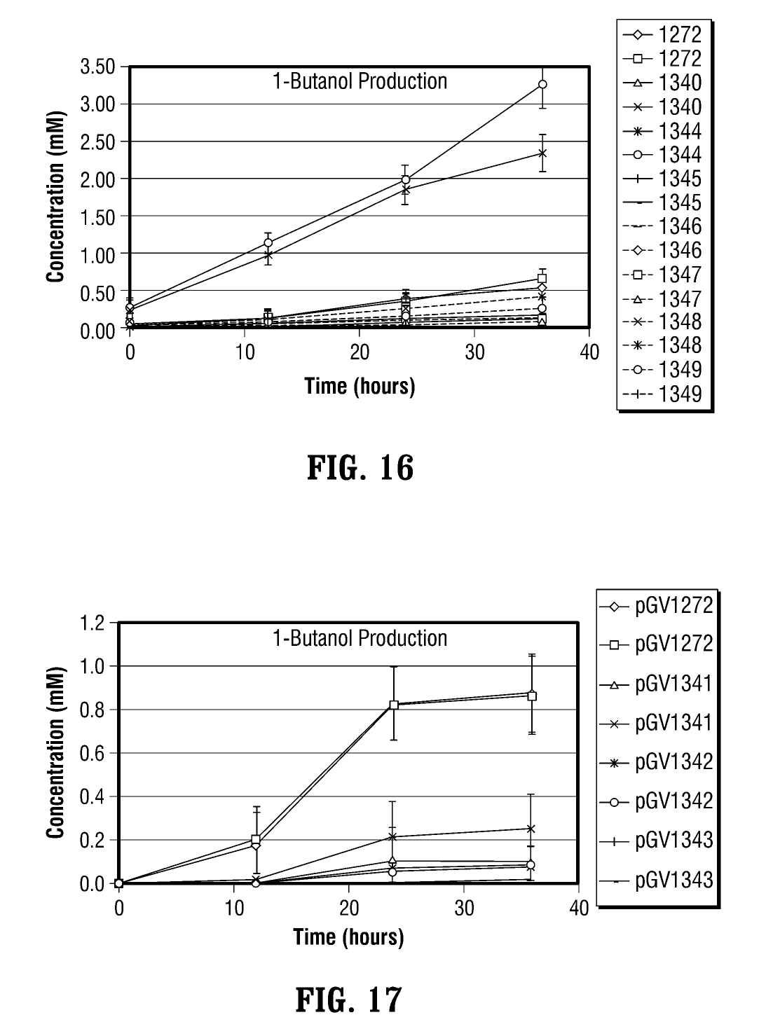

The results illustrated in FIGS. 16 and 17, showed that the bacterial homologue from Treponema denticola (pGV1344) increased the final titre of the fermentation 4 fold and improved the productivity of the fermentation more than 4 fold relative to the fermentation done with Euglena gracilis TER. (FIG. 16). All other bacterial homologues tested showed lower productivity relative to the fermentation done with Euglena gracilis TER. With the TER from Treponema denticola a titre of 0.81 g/L and a productivity of 0.022 g/L/h were reached. With the TER from Euglena gracilis a titre of 0.2 g/L and a productivity of 0.005 g/L/h were reached. The TER from Treponema denticola ensures that enough enzymatic activity is expressed to ensure that the reduction of crotonyl-CoA is not the limiting step within the pathway, when the gene is expressed in the regular 2-plasmid system (pGV1113 derivative+pGV1190).

Further experiments additionally showed that for thiolase, hydroxyl butyryl CoA dehydrogenase and crotonase the codon optimized genes from Clostridium acetobutylicum have the highest in vitro activity of all tested homologues of these genes.

In particular, homologues of the pathway enzymes hydroxyl butyryl CoA dehydrogenase (Hbd), crotonase (Crt) and thiolase (Th1) were expressed and compared by in-vitro activity assay. The hydroxyl butyryl CoA dehydrogenase homologues tested were pGV1037 (Hbd from Clostridium acetobutylicum), pGV1041 (Hbd from Butyrivibrio fibrisolvens), pGV1050 (Hbd from Clostridium beijerinkii), and pGV1154 (Hbd from Clostridium acetobutylicum, codon optimized gene sequence). The crotonase homologues tested were pGV1040 (Crt from Butyrivibrio fibrisolvens), pGV1049 (Crt from Clostridium beijerinkii), pGV1094 (Crt from Clostridium acetobutylicum) and pGV1189 (Crt from Clostridium acetobutylicum, codon optimized gene sequence). The thiolase homologues tested were pGV1035 (Th1 from Clostridium acetobutylicum), pGV1039 (Th1 from Butyrivibrio fibrisolvens), and pGV1188 (Th1 from Clostridium acetobutylicum, codon optimized gene sequence). The genes were expressed and assayed as per the following outlined protocol

GEVO768 (E. coli W3110Z1) was transformed with each of the plasmids and the transformants were plated on LB media with 100 μg/mL of chloramphenicol. The plates were incubated at 37° C. for 14-16 hours. Single colonies of the clones were used to inoculate 3 mL of LB media with 100 μg/mL of chloramphenicol. The cultures were incubated overnight at 37° C. at 250 rpm. The overnight cultures were used to inoculate 50 mL of EZ-rich medium in shake flasks with 100 μg/mL of chloramphenicol. The cultures were incubated at 37° C. at 250 rpm. At mid-exponential growth phase (OD600 0.6-0.8) the cultures were induced with 1 mM IPTG. This activated the expression of the genes cloned under the control of the lac promoter. After 4 hours the cells were centrifuged at 4000 g for 10 minutes. The cells were re-suspended in 100 mM Tris buffer pH 7.5 and lysed using a bead beater. The cells were centrifuged at 22000 g for 5 minutes to separate the lysate. The lysates were carefully transferred to a fresh tube and tested for enzyme activity and overall protein amounts.

To test the activity of Hbd, 10 μL of the lysate was added to 190 μL of 50 mM MOPS pH 7.0 buffer containing 0.1 mM acetoacetyl CoA, and 0.2 mM NADH. The activity of Hbd was measured by monitoring the consumption of NADH at 340 nm. To test the activity of Crt, 10 μL of lysate was added to 190 μL of 100 mM Tris pH 7.6 buffer containing 30 μM crotonyl CoA. Enzyme activity was measured by monitoring the consumption of crotonyl CoA at 263 nm. To test the activity of Th1, 10 μL of lysate was added to 190 μL of Tris pH 8.0 buffer containing 10 mM MgCl₂, 250 μM acetoacetyl CoA and 200 μM of CoA. Enzyme activity was measured by monitoring the consumption of acetoacetyl CoA at 303 nm. All clones were tested with biological replicates and each assay was done in duplicate.

The enzymes from codon-optimized genes had the highest expression and hence highest activity amongst the clones tested. The highest specific activity (normalized to total cellular protein) for these three conversions of the n-butanol pathway are 11.6 nmol/min/μg total cell protein for Hbd (Table 9), 1178 nmol/min/μg total cell protein for crotonase (Table 10), and 2.96 nmol/min/μg total cell protein for thiolase (Table 11). The codon-optimized genes for the thiolase, crotonase and hydroxy-butyryl dehydrogenase result in the highest in vitro enzyme activity and are likely the genes that will yield the highest productivity of the pathway.

Table 9: Specific activities of homologues of the n-butanol pathway enzyme Hbd

TABLE 9
Specific activities of homologues of
the n-butanol pathway enzyme Hbd

| | | Specific activity |
|---|---|---|
| hbd | Source Organism | (nmol/min/μg total cell protein) |
| pGV1037 | C. acetobutylicum | 3.51 |
| pGV1041 | B. fibrisolvens | 0.85 |
| pGV1050 | C. beijerinkii | 2.91 |
| pGV1154 | C. acetobutylicum, codon | 11.69 |
| | optimized | |
| pGV1111 | Vector control | 0.20 |

TABLE 10
Specific activity of Crt homologues

| | | Specific activity |
|---|---|---|
| crt | Source Organism | (nmol/min/μg total cell protein) |
| pGV1094 | C. acetobutylicum | 83.39 |
| pGV1040 | B. fibrisolvens | 0.04 |
| pGV1049 | C. beijerinkii | 10.84 |
| GV1189 | C. acetobutylicum, codon | 916.99 |
| | optimized | |
| pGV1111 | Vector control | 0.17 |

TABLE 11
Specific activity of Thl homologues.

| | | Specific activity |
|---|---|---|
| thl | Source Organism | (nmol/min/μg total cell protein) |
| pGV1035 | C. acetobutylicum | 0.36 |
| pGV1039 | B. fibrisolvens | 2.44 |
| pGV1188 | C. acetobutylicum, codon | 2.50 |
| | optimized | |
| pGV1111 | Vector control | 0.18 |

### Example 16

### Recombinant Microorganism Engineered to Balance N-Butanol Production with Respect to Carbon Production and Consumption—MgsA

A strain GEVO1083 with an additional deletion in the mgsA gene (GEVO1121) showed increased n-butanol yield and was described elsewhere.

GEVO1083 (E. coli W3110,Δndh,Δldh,ΔadhE,Δfrd,attB::(Sp+ lacIq+ tetR+)), pGV1191, pGV1113 (A) and GEVO1121 (GEVO1083, ΔmgsA), pGV1191, pGV1113 (B) were compared by n-butanol bottle fermentation.

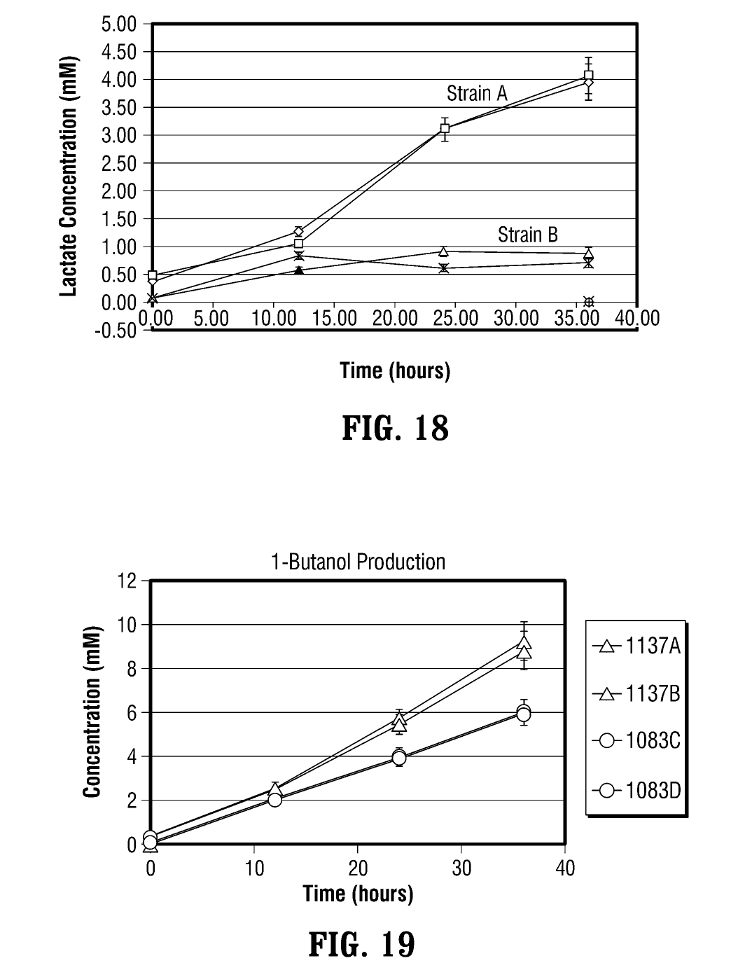

The results are illustrated in FIG. 18. Strain A produced 0.32 g/L lactate in 36 h despite the ldhA knock out which eliminates the fermentative pathway to lactate. Strain B produced only 0.065 g/L lactate in 36 h (FIG. 5). Strain B produced n-butanol as the main reduced fermentation product. Strain A reached a titer of 0.21 g/L, a yield of 0.048 μg, and a productivity of 0.006 g/L/h. Strain B reached a titer of 0.22 g/L, a yield of 0.057 μg, and a productivity of 0.006 g/L/h.

These experiments show that the deletion of mgsA in the n-butanol production strain leads to higher yield in n-butanol fermentations. In particular, these experiments show that the deletion of mgsA leads to 5 times lower lactate production which results in a 19% improvement of the n-butanol yield.

### Example 17

### Recombinant E. Coli Engineered to Balance the N-Butanol Production with Respect to Carbon Production and Consumption—Acetate Pathways

The main fermentative pathway to acetate was deleted by deletion of ackA. The effect of this knock out was investigated with the following experiment:

GEVO 1083 (E. coli W3110, Δndh, Δldh, ΔadhE, Δfrd, attB::(Sp+ lacIq+ tetR+)), pGV1190, pGV1113 (A) and GEVO 1137 (GEVO 1083, ΔackA), pGV1190, pGV1113 (B) were compared by n-butanol bottle fermentation.

The strains were grown aerobically in medium B (EZ-Rich medium containing 0.4% glucose, 100 mg/L Cm, and 200 mg/L Amp) in tubes overnight at 37° C. and 250 rpm. 60 mL of Medium B in shake flasks was inoculated at 2% from the overnight cultures and the cultures were grown to an OD600 of 0.6. The cultures were induced with IPTG and aTc and were incubated at 30° C., 250 rpm for 12 h. 50 mL of the culture were transferred into anaerobic flasks and incubated at 30° C., 250 rpm for 36 h. Samples were taken at different time points and the cultures were fed with glucose and neutralized with NaOH if necessary. The samples were analyzed with GC and HPLC.

The results of the analysis illustrated in FIG. 19 and Table 12 show that the strain with the deletion in ackA reached a 10% higher yield, and 50% higher productivity and titer (Table 13)(FIG. 19). Acetate production was reduced 5 fold in the strain that had the gene deletion in ackA when compared to the same strain without the deletion in ackA FIG. 19).

TABLE 12
process parameter for the comparison
of GEVO1083 and GEVO1137.

| | Yield | | |
|---|---|---|---|
| | g n-butanol/g | Productivity | Titer |
| Sample | Glucose | g/L/h | g/L |
| 1137A | 0.1011 | 0.0174 | 0.627 |
| 1137B | 0.1034 | 0.0183 | 0.660 |
| 1083C | 0.0921 | 0.0117 | 0.422 |
| 1083D | 0.0921 | 0.0123 | 0.442 |

In conclusion the ackA knock out reduces acetate production and increases yield, productivity and titer. This shows that the deletion of native E. coli pathways that compete with the n-butanol pathway for carbon improves the process parameters of a n-butanol production process.

These experiments show that the deletion of the acetate fermentative pathway increases yield, productivity and titer of the production strain in n-butanol fermentations

### Example 18

### Recombinant Microorganism Engineered to Balance the N-Butanol Production with Respect to NADH Production and Consumption—fdh in E. Coli.

The gene fdh was cloned into pGV1113 in an operon behind TER to allow co expression of fdh and the n-butanol pathway (pGV1281). GEVO 1083 (E. coli W3110, Δndh, Δldh, ΔadhE, Δfrd, attB::(Sp+ lacIq+ tetR+)) was transformed with pGV1113 and pGV1190 (1) and with pGV1281 and pGV1190 (2). The strains 1 and 2 were compared by n-butanol bottle fermentation. The strains were grown aerobically in medium B (EZ-Rich medium containing 0.4% glucose, 100 mg/L Cm, and 200 mg/L Amp) in tubes overnight at 37° C. and 250 rpm. 60 mL of Medium B in shake flasks was inoculated at 2% from the overnight cultures and the cultures were grown to an OD600 of 0.6. The cultures were induced with IPTG and aTc and were incubated at 30° C., 250 rpm for 12 h. 50 mL of the culture were transferred into anaerobic flasks and incubated at 30° C., 250 rpm for 36 h. Samples were taken at different time points and the cultures were fed with glucose and neutralized with NaOH if necessary. The samples were analyzed with GC and HPLC.

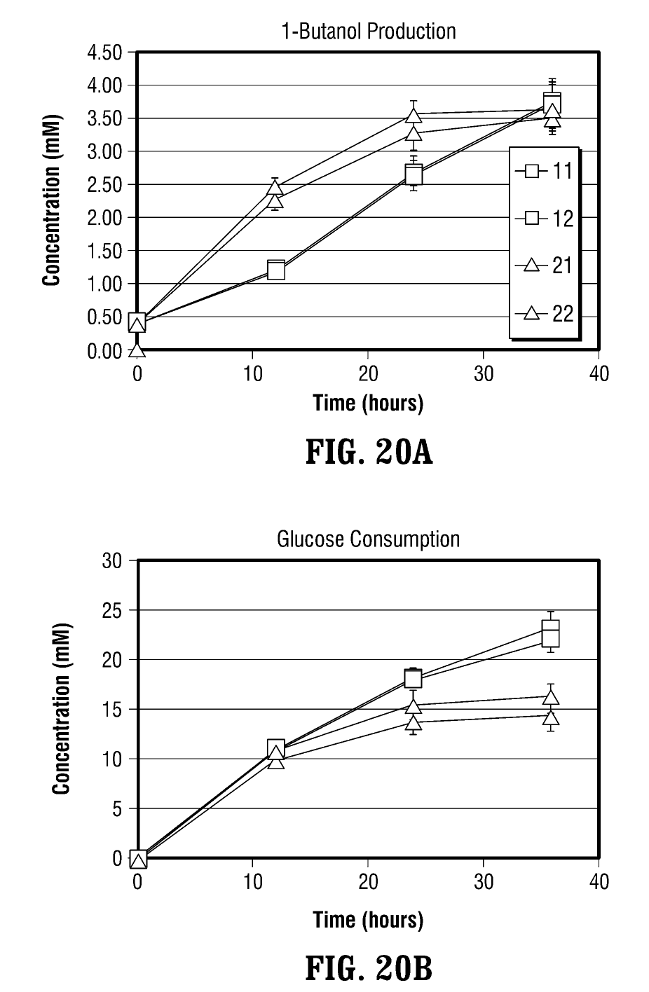

The results illustrated in FIGS. 20A and 20B show that strain 1 which expressed NADH dependent Fdh in addition to the n-butanol pathway produced n-butanol at a yield of 0.086 μg, which was 42% higher than the n-butanol yield of the comparison strain 2 that only expressed the n-butanol pathway (FIGS. 20A and 20B;).

This result shows that the expression of NADH dependent Fdh in the n-butanol production strain increases the yield of n-butanol fermentation.

### Example 19

### Method to Produce N-Butanol—Use of Culture Neutralization and Anaerobic Conditions

The strains listed in Table I above were tested for their n-butanol yield, their productivity and for the maximum titer achievable. In particular the culture conditions were changed from an all anaerobic growth and biocatalysis to an aerobic growth phase and an anaerobic biocatalysis phase according to the following procedure.

The strain to be tested was freshly transformed with the appropriate plasmids for the n-butanol pathway. The single colonies were then picked to inoculate overnight cultures in duplicates using 3 ml EZ-Rich Medium+0.4% glucose and add 3 μl of Amp (100 mg/ml) and 3 μl of Cm (50 mg/ml) diluted in acetone. Since the EZ-Rich Media is easily contaminated the media was used in the sterile hood. The antibiotics used were diluted in solvents other than ethanol (i.e. Cm).

O.D. readings of the overnight cultures were then taken to normalize the amount of inoculum needed. 2% inoculum of overnight culture was used in 60 ml EZ-Rich Media+0.4% glucose and add 60 μl of Amp (100 mg/ml) and 60 μl of Cm (50 mg/ml) diluted in acetone and incubate at 37° C./250 rpm. Again, the media was used in a sterile hood to avoid contamination of the EZ-Rich Media.

At an O.D. ˜0.600 the cultures were induced by adding 60 μl of 1M IPTG and 6 μl of 10,000×ATC[diluted in methanol], making sure that after adding the inducers the cultures were kept away from light in view of light sensitivity of ATC. Methanol was used to mask ethanol peaks in the GC. The cultures were then incubated at 30° C./250 rpm for 6-8 hours. A 100 μl sample of each culture was then taken keeping samples on ice. Reading of the pH, and glucose were also made, with O.D. readings taken at absorbance of 600 nm using water as a reference. In particular, pH paper strips with 5-10 pH range were used to take pH readings. OneTouch Ultra glucose monitor was used to take glucose readings.

The pH was adjusted to 7.5 when necessary by adding 2M NaOH and 40% glucose to maintain ˜0.2% glucose (˜500-600 mg/dl on the glucose meter). A 2 ml sample, was then taken spun down at 25000 g for 5 min at 4° C. The supernatant was then removed for GC/LC analysis and the pellet saved in a box in the freezer. This sample has been labeled as zero hour time point.

50 mL of culture were transferred into an 100 mL anaerobic air filled crimp seal flask and the cultures were put back into the incubator. The cultures were incubated at 30° C./250 rpm, 50 μl of Amp (100 mg/ml) and 50 μl of Cm (50 mg/ml) diluted in acetone were added. Dilution of the Cm in acetone was done to avoid use of antibiotics diluted in ethanol.

Approximately every 12 hours, 2 ml samples were taken in the anaerobic chamber using a syringe. Using the 2 ml sample, O.D., pH, glucose readings were taken, and the rest of the sample was used for GC/LC analysis. Every 24 h 25 μl of Amp (100 mg/ml) and 25 μl of Cm (50 mg/ml) diluted in acetone were added to the cultures to avoid the use of antibiotics diluted in ethanol.

The pH was adjusted to 7.5 when necessary by adding 2M NaOH and 40% glucose to maintain ˜0.2% glucose (˜500-600 mg/dl on the glucose meter).

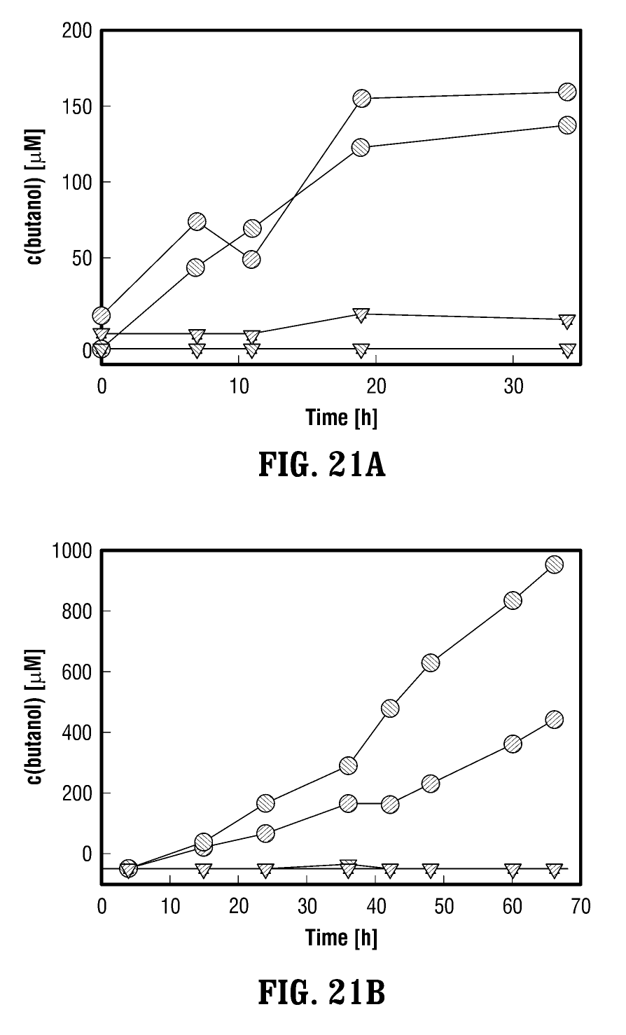

The results of these experiments illustrated in FIGS. 21A and 21B show that by extending the fermentation time and by shortening the intervals between feeding and neutralization events the titer was improved 4.7 fold from 0.011 g/L to 0.0525 g/L. The productivity was improved more than 2 fold from 0.000323 g/L/h to 0.000795 g/L/h and the yield was improved 4 fold from 0.001373 μg to 0.005831 μg (butanol/glucose) (TB002-74). These fermentations were done with strain GEVO768 (W3110Z1).

These experiments show that modification of the fermentation conditions increases productivity, yield and titer of the n-butanol production process

### Example 20

### Method to Produce N-Butanol—Optimization of Fermentation Conditions

Optimization of the transition from growth to biocatalysis in the fermenter improved n-butanol productivity and titer. N-butanol fermentations under different aerobic to anaerobic transitions were performed using GEVO1083 (E. coli W3110 ndh, ldhA, adhE, frd) transformed with the plasmids pGV1190 and pGV1113. Overnight culture of the transformed strain was used to inoculate 4 fermenter vessels, 1, 2, 3, and 4 each filled with 200 mL of EZ-rich medium containing the appropriate antibiotics. The fermenters were maintained at 37° C. during the growth phase and the pH was controlled at 7.0. The fermenters were set to a stirrer speed of 400 rpm and they were gassed at 1 sL/h with 100% air. At mid-exponential phase the cultures were induced with 1 mM IPTG and 100 ng/mL of anhydrotetracycline. The fermenter temperature was reduced to 30° C. subsequent to induction. After 6 hrs of induction, fermenters 1, 2, and 3 were programmed to lower the percent dissolved oxygen concentration from 10% to 0% by controlling the percentage of oxygen in the gas inlet.

The time required for this transition was 2 hours for fermenter 1, 6 hours for fermenter 2 and 12 hours for fermenter 3. Once the dissolved oxygen concentration was at 0% the inlet gas mix was switched to 100% nitrogen at a gas flow rate of 5 sL/h. In fermenter 4, the gas flow was turned off completely 6 hours after induction to let the culture consume the left over oxygen in the fermenter until anaerobic conditions were reached. After 2 hours, the gas mix was switched to 100% nitrogen at a flow rate of 5 sL/h. All fermentations were run for 40 hours and samples were taken at various time points. The samples were analyzed by HPLC and GC to determine the concentrations of organic acids, glucose, ethanol and n-butanol in the fermenters.

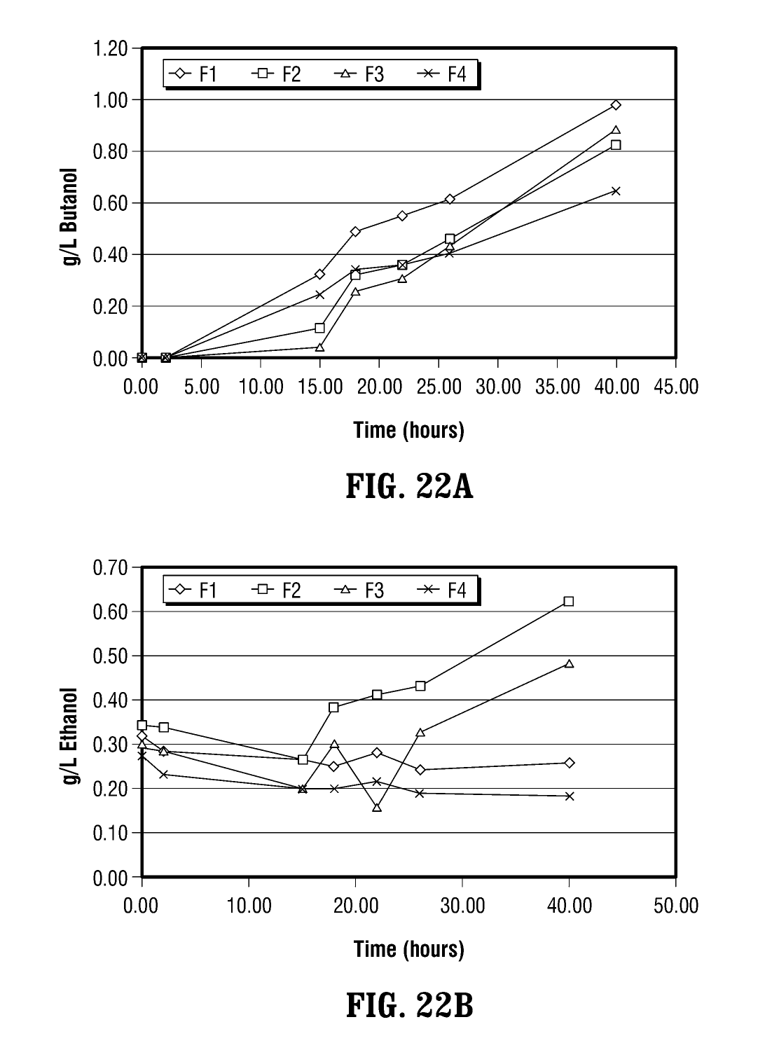

The results are illustrated in FIGS. 22A and 22B and in table 1 below. The highest titer of 0.88 g/L was reached in fermenter 1 with the 2 hour transition from aerobic to anaerobic conditions. Fermenter 1 also had the highest productivity of 0.022 g/L/h (Table 13).

TABLE 13
Titers and productivities reached in the fermentations with different
transitions from aerobic to anaerobic culture conditions

| | Titer | Productivity |
|---|---|---|
| Fermenter | g/L | g/L/h |
| F1 | 0.88 | 0.022 |
| F2 | 0.73 | 0.018 |
| F3 | 0.79 | 0.02 |
| F4 | 0.58 | 0.015 |

These results show how optimization of the fermentation process conditions improves yield, productivity and titer of the n-butanol production process.

### Example 21

### Recombinant Microorganism Engineered to Balance the N-Butanol Production with Respect to NADH Production and Consumption—Fdh Mutant in E. Coli Wild Type Strain

NADH dependent formate dehydrogenase from Candida boidinii was overexpressed in GEVO1034 (E. coli W3110, ΔfdhF) of NADH dependent Fdh in an E. coli strain that has a deletion in its native fdhF gene.

GEVO1034 (E. coli W3110, ΔfdhF), pGV1248 (fdh1 from C. boidinii expressed from medium copy plasmid) (A), and GEVO 1034, pGV1111 (vector only control (B), were compared by n-butanol bottle fermentation according to the SOP “butanol fermentation in anaerobic flasks”. The strains were grown aerobically in medium B (EZ-Rich medium containing 0.4% glucose, 100 mg/L Cm, and 200 mg/L Amp) in tubes overnight at 37° C. and 250 rpm. 60 mL of Medium B in shake flasks was inoculated at 2% from the overnight cultures and the cultures were grown to an OD600 of 0.6.

The cultures were induced with IPTG and aTc and were incubated at 30° C., 250 rpm for 12 h. 50 mL of the culture were transferred into anaerobic flasks and incubated at 30° C., 250 rpm for 36 h. Samples were taken at different time points and the cultures were fed with glucose and neutralized with NaOH if necessary. The samples were analyzed with GC and HPLC.

The results illustrated in FIGS. 23A, 23B 23C 23 D, 24 A and 24B show that Strain A produced ethanol and acetate at a ratio of 0.6+/−0.15. Strain A produced ethanol and acetate at a ratio of 3.43. Strain B produced ethanol and acetate at a ratio of 0.63. Strain A produced 2.97 NADH per glucose and Strain B produced 1.91 NADH per glucose.

In conclusion this result indicates that expression of fdh1 from Candida boidinii increases the available NADH in the cell Updated numbers:

These experiments show that expression of NADH dependent Fdh increases the ratio of NADH per glucose produced by the cell

### Example 22

### Recombinant Microorganisms Engineered to Balance the N-Butanol Production with Respect to NADH Production and Consumption—Pdh Mutant in E. Coli Wild Type Strain

The strains GEVO992 (E. coli W3110, ΔldhA, Δfrd) pGV1278 (PLtet::lpdA mutant) (A), GEVO 992, pGV1279 (PLtet::lpdA mutant) (B), GEVO992, pGV772 (vector only control) (C), were compared by n-butanol bottle fermentation. The strains were grown aerobically in medium B (EZ-Rich medium containing 0.4% glucose, 100 mg/L Cm, and 200 mg/L Amp) in tubes overnight at 37° C. and 250 rpm. 60 mL of Medium B in shake flasks was inoculated at 2% from the overnight cultures and the cultures were grown to an OD600 of 0.6.

The cultures were induced with IPTG and aTc and were incubated at 30° C., 250 rpm for 12 h. 50 mL of the culture were transferred into anaerobic flasks and incubated at 30° C., 250 rpm for 36 h. Samples were taken at different time points and the cultures were fed with glucose and neutralized with NaOH if necessary. The samples were analyzed with GC and HPLC.

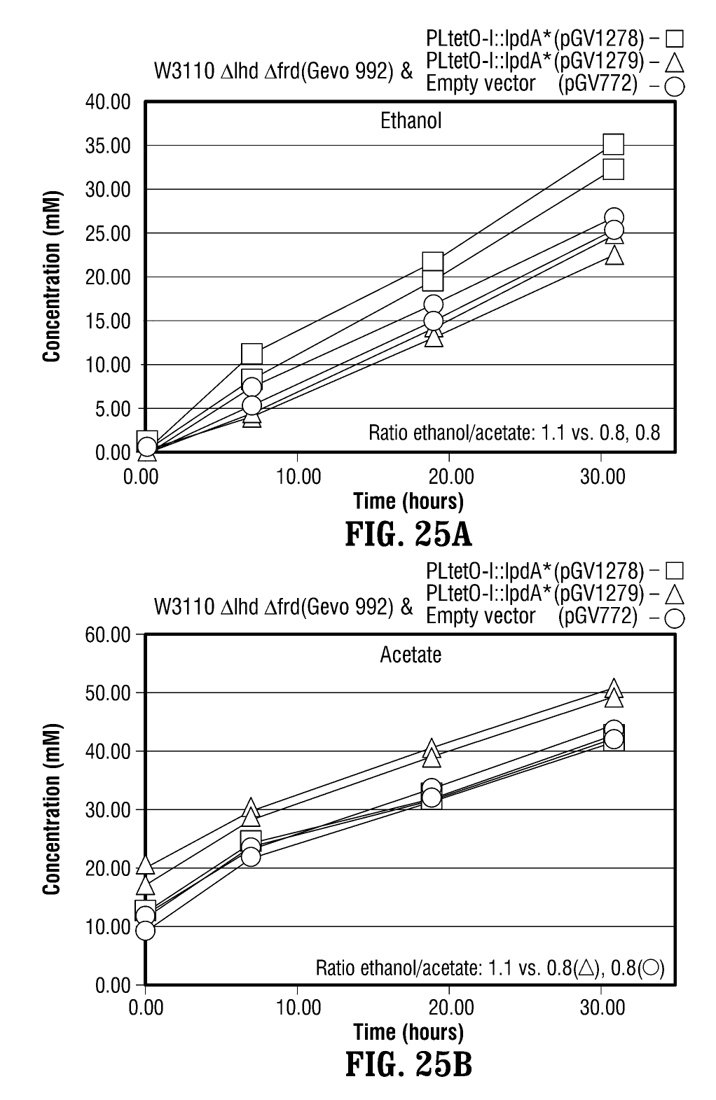

The results illustrated in FIGS. 25A and 25B show that Strain A produced ethanol and acetate at a ratio of 1.1. Strain B produced ethanol and acetate at a ratio of 0.8. Strain C produced ethanol and acetate at a ratio of 0.8. The ratio of strain A expressing the mutant lpdA is 1.4 fold higher than the ratio of strain B and strain C.

These results indicate that expression of the mutant LpdA increases the available NADH in the cell. In particular, these results show that the expression of Pdh that is mutated to avoid inhibition by high NADH/NAD levels increases the ratio of NADH per glucose produced by the cell under anaerobic conditions.

### Example 23

### (Prophetic): Production of N-Butanolat Yields Higher than 50% of Theoretical

The strains GEVO 1510 (E. coli W3110, ΔldhA, ΔpflB, ΔpflDC, ΔadhE, Δfrd, ΔackA, ΔmgsA) pGV1191, pGV1113 (A), and GEVO 1511 (E. coli W3110, ΔldhA, ΔpflB, ΔpflDC, ΔadhE, Δfrd, ΔackA, ΔmgsA) pGV1191, pGV1113 (B), were compared by n-butanol bottle fermentation. GEVO1510 is evolved for expressing Pdh under anaerobic conditions. The strains are grown aerobically in medium B (EZ-Rich medium containing 0.4% glucose, 100 mg/L Cm, and 200 mg/L Amp) in tubes overnight at 37° C. and 250 rpm. 60 mL of Medium B in shake flasks is inoculated at 2% from the overnight cultures and the cultures are grown to an OD600 of 0.6. The cultures are induced with 1 mM IPTG and 100 ng/mL aTc and are incubated at 30° C., 250 rpm for 12 h. 50 mL of the culture are transferred into anaerobic flasks and incubated at 30° C., 250 rpm for 36 h. Samples are taken at different time points and the cultures are fed with glucose and neutralized with NaOH if necessary. The samples are analyzed with GC and HPLC.

Strain A which is evolved as described supra for increased NADH production produces n-butanol at a yield of 0.3 g/g, which corresponds to 73.2% of the theoretical yield. Strain B reaches a yield of 0.1 g/g (24.4% of the theoretical yield) This result shows that evolving a n-butanol production strain for higher NADH production increases the yield of n-butanol fermentation above 50% of the theoretical yield.

These results show that a strain that produces more than 2 moles of NADH per mole of glucose anaerobically allows for n-butanol yields of higher than 50%.

### Example 24

### (Prophetic): Recombinant Microorganism Engineered to Balance the N-Butanol Production with Respect to NADH Production and Consumption—Fdh in E. Coli.

Gevo 768 (E. coli W3110, attB::(Sp+ lacIq+ tetR+)) was transformed with pGV1583 and pGV1191 (1) and with pGV1435 and pGV1191 (2). The strains 1 and 2 were compared by n-butanol bottle fermentation. The strains were grown aerobically in medium B (EZ-Rich medium containing 0.4% glucose, 100 mg/L Cm, and 200 mg/L Amp) in tubes overnight at 37° C. and 250 rpm. 60 mL of Medium B in shake flasks was inoculated at 2% from the overnight cultures and the cultures were grown to an OD600 of 0.6. The cultures were induced with IPTG and aTc and were incubated at 30° C., 250 rpm for 12 h. 50 mL of the culture were transferred into anaerobic flasks and incubated at 30° C., 250 rpm for 36 h. Samples were taken at different time points and the cultures were fed with glucose and neutralized with NaOH if necessary. The samples were analyzed with GC and HPLC.

The results show that strain 1 which expressed NADH dependent Fdh in addition to the n-butanol pathway produced n-butanol at a yield of 1.82% of theoretical, which was 30% higher than the n-butanol yield of the comparison strain 2 that only expressed the n-butanol pathway.

This result shows that the expression of NADH dependent Fdh in the n-butanol production strain increases the yield of n-butanol fermentation.

### Example 25

### (Prophetic): Production of N-Butanol at Yields Higher than 50% of Theoretical

The strains Gevo1083, pGV1191, pGV1583(A), and Gevo 1083, pGV1191, pGV1435 (B), were compared by n-butanol bottle fermentation. The strains were grown aerobically in medium B (EZ-Rich medium containing 0.4% glucose, 100 mg/L Cm, and 200 mg/L Amp) in tubes overnight at 37° C. and 250 rpm. 60 mL of Medium B in shake flasks was inoculated at 2% from the overnight cultures and the cultures were grown to an OD600 of 0.6. The cultures were induced with 1 mM IPTG and 100 ng/mL aTc and were incubated at 30° C., 250 rpm for 12 h. 50 mL of the culture were transferred into anaerobic flasks and incubated at 30° C., 250 rpm for 36 h. Samples were taken at different time points and the cultures were fed with glucose and neutralized with NaOH if necessary. The samples were analyzed with GC and HPLC.

Strain A which expresses NADH dependent Fdh from C. boidinii from a high copy plasmid produced n-butanol at a yield of 0.29 μg, which corresponds to 70.7% of the theoretical yield. Strain B reached a yield of 0.1 μg (29% of the theoretical yield).

### Example 26

### (Prophetic) Recombinant Microorganism Engineered to Balance the N-Butanol Production with Respect to NADH Production and Consumption—Fdh Mutant in E. Coli Wild Type Strain

NADH dependent formate dehydrogenase from Candida boidinii was overexpressed in Gevo1034 (E. coli W3110, ΔfdhF) of NADH dependent Fdh in an E. coli strain that has a deletion in its native fdhF gene.

Gevo1034 (E. coli W3110, ΔfdhF), pGV1582 (fdh1 from C. boidinii expressed with the strong tac promotor) (A), and Gevo1034, pGV1569 (vector only control (B), were compared by n-butanol bottle fermentation according to the SOP “butanol fermentation in anaerobic flasks”. The strains were grown aerobically in medium B (EZ-Rich medium containing 0.4% glucose, 100 mg/L Cm, and 200 mg/L Amp) in tubes overnight at 37° C. and 250 rpm. 60 mL of Medium B in shake flasks was inoculated at 2% from the overnight cultures and the cultures were grown to an OD600 of 0.6.

The cultures were induced with IPTG and aTc and were incubated at 30° C., 250 rpm for 12 h. 50 mL of the culture were transferred into anaerobic flasks and incubated at 30° C., 250 rpm for 36 h. Samples were taken at different time points and the cultures were fed with glucose and neutralized with NaOH if necessary. The samples were analyzed with GC and HPLC.

The results show that Strain A produced 4 NADH per glucose and Strain B produced 2 NADH per glucose. In conclusion this result indicates that expression of fdh1 from Candida boidinii increases the available NADH in the cell.

These experiments show that expression of NADH dependent Fdh increases the ratio of NADH per glucose produced by the cell

### Example 27

### (Prophetic): Recombinant Microorganism Engineered to Balance the N-Butanol Production with Respect to NADH Production and Consumption—fdh in E. Coli.

Several E. coli strains were transformed with plasmids for the expression of a butanol pathway and for the expression of NADH dependent Fdh from C. boidinii. The strains GEVO1082 (E. coli W3110, Δldh, attB::(Sp+ lacIq+tetR+)) (Strain A), GEVO1054 (E. coli W3110, ΔadhE, attB::(Sp+ lacIq+ tetR+)) (Strain B), GEVO1084 (E. coli W3110, Δldh, ΔadhE, attB::(Sp+ lacIq+tetR+)) (Strain C), GEVO1508 (E. coli W3110, Δldh, ΔadhE, Δfrd, attB::(Sp+ lacIq+ tetR+)) (Strain D), GEVO1509 (E. coli W3110, Δldh, ΔadhE, Δfrd, ΔmgsA, attB::(Sp+ lacIq+ tetR+)) (Strain E), GEVO1085 (E. coli W3110, Δldh, ΔadhE, Δfrd, ΔackA, attB::(Sp+ lacIq+ tetR+)) (Strain F), GEVO1507 (E. coli W3110, Δldh, ΔadhE, Δfrd, ΔackA, ΔmgsA, attB::(Sp+ lacIq+ tetR+)) (Strain G) were transformed with pGV1191 and pGV1583. (2). Strains A-F containing these plasmids were compared by n-butanol bottle fermentation. The strains were grown aerobically in medium B (EZ-Rich medium containing 0.4% glucose, 100 mg/L Cm, and 200 mg/L Amp) in tubes overnight at 37° C. and 250 rpm. 60 mL of Medium B in shake flasks was inoculated at 2% from the overnight cultures and the cultures were grown to an OD600 of 0.6. The cultures were induced with IPTG and aTc and were incubated at 30° C., 250 rpm for 12 h. 50 mL of the culture were transferred into anaerobic flasks and incubated at 30° C., 250 rpm for 36 h. Samples were taken at different time points and the cultures were fed with glucose and neutralized with NaOH if necessary. The samples were analyzed with GC and HPLC.

The results show that Strain A produces butanol with a yield of 5%, Strain B produces butanol with a yield of 40%, Strain C produces butanol with a yield of 50%, Strain D produces butanol with a yield of 55%, Strain E produces butanol with a yield of 60%, Strain F produces butanol with a yield of 65%, Strain G produces butanol with a yield of 70%.

The examples set forth above are provided to give those of ordinary skill in the art a complete disclosure and description of how to make and use the embodiments of the devices, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The entire disclosure of each document cited (including patents, patent applications, journal articles, abstracts, laboratory manuals, books, or other disclosures) in the Background, Detailed Description, and Examples is hereby incorporated herein by reference. Further, the hard copy of the sequence listing submitted herewith and the corresponding computer readable form are both incorporated herein by reference in their entireties.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. Thus, for example, reference to “a biosynthetic intermediate” includes a plurality of such intermediates, reference to “a nucleic acid” includes a plurality of such nucleic acids and reference to “the genetically modified host cell” includes reference to one or more genetically-modified host cells and equivalents thereof known to those skilled in the art and so forth. As used in this specification the term a “plurality” refers to two or more references as indicated unless the content clearly dictates otherwise.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the disclosure(s), specific examples of appropriate materials and methods are described herein. All publications mentioned herein are incorporated herein by reference to disclose and describe the methods and/or materials in connection with which the publications are cited.

While specific embodiments of the subject disclosures are explicitly disclosed herein, the above specification and examples herein are illustrative and not restrictive. It will be understood that various modifications may be made without departing from the spirit and scope of the disclosure. Many variations of the disclosures will become apparent to those skilled in the art upon review of this specification and the embodiments below. The full scope of the disclosures should be determined by reference to the embodiments, along with their full scope of equivalents and the specification, along with such variations. Accordingly, other embodiments are within the scope of the following claims.

1. A recombinant microorganism capable of producing n-butanol at a yield of at least 5 percent of theoretical, the recombinant microorganism obtainable by:
engineering the microorganism to activate an heterologous enzyme of an NADH-dependent pathway for conversion of a carbon source to n-butanol through production of one or more metabolic intermediates;
engineering the microorganism to inactivate a native enzyme of one or more pathways for the conversion of a substrate to a product wherein the substrate is one of the one or more metabolic intermediates; and
engineering the microorganism to activate at least one of an NADH-producing enzyme and an NADH-producing pathway to balance said NADH-dependent heterologous pathway.

2. The recombinant microorganisms of claim 1, wherein the one or more native pathways is an NADH-dependent pathway.

3. The recombinant microorganism of claim 1, wherein the heterologous enzyme is selected from the group consisting of an anaerobically active pyruvate dehydrogenase, NADH-dependent formate dehydrogenase, acetyl-CoA-acetyltransferase (thiolase), hydroxybutyryl-CoA dehydrogenase, crotonase, butyryl-CoA dehydrogenase, butyraldehyde dehydrogenase and n-butanol dehydrogenase.

4. The recombinant microorganisms of claim 3, wherein the native enzyme comprises an alcohol dehydrogenase catalyzing conversion of acetyl-CoA to ethanol and the recombinant microorganism is capable of producing n-butanol at a yield of at least 30% of theoretical.

5. The recombinant microorganisms of claim 4, wherein the NADH-producing enzyme is an NADH dependent formate dehydrogenase.

6. The recombinant microorganisms of claim 4, wherein the NADH-producing enzyme is a pyruvate dehydrogenase active under anaerobic condition.

7. The recombinant microorganisms of claim 4, wherein the NADH-producting pathway is a pathway for the conversion glycerol to pyruvate, the recombinant microorganism capable of producing n-butanol at a yield of at least 50% of theoretical.

8. The recombinant microorganism of claim 1, wherein the native enzymes is selected from the group consisting of D-lactate dehydrogenase, pyruvate formate lyase, acetaldehyde/alcohol dehydrogenase, phosphate acetyl transferase, acetate kinase A, fumarate reductase, pyruvate oxidase, and methylglyoxal synthase.

9. The recombinant microorganism of claim 4, wherein the native enzyme further comprises a lactate dehydrogenase and the recombinant microorganism is capable of producing n-butanol at a yield of at least 50% of theoretical.

10. The recombinant microorganism of claim 9, wherein the native enzyme further comprises a fumarate reductase and the recombinant microorganism is capable of producing n-butanol at a yield of at least 55% of theoretical.

11. The recombinant microorganism of claim 10, wherein the native enzyme further comprises a methylglyoxal synthase and the recombinant microorganism is capable of producing n-butanol at a yield of at least 60% of theoretical.

12. The recombinant microorganism of claim 11, wherein the native enzyme further comprises a acetate kinase and the recombinant microorganism is capable of producing n-butanol at a yield of at least 65% of theoretical.

13. The recombinant microorganism of claim 12, wherein the NADH-producing enzyme is a pyruvate dehydrogenase active under anaerobic condition and the recombinant microorganism is capable of producing n-butanol at a yield of at least 73% of theoretical.

14. A recombinant microorganism capable of producing n-butanol at a yield of at least 2% percent of theoretical, the recombinant microorganism obtainable by:
engineering the microorganism to activate an heterologous enzyme of an NADH-dependent pathway for conversion of a carbon source to n-butanol through production of one or more metabolic intermediates; and
engineering the microorganism to inactivate a native enzyme of one or more pathways for the conversion of a substrate to a product wherein the substrate is one of the one or more metabolic intermediates.

15. The recombinant microorganisms of claim 14, wherein the one or more native pathways is an NADH-dependent pathways.

16. The recombinant microorganism of claim 14, wherein the heterologous enzyme is selected from the group consisting of an anaerobically active pyruvate dehydrogenase, NADH-dependent formate dehydrogenase, acetyl-CoA-acetyltransferase (thiolase), hydroxybutyryl-CoA dehydrogenase, crotonase, butyryl-CoA dehydrogenase, butyraldehyde dehydrogenase and n-butanol dehydrogenase.

17. The recombinant microorganisms of claim 16, wherein the native enzyme comprises an alcohol dehydrogenase catalyzing the conversion of acetyl-CoA to ethanol and the recombinant microorganism is capable of producing n-butanol at a yield of at least 5% of theoretical.

18. The recombinant microorganism of claim 17, wherein the native enzyme further comprises a lactate dehydrogenase and the recombinant microorganism is capable of producing n-butanol at a yield of at least 7% of theoretical.

19. The recombinant microorganism of claim 18, wherein the native enzyme further comprises a fumarate reductase and the recombinant microorganism is capable of producing n-butanol at a yield of at least 20% of theoretical.

20. The recombinant microorganism of claim 19, wherein the native enzyme further comprises a methylglyoxal synthase and the recombinant microorganism is capable of producing n-butanol at a yield of at least 25% of theoretical.

21. The recombinant microorganism of claim 19, wherein the native enzyme further comprises a acetate kinase and the recombinant microorganism is capable of producing n-butanol at a yield of at least 25% of theoretical.

22. A recombinant microorganism expressing a heterologous pathway for the conversion of a carbon source to n-butanol, the heterologous pathway comprising the following substrate to product conversions:
acetyl-CoA to acetoacetyl-CoA, acetoacetyl-CoA to hydroxybutyryl-CoA, hydroxybutyryl-CoA to crotonoyl-CoA, crotonyl-CoA to butyryl-CoA, butyryl-CoA to butyraldehyde, and butyraldehyde to n-butanol,
the recombinant microorganism engineered to inactivate one or more native pathways for the conversion of a substrate to a product wherein the substrate is pyruvate or acetylCoA,
the recombinant microorganism further engineered to activate at least one of an anaerobically active pyruvate dehydrogenase, a NADH dependent formate dehydrogenase, and a heterologous pathway for the conversion of glycerol to pyruvate, and
the recombinant microorganism capable of producing n-butanol at a yield of at least 5 percent of theoretical.

23. The recombinant microorganism of claim 22, wherein said one or more native pathways are NADH-dependent pathways.

24. The recombinant microorganisms of claim 25, wherein the inactivated pathways comprises at least one of conversion of acetylcoA to ethanol, conversion of pyruvate to lactate, conversion of pyruvate to succinate and conversion of dihydroxyacetonephosphate to methylglyoxal, conversion of acetyl-CoA to acetate, and conversion of pyruvate to acetate.

25. The recombinant microorganisms of claim 22, wherein the one or more native pathways comprise the conversion of acetyl-CoA to ethanol and the recombinant microorganism is capable of producing n-butanol at a yield of at least 30% of theoretical.

26. The recombinant microorganisms of claim 25, wherein the NADH-producting pathway is a pathway for the conversion glycerol to pyruvate, and the recombinant microorganism capable of producing n-butanol at a yield of at least 50% of theoretical.

27. The recombinant microorganism of claim 25, wherein the inactivated pathways further comprises conversion of pyruvate to lactate and the recombinant microorganism is capable of producing n-butanol at a yield of at least 50% of theoretical.

28. The recombinant microorganism of claim 27, wherein the inactivated pathways further comprises the conversion of pyruvate to succinate, and the recombinant microorganism is capable of producing n-butanol at a yield of at least 55% of theoretical.

29. The recombinant microorganism of claim 28, wherein the inactivated pathways further comprises the conversion of pyruvate to methylglyoxal, and the recombinant microorganism is capable of producing n-butanol at a yield of at least 60% of theoretical.

30. The recombinant microorganism of claim 29, wherein the inactivated pathways further comprises the conversion of acetyl-CoA to acetate and the recombinant microorganism is capable of producing n-butanol at a yield of at least 65% of theoretical.

31. The recombinant microorganism of claim 20, wherein the NADH-producing enzyme is a pyruvate dehydrogenase active under anaerobic condition, and the recombinant microorganism is capable of producing n-butanol at a yield of at least 73% of theoretical.

32. A recombinant microorganism expressing a heterologous pathway for the conversion of a carbon source to n-butanol, the heterologous pathway comprising the following substrate to product conversions:
acetyl-CoA to acetoacetyl-CoA; acetoacetyl-CoA to hydroxybutyryl-CoA; hydroxybutyryl-CoA to crotonoyl-CoA; crotonyl-CoA to butyryl-CoA; butyryl-CoA to butyraldehyde, and butyraldehyde to n-butanol,
the recombinant microorganism engineered to inactivate one or more native pathways for the conversion of a substrate to a product wherein the substrate is pyruvate or acetylCoA, the recombinant microorganism capable of producing n-butanol at a yield of at least 2% percent of theoretical.

33. The recombinant microorganisms of claim 32, wherein the inactivated pathways comprises at least one of conversion of acetyl-CoA to ethanol, conversion of pyruvate to lactate, conversion of pyruvate to succinate and conversion of pyruvate to methylglyoxal, conversion of acetyl-CoA to acetate and conversion of pyruvate to acetate.

34. The recombinant microorganisms of claim 32, wherein the one or more native pathways comprise conversion of acetyl-CoA to ethanol and the recombinant microorganism is capable of producing n-butanol at a yield of at least 5% of theoretical.

35. The recombinant microorganism of claim 34, wherein the one or more native pathways further comprises conversion of pyruvate to lactate and the recombinant microorganism is capable of producing n-butanol at a yield of at least 7% of theoretical.

36. The recombinant microorganism of claim 35, wherein the inactivated pathways further comprises conversion of pyruvate to succinate and the recombinant microorganism is capable of producing n-butanol at a yield of at least 20% of theoretical.

37. The recombinant microorganism of claim 36, wherein the inactivated pathways further comprises conversion of pyruvate to methylglyoxal, and the recombinant microorganism is capable of producing n-butanol at a yield of at least 25% of theoretical.

38. The recombinant microorganism of claim 36, wherein the inactivated pathways further comprises conversion of acetyl-CoA to acetate and the recombinant microorganism is capable of producing n-butanol at a yield of at least 35% of theoretical.

39. A method for producing n-butanol the method comprising
providing a recombinant microorganism according to claim 1,
contacting the recombinant microorganism with a carbon source for a time and under conditions sufficient to allow n-butanol production, until a recoverable quantity of n-butanol is produced and
recovering the recoverable amount of n-butanol.

40. A method according to claim 39 wherein the microorganism is grown under aerobic conditions and wherein the biocatalysis is conducted under anaerobic conditions.

41. A method according to claim 32 wherein the microorganism is cultivated with control of pH at pH5-7 and wherein the cultivation temperature is controlled at 25-37C.

42. A recombinant microorganism capable of producing butyrate at a yield of at least 5 percent of theoretical, the recombinant microorganism obtainable by:
engineering the microorganism to activate an NADH-dependent heterologous pathway for conversion of a carbon source to butyrate through production of one or more metabolic intermediates; and
engineering the microorganism to inactivate a native pathway for the conversion of a substrate to a product wherein the substrate is one of the one or more metabolic intermediates.

43. Recombinant microorganism capable of producing mixtures of butyrate and n-butanol at a yield of at least 5 percent of theoretical, the recombinant microorganism obtainable by:
engineering the microorganism to activate an NADH-dependent heterologous pathway for conversion of a carbon source to butyrate through production of one or more metabolic intermediates;
engineering the microorganism to activate an NADH-dependent heterologous pathway for conversion of a carbon source to n-butanol through production of one or more metabolic intermediates; and
engineering the microorganism to inactivate a native pathway for the conversion of a substrate to a product wherein the substrate is one of the one or more metabolic intermediates.

44. The recombinant microorganism of claim 43, the recombinant microorganism obtainable by further engineering the microorganism to activate at least one of an NADH-producing enzyme and an NADH-producing pathway to balance said NADH-dependent heterologous pathway.
